(19) United States
(12) Patent Application Publication
(10) Pub. No.: US 2025/0121049 A1
(43) Pub. Date: Apr. 17, 2025

(54) INFLUENZA VIRUS HEMAGGLUTININ PROTEINS AND USES THEREOF

(71) Applicant: ICAHN SCHOOL OF MEDICINE AT MOUNT SINAI, New York, NY (US)

(72) Inventors: Peter PALESE, New York, NY (US); Adolfo GARCIA-SASTRE, New York, NY (US); Megan ERMLER, New York, NY (US); Florian KRAMMER, New York, NY (US)

(73) Assignee: ICAHN SCHOOL OF MEDICINE AT MOUNT SINAI, New York, NY (US)

(21) Appl. No.: 18/983,218

(22) Filed: Dec. 16, 2024

Related U.S. Application Data
(62) Division of application No. 18/512,751, filed on Nov. 17, 2023, now Pat. No. 12,233,123.
Application No. 18/512,751 is a division of application No. 17/583,088, filed on Jan. 24, 2022, now Pat. No. 11,865,173.
Application No. 17/583,088 is a division of application No. 16/305,845, filed on Nov. 29, 2018, now Pat. No. 11,266,734.
(60) Provisional application No. 62/355,679, filed on Jun. 28, 2016.
(60) Provisional application No. 62/350,701, filed on Jun. 15, 2016.

(51) Int. Cl. A61K 39/145; A61K 39/00; C07K 14/005

(52) U.S. Cl. CPC A61K 39/145; C07K 14/005; A61K 2039/525; A61K 2039/53; A61K 2039/545; A61K 2039/70; C12N 2760/16122; C12N 2760/16134; C12N 2760/16171; C12N 2760/16222; C12N 2760/16234; C12N 2760/16271

(57) ABSTRACT

Provided herein are chimeric hemagglutinin (HA) polypeptides and uses thereof for inducing an immune response (e.g., an antibody response) against influenza virus. Also provided herein are methods of generating antibodies to the chimeric HA polypeptides in a subject.

This application claims the benefit of U.S. Provisional Application Nos. 62/350,701, filed Jun. 15, 2016, and 62/355,679, filed Jun. 28, 2016, each of which is incorporated by reference herein in its entirety.

This invention was made with government support under grant number HHSO100201500010C, awarded by Biomedical Advanced Research and Development Authority, grant numbers 5T32AI007647, U19 AI109946, and 1P01AI097092, awarded by National Institutes of Health, and grant number HHSN272201400008C, awarded by Centers for Excellence for Influenza Research and Surveillance. The government has certain rights in the invention.

This application incorporates by reference a Sequence Listing submitted with this application as a text file entitled “Sequence_Listing_6923_261_228.txt” created on Jun. 12, 2017 and having a size of 206 kilobytes.

### 1. INTRODUCTION

Provided herein are chimeric influenza virus hemagglutinin proteins.

### 2. BACKGROUND

Influenza viruses are enveloped RNA viruses that belong to the family of Orthomyxoviridae (Palese and Shaw (2007) Orthomyxoviridae: The Viruses and Their Replication, 5th ed. Fields' Virology, edited by B. N. Fields, D. M. Knipe and P. M. Howley. Wolters Kluwer Health/Lippincott Williams & Wilkins, Philadelphia, USA, p 1647-1689). The natural host of influenza A viruses are mainly avians, but influenza A viruses (including those of avian origin) also can infect and cause illness in humans and other animal hosts (bats, canines, pigs, horses, sea mammals, and mustelids). For example, the H5N1 avian influenza A virus circulating in Asia has been found in pigs in China and Indonesia and has also expanded its host range to include cats, leopards, and tigers, which generally have not been considered susceptible to influenza A (CIDRAP—Avian Influenza: Agricultural and Wildlife Considerations). The occurrence of influenza virus infections in animals could potentially give rise to human pandemic influenza strains.

Influenza A and B viruses are major human pathogens, causing a respiratory disease that ranges in severity from sub-clinical infection to primary viral pneumonia which can result in death. The clinical effects of infection vary with the virulence of the influenza strain and the exposure, history, age, and immune status of the host. The cumulative morbidity and mortality caused by seasonal influenza is substantial due to the relatively high attack rate. In a normal season, influenza can cause between 3-5 million cases of severe illness and up to 500,000 deaths worldwide (World Health Organization (2003) Influenza: Overview; March 2003). In the United States, influenza viruses infect an estimated 10-15% of the population (Glezen and Couch R B (1978) Interpandemic influenza in the Houston area, 1974-76. N Engl J Med 298:587-592; Fox et al. (1982) Influenza virus infections in Seattle families, 1975-1979. II. Pattern of infection in invaded households and relation of age and prior antibody to occurrence of infection and related illness. Am J Epidemiol 116:228-242) and are associated with approximately 30,000 deaths each year (Thompson W W et al. (2003) Mortality Associated with Influenza and Respiratory Syncytial Virus in the United States. JAMA 289:179-186; Belshe (2007) Translational research on vaccines: influenza as an example. Clin Pharmacol Ther 82:745-749).

An effective way to protect against influenza virus infection is through vaccination; however, current vaccination approaches rely on achieving a good match between circulating strains and the isolates included in the vaccine. Such a match is often difficult to attain due to a combination of factors. First, influenza viruses are constantly undergoing change: every 3-5 years the predominant strain of influenza A virus is replaced by a variant that has undergone sufficient antigenic drift to evade existing antibody responses. Isolates to be included in vaccine preparations must therefore be selected each year based on the intensive surveillance efforts of the World Health Organization (WHO) collaborating centers. Second, to allow sufficient time for vaccine manufacture and distribution, strains must be selected approximately six months prior to the initiation of the influenza season. Often, the predictions of the vaccine strain selection committee are inaccurate, resulting in a substantial drop in the efficacy of vaccination.

The possibility of a novel subtype of influenza virus entering the human population also presents a significant challenge to current vaccination strategies. Since it is impossible to predict what subtype and strain of influenza virus will cause the next pandemic, current, strain-specific approaches cannot be used to prepare a pandemic influenza vaccine in advance of a pandemic. Thus, there is a need for vaccines that cross-protect subjects against different strains and/or subtypes of influenza virus.

### 3. SUMMARY

In one aspect, provided herein are chimeric hemagglutinin (HA) polypeptides comprising an HA ectodomain of an influenza B virus comprising 2, 3, 4, 5, 6, 7, 8, 9, 10, 11, 12 or more amino acid substitutions within an antigenic loop of the globular head domain of the influenza B virus HA, wherein the amino acid substitutions substitute 2, 3, 4, 5, 6, 7, 8, 9, 10, 11, 12 or more amino acid residues in the loop of the globular head of the influenza B virus HA with amino acid residues found in a corresponding region of the globular domain of an influenza A virus HA. In another aspect, provided herein are chimeric hemagglutinin (HA) polypeptides comprising an HA ectodomain of an influenza B virus comprising 2, 3, 4, 5, 6, 7, 8, 9, 10, 11, 12 or more amino acid substitutions within the 120 loop, 150 loop, 160 loop or 190 helix of the globular head domain of the influenza B virus HA, wherein the amino acid substitutions substitute 2, 3, 4, 5, 6, 7, 8, 9, 10, 11, 12 or more amino acid residues in the 120 loop, 150 loop, 160 loop or 190 helix of the globular head of the influenza B virus HA with amino acid residues found in a corresponding region of the globular domain of an influenza A virus HA. In one embodiment, provided herein are chimeric hemagglutinin (HA) polypeptides comprising an HA ectodomain of an influenza B virus comprising 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTFNVINAETAPGGPYRL (SEQ ID NO: 63). In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In another embodiment, provided herein are chimeric hemagglutinin (HA) polypeptides comprising an HA ectodomain of an influenza B virus comprising 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA. In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60). In another embodiment, provided herein are chimeric hemagglutinin (HA) polypeptides comprising an HA ectodomain of an influenza B virus comprising 2, 3, 4, 5 or more amino acid substitutions within 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA. In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In another embodiment, provided herein are chimeric hemagglutinin (HA) polypeptides comprising an HA ectodomain of an influenza B virus comprising 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA. In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMOT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In some embodiments, the chimeric HA polypeptide may also comprise the signal peptide, transmembrane domain, and cytoplasmic tail domain from the influenza B virus HA. In other embodiments, the chimeric HA polypeptide comprises the signal peptide from the influenza B virus HA but lacks the transmembrane and cytoplasmic tail domains. In some embodiments, the chimeric HA polypeptide may also comprise the signal peptide, transmembrane domain, and cytoplasmic tail domain from an influenza A virus HA. In other embodiments, the chimeric HA polypeptide comprises the signal peptide from an influenza A virus HA but lacks the transmembrane and cytoplasmic tail domains. In some embodiments, the chimeric HA comprises the signal peptide of the HA of the influenza virus backbone of the chimeric HA. For example, if the chimeric HA is engineered for an influenza A virus backbone (e.g., the influenza virus comprising or engineered to express the chimeric HA is an influenza A virus), then the chimeric HA comprises the signal peptide of the influenza A virus. In some embodiments, the chimeric HA comprises the signal peptide, transmembrane domain, and cytoplasmic domain of the HA of the influenza virus backbone of the chimeric HA. In some embodiments, the chimeric HA polypeptide may also comprise the signal peptide from the HA of the influenza virus that is engineered to express the chimeric HA. In some embodiments, the chimeric HA polypeptide may also comprise the signal peptide, transmembrane domain, and cytoplasmic tail domain from the HA of the influenza virus that is engineered to express the chimeric HA. Also provided herein are nucleic acids comprising nucleotide sequences encoding such a chimeric HA. In some embodiments, the nucleic acids comprise nucleotide sequences encoding such a chimeric HA and the 5′ and 3′ non-coding regions from the influenza B virus HA. In some embodiments, the nucleic acids comprise nucleotide sequences encoding such a chimeric HA and the 5′ and 3′ non-coding regions from an influenza A virus HA. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA. In specific embodiments, the chimeric HA polypeptide is soluble. In certain embodiments, the chimeric HA polypeptides comprise 1, 2, 3, 4, 5 or more amino acid substitutions in the globular head domain of the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid).

In another aspect, provided herein are chimeric hemagglutinin (HA) polypeptides comprising an HA ectodomain of an influenza B virus comprising one, two, three or all of the following: (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A/virus HA; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNOMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In some embodiments, the chimeric HA polypeptide may also comprise the signal peptide, transmembrane domain, and cytoplasmic tail domain from the influenza B virus HA. In other embodiments, the chimeric HA polypeptide comprises the signal peptide from the influenza B virus HA but lacks the transmembrane and cytoplasmic tail domains. In some embodiments, the chimeric HA polypeptide may also comprise the signal peptide, transmembrane domain, and cytoplasmic tail domain from an influenza A virus HA. In other embodiments, the chimeric HA polypeptide comprises the signal peptide from an influenza A virus HA but lacks the transmembrane and cytoplasmic tail domains. In some embodiments, the chimeric HA comprises the signal peptide of the HA of the influenza virus backbone of the chimeric HA. For example, if the chimeric HA is engineered for an influenza A virus backbone (e.g., the influenza virus comprising or engineered to express the chimeric HA is an influenza A virus), then the chimeric HA comprises the signal peptide of the influenza A virus. In some embodiments, the chimeric HA comprises the signal peptide, transmembrane domain, and cytoplasmic domain of the HA of the influenza virus backbone of the chimeric HA. In some embodiments, the chimeric HA polypeptide may also comprise the signal peptide from the HA of the influenza virus that is engineered to express the chimeric HA. In some embodiments, the chimeric HA polypeptide may also comprise the signal peptide, transmembrane domain, and cytoplasmic tail domain from the HA of the influenza virus that is engineered to express the chimeric HA. Also provided herein are nucleic acids comprising nucleotide sequences encoding such a chimeric HA. In some embodiments, the nucleic acids comprise nucleotide sequences encoding such a chimeric HA and the 5′ and 3′ non-coding regions from the influenza B virus HA. In some embodiments, the nucleic acids comprise nucleotide sequences encoding such a chimeric HA and the 5′ and 3′ non-coding regions from an influenza A virus HA. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA. In specific embodiments, the chimeric HA polypeptide is soluble. In certain embodiments, the chimeric HA polypeptides comprise 1, 2, 3, 4, 5 or more amino acid substitutions in the globular head domain of the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid).

In another aspect, provided herein are chimeric hemagglutinin (HA) polypeptides comprising (i) a hemagglutinin ectodomain from a first influenza B virus strain with 2, 3, 4, 5, 6, 7, 8, 9, 10, 11, 12 or more amino acid substitutions within the 120 loop, 150 loop, 160 loop or 190 helix of the globular head domain of the first influenza B virus strain HA and (ii) a signal peptide, a transmembrane domain and a cytoplasmic tail domain from a second influenza B virus strain, wherein the amino acid substitutions substitute 2, 3, 4, 5, 6, 7, 8, 9, 10, 11, 12 or more amino acid residues in the 120 loop, 150 loop, 160 loop or 190 helix of the globular head of the first influenza B virus strain HA with amino acid residues found in a corresponding region of the globular domain of an influenza A virus HA. In one embodiment, provided herein are chimeric hemagglutinin (HA) polypeptides comprising (i) a hemagglutinin ectodomain from a first influenza B virus strain with 2, 3, 4, 5, 6, 7, 8, 9, 10, 11, 12, 13, 14, 15, 16, 17, 18, 19, 20 or more amino acid substitutions within the 120 loop of the globular head domain of the first influenza B virus strain HA and (ii) a signal peptide, a transmembrane domain and a cytoplasmic tail domain from a second influenza B virus strain, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the first influenza B virus strain HA with amino acid residues found in a corresponding region of the globular domain of an influenza A virus HA. In another embodiment, provided herein are chimeric hemagglutinin (HA) polypeptides comprising (i) a hemagglutinin ectodomain from a first influenza B virus strain with 2, 3, 4, 5, 6, 7, 8, 9 or more amino acid substitutions within the 150 loop of the globular head domain of the first influenza B virus strain HA and (ii) a signal peptide, a transmembrane domain and a cytoplasmic tail domain from a second influenza B virus strain, wherein the amino acid substitutions substitute 2, 3, 4, 5, 6, 7, 8, 9 or more amino acid residues in the 150 loop of the globular head of the first influenza B virus strain HA with amino acid residues found in a corresponding region of the globular domain of an influenza A virus HA. In another embodiment, provided herein are chimeric hemagglutinin (HA) polypeptides comprising (i) a hemagglutinin ectodomain from a first influenza B virus strain with 2, 3, 4, 5 or more amino acid substitutions within the 160 loop of the globular head domain of the first influenza B virus strain HA and (ii) a signal peptide, a transmembrane domain and a cytoplasmic tail domain from a second influenza B virus strain, wherein the amino acid substitutions substitute 2, 3, 4, 5 or more amino acid residues in the 160 loop of the globular head of the first influenza B virus strain HA with amino acid residues found in a corresponding region of the globular domain of an influenza A virus HA. In another embodiment, provided herein are chimeric hemagglutinin (HA) polypeptides comprising (i) a hemagglutinin ectodomain from a first influenza B virus strain with 2, 3, 4, 5, 6, 7, 8 or more amino acid substitutions within the 190 helix of the globular head domain of the first influenza B virus strain HA and (ii) a signal peptide, a transmembrane domain and a cytoplasmic tail domain from a second influenza B virus strain, wherein the amino acid substitutions substitute 2, 3, 4, 5, 6, 7, 8 or more amino acid residues in the 190 helix of the globular head of the first influenza B virus strain HA with amino acid residues found in a corresponding region of the globular domain of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMOT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMQT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and

NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the globular head domain of the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the first influenza B virus strain and the second influenza B virus strain are from the same lineage. In some embodiments, the first influenza B virus strain and the second influenza B virus strain are from the same lineage but are different strains. In a specific embodiment, the first influenza B virus strain is the same strain as the second influenza B virus strain. In another embodiment, the first influenza B virus strain is a different strain than the second influenza B virus strain. In some embodiments, the first influenza B virus strain and the second influenza B virus strain are from different lineages. In some embodiments, the first influenza B virus strain is from the Yamagata lineage. In other embodiments, the first influenza B virus is from the Victoria lineage. In some embodiments, the second influenza B virus strain is from the Yamagata lineage. In other embodiments, the second influenza B virus is from the Victoria lineage. In a specific embodiment, the second influenza B virus strain is the same strain as the influenza virus backbone of an influenza virus either containing, expressing, or both the chimeric HA. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska 7MP0167_2007), or H13 strain (e.g., A/black headed gull/Sweden/1/99).

In another aspect, provided herein are chimeric hemagglutinin (HA) polypeptides comprising: (a) a hemagglutinin ectodomain from a first influenza B virus strain with one, two, three or all of the following (i) 2, 3, 4, 5, 6, 7, 8, 9, 10, 11, 12, 13, 14, 15, 16, 17, 18, 19, 20 or more amino acid substitutions within the 120 loop of the globular head domain of the first influenza B virus strain HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the first influenza B virus strain HA with amino acid residues found in a corresponding region of the globular domain of an influenza A virus HA; (ii) 2, 3, 4, 5, 6, 7, 8, 9 or more amino acid substitutions within the 150 loop of the globular head domain of the first influenza B virus strain HA, wherein the amino acid substitutions substitute 2, 3, 4, 5, 6, 7, 8, 9 or more amino acid residues in the 150 loop of the globular head of the first influenza B virus strain HA with amino acid residues found in a corresponding region of the globular domain of an influenza A virus HA; (iii) 2, 3, 4, 5 or more amino acid substitutions within the 160 loop of the globular head domain of the first influenza B virus strain HA, wherein the amino acid substitutions substitute 2, 3, 4, 5 or more amino acid residues in the 160 loop of the globular head of the first influenza B virus strain HA with amino acid residues found in a corresponding region of the globular domain of an influenza A virus HA; and (iv) 2, 3, 4, 5, 6, 7, 8 or more amino acid substitutions within the 190 helix of the globular head domain of the first influenza B virus strain HA, wherein the amino acid substitutions substitute 2, 3, 4, 5, 6, 7, 8 or more amino acid residues in the 190 helix of the globular head of the first influenza B virus strain HA with amino acid residues found in a corresponding region of the globular domain of an influenza A virus HA; and (b) a signal peptide, a transmembrane domain and a cytoplasmic tail domain from a second influenza B virus. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 13. In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 15. In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 17. In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTFNVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the globular head domain of the influenza B virus HA which are outside of one, two, three, or all of the following: the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the first influenza B virus strain and the second influenza B virus strain are from the same lineage. In some embodiments, the first influenza B virus strain and the second influenza B virus strain are from the same lineage but are different strains. In a specific embodiment, the first influenza B virus strain is the same strain as the second influenza B virus strain. In another embodiment, the first influenza B virus strain is a different strain than the second influenza B virus strain. In some embodiments, the first influenza B virus strain and the second influenza B virus strain are from different lineages. In some embodiments, the first influenza B virus strain is from the Yamagata lineage. In other embodiments, the first influenza B virus is from the Victoria lineage. In some embodiments, the second influenza B virus strain is from the Yamagata lineage. In other embodiments, the second influenza B virus is from the Victoria lineage. In a specific embodiment, the second influenza B virus strain is the same strain as the influenza virus backbone of an influenza virus either containing, expressing, or both the chimeric HA. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska 7MP0167_2007), or H13 strain (e.g., A/black headed gull/Sweden/1/99).

In another aspect, provided herein are chimeric hemagglutinin (HA) polypeptides comprising (i) a hemagglutinin ectodomain from an influenza B virus with 2, 3, 4, 5, 6, 7, 8, 9, 10, 11, 12 or more amino acid substitutions within the 120 loop, 150 loop, 160 loop or 190 helix of the globular head domain of the influenza B virus HA and (ii) a signal peptide, a transmembrane domain and a cytoplasmic tail domain from an influenza A virus, wherein the amino acid substitutions substitute 2, 3, 4, 5, 6, 7, 8, 9, 10, 11, 12 or more amino acid residues in the 120 loop, 150 loop, 160 loop or 190 helix of the globular head of the influenza B virus HA with amino acid residues found in a corresponding region of the globular domain of an influenza A virus HA. In one embodiment, provided herein are chimeric hemagglutinin (HA) polypeptides comprising (i) a hemagglutinin ectodomain from an influenza B virus with 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA and (ii) a signal peptide, a transmembrane domain and a cytoplasmic tail domain from an influenza A virus, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA. In another embodiment, provided herein are chimeric hemagglutinin (HA) polypeptides comprising (i) a hemagglutinin ectodomain from an influenza B virus with 2, 3, 4, 5, 6, 7, 8, 9 or more amino acid substitutions within the 150 loop of the globular head domain of the influenza B virus HA and (ii) a signal peptide, a transmembrane domain and a cytoplasmic tail domain from an influenza A virus, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA. In another embodiment, provided herein are chimeric hemagglutinin (HA) polypeptides comprising (i) a hemagglutinin ectodomain from an influenza B virus with 2, 3, 4, 5 or more amino acid substitutions within the 160 loop of the globular head domain of the influenza B virus HA and (ii) a signal peptide, a transmembrane domain and a cytoplasmic tail domain from an influenza A virus, wherein the amino acid substitutions substitute 2, 3, 4, 5 or more amino acid residues in the 160 loop of the globular head of the influenza B virus HA with amino acid residues found in a corresponding region of the globular domain of an influenza A virus HA. In another embodiment, provided herein are chimeric hemagglutinin (HA) polypeptides comprising (i) a hemagglutinin ectodomain from an influenza B virus with 2, 3, 4, 5, 6, 7, 8 or more amino acid substitutions within the 190 helix of the globular head domain of the influenza B virus HA and (ii) a signal peptide, a transmembrane domain and a cytoplasmic tail domain from an influenza A virus, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the globular head domain of the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the influenza B virus is from the Yamagata lineage. In other embodiments, the influenza B virus is from the Victoria lineage. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99). Also provided herein are nucleic acids comprising nucleotide sequences encoding said chimeric HA polypeptides. In some embodiments, the nucleic acids comprise nucleotide sequences encoding a chimeric HA polypeptide and the 5′ non-coding region and 3′ non-coding region from an influenza A virus. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA.

In another aspect, provided herein are chimeric hemagglutinin (HA) polypeptides comprising: (a) a hemagglutinin ectodomain from an influenza B virus with one, two, three or all of the following (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA; and (b) a signal peptide, a transmembrane domain and a cytoplasmic tail domain from an influenza A virus. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMOT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTENVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the globular head domain of the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the influenza B virus is from the Yamagata lineage. In other embodiments, the influenza B virus is from the Victoria lineage. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99). Also provided herein are nucleic acids comprising nucleotide sequences encoding said chimeric HA polypeptides. In some embodiments, the nucleic acids comprise nucleotide sequences encoding a chimeric HA polypeptide and the 5′ non-coding region and 3′ non-coding region from an influenza A virus. In some embodiments, the nucleic acids comprise nucleotide sequences encoding a chimeric HA polypeptide and the 5′ non-coding region and 3′ non-coding region from an influenza B virus. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA. In a specific embodiment, the nucleic acid comprises the nucleotide sequence set forth in FIG. 12A and FIG. 12B or the complement thereof. In a specific embodiment, the nucleic acid comprises the nucleotide sequence set forth in FIG. 14A and FIG. 14B or the complement thereof. In a specific embodiment, the nucleic acid comprises the nucleotide sequence set forth in FIG. 16A and FIG. 16B or the complement thereof. In a specific embodiment, the nucleic acid comprises the nucleotide sequence set forth in FIG. 18A and FIG. 18B or the complement thereof.

In another aspect, provided herein are chimeric hemagglutinin (HA) polypeptides comprising an HA ectodomain of an influenza B virus comprising 2, 3, 4, 5, 6, 7, 8, 9, 10, 11, 12 or more amino acid substitutions within an antigenic loop of the globular head domain of the influenza B virus HA (e.g., 120 loop, 150 loop, 160 loop and/or 190 helix), wherein the amino acid substitutions substitute 2, 3, 4, 5, 6, 7, 8, 9, 10, 11, 12 or more amino acid residues in the loop of the globular head of the influenza B virus HA with random amino acid residues that do not affect the conformation/structure of the HA. In addition to these amino acid residue substitutions, one or more substitutions outside of the antigenic loops may be introduced. The amino acid substitutions are selected such that the folding of the chimeric HA is not significantly impacted as determined by techniques known to one of skill in the art or described herein. In addition, the amino acid substitutions may be selected such that the coding sequence for N-linkd glycosylation sites (N—X—S/T) is not altered or not significantly altered. The effect of amino acid substitutions on the conformation/structure may be determined by assays known to one of skill in the art, e.g., structure programs, crystallography, or functional assays. See, e.g., Section 5.11, infra, and Section 6, infra. For example, the chimeric HA polypeptides may be evaluated for antigenic conservation using a panel of monoclonal antibodies that bind to conserved epitopes in the globular head domain of HA and the stem domain of HA. In a specific embodiment, the methods described in Section 6, infra, are used to evaluate antigenic conservation of the chimeric HA. In addition, the chimeric HA polypeptides described herein may be evaluated to determine whether the antigenic loops of the influenza B virus HA were mutated using techniques known to one of skill in the art or described herein (see, e.g., Section 6, infra, including the HI assay described therein). In particular, the chimeric HA polypeptides described herein may be evaluated to determine if the amino acid substitutions in the antigenic loop(s) of the influenza B virus HA result in loss of a variable region(s) of the influenza B virus HA using techniques known to one of skill in the art or described herein (see, e.g., Section 6, infra, including the HI assay described therein). In a specific embodiment, the chimeric HA polypeptides described herein may be evaluated to determine if the amino acid substitutions in the antigenic loop(s) of the influenza B virus HA reduce or eliminate the immunodominant epitopes of the influenza B virus HA using techniques known to one of skill in the art or described herein (see, e.g., Section 6, infra, including the HI assay described therein). In a specific embodiment, a chimeric HA polypeptide described herein is assessed in an HI assay, such as described in Section 6, infra, to evaluate the replacement of the antigenic loop(s) in the influenza B virus HA.

In another aspect, provided herein are chimeric hemagglutinin (HA) polypeptides comprising an HA ectodomain of an influenza B virus comprising 2, 3, 4, 5, 6, 7, 8, 9, 10, 11, 12 or more amino acid substitutions within an antigenic loop of the influenza B virus HA, wherein the amino acid substitutions substitute 2, 3, 4, 5, 6, 7, 8, 9, 10, 11, 12 or more amino acid residues in the loop of the influenza B virus HA with amino acid residues found in a corresponding region of an influenza A virus HA. In another aspect, provided herein are chimeric hemagglutinin (HA) polypeptides comprising an HA ectodomain of an influenza B virus comprising 2, 3, 4, 5, 6, 7, 8, 9, 10, 11, 12 or more amino acid substitutions within the 120 loop, 150 loop, 160 loop or 190 helix of the influenza B virus HA, wherein the amino acid substitutions substitute 2, 3, 4, 5, 6, 7, 8, 9, 10, 11, 12 or more amino acid residues in the 120 loop, 150 loop, 160 loop or 190 helix of the influenza B virus HA with amino acid residues found in a corresponding region of an influenza A virus HA. In one embodiment, provided herein are chimeric hemagglutinin (HA) polypeptides comprising an HA ectodomain of an influenza B virus comprising 2, 3, 4, 5, 6, 7, 8, 9, 10, 11, 12, 13, 14, 15, 16, 17, 18, 19, 20 or more amino acid substitutions within the 120 loop of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the influenza B virus HA with amino acid residues found in a corresponding region of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In another embodiment, provided herein are chimeric hemagglutinin (HA) polypeptides comprising an HA ectodomain of an influenza B virus comprising 2, 3, 4, 5, 6, 7, 8, 9 or more amino acid substitutions within the 150 loop of the influenza B virus HA, wherein the amino acid substitutions substitute 2, 3, 4, 5, 6, 7, 8, 9 or more amino acid residues in the 150 loop of the influenza B virus HA with amino acid residues found in a corresponding region of an influenza A virus HA. In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO: 19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60). In another embodiment, provided herein are chimeric hemagglutinin (HA) polypeptides comprising an HA ectodomain of an influenza B virus comprising 2, 3, 4, 5 or more amino acid substitutions within the 160 loop of the influenza B virus HA, wherein the amino acid substitutions substitute 2, 3, 4, 5 or more amino acid residues in the 160 loop of the influenza B virus HA with amino acid residues found in a corresponding region of an influenza A virus HA. In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In another embodiment, provided herein are chimeric hemagglutinin (HA) polypeptides comprising an HA ectodomain of an influenza B virus comprising 2, 3, 4, 5, 6, 7, 8 or more amino acid substitutions within the 190 helix of the influenza B virus HA, wherein the amino acid substitutions substitute 2, 3, 4, 5, 6, 7, 8 or more amino acid residues in the 190 helix of the influenza B virus HA with amino acid residues found in a corresponding region of an influenza A virus HA. In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In some embodiments, the chimeric HA polypeptide may also comprise the signal peptide, transmembrane domain, and cytoplasmic tail domain from the influenza B virus HA. In other embodiments, the chimeric HA polypeptide comprises the signal peptide from the influenza B virus HA but lacks the transmembrane and cytoplasmic tail domains. In some embodiments, the chimeric HA polypeptide may also comprise the signal peptide, transmembrane domain, and cytoplasmic tail domain from an influenza A virus HA. In other embodiments, the chimeric HA polypeptide comprises the signal peptide from an influenza A virus HA but lacks the transmembrane and cytoplasmic tail domains. In some embodiments, the chimeric HA comprises the signal peptide of the HA of the influenza virus backbone of the chimeric HA. For example, if the chimeric HA is engineered for an influenza A virus backbone (e.g., the influenza virus comprising or engineered to express the chimeric HA is an influenza A virus), then the chimeric HA comprises the signal peptide of the influenza A virus. In some embodiments, the chimeric HA comprises the signal peptide, transmembrane domain, and cytoplasmic domain of the HA of the influenza virus backbone of the chimeric HA. In some embodiments, the chimeric HA polypeptide may also comprise the signal peptide from the HA of the influenza virus that is engineered to express the chimeric HA. In some embodiments, the chimeric HA polypeptide may also comprise the signal peptide, transmembrane domain, and cytoplasmic tail domain from the HA of the influenza virus that is engineered to express the chimeric HA. Also provided herein are nucleic acids comprising nucleotide sequences encoding such a chimeric HA. In some embodiments, the nucleic acids comprise nucleotide sequences encoding such a chimeric HA and the 5′ and 3′ non-coding regions from the influenza B virus HA. In some embodiments, the nucleic acids comprise nucleotide sequences encoding such a chimeric HA and the 5′ and 3′ non-coding regions from an influenza A virus HA. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA. In specific embodiments, the chimeric HA polypeptide is soluble. In certain embodiments, the chimeric HA polypeptides comprise 1, 2, 3, 4, 5 or more amino acid substitutions in the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid).

In another aspect, provided herein are chimeric hemagglutinin (HA) polypeptides comprising an HA ectodomain of an influenza B virus comprising one, two, three or all of the following: (i) 2, 3, 4, 5, 6, 7, 8, 9, 10, 11, 12, 13, 14, 15, 16, 17, 18, 19, 20 or more amino acid substitutions within the 120 loop of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the influenza B virus HA with amino acid residues found in a corresponding region of an influenza A virus HA; (ii) 2, 3, 4, 5, 6, 7, 8, 9 or more amino acid substitutions within the 150 loop of the influenza B virus HA, wherein the amino acid substitutions substitute 2, 3, 4, 5, 6, 7, 8, 9 or more amino acid residues in the 150 loop of the influenza B virus HA with amino acid residues found in a corresponding region of an influenza A virus HA; (iii) 2, 3, 4, 5 or more amino acid substitutions within the 160 loop of the influenza B virus HA, wherein the amino acid substitutions substitute 2, 3, 4, 5 or more amino acid residues in the 160 loop of the influenza B virus HA with amino acid residues found in a corresponding region of an influenza A virus HA; and (iv) 2, 3, 4, 5, 6, 7, 8 or more amino acid substitutions within the 190 helix of the influenza B virus HA, wherein the amino acid substitutions substitute 2, 3, 4, 5, 6, 7, 8 or more amino acid residues in the 190 helix of the influenza B virus HA with amino acid residues found in a corresponding region of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In some embodiments, the chimeric HA polypeptide may also comprise the signal peptide, transmembrane domain, and cytoplasmic tail domain from the influenza B virus HA. In other embodiments, the chimeric HA polypeptide comprises the signal peptide from the influenza B virus HA but lacks the transmembrane and cytoplasmic tail domains. In some embodiments, the chimeric HA polypeptide may also comprise the signal peptide, transmembrane domain, and cytoplasmic tail domain from an influenza A virus HA. In other embodiments, the chimeric HA polypeptide comprises the signal peptide from an influenza A virus HA but lacks the transmembrane and cytoplasmic tail domains. In some embodiments, the chimeric HA comprises the signal peptide of the HA of the influenza virus backbone of the chimeric HA. For example, if the chimeric HA is engineered for an influenza A virus backbone (e.g., the influenza virus comprising or engineered to express the chimeric HA is an influenza A virus), then the chimeric HA comprises the signal peptide of the influenza A virus. In some embodiments, the chimeric HA comprises the signal peptide, transmembrane domain, and cytoplasmic domain of the HA of the influenza virus backbone of the chimeric HA. In some embodiments, the chimeric HA polypeptide may also comprise the signal peptide from the HA of the influenza virus that is engineered to express the chimeric HA. In some embodiments, the chimeric HA polypeptide may also comprise the signal peptide, transmembrane domain, and cytoplasmic tail domain from the HA of the influenza virus that is engineered to express the chimeric HA. Also provided herein are nucleic acids comprising nucleotide sequences encoding such a chimeric HA. In some embodiments, the nucleic acids comprise nucleotide sequences encoding such a chimeric HA and the 5′ and 3′ non-coding regions from the influenza B virus HA. In some embodiments, the nucleic acids comprise nucleotide sequences encoding such a chimeric HA and the 5′ and 3′ non-coding regions from an influenza A virus HA. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA. In specific embodiments, the chimeric HA polypeptide is soluble. In certain embodiments, the chimeric HA polypeptides comprise 1, 2, 3, 4, 5 or more amino acid substitutions in the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid).

In another aspect, provided herein are chimeric hemagglutinin (HA) polypeptides comprising (i) a hemagglutinin ectodomain from an influenza B virus with 2, 3, 4, 5, 6, 7, 8, 9, 10, 11, 12 or more amino acid substitutions within the 120 loop, 150 loop, 160 loop or 190 helix of the influenza B virus HA and (ii) a signal peptide, a transmembrane domain and a cytoplasmic tail domain from an influenza A virus, wherein the amino acid substitutions substitute 2, 3, 4, 5, 6, 7, 8, 9, 10, 11, 12 or more amino acid residues in the 120 loop, 150 loop, 160 loop or 190 helix of the influenza B virus HA with amino acid residues found in a corresponding region of an influenza A virus HA. In one embodiment, provided herein are chimeric hemagglutinin (HA) polypeptides comprising (i) a hemagglutinin ectodomain from an influenza B virus with 2, 3, 4, 5, 6, 7, 8, 9, 10, 11, 12, 13, 14, 15, 16, 17, 18, 19, 20 or more amino acid substitutions within the 120 loop of the influenza B virus HA and (ii) a signal peptide, a transmembrane domain and a cytoplasmic tail domain from an influenza A virus, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the influenza B virus HA with amino acid residues found in a corresponding region of an influenza A virus HA. In another embodiment, provided herein are chimeric hemagglutinin (HA) polypeptides comprising (i) a hemagglutinin ectodomain from an influenza B virus with 2, 3, 4, 5, 6, 7, 8, 9 or more amino acid substitutions within the 150 loop of the influenza B virus HA and (ii) a signal peptide, a transmembrane domain and a cytoplasmic tail domain from an influenza A virus, wherein the amino acid substitutions substitute 2, 3, 4, 5, 6, 7, 8, 9 or more amino acid residues in the 150 loop of the influenza B virus HA with amino acid residues found in a corresponding region of an influenza A virus HA. In another embodiment, provided herein are chimeric hemagglutinin (HA) polypeptides comprising (i) a hemagglutinin ectodomain from an influenza B virus with 2, 3, 4, 5 or more amino acid substitutions within the 160 loop of the influenza B virus HA and (ii) a signal peptide, a transmembrane domain and a cytoplasmic tail domain from an influenza A virus, wherein the amino acid substitutions substitute 2, 3, 4, 5 or more amino acid residues in the 160 loop of the influenza B virus HA with amino acid residues found in a corresponding region of an influenza A virus HA. In another embodiment, provided herein are chimeric hemagglutinin (HA) polypeptides comprising (i) a hemagglutinin ectodomain from an influenza B virus with 2, 3, 4, 5, 6, 7, 8 or more amino acid substitutions within the 190 helix of the influenza B virus HA and (ii) a signal peptide, a transmembrane domain and a cytoplasmic tail domain from an influenza A virus, wherein the amino acid substitutions substitute 2, 3, 4, 5, 6, 7, 8 or more amino acid residues in the 190 helix of the influenza B virus HA with amino acid residues found in a corresponding region of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the influenza B virus is from the Yamagata lineage. In other embodiments, the influenza B virus is from the Victoria lineage. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska 7MP0167_2007), or H13 strain (e.g., A/black headed gull/Sweden/1/99). Also provided herein are nucleic acids comprising nucleotide sequences encoding such a chimeric HA. In some embodiments, the nucleic acids comprise nucleotide sequences encoding such a chimeric HA and the 5′ and 3′ non-coding regions from an influenza A virus HA. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA.

In another aspect, provided herein are chimeric hemagglutinin (HA) polypeptides comprising (i) a hemagglutinin ectodomain from a first influenza B virus strain with 2, 3, 4, 5, 6, 7, 8, 9, 10, 11, 12 or more amino acid substitutions within the 120 loop, 150 loop, 160 loop or 190 helix of the first influenza B virus strain HA and (ii) a signal peptide, a transmembrane domain and a cytoplasmic tail domain from a second influenza B virus strain, wherein the amino acid substitutions substitute 2, 3, 4, 5, 6, 7, 8, 9, 10, 11, 12 or more amino acid residues in the 120 loop, 150 loop, 160 loop or 190 helix of the first influenza B virus strain HA with amino acid residues found in a corresponding region of an influenza A virus HA. In one embodiment, provided herein are chimeric hemagglutinin (HA) polypeptides comprising (i) a hemagglutinin ectodomain from a first influenza B virus strain with 2, 3, 4, 5, 6, 7, 8, 9, 10, 11, 12, 13, 14, 15, 16, 17, 18, 19, 20 or more amino acid substitutions within the 120 loop of the first influenza B virus strain HA and (ii) a signal peptide, a transmembrane domain and a cytoplasmic tail domain from a second influenza B virus strain, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the first influenza B virus strain HA with amino acid residues found in a corresponding region of an influenza A virus HA. In another embodiment, provided herein are chimeric hemagglutinin (HA) polypeptides comprising (i) a hemagglutinin ectodomain from a first influenza B virus strain with 2, 3, 4, 5, 6, 7, 8, 9 or more amino acid substitutions within the 150 loop of the first influenza B virus strain HA and (ii) a signal peptide, a transmembrane domain and a cytoplasmic tail domain from a second influenza B virus, wherein the amino acid substitutions substitute 2, 3, 4, 5, 6, 7, 8, 9 or more amino acid residues in the 150 loop of the first influenza B virus strain HA with amino acid residues found in a corresponding region of an influenza A virus HA. In another embodiment, provided herein are chimeric hemagglutinin (HA) polypeptides comprising (i) a hemagglutinin ectodomain from a first influenza B virus strain with 2, 3, 4, 5 or more amino acid substitutions within the 160 loop of the first influenza B virus strain HA and (ii) a signal peptide, a transmembrane domain and a cytoplasmic tail domain from a second influenza B virus strain, wherein the amino acid substitutions substitute 2, 3, 4, 5 or more amino acid residues in the 160 loop of the first influenza B virus strain HA with amino acid residues found in a corresponding region of an influenza A virus HA. In another embodiment, provided herein are chimeric hemagglutinin (HA) polypeptides comprising (i) a hemagglutinin ectodomain from a first influenza B virus strain with 2, 3, 4, 5, 6, 7, 8 or more amino acid substitutions within the 190 helix of the first influenza B virus strain HA and (ii) a signal peptide, a transmembrane domain and a cytoplasmic tail domain from a second influenza B virus strain, wherein the amino acid substitutions substitute 2, 3, 4, 5, 6, 7, 8 or more amino acid residues in the 190 helix of the first influenza B virus strain HA with amino acid residues found in a corresponding region of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 13. In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 15. In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 17. In specific embodiments, a chimeric HA polypeptide described herein comprises a one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTFNVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the influenza B virus HA which are outside of one, two, three, or all of the following: the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the first influenza B virus strain and the second influenza B virus strain are from the same lineage. In some embodiments, the first influenza B virus strain and the second influenza B virus strain are from the same lineage but are different strains. In a specific embodiment, the first influenza B virus strain is the same strain as the second influenza B virus strain. In another embodiment, the first influenza B virus strain is a different strain than the second influenza B virus strain. In some embodiments, the first influenza B virus strain and the second influenza B virus strain are from different lineages. In some embodiments, the first influenza B virus strain is from the Yamagata lineage. In other embodiments, the first influenza B virus is from the Victoria lineage. In some embodiments, the second influenza B virus strain is from the Yamagata lineage. In other embodiments, the second influenza B virus is from the Victoria lineage. In a specific embodiment, the second influenza B virus strain is the same strain as the influenza virus backbone of an influenza virus either containing, expressing, or both the chimeric HA. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99). Also provided herein are nucleic acids comprising nucleotide sequences encoding said chimeric HA polypeptides. In some embodiments, the nucleic acids comprise nucleotide sequences encoding such a chimeric HA and the 5′ non-coding region and 3′ non-coding region from an influenza B virus. In some embodiments, the nucleic acids comprise nucleotide sequences encoding such a chimeric HA and the 5′ and 3′ non-coding regions of the HA of the influenza virus backbone of an influenza virus either comprising, containing, or both the chimeric HA.

In another aspect, provided herein are chimeric hemagglutinin (HA) polypeptides comprising: (a) a hemagglutinin ectodomain from an influenza B virus with one, two, three or all of the following (i) 2, 3, 4, 5, 6, 7, 8, 9, 10, 11, 12, 13, 14, 15, 16, 17, 18, 19, 20 or more amino acid substitutions within the 120 loop of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the influenza B virus HA with amino acid residues found in a corresponding region of an influenza A virus HA; (ii) 2, 3, 4, 5, 6, 7, 8, 9 or more amino acid substitutions within the 150 loop of the influenza B virus HA, wherein the amino acid substitutions substitute 2, 3, 4, 5, 6, 7, 8, 9 or more amino acid residues in the 150 loop of the influenza B virus HA with amino acid residues found in a corresponding region of an influenza A virus HA; (iii) 2, 3, 4, 5 or more amino acid substitutions within the 160 loop of the influenza B virus HA, wherein the amino acid substitutions substitute 2, 3, 4, 5 or more amino acid residues in the 160 loop of the influenza B virus HA with amino acid residues found in a corresponding region of an influenza A virus HA; and (iv) 2, 3, 4, 5, 6, 7, 8 or more amino acid substitutions within the 190 helix of the influenza B virus HA, wherein the amino acid substitutions substitute 2, 3, 4, 5, 6, 7, 8 or more amino acid residues in the 190 helix of the influenza B virus HA with amino acid residues found in a corresponding region of an influenza A virus HA; and (b) a signal peptide, a transmembrane domain and a cytoplasmic tail domain from an influenza A virus. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMOT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTENVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the influenza B virus is from the Yamagata lineage. In other embodiments, the influenza B virus is from the Victoria lineage. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska 7MP0167_2007), or H13 strain (e.g., A/black headed gull/Sweden/1/99). Also provided herein are nucleic acids comprising nucleotide sequences encoding said chimeric HA polypeptides. In some embodiments, the nucleic acids comprise nucleotide sequences encoding such a chimeric HA and the 5′ non-coding region and 3′ non-coding region from an influenza A virus. In some embodiments, the nucleic acids comprise nucleotide sequences encoding a chimeric HA polypeptide and the 5′ non-coding region and 3′ non-coding region from an influenza B virus. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA. In a specific embodiment, the nucleic acid comprises the nucleotide sequence set forth in FIG. 12A and FIG. 12B or the complement thereof. In a specific embodiment, the nucleic acid comprises the nucleotide sequence set forth in FIG. 14A and FIG. 14B or the complement thereof. In a specific embodiment, the nucleic acid comprises the nucleotide sequence set forth in FIG. 16A and FIG. 16B or the complement thereof. In a specific embodiment, the nucleic acid comprises the nucleotide sequence set forth in FIG. 18A and FIG. 18B or the complement thereof.

In another aspect, provided herein are chimeric hemagglutinin (HA) polypeptides comprising: (a) a hemagglutinin ectodomain from a first influenza B virus strain with one, two, three or all of the following (i) 2, 3, 4, 5, 6, 7, 8, 9, 10, 11, 12, 13, 14, 15, 16, 17, 18, 19, 20 or more amino acid substitutions within the 120 loop of the first influenza B virus strain HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the first influenza B virus strain HA with amino acid residues found in a corresponding region of an influenza A virus HA; (ii) 2, 3, 4, 5, 6, 7, 8, 9 or more amino acid substitutions within the 150 loop of the first influenza B virus strain HA, wherein the amino acid substitutions substitute 2, 3, 4, 5, 6, 7, 8, 9 or more amino acid residues in the 150 loop of the first influenza B virus strain HA with amino acid residues found in a corresponding region of an influenza A virus HA; (iii) 2, 3, 4, 5 or more amino acid substitutions within the 160 loop of the first influenza B virus strain HA, wherein the amino acid substitutions substitute 2, 3, 4, 5 or more amino acid residues in the 160 loop of the first influenza B virus strain HA with amino acid residues found in a corresponding region of an influenza A virus HA; and (iv) 2, 3, 4, 5, 6, 7, 8 or more amino acid substitutions within the 190 helix of the first influenza B virus strain HA, wherein the amino acid substitutions substitute 2, 3, 4, 5, 6, 7, 8 or more amino acid residues in the 190 helix of the first influenza B virus strain HA with amino acid residues found in a corresponding region of an influenza A virus HA; and (b) a signal peptide, a transmembrane domain and a cytoplasmic tail domain from a second influenza B virus strain. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 13. In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMQT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 15. In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 17. In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTFNVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises one, two, three, or all of the following: a 120 loop, a 150 loop, a 160 loop, and a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the influenza B virus HA which are outside of one, two, three, or all of the following: the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the first influenza B virus strain and the second influenza B virus strain are from the same lineage. In some embodiments, the first influenza B virus strain and the second influenza B virus strain are from the same lineage but are different strains. In a specific embodiment, the first influenza B virus strain is the same strain as the second influenza B virus strain. In another embodiment, the first influenza B virus strain is a different strain than the second influenza B virus strain. In some embodiments, the first influenza B virus strain and the second influenza B virus strain are from different lineages. In some embodiments, the first influenza B virus strain is from the Yamagata lineage. In other embodiments, the first influenza B virus is from the Victoria lineage. In some embodiments, the second influenza B virus strain is from the Yamagata lineage. In other embodiments, the second influenza B virus is from the Victoria lineage. In a specific embodiment, the second influenza B virus strain is the same strain as the influenza virus backbone of an influenza virus either comprising, containing, or both the chimeric HA. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99). Also provided herein are nucleic acids comprising nucleotide sequences encoding said chimeric HA polypeptides. In some embodiments, the nucleic acids comprise nucleotide sequences encoding such a chimeric HA and the 5′ and 3′ non-coding regions of the HA of the influenza virus backbone of an influenza virus either comprising, containing, or both the chimeric HA. In a specific embodiment, the nucleic acid comprises the nucleotide sequence set forth in FIG. 29 or the complement thereof. In a specific embodiment, the nucleic acid comprises the nucleotide sequence set forth in FIG. 31 or the complement thereof. In a specific embodiment, the nucleic acid comprises the nucleotide sequence set forth in FIG. 33 or the complement thereof. In a specific embodiment, the nucleic acid comprises the nucleotide sequence set forth in FIG. 35 or the complement thereof.

In a specific embodiment, the influenza A virus HA utilized in the generation of a chimeric HA polypeptide described herein is the HA from an influenza virus of the H1, H2, H3, H4, H5, H6, H7, H8, H9, H10, H11, H12, H13, H14, H15, H16, H17, or H18 subtype. In a specific embodiment, the influenza A virus HA utilized in the generation of a chimeric HA polypeptide described herein is the HA from an influenza virus of the H2, H4, H5, H6, H7, H8, H9, H10, H11, H12, H13, H14, H15, H16, H17, or H18 subtype. In a specific embodiment, the influenza A virus HA utilized in the generation of a chimeric HA polypeptide described herein is the HA from an influenza virus of the H5, H8, H11, H12, or H13 subtype. In a specific embodiment, the influenza A virus HA utilized in the generation of a chimeric HA polypeptide described herein is the HA from an influenza virus of the H5 subtype. In a specific embodiment, the influenza A virus HA utilized in the generation of a chimeric HA polypeptide described herein is the HA from an influenza virus of the H8 subtype. In a specific embodiment, the influenza A virus HA utilized in the generation of a chimeric HA polypeptide described herein is the HA from an influenza virus of the H11 subtype. In a specific embodiment, the influenza A virus HA utilized in the generation of a chimeric HA polypeptide described herein is the HA from an influenza virus of the H12 subtype. In a specific embodiment, the influenza A virus HA utilized in the generation of a chimeric HA polypeptide described herein is the HA from an influenza virus of the H13 subtype. In a specific embodiment, the influenza A virus HA utilized in the generation of a chimeric HA polypeptide described herein is the HA from an avian influenza virus. In a specific embodiment, the influenza A virus HA utilized in the generation of a chimeric HA polypeptide described herein is the HA from influenza A/mallard/Sweden/24/2002 virus (GenBank Accession No. CY060249.1; GenBank GI No. 294441479; see, also, FIG. 21A and FIG. 21B). In a specific embodiment, the influenza A virus HA utilized in the generation of a chimeric HA polypeptide described herein is the HA from influenza A/Vietnam/1203/04 virus (GenBank Accession No. EF541403.1; GenBank GI No. 145284465; see, also, FIG. 22A and FIG. 22B and Steel et al., 2009, Journal of Virology, 83 (4): 1742-1753 for the HA of influenza A/Vietnam/1203/04 (HALo) virus). In a specific embodiment, the influenza A virus HA utilized in the generation of a chimeric HA polypeptide described herein is the HA from influenza A/northern shoveler/Netherlands/18/99 virus (GenBank Accession No. CY060417.1; GenBank GI No. 294441876; see, also, FIG. 23A and FIG. 23B). In a specific embodiment, the influenza A virus HA utilized in the generation of a chimeric HA polypeptide described herein is the HA from influenza A_mallard_interior Alaska 7MP0167_2007 virus (GenBank Accession No. CY077198.1; GenBank GI No. 312652817; see, also, FIG. 24A and FIG. 24B). In a specific embodiment, the influenza A virus HA utilized in the generation of a chimeric HA polypeptide described herein is the HA from influenza A/black headed gull/Sweden/1/99 (GenBank Accession No. AY684887.1; see, also, FIG. 41A and FIG. 41B). In a specific embodiment, the influenza A virus HA utilized in the generation of a chimeric HA polypeptide described herein is the HA from influenza A/Puerto Rico/8/34 virus (GenBank Accession No. AF389118.1; GenBank GI No. 21693168; see, also, FIG. 25A and FIG. 25B). In a specific embodiment, the influenza B virus HA utilized in the generation of a chimeric HA polypeptide described herein is the HA from influenza B/Yamagata/16/88 virus (see, FIG. 26A and FIG. 26B). In a specific embodiment, the influenza B virus HA utilized in the generation of a chimeric HA polypeptide described herein is the HA from a mouse-adapted influenza B/Malaysia/2506/04 virus (see, e.g., SEQ ID NO: 73 or 83). In a specific embodiment, the influenza B virus HA utilized in the generation of a chimeric HA polypeptide described herein is the HA from influenza B/Malaysia/2506/04 virus (see, e.g., GenBank Accession No. CY040449.1).

In a specific embodiment, a chimeric HA polypeptide is a chimeric HA polypeptide described in Section 6, infra.

In another aspect, provided herein are chimeric hemagglutinin (HA) polypeptides comprising an HA ectodomain of an influenza B virus comprising 2, 3, 4, 5, 6, 7, 8, 9, 10, 11, 12 or more amino acid substitutions within an antigenic loop of the influenza B virus HA (e.g., 120 loop, 150 loop, 160 loop and/or 190 helix), wherein the amino acid substitutions substitute 2, 3, 4, 5, 6, 7, 8, 9, 10, 11, 12 or more amino acid residues in the loop of the influenza B virus HA with random amino acid residues that do not affect the conformation/structure of the HA. In addition to these amino acid residue substitutions, one or more substitutions outside of the antigenic loops may be introduced. The amino acid substitutions are selected such that the folding of the chimeric HA is not significantly impacted as determined by techniques known to one of skill in the art or described herein. In addition, the amino acid substitutions may be selected such that the coding sequence for N-linkd glycosylation sites (N—X—S/T) is not altered or not significantly altered. The effect of amino acid substitutions on the conformation/structure may be determined by assays known to one of skill in the art, e.g., structure programs, crystallography, or functional assays. See, e.g., Section 5.11, infra, and Section 6, infra. For example, the chimeric HA polypeptides may be evaluated for antigenic conservation using a panel of monoclonal antibodies that bind to conserved epitopes in the globular head domain of HA and the stem domain of HA. In a specific embodiment, the methods described in Section 6, infra, are used to evaluate antigenic conservation of the chimeric HA. In addition, the chimeric HA polypeptides described herein may be evaluated to determine whether the antigenic loops of the influenza B virus HA were mutated using techniques known to one of skill in the art or described herein (see, e.g., Section 6, infra, including the HI assay described therein). In particular, the chimeric HA polypeptides described herein may be evaluated to determine if the amino acid substitutions in the antigenic loop(s) of the influenza B virus HA result in loss of a variable region(s) of the influenza B virus HA using techniques known to one of skill in the art or described herein (see, e.g., Section 6, infra, including the HI assay described therein). In a specific embodiment, the chimeric HA polypeptides described herein may be evaluated to determine if the amino acid substitutions in the antigenic loop(s) of the influenza B virus HA reduce or eliminate the immunodominant epitopes of the influenza B virus HA using techniques known to one of skill in the art or described herein (see, e.g., Section 6, infra, including the HI assay described therein). In a specific embodiment, a chimeric HA polypeptide described herein is assessed in an HI assay, such as described in Section 6, infra, to evaluate the replacement of the antigenic loop(s) in the influenza B virus HA.

In another aspect, provided herein are nucleic acid sequences comprising a nucleotide sequence encoding a chimeric HA polypeptide described herein. In a specific embodiment, provided herein is a nucleic acid sequence comprising a nucleotide sequence encoding a chimeric HA polypeptide described herein. In another specific embodiment, provided herein is an HA segment comprising the nucleic acid sequence encoding a chimeric HA polypeptide described herein. In a specific embodiment, the nucleic acid sequence comprises the nucleotide sequence set forth in FIG. 12, FIG. 14, FIG. 16, FIG. 18, FIG. 29, FIG. 31, FIG. 33, or FIG. 35. In certain embodiments, the HA segment or the nucleic acid sequence comprising the nucleotide sequence encoding a chimeric HA polypeptide described herein are transfected into cells (e.g., cell lines).

In another aspect, provided herein are host cells comprising or engineered to express a chimeric HA polypeptide described herein. In one embodiment, provided herein are host cells comprising a chimeric HA polypeptide described herein. In another embodiment, provided herein are host cells comprising an HA segment comprising the nucleic acid sequence encoding a chimeric HA polypeptide described herein. In another embodiment, provided herein are embryonated eggs (e.g., chicken embryonated eggs) comprising a chimeric HA polypeptide described herein. In another embodiment, provided herein are embryonated eggs (e.g., chicken embryonated eggs) comprising an HA segment comprising the nucleic acid sequence encoding a chimeric HA polypeptide described herein. In another embodiment, provided herein are embryonated eggs (e.g., chicken embryonated eggs) comprising an influenza virus, wherein said influenza virus comprises an HA segment comprising the nucleic acid sequence encoding a chimeric HA polypeptide described herein. In another embodiment, provided herein are embryonated eggs (e.g., chicken embryonated eggs) comprising an influenza virus, wherein the influenza virus comprises a chimeric HA polypeptide described herein.

In another aspect, provided herein are influenza viruses engineered to express a chimeric HA polypeptide described herein. In a specific embodiment, provided herein is an influenza A virus engineered to express a chimeric HA polypeptide described herein. In accordance with this embodiment, the signal peptide, transmembrane domain and cytoplasmic tail domain of the chimeric HA polypeptide are preferably derived from the same influenza A virus as the influenza A virus engineered to express the chimeric HA polypeptide. Thus, in accordance with this embodiment, a nucleic acid comprising nucleotide sequences encoding said chimeric HA polypeptide preferably comprises the 5′ non-coding region, 3′ non-coding region and nucleotide sequences encoding the signal peptide, transmembrane domain, and cytoplasmic domain derived from the same influenza A virus as the influenza A virus engineered to express the chimeric HA polypeptide. In specific embodiments, provided herein is an influenza A virus comprising 7 non-HA segments of an influenza A virus segments and an HA segment comprising the nucleic acid sequence encoding a chimeric HA polypeptide described herein. In accordance with these embodiments, the signal peptide, transmembrane domain and cytoplasmic tail domain of the chimeric HA polypeptide are preferably derived from the same influenza A virus as influenza A virus comprising the non-HA segments. In accordance with these embodiments, a nucleic acid comprising nucleotide sequences encoding the chimeric HA polypeptide preferably comprises the 5′ non-coding region and the 3′ non-coding region, and the nucleic sequences encoding the signal peptide, transmembrane domain, and cytoplasmic domain derived from the same influenza A virus as the influenza A virus comprising the non-HA segments. In another embodiment, provided herein is an influenza A virus engineered to express and contain a chimeric HA polypeptide described herein.

In a specific embodiment, provided herein is an influenza B virus engineered to express a chimeric HA polypeptide described herein. In specific embodiments, provided herein is an influenza B virus comprising 7 non-HA segments of an influenza B virus segments and an HA segment comprising the nucleic acid sequence encoding a chimeric HA polypeptide described herein. In certain embodiments, the 7 non-HA segments are from the same influenza B virus as the ectodomain of the chimeric HA polypeptide. In some embodiments, the 7 non-HA segments are from the same influenza B virus as the globular head domain of the chimeric HA polypeptide. In another embodiment, provided herein is an influenza B virus engineered to express and contain a chimeric HA polypeptide described herein.

In a specific embodiment, provided herein is an influenza B virus engineered to express a chimeric HA polypeptide described herein. In accordance with this embodiment, the signal peptide, transmembrane domain and cytoplasmic tail domain of the chimeric HA polypeptide are preferably derived from the same influenza B virus as the influenza B virus engineered to express the chimeric HA polypeptide. Thus, in accordance with this embodiment, a nucleic acid comprising nucleotide sequences encoding said chimeric HA polypeptide preferably comprises the 5′ non-coding region, 3′ non-coding region and nucleotide sequences encoding the signal peptide, transmembrane domain, and cytoplasmic domain derived from the same influenza B virus as the influenza B virus engineered to express the chimeric HA polypeptide. In specific embodiments, provided herein is an influenza B virus comprising 7 non-HA segments of an influenza B virus segment and an HA segment comprising the nucleic acid sequence encoding a chimeric HA polypeptide described herein. In accordance with these embodiments, the signal peptide, transmembrane domain and cytoplasmic tail domain of the chimeric HA polypeptide are preferably derived from the same influenza B virus as the influenza B virus comprising the non-HA segments. In accordance with these embodiments, a nucleic acid comprising nucleotide sequences encoding the chimeric HA polypeptide preferably comprises the 5′ non-coding region and the 3′ non-coding region, and the nucleic sequences encoding the signal peptide, transmembrane domain, and cytoplasmic domain derived from the same influenza B virus as the influenza B virus comprising the non-HA segments. In another embodiment, provided herein is an influenza B virus engineered to express and contain a chimeric HA polypeptide described herein.

In another aspect, provided herein are influenza viruses containing a chimeric HA polypeptide described herein. In a specific embodiment, provided herein is an influenza A virus containing a chimeric HA polypeptide described herein. In another embodiment, provided herein is an influenza B virus containing a chimeric HA polypeptide described herein. In certain embodiments, the influenza B virus containing the chimeric HA polypeptide is from the Yamagata lineage. In other embodiments, the influenza B virus containing the chimeric HA polypeptide is from the Victoria lineage. In specific embodiments, the influenza B virus containing the chimeric HA polypeptide is B/Malaysia/2506/04.

In a specific embodiment, provided herein is an influenza virus described in Section 6, infra.

In one embodiment, provided herein is a virus-like particle comprising a chimeric HA polypeptide described herein. In another embodiment, provided herein is a virosome comprising a chimeric HA polypeptide described herein.

In another aspect, provided herein are compositions (e.g., immunogenic compositions) comprising a chimeric HA polypeptide described herein, a nucleic acid sequence comprising a nucleotide sequence encoding a chimeric HA polypeptide described herein, an HA segment comprising the nucleic acid sequence encoding a chimeric HA polypeptide described herein, an influenza virus comprising/containing a chimeric HA polypeptide described herein, an influenza virus engineered to express a chimeric HA polypeptide described herein, a virus-like particle comprising a chimeric HA polypeptide described herein, or a virosome comprising a chimeric HA polypeptide described herein. In one embodiment, provided herein is an immunogenic composition comprising a chimeric HA polypeptide described herein. In another embodiment, provided herein is an immunogenic composition comprising a nucleic acid sequence comprising a nucleotide sequence encoding a chimeric HA polypeptide described herein or an HA segment comprising the nucleic acid sequence encoding a chimeric HA polypeptide described herein. In another embodiment, provided herein is an immunogenic composition comprising an influenza virus comprising a chimeric HA polypeptide described herein. In another embodiment, provided herein is an immunogenic composition comprising an influenza virus engineered to express a chimeric HA polypeptide described herein. In another embodiment, provided herein is an immunogenic composition comprising an influenza virus engineered to express and contain a chimeric HA polypeptide described herein. In certain embodiments, the influenza virus is a live attenuated influenza virus. In other embodiments, the influenza virus is an inactivated virus. In one embodiment, the composition is a subunit vaccine. In another embodiment, the composition is a split vaccine. In another embodiment, provided herein is an immunogenic composition comprising a virus-like particle described herein. In another embodiment, provided herein is an immunogenic composition comprising a virosome described herein. In some embodiments, the compositions further comprise one or more adjuvants (see, e.g., Section 5.7, infra, regarding adjuvants, such as AS03 or MF59).

In another aspect, provided herein are methods for immunizing a subject against influenza virus, comprising administering to the subject a composition described herein. In a specific embodiment, provided herein is a method for immunizing a subject against influenza virus, comprising administering to the subject an effective amount of a composition described herein. In a specific embodiment, provided herein is a method for inducing an immune response (e.g., an antibody response) to an influenza B virus HA stem domain in a subject, comprising administering the subject a composition described herein. In another specific embodiment, provided herein is a method for inducing an immune response (e.g., an antibody response) to an influenza B virus HA globular head domain and stem domain in a subject, comprising administering to the subject a composition described herein. In another specific embodiment, provided herein is a method for inducing an immune response (e.g., an antibody response) to an influenza B virus HA stem domain and an influenza A virus globular head domain in a subject, comprising administering the subject a composition described herein. In another specific embodiment, provided herein is a method for inducing an immune response (e.g., an antibody response) to an influenza B virus HA globular head domain and stem domain, and an influenza A virus globular head domain in a subject, comprising administering to the subject a composition described herein. In particular embodiments, the subject is human.

In another embodiment, provided herein is a method of immunizing a subject against an influenza virus disease or infection comprising (i) a first administration of a first chimeric HA polypeptide, a first nucleic acid encoding such a polypeptide(s), a first vector either containing, expressing, or both such a polypeptide(s), or cells described herein to the subject; and (ii) a second administration of a second chimeric HA polypeptide, a second nucleic acid encoding such a polypeptide(s), a second vector either containing, expressing, or both such a polypeptide(s), or cells described herein to the subject, wherein the first and second chimeric HA polypeptides comprise an ectodomain with the same stem domains but have different 120 loops, 150 loops, 160 loops, and/or 190 helices. In specific embodiments, the first and second chimeric HA have either one, two, three, or four of the following: (i) different 120 loops, (ii) different 150 loops, (iii) different 160 loops, and (iv) different 190 helices. The first and second administrations may be separated by at least 1 day, 2 days, 3 days, 5 days, 10 days, 15 days, 30 days, 45 days, 2 months, 75 days, 3 months, or at least 6 months. In specific embodiments, the first and second administrations may be separated by 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months. In certain embodiments, booster inoculations may be administered to the subject at 6 to 12 month intervals following the second inoculation. In certain embodiments, the booster inoculation comprises a third chimeric HA polypeptide, a third nucleic acid encoding such a polypeptide(s), a third vector either containing, expressing, or both such a polypeptide(s), or cells described herein, wherein the first, second, and third chimeric HA polypeptides comprise an ectodomain with the same stem but have different 120 loops, 150 loops, 160 loops, and/or 190 helices. In specific embodiments, the first, second, and third chimeric HA have either one, two, three, or four of the following: (i) different 120 loops, (ii) different 150 loops, (iii) different 160 loops, and (iv) different 190 helices.

In another aspect, provided herein is a composition described herein for use in a method for immunizing a subject against influenza virus, wherein the method comprises administering to the subject the composition. In a specific embodiment, provided herein is a composition described herein for use in a method for immunizing a subject against influenza virus, wherein the method comprises administering to the subject an effective amount of the composition. In a specific embodiment, provided herein is a composition described herein for use in a method for inducing an immune response (e.g., an antibody response) to an influenza B virus HA stem domain in a subject, wherein the method comprises administering the subject the composition. In another specific embodiment, provided herein is a composition described herein for use in a method for inducing an immune response (e.g., an antibody response) to an influenza B virus HA globular head domain and stem domain in a subject, wherein the method comprises administering to the subject the composition. In another specific embodiment, provided herein is a composition described herein for use in a method for inducing an immune response (e.g., an antibody response) to an influenza B virus HA stem domain and an influenza A virus globular head domain in a subject, wherein the method comprises administering the subject the composition. In another specific embodiment, provided herein is a composition described herein for use in a method for inducing an immune response (e.g., an antibody response) to an influenza B virus HA globular head domain and stem domain, and an influenza A virus globular head domain in a subject, wherein the method comprises administering to the subject the composition. In particular embodiments, the subject is human.

In another embodiment, provided herein is a first immunogenic composition for use in a method of immunizing a subject against an influenza virus disease or infection, wherein the method comprises (i) a first administration of the first immunogenic composition to the subject, wherein the first immunogenic composition comprises a first chimeric HA polypeptide, a first nucleic acid encoding such a polypeptide(s), a first vector either containing, expressing, or both such a polypeptide(s), or cells described herein; and (ii) a second administration of a second immunogenic composition to the subject, wherein the second immunogenic composition comprises chimeric HA polypeptide, a second nucleic acid encoding such a polypeptide(s), a second vector either containing, expressing, or both such a polypeptide(s), or cells described herein, and wherein the first and second chimeric HA polypeptides comprise an ectodomain with the same stem domains but have different 120 loops, 150 loops, 160 loops, and/or 190 helices. In specific embodiments, the first and second chimeric HA have either one, two, three, or four of the following: (i) different 120 loops, (ii) different 150 loops, (iii) different 160 loops, and (iv) different 190 helices. The first and second administrations may be separated by at least 1 day, 2 days, 3 days, 5 days, 10 days, 15 days, 30 days, 45 days, 2 months, 75 days, 3 months, or at least 6 months. In specific embodiments, the first and second administrations may be separated by 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months. In certain embodiments, booster inoculations may be administered to the subject at 6 to 12 month intervals following the second inoculation. In certain embodiments, the booster inoculation comprises a third chimeric HA polypeptide, a third nucleic acid encoding such a polypeptide(s), a third vector either containing, expressing, or both such a polypeptide(s), or cells described herein, wherein the first, second, and third chimeric HA polypeptides comprise an ectodomain with the same stem but have different 120 loops, 150 loops, 160 loops, and/or 190 helices. In specific embodiments, the first, second, and third chimeric HA have either one, two, three, or four of the following: (i) different 120 loops, (ii) different 150 loops, (iii) different 160 loops, and (iv) different 190 helices.

Without being bound by any theory, the chimeric HA polypeptides described herein, when administered to a subject, focuses the immune response towards conserved epitopes in the globular head domain as well as the stem domain of an influenza B virus HA. Thus, in specific embodiments, provided herein is a method for inducing an immune response (e.g., an antibody response) to conserved epitopes in the globular head domain and stem domain of an influenza B virus HA in a subject, comprising administering to the subject a chimeric HA polypeptide described herein or a composition thereof.

In another aspect, provided herein are methods for preventing and/or treating an influenza virus infection or influenza virus disease, comprising administering to a subject in need thereof a composition described herein. In a specific embodiment, provided herein is a method for preventing an influenza virus disease, comprising administering to a subject in need thereof a composition described herein. In another specific embodiment, provided herein is a method for treating an influenza virus infection, comprising administering to a subject in need thereof a composition described herein. In another specific embodiment, provided herein is a method for treating an influenza virus disease, comprising administering to a subject in need thereof a composition described herein. In particular embodiments, the subject is human.

In another aspect, provided herein is a composition described herein for use in a method for preventing and/or treating an influenza virus infection or influenza virus disease, wherein the method comprises administering to a subject in need thereof the composition. In a specific embodiment, provided herein is a composition described herein for use in a method for preventing an influenza virus disease, wherein the method comprises administering to a subject in need thereof the composition. In another specific embodiment, provided herein is a composition described herein for use in a method for treating an influenza virus infection, wherein the method comprises administering to a subject in need thereof the composition. In another specific embodiment, provided herein is a composition described herein for use in a method for treating an influenza virus disease, wherein the method comprises administering to a subject in need thereof the composition. In particular embodiments, the subject is human.

The chimeric HA polypeptides, nucleic acids encoding such polypeptides, or vectors comprising such nucleic acids or polypeptides described herein may be used to elicit neutralizing antibodies against influenza, for example, against the stalk region of an influenza virus hemagglutinin polypeptide and/or subdominant epitopes in the globular head domain of an influenza virus hemagglutinin polypeptide. In a specific embodiment, the chimeric HA polypeptide, nucleic acids encoding such polypeptides, or vectors comprising such nucleic acids or polypeptides described herein may be administered to a non-human subject (e.g., a mouse, rabbit, rat, guinea pig, etc.) to induce an immune response that includes the production of antibodies which may be isolated using techniques known to one of skill in the art (e.g., immunoaffinity chromatography, centrifugation, precipitation, etc.).

### 3.1 Terminology

As used herein, the term “120 loop” refers to an antigenic region in an influenza B virus HA. In a specific embodiment, the term “120 loop” refers to amino acid residues 116 to 137 of the HA1 domain of influenza B virus B/Hong Kong/8/73 or amino acid residues in the HA1 domain of an influenza B virus other than B/Hong Kong/8/73 that correspond to amino acid residues 116 to 137 of the HA1 domain of influenza B virus B/Hong Kong/8/73 (wherein the amino acid residues 116-137 correspond to the numbered positions of the influenza B virus B/Hong Kong/8/73 HA not including the signal peptide, i.e., the numbering of the mature HA). In another specific embodiment, the term “120 loop” refers to amino acid residues 75 to 77, and 116 to 137 of the HA1 domain of influenza B virus B/Hong Kong/8/73 or amino acid residues in the HA1 domain of an influenza B virus other than B/Hong Kong/8/73 that correspond to amino acid residues 75 to 77, and 116 to 137 of the HA1 domain of influenza B virus B/Hong Kong/8/73 (wherein the amino acid residues 75 to 77 and 116-137 correspond to the numbered positions of the influenza B virus B/Hong Kong/8/73 HA not including the signal peptide, i.e., the numbering of the mature HA). In another specific embodiment, the term “120 loop” refers to amino acid residues 75, 77, and 116 to 137 of the HA1 domain of influenza B virus B/Hong Kong/8/73 or amino acid residues in the HA1 domain of an influenza B virus other than B/Hong Kong/8/73 that correspond to amino acid residues 75, 77, and 116 to 137 of the HA1 domain of influenza B virus B/Hong Kong/8/73 (wherein the amino acid residues 75, 77, and 116-137 correspond to the numbered positions of the influenza B virus B/Hong Kong/8/73 HA not including the signal peptide, i.e., the numbering of the mature HA). In another specific embodiment, the term “120 loop” refers to amino acid residues 75, 77, 116, 118, 122, 129, and 137 of the HA1 domain of influenza B virus B/Hong Kong/8/73 or amino acid residues in the HA1 domain of an influenza B virus other than B/Hong Kong/8/73 that correspond to amino acid residues 75, 77, 116, 118, 122, 129, and 137 of the HA1 domain of influenza B virus B/Hong Kong/8/73 (wherein the amino acid residues 75, 77, 116, 118, 122, 129, and 137 correspond to the numbered positions of the influenza B virus B/Hong Kong/8/73 HA not including the signal peptide, i.e., the numbering of the mature HA). In another specific embodiment, the term “120 loop” refers to the antigenic region defined by Wang et al., 2008, Journal of Virology 82:3011-3020 as 120 loop or the equivalent thereof in other influenza B viruses.

As used herein, the term “150 loop” refers to an antigenic region in an influenza B virus HA. In a specific embodiment, the term “150 loop” refers to amino acid residues 141 to 150 of the HA1 domain of influenza B virus B/Hong Kong/8/73 or amino acid residues in the HA1 domain of an influenza B virus other than B/Hong Kong/8/73 that correspond to amino acid residues 141 to 150 of the HA1 domain of influenza B virus B/Hong Kong/8/73 (wherein the amino acid residues 141 to 150 correspond to the numbered positions of the influenza B virus B/Hong Kong/8/73 HA not including the signal peptide, i.e., the numbering of the mature HA). In another specific embodiment, the term “150 loop” refers to amino acid residues 141 and 144 to 150 of the HA1 domain of influenza B virus B/Hong Kong/8/73 or amino acid residues in the HA1 domain of an influenza B virus other than B/Hong Kong/8/73 that correspond to amino acid residues 141 and 144 to 150 of the HA1 domain of influenza B virus B/Hong Kong/8/73 (wherein the amino acid residues 141 and 144 to 150 correspond to the numbered positions of the influenza B virus B/Hong Kong/8/73 HA not including the signal peptide, i.e., the numbering of the mature HA). In another specific embodiment, the term “150 loop” refers to the antigenic region defined by Wang et al., 2008, Journal of Virology 82:3011-3020 as 150 loop or the equivalent thereof in other influenza B viruses.

As used herein, the term “160 loop” refers to an antigenic region in an influenza B virus HA. In a specific embodiment, the term “160 loop” refers to amino acid residues 162 to 167 of the HA1 domain of influenza B virus B/Hong Kong/8/73 or amino acid residues in the HA1 domain of an influenza B virus other than B/Hong Kong/8/73 that correspond to amino acid residues 162 to 167 of the HA1 domain of influenza B virus B/Hong Kong/8/73 (wherein the amino acid residues 162 to 167 correspond to the numbered positions of the influenza B virus B/Hong Kong/8/73 HA not including the signal peptide, i.e., the numbering of the mature HA). In another specific embodiment, the term “160 loop” refers to the antigenic region defined by Wang et al., 2008, Journal of Virology 82:3011-3020 as 160 loop or the equivalent thereof in other influenza B viruses.

As used herein, the term “190 helix” refers to an antigenic region in an influenza B virus HA. In a specific embodiment, the term “190 helix” refers to amino acid residues 194 to 202 of the HA1 domain of influenza B virus B/Hong Kong/8/73 or amino acid residues in the HA1 domain of an influenza B virus other than B/Hong Kong/8/73 that correspond to amino acid residues 194 to 202 of the HA1 domain of influenza B virus B/Hong Kong/8/73 (wherein the amino acid residues 194 to 202 correspond to the numbered positions of the influenza B virus B/Hong Kong/8/73 HA not including the signal peptide, i.e., the numbering of the mature HA). In a specific embodiment, the term “190 helix” refers to amino acid residues 194 to 200 of the HA1 domain of influenza B virus B/Hong Kong/8/73 or amino acid residues in the HA1 domain of an influenza B virus other than B/Hong Kong/8/73 that correspond to amino acid residues 194 to 200 of the HA1 domain of influenza B virus B/Hong Kong/8/73 (wherein the amino acid residues 194 to 200 correspond to the numbered positions of the influenza B virus B/Hong Kong/8/73 HA not including the signal peptide, i.e., the numbering of the mature HA). In a specific embodiment, the term “190 helix” refers to amino acid residues 194 to 200, 205 and 238 of the HA1 domain of influenza B virus B/Hong Kong/8/73 or amino acid residues in the HA1 domain of an influenza B virus other than B/Hong Kong/8/73 that correspond to amino acid residues 194 to 200, 205 and 238 of the HA1 domain of influenza B virus B/Hong Kong/8/73 (wherein the amino acid residues 194 to 200, 205 and 238 correspond to the numbered positions of the influenza B virus B/Hong Kong/8/73 HA not including the signal peptide, i.e., the numbering of the mature HA). In another specific embodiment, the term “190 helix” refers to amino acid residues 194 to 205 and 238 of the HA1 domain of influenza B virus B/Hong Kong/8/73 or amino acid residues in the HA1 domain of an influenza B virus other than B/Hong Kong/8/73 that correspond to amino acid residues 194 to 205 and 238 of the HA1 domain of influenza B virus B/Hong Kong/8/73 (wherein the amino acid residues 194 to 205 and 238 correspond to the numbered positions of the influenza B virus B/Hong Kong/8/73 HA not including the signal peptide, i.e., the numbering of the mature HA). In another specific embodiment, the term “190 helix” refers to the antigenic region defined by Wang et al., 2008, Journal of Virology 82:3011-3020 as 190 helix or the equivalent thereof in other influenza B viruses.

The terms “about” or “approximate,” when used in reference to an amino acid position refer to the particular amino acid position in a sequence or any amino acid that is within five, four, three, two, or one residues of that amino acid position, either in an N-terminal direction or a C-terminal direction. As used herein, the term “about” or “approximately” when used in conjunction with a number refers to any number within 1, 5 or 10% of the referenced number. In certain embodiments, the term “about” encompasses the exact number recited.

The term “amino acid sequence identity” refers to the degree of identity or similarity between a pair of aligned amino acid sequences, usually expressed as a percentage. Percent identity is the percentage of amino acid residues in a candidate sequence that are identical (i.e., the amino acid residues at a given position in the alignment are the same residue) or similar (i.e., the amino acid substitution at a given position in the alignment is a conservative substitution, as discussed below), to the corresponding amino acid residue in the peptide after aligning the sequences and introducing gaps, if necessary, to achieve the maximum percent sequence homology. Sequence homology, including percentages of sequence identity and similarity, may be determined using sequence alignment techniques well-known in the art, preferably computer algorithms designed for this purpose, using the default parameters of said computer algorithms or the software packages containing them. Non-limiting examples of computer algorithms and software packages incorporating such algorithms include the following. The BLAST family of programs exemplify a particular, non-limiting example of a mathematical algorithm utilized for the comparison of two sequences (e.g., Karlin & Altschul, 1990, Proc. Natl. Acad. Sci. USA 87:2264-2268 (modified as in Karlin & Altschul, 1993, Proc. Natl. Acad. Sci. USA 90:5873-5877), Altschul et al., 1990, J. Mol. Biol. 215:403-410, (describing NBLAST and XBLAST), Altschul et al., 1997, Nucleic Acids Res. 25:3389-3402 (describing Gapped BLAST, and PSI-Blast). Another particular example is the algorithm of Myers and Miller (1988 CABIOS 4:11-17) which is incorporated into the ALIGN program (version 2.0) and is available as part of the GCG sequence alignment software package. Also particular is the FASTA program (Pearson W. R. and Lipman D. J., Proc. Nat. Acad. Sci. USA, 85:2444-2448, 1988), available as part of the Wisconsin Sequence Analysis Package. Additional examples include BESTFIT, which uses the “local homology” algorithm of Smith and Waterman (Advances in Applied Mathematics, 2:482-489, 1981) to find best single region of similarity between two sequences, and which is preferable where the two sequences being compared are dissimilar in length; GAP, which aligns two sequences by finding a “maximum similarity” according to the algorithm of Neddleman and Wunsch (J. Mol. Biol. 48:443-354, 1970), and is preferable where the two sequences are approximately the same length and an alignment is expected over the entire length; and BioEdit.

“Conservative substitution” refers to replacement of an amino acid of one class is with another amino acid of the same class. In particular embodiments, a conservative substitution does not alter the structure or function, or both, of a polypeptide. Classes of amino acids for the purposes of conservative substitution include hydrophobic (Met, Ala, Val, Leu, Ile), neutral hydrophilic (Cys, Ser, Thr), acidic (Asp, Glu), basic (Asn, Gln, His, Lys, Arg), conformation disrupters (Gly, Pro) and aromatic (Trp, Tyr, Phe).

As used herein, the terms “disease” and “disorder” are used interchangeably to refer to a condition in a subject. In some embodiments, the condition is a viral infection. In specific embodiments, a term “disease” refers to the pathological state resulting from the presence of the virus in a cell or a subject, or by the invasion of a cell or subject by the virus. In certain embodiments, the condition is a disease in a subject, the severity of which is decreased by inducing an immune response in the subject through the administration of an immunogenic composition.

As described herein, the term “ectodomain” in reference to an influenza virus HA polypeptide would be understood by one of skill in the art. In specific embodiment, the ectodomain does not include the signal peptide, the transmembrane domain, and the cytoplasmic tail domain of an influenza virus HA. See, e.g., Table 1A, Table 1B and Table 1C, below, for an exemplary influenza B virus ectodomain sequence and location. In certain embodiments, the ectodomain of an influenza B virus HA polypeptide is a region of the influenza B virus HA polypeptide that aligns with the ectodomain of influenza B/Hong Kong/8/73 virus HA ectodomain set forth in Table 1A, below. In some embodiments, the ectodomain of an influenza B virus HA polypeptide is a region of the influenza B virus HA polypeptide that aligns with the ectodomain of influenza B/Malaysia/2506/04 virus HA ectodomain set forth in Table 1C, below.

As used herein, the term “effective amount” in the context of administering a therapy to a subject refers to the amount of a therapy which has a prophylactic and/or therapeutic effect(s). In certain embodiments, an “effective amount” in the context of administration of a therapy to a subject refers to the amount of a therapy which is sufficient to achieve one, two, three, four, or more of the following effects: (i) reduce or ameliorate the severity of an influenza virus infection, disease or symptom associated therewith; (ii) reduce the duration of an influenza virus infection, disease or symptom associated therewith; (iii) prevent the progression of an influenza virus infection, disease or symptom associated therewith; (iv) cause regression of an influenza virus infection, disease or symptom associated therewith; (v) prevent the development or onset of an influenza virus infection, disease or symptom associated therewith; (vi) prevent the recurrence of an influenza virus infection, disease or symptom associated therewith; (vii) reduce or prevent the spread of an influenza virus from one cell to another cell, one tissue to another tissue, or one organ to another organ; (viii) prevent or reduce the spread of an influenza virus from one subject to another subject; (ix) reduce organ failure associated with an influenza virus infection; (x) reduce hospitalization of a subject; (xi) reduce hospitalization length; (xii) increase the survival of a subject with an influenza virus infection or disease associated therewith; (xiii) eliminate an influenza virus infection or disease associated therewith; (xiv) inhibit or reduce influenza virus replication; (xv) inhibit or reduce the entry of an influenza virus into a host cell(s); (xvi) inhibit or reduce replication of the influenza virus genome; (xvii) inhibit or reduce synthesis of influenza virus proteins; (xviii) inhibit or reduce assembly of influenza virus particles; (xix) inhibit or reduce release of influenza virus particles from a host cell(s); (xx) reduce influenza virus titer; and/or (xxi) enhance or improve the prophylactic or therapeutic effect(s) of another therapy.

In certain embodiments, the effective amount does not result in complete protection from an influenza virus disease, but results in a lower titer or reduced number of influenza viruses compared to an untreated subject with an influenza virus infection. In certain embodiments, the effective amount results in a 0.5 fold, 1 fold, 1.5 fold, 2 fold, 3 fold, 4 fold, 5 fold, 6 fold, 7 fold, 8 fold, 9 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titer of influenza virus relative to an untreated subject with an influenza virus infection. In some embodiments, the effective amount results in a reduction in titer of influenza virus relative to an untreated subject with an influenza virus infection of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. Benefits of a reduction in the titer, number or total burden of influenza virus include, but are not limited to, less severe symptoms of the infection, fewer symptoms of the infection and a reduction in the length of the disease associated with the infection.

“HA” and “hemagglutinin” refer to any hemagglutinin known to those of skill in the art. In certain embodiments, the hemagglutinin is influenza hemagglutinin, such as an influenza A hemagglutinin, an influenza B hemagglutinin, or an influenza C hemagglutinin. A typical hemagglutinin comprises domains known to those of skill in the art including a signal peptide (optional herein), a stem domain, a globular head domain, a luminal domain (optional herein), a transmembrane domain (optional herein) and a cytoplasmic domain (optional herein). In certain embodiments, a hemagglutinin consists of a single polypeptide chain, such as HA0. In certain embodiments, a hemagglutinin consists of more than one polypeptide chain in quaternary association, e.g. HA1 and HA2. Those of skill in the art will recognize that an immature HA0 might be cleaved to release a signal peptide (approximately 20 amino acids) yielding a mature hemagglutinin HA0. In another aspect, those of skill in the art will recognize that an immature HA0 might be cleaved to release a signal peptide (approximately 15 amino acids) yielding a mature hemagglutinin HA0. A hemagglutinin HA0 might be cleaved at another site to yield HA1 polypeptide (approximately 342 amino acids of influenza B/Hong Kong/8/73 virus, including the globular head domain and a portion of the stem domain) and HA2 polypeptide (approximately 169 amino acids of influenza B/Hong Kong/8/73 virus, including the remainder of the stem domain, a luminal domain, a transmembrane domain and a cytoplasmic domain). In another embodiment, a hemagglutinin HA0 might be cleaved at another site to yield HA1 polypeptide (approximately 344 amino acids of influenza B/Hong Kong/8/73 virus, including the globular head domain and a portion of the stem domain) and HA2 polypeptide (approximately 223 amino acids of influenza B/Hong Kong/8/73 virus, including the remainder of the stem domain, a luminal domain, a transmembrane domain and a cytoplasmic domain). Those of skill in the art will recognize that an influenza B virus has an elongated fusion domain (composed of the central coiled-coil structure from the HA2 domain), the extended regions from HA1 (amino acid residues 1-42), and HA1 (amino acid residues 288-342), a globular membrane-distal domain containing the receptor-binding subdomain, HA1 (amino acid residues 116-274), and a vestigial esterase subdomain, HA1 (amino acid residues 43-115) and HA1 (amino acid residues 275-287) (see Wang et al., 2008, Journal of Virology, 82 (6): 3011-3020). Those of skill in the art will recognize that the delineation of the domains of an influenza B virus HA may be determined from, e.g., crystal structure and/or by using structure prediction software (for example, the website for the Center for Biological Sequence Analysis, Technical University of Denmark DTU, or Pymol) in conjunction with protein alignments. Thus, in one aspect, one skilled in the art will recognize that the delineation of the domains of influenza B/Hong Kong/8/73 virus HA are as set forth in Table 1A, below. In another aspect, one skilled in the art will recognize the delineation of domains of the mouse adapted influenza B/Malaysia/2506/20/03 virus HA. See, e.g., Table 1B, infra, for exemplary domains for the mouse adapted influenza B/Malaysia/2506/20/03 virus HA. In another aspect, one skilled in the art will recognize the delineation of domain of the influenza B/Malaysia/2506/04 virus HA. See, e.g., Table 1C, infra, for exemplary domains of the influenza B/Malaysia/2506/04 virus HA.

TABLE 1A
Exemplary domains for influenza B/Hong Kong/8/73 virus HA.

| | | | aa residues | | |
|---|---|---|---|---|---|
| | | | (positions in | | |
| | | | influenza | aa residues | |
| | | | B/Hong | (positions in | |
| | | | Kong/8/73, | influenza B/ | |
| | | | inclusive | Hong Kong/ | |
| | | | of the | 8/73, not | |
| Domain for | | aa | signal peptide | including | |
| influenza | | resi- | with exception | the signal | |
| B/Hong | nt | dues | of loops; | peptide; | |
| Kong/8/73 | length | (length) | immature HA) | mature HA) | aa residues |
| Signal | 45 | 15 | 1-15 | N/A | MKAIIVLLMVVTSNA |
| peptide | | | | | (SEQ ID NO: 9) |
| yielding a | | | | | |
| mature HA | | | | | |
| HA0 | | | | | |
| HA1 | 1032 | 344 | 16-359 | 1-344 | DRICTGITSSNSPHVVKTATQGEVNVT |
| polypeptide | | | | | GVIPLTTTPTKSHFANLKGTQTRGKLC |
| (does not | | | | | PNCLNCTDLDVALGRPKCMGTIPSAKA |
| include | | | | | SILHEVKPVTSGCFPIMHDRTKIRQLP |
| signal | | | | | NLLRGYENIRLSARNVTNAETAPGGPY |
| peptide) | | | | | IVGTSGSCPNVTNGNGFFATMAWAVPK |
| | | | | | NKTATNPLTVEVPYICTKGEDQITVWG |
| | | | | | FHSDDETQMVKLYGDSKPQKFTSSANG |
| | | | | | VTTHYVSQIGGFPNQAEDEGLPQSGRI |
| | | | | | VVDYMVQKPGKTGTIAYQRGVLLPQKV |
| | | | | | WCASGRSKVIKGSLPLIGEADCLHEKY |
| | | | | | GGLNKSKPYYTGEHAKAIGNCPIWVKT |
| | | | | | PLKLANGTKYRPPAKLLKER |
| | | | | | (SEQ ID NO: 10) |
| HA2 | 669 | 223 | 360-582 | 345-567 | GFFGAIAGFLEGGWEGMIAGWHGYTSH |
| polypeptide | | | | | GAHGVAVAADLKSTQEAINKITKNLNS |
| (not | | | | | LSELEVKNLQRLSGAIVIDELHNEILE |
| including | | | | | LDEKVDDLRADTISSQIELAVLLSNEG |
| stop codon) | | | | | IINSEDEHLLALERKLKKMLGPSAVDI |
| | | | | | GNGCFETKHKCNQTCLDRIAAGTFNAG |
| | | | | | EFSLPTFDSLNITAASLNDDGLDNHTI |
| | | | | | LLYYSTAASSLAVTLMIAIFIVYMVSR |
| | | | | | DNVSCSICL (SEQ ID NO: 11) |
| Ectodomain | 1590 | 530 | 16-545 | 1-530 | DRICTGITSSNSPHVVKTATQGEVNVT |
| (excludes | | | | | GVIPLTTTPTKSHFANLKGTQTRGKLC |
| signal | | | | | PNCLNCTDLDVALGRPKCMGTIPSAKA |
| peptide, | | | | | SILHEVKPVTSGCFPIMHDRTKIRQLP |
| transmembrane | | | | | NLLRGYENIRLSARNVTNAETAPGGPY |
| domain, | | | | | IVGTSGSCPNVTNGNGFFATMAWAVPK |
| cytoplasmic | | | | | NKTATNPLTVEVPYICTKGEDQITVWG |
| tail domain) | | | | | FHSDDETQMVKLYGDSKPQKFTSSANG |
| | | | | | VTTHYVSQIGGFPNQAEDEGLPQSGRI |
| | | | | | VVDYMVQKPGKTGTIAYQRGVLLPQKV |
| | | | | | WCASGRSKVIKGSLPLIGEADCLHEKY |
| | | | | | GGLNKSKPYYTGEHAKAIGNCPIWVKT |
| | | | | | PLKLANGTKYRPPAKLLKERGFFGAIA |
| | | | | | GFLEGGWEGMIAGWHGYTSHGAHGVAV |
| | | | | | AADLKSTQEAINKITKNLNSLSELEVK |
| | | | | | NLQRLSGAMDELHNEILELDEKVDDLR |
| | | | | | ADTISSQIELAVLLSNEGIINSEDEHL |
| | | | | | LALERKLKKMLGPSAVDIGNGCFETKH |
| | | | | | KCNQTCLDRIAAGTFNAGEFSLPTFDS |
| | | | | | LNITAASLNDDGLDNHT(SEQ ID |
| | | | | | NO: 12) |
| Transmembrane | 81 | 27 | 546-572 | 531-557 | ILLYYSTAASSLAVTLMIAIFIVYMVS |
| domain | | | | | (SEQ ID NO: 13) |
| Cytoplasmic | 30 | 10 | 573-582 | 558-567 | RDNVSCSICL (SEQ ID NO: 14) |
| domain | | | | | |
| (not | | | | | |
| including | | | | | |
| stop codon) | | | | | |
| Stem | 126 | 42 | 16-57 | 1-42 | DRICTGITSSNSPHVVKTATQGEVNVT |
| domain | | | | | GVIPLTTTPTKSHFA (SEQ ID NO: |
| (encompasses | | | | | 15) |
| alanines | | | | | |
| at interface; | | | | | |
| does not | | | | | |
| include | | | | | |
| signal | | | | | |
| peptide or | | | | | |
| stop codon) | | | | | |
| Piece 1: | | | | | |
| Stem | 723 | 241 | 305-545 | 290-530 | ADCLHEKYGGLNKSKPYYTGEHAKAIG |
| domain | | | | | NCPIWVKTPLKLANGTKYRPPAKLLKE |
| (encompasses | | | | | RGFFGAIAGFLEGGWEGMIAGWHGYTS |
| alanines | | | | | HGAHGVAVAADLKSTQEAINKITKNLN |
| at interface; | | | | | SLSELEVKNLQRLSGAMDELHNEILEL |
| does not | | | | | DEKVDDLRADTISSQIELAVLLSNEGI |
| include | | | | | INSEDEHLLALERKLKKMLGPSAVDIG |
| signal | | | | | NGCFETKHKCNQTCLDRIAAGTFNAGE |
| peptide or | | | | | FSLPTFDSLNITAASLNDDGLDNHT |
| stop codon) | | | | | (SEQ ID NO: 16) |
| Piece 2: | | | | | |
| Globular | 741 | 247 | 58-304 | 43-289 | NLKGTQTRGKLCPNCLNCTDLDVALGR |
| head | | | | | PKCMGTIPSAKASILHEVKPVTSGCFP |
| domain | | | | | IMHDRTKIRQLPNLLRGYENIRLSARN |
| (not | | | | | VTNAETAPGGPYIVGTSGSCPNVTNGN |
| including | | | | | GFFATMAWAVPKNKTATNPLTVEVPYI |
| alanines) | | | | | CTKGEDQITVWGFHSDDETQMVKLYGD |
| | | | | | SKPQKFTSSANGVTTHYVSQIGGFPNQ |
| | | | | | AEDEGLPQSGRIVVDYMVQKPGKTGTI |
| | | | | | AYQRGVLLPQKVWCASGRSKVIKGSLP |
| | | | | | LIGE(SEQ ID NO: 17) |

Abbreviations:
nt = nucleotide;
aa = amino acid;
N/A = not applicable.

TABLE 1B
Exemplary domains for mouse adapted influenza
B/Malaysia/2506/20/03 HA. The full length
amino acid sequence for mouse adapted influenza
B/Malaysia/2506/20/03 HA may be found in
SEQ ID NO: 73.

| Domain | Amino acid sequence |
|---|---|
| Signal | MKAIIVLLMVVTSNA (SEQ ID NO: 74) |
| Peptide | |
| HA1 Domain | DRICTGITSSNSPHVVKTATQGEVNVTGVIPLTTT |
| | PTKSHFANLKGTETRGKLCPKCLNCTDLDVALGRP |
| | KCTGNIPSARVSILHEVRPVTSGCFPIMHDRTKIR |
| | QLPNLLRGYEHIRLSTHNVINAENAPGGPYKIGTS |
| | GSCPNVINGNGFFATMAWAVPKNDNNKTATNSLTI |
| | EVPYICTEGEDQITVWGFHSDX₁EX₂QMAKLYGDS |
| | KPQKFTSSANGVTTHYVSQIGGFPNQTEDGGLPQS |
| | GRIVVDYMVQKSGKTGTITYQRGILLPQKVWCASG |
| | RSKVIKGSLPLIGEADCLHEKYGGLNKSKPYYTGE |
| | HAKAIGNCPIWVKTPLKLANGTKYRPPAKLLKER |
| | (SEQ ID NO: 75) |
| | X₁ = N or S; X₂ = N, I, T or S |
| HA2 Domain | GFFGAIAGFLEGGWEGMIAGWHGYTSHGAHGVAVA |
| | ADLKSTQEAINKITKNLNSLSELEVKNLQRLSGAM |
| | DELHNEILELDEKVDDLRADTISSQIELAVLLSNE |
| | GIINSEDEHLLALERKLKKMLGPSAVEIGNGCFET |
| | KHKCNQTCLDRIAAGTFDAGEFSLPTFDSLNITAA |
| | SLNDDGLDNHTILLYYSTAASSLAVTLMIAIFVVY |
| | MVSRDNVSCSICL (SEQ ID NO: 76) |
| N-Terminal | DRICTGITSSNSPHVVKTATQGEVNVTGVIPLTTT |
| Segment of | 50 PTKSHFA (SEQ ID NO: 78) |
| Stalk | |
| (encompasses | |
| alanine at | |
| interface | |
| with globular | |
| head domain) | |
| C-Terminal | ADCLHEKYGGLNKSKPYYTGEHAKAIGNCPIWVKT |
| Segment of | PLKLANGTKYRPPAKLLKERGFFGAIAGFLEGGWE |
| Stalk | GMIAGWHGYTSHGAHGVAVAADLKSTQEAINKITK |
| (encompasses | NLNSLSELEVKNLQRLSGAMDELHNEILELDEKVD |
| alanine | DLRADTISSQIELAVLLSNEGIINSEDEHLLALER |
| at interface | KLKKMLGPSAVEIGNGCFETKHKCNQTCLDRIAAG |
| with globular | TFDAGEFSLPTFDSLNITAASLNDDGLDNHT |
| head domain) | (SEQ ID NO: 79) |
| Globular Head | NLKGTETRGKLCPKCLNCTDLDVALGRPKCTGNIP |
| Domain | SARVSILHEVRPVTSGCFPIMHDRTKIRQLPNLLR |
| | GYEHIRLSTHNVINAENAPGGPYKIGTSGSCPNVT |
| | NGNGFFATMAWAVPKNDNNKTATNSLTIEVPYICT |
| | EGEDQITVWGFHSDX₁EX₂QMAKLYGDSKPQKFTS |
| | SANGVTTHYVSQIGGFPNQTEDGGLPQSGRIVVDY |
| | MVQKSGKTGTITYQRGILLPQKVWCASGRSKVIKG |
| | SLPLIGE (SEQ ID NO: 77) |
| | X₁ = N or S; X₂ = N, I, T or S |

TABLE 1C
Exemplary domains for influenza
B/Malaysia/2506/20/03 HA. The full length
amino acid sequence for influenza
B/Malaysia/2506/20/03 HA may be found in
SEQ ID NO: 88.

| Domain | Amino acid sequence |
|---|---|
| Signal | MKAIIVLLMVVTSNA(SEQ ID NO: 89) |
| Peptide | |
| HA1 Domain | DRICTGITSSNSPHVVKTATQGEVNVTGVIPLTTT |
| | PTKSHFANLKGTETRGKLCPKCLNCTDLDVALGRP |
| | KCTGNIPSARVSILHEVRPVTSGCFPIMHDRTKIR |
| | QLPNLLRGYEHIRLSTHNVINAENAPGGPYKIGTS |
| | GSCPNVTNGNGFFATMAWAVPKNDNNKTATNSLTI |
| | EVPYICTEGEDQITVWGFHSDNEX₁QMAKLYGDSK |
| | PQKFTSSANGVTTHYVSQIGGFPNQTEDGGLPQSG |
| | RIVVDYMVQKSGKTGTITYQRGILLPQKVWCASGR |
| | SKVIKGSLPLIGEADCLHEKYGGLNKSKPYYTGEH |
| | AKAIGNCPIWVKTPLKLANGTKYRPPAKLLKER |
| | (SEQ ID NO: 90) X₁ = I or T |
| HA2 Domain | GFFGAIAGFLEGGWEGMIAGWHGYTSHGAHGVAVA |
| | ADLKSTQEAINKITKNLNSLSELEVKNLQRLSGAM |
| | DELHNEILELDEKVDDLRADTISSQIELAVLLSNE |
| | GIINSEDEHLLALERKLKKMLGPSAVEIGNGCFET |
| | KHKCNQTCLDRIAAGTFDAGEFSLPTFDSLNITAA |
| | SLNDDGLDNHTILLYYSTAASSLAVTLMIAIFVVY |
| | MVSRDNVSCSICL (SEQ ID NO: 91) |
| N-Terminal | DRICTGITSSNSPHVVKTATQGEVNVTGVIPLTTT |
| Segment of | PTKSHFA(SEQ ID NO: 92) |
| Stalk | |
| (encompasses | |
| alanine at | |
| interface | |
| with globular | |
| head domain) | |
| C-Terminal | ADCLHEKYGGLNKSKPYYTGEHAKAIGNCPIWVKT |
| Segment of | PLKLANGTKYRPPAKLLKERGFFGAIAGFLEGGWE |
| Stalk | GMIAGWHGYTSHGAHGVAVAADLKSTQEAINKITK |
| (encompasses | NLNSLSELEVKNLQRLSGAMDELHNEILELDEKVD |
| alanine at | DLRADTISSQIELAVLLSNEGIINSEDEHLLALER |
| interface | KLKKMLGPSAVEIGNGCFETKHKCNQTCLDRIAAG |
| with globular | TFDAGEFSLPTFDSLNITAASLNDDGLDNHT |
| head domain) | (SEQ ID NO: 93) |
| Globular Head | NLKGTETRGKLCPKCLNCTDLDVALGRPKCTGNIP |
| Domain | SARVSILHEVRPVTSGCFPIMHDRTKIRQLPNLLR |
| | GYEHIRLSTHNVINAENAPGGPYKIGTSGSCPNVT |
| | NGNGFFATMAWAVPKNDNNKTATNSLTIEVPYICT |
| | EGEDQITVWGFHSDNE_{X1}QMAKLYGDSKPQKFTSS |
| | ANGVTTHYVSQIGGFPNQTEDGGLPQSGRIVVDYM |
| | VQKSGKTGTITYQRGILLPQKVWCASGRSKVIKGS |
| | LPLIGE (SEQ ID NO: 94) X₁ = I or T |
| Ectodomain | DRICTGITSSNSPHVVKTATQGEVNVTGVIPLTTT |
| | PTKSHFANLKGTETRGKLCPKCLNCTDLDVALGRP |
| | KCTGNIPSARVSILHEVRPVTSGCFPIMHDRTKIR |
| | QLPNLLRGYEHIRLSTHNVINAENAPGGPYKIGTS |
| | GSCPNVTNGNGFFATMAWAVPKNDNNKTATNSLTI |
| | EVPYICTEGEDQITVWGFHSDNE_{X1}QMAKLYGDSK |
| | PQKFTSSANGVTTHYVSQIGGFPNQTEDGGLPQSG |
| | RIVVDYMVQKSGKTGTITYQRGILLPQKVWCASGR |
| | SKVIKGSLPLIGEADCLHEKYGGLNKSKPYYTGEH |
| | AKAIGNCPIWVKTPLKLANGTKYRPPAKLLKERGE |
| | FGAIAGFLEGGWEGMIAGWHGYTSHGAHGVAVAAD |
| | LKSTQEAINKITKNLNSLSELEVKNLQRLSGAMDE |
| | LHNEILELDEKVDDLRADTISSQIELAVLLSNEGI |
| | INSEDEHLLALERKLKKMLGPSAVEIGNGCFETKH |
| | KCNQTCLDRIAAGTFDAGEFSLPTFDSLNITAASL |
| | NDDGLDNHT (SEQ ID NO: 95) |
| | X₁ = I or T |

In certain embodiments, a hemagglutinin protein comprises a signal peptide, a transmembrane domain and a cytoplasmic domain. In certain embodiments, a hemagglutinin protein comprises a signal peptide, an ectodomain, a transmembrane domain and a cytoplasmic domain. In certain embodiments, a hemagglutinin lacks a signal peptide, i.e. the hemagglutinin is a mature hemagglutinin. In certain embodiments, a hemagglutinin lacks a transmembrane domain or cytoplasmic domain, or both. As used herein, the terms “hemagglutinin” and “HA” encompass hemagglutinin polypeptides that are modified by post-translational processing such as signal peptide cleavage, disulfide bond formation, glycosylation (e.g., N-linked glycosylation), protease cleavage and lipid modification (e.g. S-palmitoylation).

“HA2” refers to a polypeptide domain that corresponds to the HA2 domain of an influenza hemagglutinin polypeptide known to those of skill in the art. In certain embodiments, an HA2 consists of a stem domain, a luminal domain, a transmembrane domain and a cytoplasmic domain (see, e.g., Scheiffle et al., 2007, EMBO J. 16 (18): 5501-5508, the contents of which are incorporated by reference in their entirety). In certain embodiments, an HA2 consists of a stem domain, a luminal domain and a transmembrane domain. In certain embodiments, an HA2 consists of a stem domain and a luminal domain; in such embodiments, the HA2 might be soluble. In certain embodiments, an HA2 consists of a stem domain; in such embodiments, the HA2 might be soluble. In certain embodiments, an HA2 consists of amino acid residues 1-169 of the HA2 domain of an influenza B/Hong Kong/8/73 virus (see Wang et al., 2008, Journal of Virology 82:3011-3020). In certain embodiments, an HA2 consists of amino acid residues 345-567 of a mature influenza B/Hong Kong/8/73 virus HA (i.e., the numbering is determined from an influenza B/Hong Kong/8/73 virus HA that does not include the signal peptide).

As used herein, the term “heterologous” in the context of a polypeptide, nucleic acid or virus refers to a polypeptide, nucleic acid or virus, respectively, that is not normally found in nature or not normally associated in nature with a polypeptide, nucleic acid or virus of interest. For example, a “heterologous polypeptide” may refer to a polypeptide derived from a different virus, e.g., a different influenza strain or subtype, or an unrelated virus or different species.

As used herein, the term “in combination,” in the context of the administration of two or more therapies to a subject, refers to the use of more than one therapy (e.g., more than one prophylactic agent and/or therapeutic agent). The use of the term “in combination” does not restrict the order in which therapies are administered to a subject. For example, a first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. As another example, a first therapy (e.g., a first prophylactic or therapeutic agent) can be administered prior to (e.g., 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months before), concomitantly with, or subsequent to (e.g., about 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after) the administration of a second therapy to a subject.

As used herein, the term “infection” means the invasion by, multiplication and/or presence of a virus in a cell or a subject. In one embodiment, an infection is an “active” infection, i.e., one in which the virus is replicating in a cell or a subject. Such an infection is characterized by the spread of the virus to other cells, tissues, and/or organs, from the cells, tissues, and/or organs initially infected by the virus. An infection may also be a latent infection, i.e., one in which the virus is not replicating. In certain embodiments, an infection refers to the pathological state resulting from the presence of the virus in a cell or a subject, or by the invasion of a cell or subject by the virus.

As used herein, the term “influenza virus disease” refers to the pathological state resulting from the presence of an influenza (e.g., influenza A or B virus) virus in a cell or subject or the invasion of a cell or subject by an influenza virus. In specific embodiments, the term refers to a respiratory illness caused by an influenza virus.

As used herein, the terms “influenza virus hemagglutinin head domain polypeptide,” “influenza virus hemagglutinin head domain,” “HA globular head domain,” and “HA head domain” refer to the globular head domain of an influenza hemagglutinin polypeptide. An influenza virus hemagglutinin head domain polypeptide or influenza virus hemagglutinin head domain may comprise or consist of a known (e.g., wild-type) influenza virus hemagglutinin head domain or may comprise or consist of a derivative, e.g. an engineered derivative, of a known (e.g., wild-type) influenza virus hemagglutinin head domain. Those of skill in the art will recognize that an influenza B virus HA globular head domain typically comprises the amino acid residues corresponding to amino acid residues 43-289 of the HA1 domain of influenza B/Hong Kong/8/73 virus (wherein the numbering of the amino acid residues is with respect to the mature HA sequence, which does not comprise the 15 amino acid signal peptide). For example, one skilled in the art will recognize that the amino acid sequence for the HA globular head domain for influenza B/Hong Kong/8/73 virus typically consists of the amino acid sequence:

(SEQ ID NO: 17)
NLKGTQTRGKLCPNCLNCTDLDVALGRPKCMGTIPSAKASILHEVKPVTS
GCFPIMHDRTKIRQLPNLLRGYENIRLSARNVTNAETAPGGPYIVGTSGS
CPNVINGNGFFATMAWAVPKNKTATNPLTVEVPYICTKGEDQITVWGFHS
DDETQMVKLYGDSKPQKFTSSANGVTTHYVSQIGGFPNQAEDEGLPQSGR
IVVDYMVQKPGKTGTIAYQRGVLLPQKVWCASGRSKVIKGSLPLIGE.

Those of skill in the art will recognize that the location of the influenza B virus HA globular head domain for a particular strain can be determined by alignment of the influenza B virus HA polypeptide for said strain to the sequence of an influenza A virus HA (FIG. 20). In specific embodiments, the influenza B virus globular head domain consists of the amino acid residues that align to amino acid residues 58-304 of the mature influenza B/Hong Kong/8/73 virus HA (i.e., wherein said numbering includes the signal peptide). See, e.g., Table 1A, above.

As used herein, the phrases “IFN deficient system” or “IFN-deficient substrate” refer to systems, e.g., cells, cell lines and animals, such as pigs, mice, chickens, turkeys, rabbits, rats, etc., which do not produce interferon (IFN) or produce low levels of IFN (i.e., a reduction in IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to IFN, and/or are deficient in the activity of one or more antiviral genes induced by IFN.

As used herein, the numeric term “log” refers to log 10.

As used herein, the phrase “multiplicity of infection” or “MOI” is the average number of infectious virus particles per infected cell. The MOI is determined by dividing the number of infectious virus particles added (ml added x PFU/ml) by the number of cells added (ml added x cells/ml).

As used herein, the term “nucleic acid” is intended to include DNA molecules (e.g., cDNA or genomic DNA) and RNA molecules (e.g., mRNA) and analogs of the DNA or RNA generated using nucleotide analogs. The nucleic acid can be single-stranded or double-stranded.

“Polypeptide” refers to a polymer of amino acids linked by amide bonds as is known to those of skill in the art. As used herein, the term can refer to a single polypeptide chain linked by covalent amide bonds. The term can also refer to multiple polypeptide chains associated by non-covalent interactions such as ionic contacts, hydrogen bonds, Van der Waals contacts and hydrophobic contacts. Those of skill in the art will recognize that the term includes polypeptides that have been modified, for example by post-translational processing such as signal peptide cleavage, disulfide bond formation, glycosylation (e.g., N-linked glycosylation), protease cleavage and lipid modification (e.g. S-palmitoylation).

As used herein, the terms “prevent,” “preventing” and “prevention” in the context of the administration of a therapy (ies) to a subject to prevent an influenza virus disease refer to one or more of the prophylactic/beneficial effects resulting from the administration of a therapy or a combination of therapies. In a specific embodiment, the terms “prevent,” “preventing” and “prevention” in the context of the administration of a therapy (ies) to a subject to prevent an influenza virus disease refer to one or more of the following effects resulting from the administration of a therapy or a combination of therapies: (i) the inhibition of the development or onset of an influenza virus disease or a symptom thereof; (ii) the inhibition of the recurrence of an influenza virus disease or a symptom associated therewith; and (iii) the reduction or inhibition in influenza virus infection and/or replication.

As used herein, the terms “purified” and “isolated” when used in the context of a polypeptide (including an antibody) that is obtained from a natural source, e.g., cells, refers to a polypeptide which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a polypeptide (including an antibody) that is chemically synthesized refers to a polypeptide which is substantially free of chemical precursors or other chemicals which are involved in the syntheses of the polypeptide. In a specific embodiment, a chimeric HA polypeptide is chemically synthesized. In another specific embodiment, an influenza hemagglutinin stem domain polypeptide, an influenza hemagglutinin head domain polypeptide, non-chimeric HA polypeptide, and/or a chimeric influenza hemagglutinin polypeptide is isolated.

As used herein, the terms “replication,” “viral replication” and “virus replication” in the context of a virus refer to one or more, or all, of the stages of a viral life cycle which result in the propagation of virus. The steps of a viral life cycle include, but are not limited to, virus attachment to the host cell surface, penetration or entry of the host cell (e.g., through receptor mediated endocytosis or membrane fusion), uncoating (the process whereby the viral capsid is removed and degraded by viral enzymes or host enzymes thus releasing the viral genomic nucleic acid), genome replication, synthesis of viral messenger RNA (mRNA), viral protein synthesis, and assembly of viral ribonucleoprotein complexes for genome replication, assembly of virus particles, post-translational modification of the viral proteins, and release from the host cell by lysis or budding and acquisition of a phospholipid envelope which contains embedded viral glycoproteins. In some embodiments, the terms “replication,” “viral replication” and “virus replication” refer to the replication of the viral genome. In other embodiments, the terms “replication,” “viral replication” and “virus replication” refer to the synthesis of viral proteins.

As used herein, the terms “stem domain polypeptide” and “influenza virus hemagglutinin stem domain polypeptide” and “stalk domain” refer to the stem domain of an influenza virus hemagglutinin polypeptide (which includes derivatives of an influenza HA polypeptide). A stem domain polypeptide might be a single polypeptide chain, two polypeptide chains or more polypeptide chains. Typically, a stem domain polypeptide is a single polypeptide chain (i.e. corresponding to the stem domain of a hemagglutinin HA0 polypeptide) or two polypeptide chains (i.e. corresponding to the stem domain of a hemagglutinin HA1 polypeptide in association with a hemagglutinin HA2 polypeptide). In a specific embodiment, the stem domain of an influenza B virus HA polypeptide is a trimer. See, e.g., Tables 1A and 1B, supra, for an example of the amino acid sequence and location of a stem domain of an influenza B virus.

As used herein, terms “subject” or “patient” are used interchangeably to refer to an animal (e.g., birds, reptiles, and mammals). In a specific embodiment, a subject is a bird. In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human. In another embodiment, a subject is a human infant. In another embodiment, a subject is a human child. In another embodiment, a subject is a human adult. In another embodiment, a subject is an elderly human. In another embodiment, a subject is a premature human infant.

As used herein, the term “seasonal influenza virus strain” refers to a strain of influenza virus to which a subject population is exposed to on a seasonal basis. In specific embodiments, the term seasonal influenza virus strain refers to a strain of influenza B virus. In specific embodiments, the term seasonal influenza virus strain refers to a strain of influenza virus that belongs to the Yamagata or the Victoria lineages, i.e., the two lineages that presently persist in the human subject population.

As used herein, the terms “therapies” and “therapy” can refer to any protocol(s), method(s), compound(s), composition(s), formulation(s), and/or agent(s) that can be used in the prevention or treatment of a viral infection or a disease or symptom associated therewith. In certain embodiments, the terms “therapies” and “therapy” refer to biological therapy, supportive therapy, and/or other therapies useful in treatment or prevention of a viral infection or a disease or symptom associated therewith known to one of skill in the art. In some embodiments, the term “therapy” refers to (i) a nucleic acid encoding a chimeric HA polypeptide, (ii) a chimeric HA polypeptide), or (iii) a vector or composition comprising a nucleic acid encoding a chimeric HA polypeptide or comprising a chimeric HA polypeptide. In some embodiments, the term “therapy” refers to an antibody that specifically binds to a chimeric influenza virus hemagglutinin polypeptide.

As used herein, the terms “treat,” “treatment,” and “treating” refer in the context of administration of a therapy (ies) to a subject to treat an influenza virus disease or infection to obtain a beneficial or therapeutic effect of a therapy or a combination of therapies. In specific embodiments, such terms refer to one, two, three, four, five or more of the following effects resulting from the administration of a therapy or a combination of therapies: (i) the reduction or amelioration of the severity of an influenza virus infection or a disease or a symptom associated therewith; (ii) the reduction in the duration of an influenza virus infection or a disease or a symptom associated therewith; (iii) the regression of an influenza virus infection or a disease or a symptom associated therewith; (iv) the reduction of the titer of an influenza virus; (v) the reduction in organ failure associated with an influenza virus infection or a disease associated therewith; (vi) the reduction in hospitalization of a subject; (vii) the reduction in hospitalization length; (viii) the increase in the survival of a subject; (ix) the elimination of an influenza virus infection or a disease or symptom associated therewith; (x) the inhibition of the progression of an influenza virus infection or a disease or a symptom associated therewith; (xi) the prevention of the spread of an influenza virus from a cell, tissue, organ or subject to another cell, tissue, organ or subject; (xii) the inhibition or reduction in the entry of an influenza virus into a host cell(s); (xiii) the inhibition or reduction in the replication of an influenza virus genome; (xiv) the inhibition or reduction in the synthesis of influenza virus proteins; (xv) the inhibition or reduction in the release of influenza virus particles from a host cell(s); and/or (xvi) the enhancement or improvement the therapeutic effect of another therapy.

As used herein, in some embodiments, the phrase “wild-type” in the context of a viral polypeptide refers to a viral polypeptide that is found in nature and is associated with a naturally occurring virus.

As used herein, in some embodiments, the phrase “wild-type” in the context of a virus refers to the types of a virus that are prevalent, circulating naturally and producing typical outbreaks of disease. In other embodiments, the term “wild-type” in the context of a virus refers to a parental virus.

### 4. BRIEF DESCRIPTION OF THE DRAWINGS

FIG. 1 depicts a strategy for generating influenza B virus chimeric HAs for use as universal influenza virus vaccine candidates and potential vaccination schematic.

FIG. 2 depicts the 120 loop, 150 loop, 160 loop, and 190 helix in the influenza B/Yamanashi/166/1998 virus globular head as was defined by Wang et al., 2008, Journal of Virology 82:3011-3020.

FIG. 3A depicts the amino acid sequence corresponding to the 150 loop of the influenza B/Yamagata/16/88 virus sequence (top, SEQ ID NO: 18) and the amino acid sequence of the modified 150 loop of the chimeric HA (bottom, SEQ ID NO: 19). FIG. 3B depicts the structure of the influenza B/Yamanashi/166/1998 virus globular head domain and points out the location of the 150 loop. FIG. 3C depicts an immunostain of a plaque assay used to visualize plaque morphology of the rescued virus expressing the chimeric HA.

FIG. 4 depicts growth curves of viruses expressing the indicated chimeric HA constructs at the indicated timepoints. The growth curves were performed in MDCK cells growth at 33 degrees Celsius.

FIG. 5 depicts immunofluorescent staining of 293T cells transfected with the indicated constructs and stained with anti-H5 serum.

FIG. 6A depicts the amino acid sequence corresponding to the 190 helix of the influenza B/Yamagata/16/88 virus sequence (top, SEQ ID NO: 7) and the amino acid sequence of the modified 190 helix of the chimeric HA (bottom, SEQ ID NO: 8). FIG. 6B depicts the structure of the influenza B/Yamanashi/166/1998 virus globular head domain and points out the location of the 190 helix. FIG. 6C depicts an immunostain of a plaque assay used to visualize plaque morphology of the rescued virus expressing the chimeric HA.

FIG. 7A depicts the amino acid sequence corresponding to the 160 loop of the influenza B/Yamagata/16/88 virus sequence (top, SEQ ID NO: 5) and the amino acid sequence of the modified 160 loop of the chimeric HA (bottom, SEQ ID NO: 6). FIG. 7B depicts the structure of the influenza B/Yamanashi/166/1998 virus globular head domain and points out the location of the 160 loop. FIG. 7C depicts an immunostain of a plaque assay used to visualize plaque morphology of the rescued virus expressing the chimeric HA.

FIG. 8A depicts the amino acid sequence corresponding to the 120 loop of the influenza B/Yamagata/16/88 virus sequence (top “B/Yam”, SEQ ID NO: 1) and the amino acid sequence of the modified 120 loop of the chimeric HA (bottom “B/Yamagata ectodomain with A/H5”, SEQ ID NO: 2). Also depicted is the mutation made at amino acid position T90 of influenza B/Yamagata/16/88 virus (wherein amino acid position T90 is with respect to the immature influenza B/Yamagata/16/88 virus HA, i.e., including the signal peptide). The amino acid numbering is with respect to the immature HA (i.e., inclusive of the signal peptide). FIG. 8B depicts the structure of the influenza B/Yamanashi/166/1998 virus globular head domain and points out the location of the 120 loop.

FIG. 9 depicts immunofluorescence of 293T with the indicated antibodies. Column 1 represents cells transfected with pDZ. Column 2 represents cells transfected with Yamagata ectodomain (influenza A/Puerto Rico/8/34 virus (“PR8”) background) HA. Column 3 represents cells transfected with the chimeric HA comprising 150 loop, 160 loop, and 190 helix modified with influenza A virus H5 sequences. Column 4 represents cells transfected with the chimeric HA comprising 120 loop, 150 loop, 160 loop, and 190 helix modified with influenza A virus H5 sequences.

FIG. 10A depicts HI assay titers performed with mouse serum. FIG. 10B depicts HI assay titers performed with ferret serum. Yamecto/PR8 refers to a PR8 virus expressing an HA that has an influenza B/Yamagata/16/88 virus HA ectodomain. 3 loop refers to a PR8 virus expressing an HA that has an influenza B/Yamagata/16/88 virus HA ectodomain, in which three antigenic loops (150 loop, 160 loop, and 190 helix) of the ectodomain have been modified based on the amino acid sequence of influenza A/Vietnam/1203/04 (HALo) virus HA. 4 loop refers to a PR8 virus expressing an HA that has an influenza B/Yamagata/16/88 virus HA ectodomain, in which four antigenic loops (120 loop, 150 loop, 160 loop, and 190 helix) of the ectodomain have been modified based on the amino acid sequence of influenza A/Vietnam/1203/04 (HALo) virus HA.

FIG. 11 demonstrates that chimeric HA may be generated in which each of the 120 loop, 150 loop, 160 loop, and 190 helix of an influenza B virus HA have been modified based on the amino acid sequence of an influenza A virus of the H5, H8, or H12 subtype.

FIG. 12A and FIG. 12B: Nucleic acid sequence encoding H5-4 loop chimeric HA (SEQ ID NO: 20). The background of the construct is Yamagata ectodomain in PR8 HA background in pDZ plasmid made by infusion cloning. Non-underlined nucleic acid sequences refer to the sequences encoding the ectodomain of Yamagata. Underlined nucleic acid sequences labeled “1” refer to the PR8 3′ non-coding region sequence. Underlined nucleic acid sequences labeled “2” refer to the sequence encoding the PR8 signal peptide. Underlined nucleic acid sequences labeled “3” refer to the mutations introduced into the sequence encoding the 120 loop. Underlined nucleic acid sequences labeled “4” refer to the mutations introduced into the sequence encoding the 150 loop. Underlined nucleic acid sequences labeled “5” refer to mutations introduced into the sequence encoding the 160 loop. Underlined nucleic acid sequences labeled “6” refer to the mutations introduced into the sequence encoding the 190 helix. Underlined nucleic acid sequences labeled “7” refer to a H→Y mutation in the Yamagata ectodomain. Underlined nucleic acid sequences labeled “8” refer to the sequence encoding the PR8 transmembrane domain. Underlined nucleic acid sequences labeled “9” refer to the sequence encoding the PR8 cytoplasmic tail domain. Underlined nucleic acid sequences labeled “10” refer to the PR8 5′ non-coding region.

FIG. 13: Amino acid sequence (SEQ ID NO: 21) of the nucleic acid of FIG. 12A and FIG. 12B. Underlined amino acid sequences labeled “1” refer to the PR8 signal peptide sequence. Underlined amino acid sequences labeled “2” refer to the mutations introduced into the 120 loop sequence. Underlined amino acid sequences labeled “3” refer to the mutations introduced into the 150 loop sequence. Underlined amino acid sequences labeled “4” refer to the mutations introduced into the 160 loop sequence. Underlined amino acid sequences labeled “5” refer to the mutations introduced into the 190 helix sequence. Underlined amino acid labeled “6” refers to a H→Y mutation in the Yamagata ectodomain, which matches influenza A virus HA at this position (determined by Flandorfer et al., Journal of Virology, 2003), which may lead to more efficient rescue. Underlined amino acid sequences labeled “7” refer to the PR8 transmembrane domain sequence. Underlined amino acid sequences labeled “8” refer to the PR8 cytoplasmic tail domain sequence.

FIG. 14A and FIG. 14B: Nucleic acid sequence encoding H8-4 loop chimeric HA (SEQ ID NO: 22). The background of the construct is Yamagata ectodomain in PR8 HA background in pDZ plasmid made by infusion cloning. Non-underlined nucleic acid sequences refer to the sequences encoding the ectodomain of Yamagata. Underlined nucleic acid sequences labeled “1” refer to the PR8 3′ non-coding region sequence. Underlined nucleic acid sequences labeled “2” refer to the sequence encoding the PR8 signal peptide. Underlined nucleic acid sequences labeled “3” refer to the mutations introduced into the sequence encoding the 120 loop. Underlined nucleic acid sequences labeled “4” refer to an E→K mutation. Underlined nucleic acid sequences labeled “5” refer to the mutations introduced into the sequence encoding the 150 loop. Underlined nucleic acid sequences labeled “6” refer to mutations introduced into the sequence encoding the 160 loop. Underlined nucleic acid sequences labeled “7” refer to the mutations introduced into the sequence encoding the 190 helix. Underlined nucleic acid sequences labeled “8” refer to a H→Y mutation in the Yamagata ectodomain. Underlined nucleic acid sequences labeled “9” refer to the sequence encoding the PR8 transmembrane domain. Underlined nucleic acid sequences labeled “10” refer to the sequence encoding the PR8 cytoplasmic tail domain. Underlined nucleic acid sequences labeled “11” refer to the PR8 5′ non-coding region.

FIG. 15: Amino acid sequence (SEQ ID NO: 23) of the nucleic acid of FIG. 14A and FIG. 14B. Underlined amino acid sequences labeled “1” refer to the PR8 signal peptide sequence. Underlined amino acid sequences labeled “2” refer to the mutations introduced into the 120 loop sequence. Underlined amino acid labeled “3” refers to an E→K mutation. Underlined amino acid sequences labeled “4” refer to the mutations introduced into the 150 loop sequence. Underlined amino acid sequences labeled “5” refer to the mutations introduced into the 160 loop sequence. Underlined amino acid sequences labeled “6” refer to the mutations introduced into the 190 helix sequence. Underlined amino acid labeled “7” refers to a H→Y mutation in the Yamagata ectodomain, which matches influenza A virus HA at this position (determined by Flandorfer et al., Journal of Virology, 2003), which may lead to more efficient rescue. Underlined amino acid sequences labeled “8” refer to the PR8 transmembrane domain sequence. Underlined amino acid sequences labeled “9” refer to the PR8 cytoplasmic tail domain sequence.

FIG. 16A and FIG. 16B: Nucleic acid sequence encoding H11-4 loop chimeric HA (SEQ ID NO: 24). The background of the construct is Yamagata ectodomain in PR8 HA background in pDZ plasmid made by infusion cloning. Non-underlined nucleic acid sequences refer to the sequences encoding the ectodomain of Yamagata. Underlined nucleic acid sequences labeled “1” refer to the PR8 3′ non-coding region sequence. Underlined nucleic acid sequences labeled “2” refer to the sequence encoding the PR8 signal peptide. Underlined nucleic acid sequences labeled “3” refer to the mutations introduced into the sequence encoding the 120 loop. Underlined nucleic acid sequences labeled “4” refer to the E→K mutation. Underlined nucleic acid sequences labeled “5” refer to the mutations introduced into the sequence encoding the 150 loop. Underlined nucleic acid sequences labeled “6” refer to mutations introduced into the sequence encoding the 160 loop. Underlined nucleic acid sequences labeled “7” refer to the mutations introduced into the sequence encoding the 190 helix. Underlined nucleic acid sequences labeled “8” refer to a H→Y mutation in the Yamagata ectodomain. Underlined nucleic acid sequences labeled “9” refer to the sequence encoding the PR8 transmembrane domain. Underlined nucleic acid sequences labeled “10” refer to the sequence encoding the PR8 cytoplasmic tail domain. Underlined nucleic acid sequences labeled “11” refer to the PR8 5′ non-coding region.

FIG. 17: Amino acid sequence (SEQ ID NO: 25) of the nucleic acid of FIG. 16A and FIG. 16B. Underlined amino acid sequences labeled “1” refer to the PR8 signal peptide sequence. Underlined amino acid sequences labeled “2” refer to the mutations introduced into the 120 loop sequence. Underlined amino acid labeled “3” refers to an E→K mutation. Underlined amino acid sequences labeled “4” refer to the mutations introduced into the 150 loop sequence. Underlined amino acid sequences labeled “5” refer to the mutations introduced into the 160 loop sequence. Underlined amino acid sequences labeled “6” refer to the mutations introduced into the 190 helix sequence. Underlined amino acid labeled “7” refers to a H→Y mutation in the Yamagata ectodomain, which matches influenza A virus HA at this position (determined by Flandorfer et al., Journal of Virology, 2003), which may lead to more efficient rescue. Underlined amino acid sequences labeled “8” refer to the PR8 transmembrane domain sequence. Underlined amino acid sequences labeled “9” refer to the PR8 cytoplasmic tail domain sequence.

FIG. 18A and FIG. 18B: Nucleic acid sequence encoding H12-4 loop chimeric HA (SEQ ID NO: 26). The background of the construct is Yamagata ectodomain in PR8 HA background in pDZ plasmid made by infusion cloning. Non-underlined nucleic acid sequences refer to the sequences encoding the ectodomain of Yamagata. Underlined nucleic acid sequences labeled “1” refer to the PR8 3′ non-coding region sequence. Underlined nucleic acid sequences labeled “2” refer to the sequence encoding the PR8 signal peptide. Underlined nucleic acid sequences labeled “3” refer to the mutations introduced into the sequence encoding the 120 loop. Underlined nucleic acid sequences labeled “4” refer to an E→K mutation. Underlined nucleic acid sequences labeled “5” refer to the mutations introduced into the sequence encoding the 150 loop. Underlined nucleic acid sequences labeled “6” refer to mutations introduced into the sequence encoding the 160 loop. Underlined nucleic acid sequences labeled “7” refer to the mutations introduced into the sequence encoding the 190 helix. Underlined nucleic acid sequences labeled “8” refer to a H→Y mutation in the Yamagata ectodomain. Underlined nucleic acid sequences labeled “9” refer to the sequence encoding the PR8 transmembrane domain. Underlined nucleic acid sequences labeled “10” refer to the sequence encoding the PR8 cytoplasmic tail domain. Underlined nucleic acid sequences labeled “11” refer to the PR8 5′ non-coding region.

FIG. 19: Amino acid sequence (SEQ ID NO: 27) of the nucleic acid of FIG. 18A and FIG. 18B. Underlined amino acid sequences labeled “1” refer to the PR8 signal peptide sequence. Underlined amino acid sequences labeled “2” refer to the mutations introduced into the 120 loop sequence. Underlined amino acid labeled “3” refers to an E→K mutation. Underlined amino acid sequences labeled “4” refer to the mutations introduced into the 150 loop sequence. Underlined amino acid sequences labeled “5” refer to the mutations introduced into the 160 loop sequence. Underlined amino acid sequences labeled “6” refer to the mutations introduced into the 190 helix sequence. Underlined amino acid labeled “7” refers to a H→Y mutation in the Yamagata ectodomain, which matches influenza A virus HA at this position (determined by Flandorfer et al., Journal of Virology, 2003), which may lead to more efficient rescue. Underlined amino acid sequences labeled “8” refer to the PR8 transmembrane domain sequence. Underlined amino acid sequences labeled “9” refer to the PR8 cytoplasmic tail domain sequence.

FIG. 20: Alignment of influenza B/Hong Kong/8/73 virus HA (SEQ ID NO: 28), influenza A/Puerto Rico/8/34 virus HA (SEQ ID NO: 29), and influenza B/Yamagata/16/88 virus HA (SEQ ID NO: 30). The locations of the signal peptide, stalk domain, head domain, start of the HA2 domain, fusion peptide, transmembrane domain, and cytoplasmic tail domain for the influenza B viruses are delineated based on the locations of the respective domains in the influenza A virus.

FIG. 21A: Nucleic acid sequence encoding influenza A/mallard/Sweden/24/2002 virus HA (SEQ ID NO 31). Underlined sequences are the 5′ and 3′ noncoding regions. FIG. 21B: Amino acid sequence (SEQ ID NO: 32) of the nucleic acid of FIG. 21A.

FIG. 22A: Nucleic acid sequence encoding influenza A/Vietnam/1203/04 (HALo) virus HA (SEQ ID NO: 33). FIG. 22B: Amino acid sequence (SEQ ID NO: 34) of the nucleic acid of FIG. 22A.

FIG. 23A: Nucleic acid sequence encoding influenza A/northern shoveler/Netherlands/18/99 virus HA (SEQ ID NO: 35). Underlined sequences are the 5′ and 3′ non-coding regions. FIG. 23B: Amino acid sequence (SEQ ID NO: 36) of the nucleic acid of FIG. 23A.

FIG. 24A: Nucleic acid sequence encoding A_mallard_interior Alaska 7MP0167_2007 virus HA (SEQ ID NO: 37). Underlined sequences are the 5′ and 3′ non-coding regions. FIG. 24B: Amino acid sequence (SEQ ID NO: 38) of the nucleic acid of FIG. 24A.

FIG. 25A: Nucleic acid sequence encoding influenza A/Puerto Rico/8/34 virus HA (SEQ ID NO: 39). Underlined sequences are the 5′ and 3′ non-coding regions. FIG. 25B: Amino acid sequence (SEQ ID NO: 40) of the nucleic acid of FIG. 25A.

FIG. 26A: Nucleic acid sequence encoding influenza B/Yamagata/16/88 virus HA (SEQ ID NO: 41). Underlined sequences are the 5′ and 3′ non-coding regions. FIG. 26B: Amino acid sequence (SEQ ID NO: 42) of the nucleic acid of FIG. 26A.

FIG. 27 depicts the four major antigenic sites of influenza B virus HA in the influenza virus B/Yamanashi/166/1988 (PDB: 4M40): 120 loop, 150 loop, 160 loop, and 190 helix.

FIG. 28: Amino acid residues in four antigenic sites (120 loop, 150 loop, 160 loop, and 190 helix) of the influenza B/Yamagata/16/88 virus HA were replaced by corresponding amino acid sequences from influenza A virus HAs of the H5, H8, H11 or H13 subtypes. The resulting constructs are referred to herein as mH5/B, mH8/B, mH11/B, and mH13/B, respectively, chimeric HAs. Viruses encoding the chimeric HAs were rescued in an influenza B/Malaysia/2506/04 MA virus backbone.

FIG. 29: Nucleic acid sequence encoding mH5/B chimeric HA based on an influenza B/Yamagata/16/88 virus HA sequence, comprising nucleic acid sequences from the influenza A/Vietnam/1203/04 (HALo) virus (H5) globular head domain (SEQ ID NO: 43). Bold, underlined, uppercase sequences correspond to mutations introduced into the 120 loop of the influenza B/Yamagata/16/88 virus HA ectodomain. Bold lowercase sequences correspond to mutations introduced into the 150 loop of the influenza B/Yamagata/16/88 virus HA ectodomain. Italicized, underlined lowercase sequences correspond to mutations introduced into the 160 loop of the influenza B/Yamagata/16/88 virus HA ectodomain. Underlined, bold, italicized, uppercase sequences correspond to mutations introduced into the 190 helix of the influenza B/Yamagata/16/88 virus HA ectodomain. Uppercase bold sequences correspond to additional mutations introduced into the influenza B/Yamagata/16/88 virus HA ectodomain.

FIG. 30: Amino acid sequence (SEQ ID NO: 44) encoded by the nucleic acid of FIG. 29. Bold, underlined, uppercase sequences correspond to mutations introduced into the 120 loop of the influenza B/Yamagata/16/88 virus HA ectodomain. Bold lowercase sequences correspond to mutations introduced into the 150 loop of the influenza B/Yamagata/16/88 virus HA ectodomain. Italicized, underlined lowercase sequences correspond to mutations introduced into the 160 loop of the influenza B/Yamagata/16/88 virus HA ectodomain. Underlined, bold, italicized, uppercase sequences correspond to mutations introduced into the 190 helix of the influenza B/Yamagata/16/88 virus HA ectodomain. Uppercase bold sequence corresponds to an additional mutation introduced into the influenza B/Yamagata/16/88 virus HA ectodomain.

FIG. 31: Nucleic acid sequence encoding mH8/B chimeric HA based on an influenza B/Yamagata/16/88 virus HA sequence, comprising nucleic acid sequences from the influenza A/Mallard/Sweden/24/2002 virus (H8) globular head domain (SEQ ID NO: 45). Bold, underlined, uppercase sequences correspond to mutations introduced into the 120 loop of the influenza B/Yamagata/16/88 virus HA ectodomain. Bold lowercase sequences correspond to mutations introduced into the 150 loop of the influenza B/Yamagata/16/88 virus HA ectodomain. Italicized, underlined lowercase sequences correspond to mutations introduced into the 160 loop of the influenza B/Yamagata/16/88 virus HA ectodomain. Underlined, bold, italicized, uppercase sequences correspond to mutations introduced into the 190 helix of the influenza B/Yamagata/16/88 virus HA ectodomain. Uppercase bold sequences correspond to additional mutations introduced into the influenza B/Yamagata/16/88 virus HA ectodomain.

FIG. 32: Amino acid sequence (SEQ ID NO: 46) encoded by the nucleic acid of FIG. 31. Bold, underlined, uppercase sequences correspond to mutations introduced into the 120 loop of the influenza B/Yamagata/16/88 virus HA ectodomain. Bold lowercase sequences correspond to mutations introduced into the 150 loop of the influenza B/Yamagata/16/88 virus HA ectodomain. Italicized, underlined lowercase sequences correspond to mutations introduced into the 160 loop of the influenza B/Yamagata/16/88 virus HA ectodomain. Underlined, bold, italicized, uppercase sequences correspond to mutations introduced into the 190 helix of the influenza B/Yamagata/16/88 virus HA ectodomain. Uppercase bold sequence corresponds to an additional mutation introduced into the influenza B/Yamagata/16/88 virus HA ectodomain.

FIG. 33: Nucleic acid sequence encoding mH11/B chimeric HA based on an influenza B/Yamagata/16/88 virus HA sequence, comprising nucleic acid sequences from the influenza A/northern shoveler/Netherlands/18/99 virus (H11) globular head domain (SEQ ID NO: 47). Bold, underlined, uppercase sequences correspond to mutations introduced into the 120 loop of the influenza B/Yamagata/16/88 virus HA ectodomain. Bold lowercase sequences correspond to mutations introduced into the 150 loop of the influenza B/Yamagata/16/88 virus HA ectodomain. Italicized, underlined lowercase sequences correspond to mutations introduced into the 160 loop of the influenza B/Yamagata/16/88 virus HA ectodomain. Underlined, bold, italicized, uppercase sequences correspond to mutations introduced into the 190 helix of the influenza B/Yamagata/16/88 virus HA ectodomain. Uppercase bold sequence corresponds to an additional mutation introduced into the influenza B/Yamagata/16/88 virus HA ectodomain.

FIG. 34: Amino acid sequence (SEQ ID NO: 48) encoded by the nucleic acid of FIG. 33. Bold, underlined, uppercase sequences correspond to mutations introduced into the 120 loop of the influenza B/Yamagata/16/88 virus HA ectodomain. Bold lowercase sequences correspond to mutations introduced into the 150 loop of the influenza B/Yamagata/16/88 virus HA ectodomain. Italicized, underlined lowercase sequences correspond to mutations introduced into the 160 loop of the influenza B/Yamagata/16/88 virus HA ectodomain. Underlined, bold, italicized, uppercase sequences correspond to mutations introduced into the 190 helix of the influenza B/Yamagata/16/88 virus HA ectodomain. Uppercase bold sequence corresponds to an additional mutation introduced into the influenza B/Yamagata/16/88 virus HA ectodomain.

FIG. 35: Nucleic acid sequence encoding mH13/B chimeric HA based on an influenza B/Yamagata/16/88 virus HA sequence, comprising nucleic acid sequences from the influenza A/black headed gull/Sweden/1/99 (H13) globular head domain (SEQ ID NO: 49). Bold, underlined, uppercase sequences correspond to mutations introduced into the 120 loop of the influenza B/Yamagata/16/88 virus HA ectodomain. Bold lowercase sequences correspond to mutations introduced into the 150 loop of the influenza B/Yamagata/16/88 virus HA ectodomain. Italicized, underlined lowercase sequences correspond to mutations introduced into the 160 loop of the influenza B/Yamagata/16/88 virus HA ectodomain. Underlined, bold, italicized, uppercase sequences correspond to mutations introduced into the 190 helix of the influenza B/Yamagata/16/88 virus HA ectodomain.

FIG. 36: Amino acid sequence (SEQ ID NO: 50) encoded by the nucleic acid of FIG. 35. Bold, underlined, uppercase sequences correspond to mutations introduced into the 120 loop of the influenza B/Yamagata/16/88 virus HA ectodomain. Bold lowercase sequences correspond to mutations introduced into the 150 loop of the influenza B/Yamagata/16/88 virus HA ectodomain. Italicized, underlined lowercase sequences correspond to mutations introduced into the 160 loop of the influenza B/Yamagata/16/88 virus HA ectodomain. Underlined, bold, italicized, uppercase sequences correspond to mutations introduced into the 190 helix of the influenza B/Yamagata/16/88 virus HA ectodomain.

FIGS. 37A-37D: depicts growth curves for influenza viruses expressing mH5/B chimeric HA (FIG. 37A), mH8/B chimeric HA (FIG. 37B), mH11/B chimeric HA (FIG. 37C), or mH13/B chimeric HA (FIG. 37D) as compared to wild type influenza B/Malaysia/2506/04 MA virus. 10-day embryonated eggs were infected with 500 PFU/egg of the influenza virus expressing mH5/B chimeric HA (mH5/B Mal), mH8/B chimeric HA (mH8/B Mal), mH11/B chimeric HA (mH11/B Mal), or mH13/B chimeric HA (mH13/B Mal), or wild type influenza B/Malaysia/2506/04 MA virus (B/Mal04 MA) in triplicates and incubated at 33 degrees Celsius. Allantoic fluids were harvested at the indicated times and plaque assays were performed on Madin Darby Canine Kidney (MDCK) cells to determine virus titers. PFU refers to plaque forming unit. B/Mal04 MA refers to wild type influenza B/Malaysia/2506/04 MA virus. mH5/B Mal refers to influenza B/Malaysia/2506/04 MA virus encoding the mH5/B chimeric HA described in FIGS. 29 and 30. mH8/B Mal refers to influenza B/Malaysia/2506/04 MA virus encoding the mH8/B chimeric HA described in FIGS. 31 and 32. mH11/B Mal refers to influenza B/Malaysia/2506/04 MA virus encoding the mH11/B chimeric HA described in FIGS. 33 and 34. mH13/B Mal refers to influenza B/Malaysia/2506/04 MA virus encoding the mH13/B chimeric HA described in FIGS. 35 and 36.

FIGS. 38A-38D demonstrate that cross-protective subdominant conserved antigenic sites within influenza B virus HA were preserved in the chimeric HA. MDCK cells were infected with influenza viruses expressing mH5/B chimeric HA (FIG. 38A), mH8/B chimeric HA (FIG. 38B), mH11/B chimeric HA (FIG. 38C), or mH13/B chimeric HA (FIG. 38D) at an MOI of 5 without TPCK-trypsin and compared to uninfected cells or cells infected with B/Mal04 MA at an MOI of 5 without TPCK-trypsin. Cells were incubated at 33 degrees Celsius and 5% CO₂. 17 hours post infection, cells were fixed with methanol free 5% paraformaldehyde for immunofluorescence surface staining using the indicated anti-influenza B virus HA cross-protective human/mouse monoclonal antibodies and anti-influenza B virus HA polyclonal mouse serum. Secondary Alexa Fluor anti-human or anti-mouse antibody were used. Images were taken using Zeiss LSM 880 confocal microscope. PFU refers to plaque forming unit. B/Mal04 MA refers to wild type influenza B/Malaysia/2506/04 MA virus. mH5/B Mal refers to influenza B/Malaysia/2506/04 MA virus encoding the mH5/B chimeric HA described in FIGS. 29 and 30. mH8/B Mal refers to influenza B/Malaysia/2506/04 MA virus encoding the mH8/B chimeric HA described in FIGS. 31 and 32. mH11/B Mal refers to influenza B/Malaysia/2506/04 MA virus encoding the mH11/B chimeric HA described in FIGS. 33 and 34. mH13/B Mal refers to influenza B/Malaysia/2506/04 MA virus encoding the mH13/B chimeric HA described in FIGS. 35 and 36. TPCK refers to L-1-Tosylamide-2-phenylethyl chloromethyl ketone.

FIGS. 39A-39D demonstrate that immunodominant epitopes on influenza B/Yamagata/88 HA head were ablated in the chimeric HAs. Mouse and ferret sera were raised against wild type influenza B virus strain B/Yamagata/16/88 to acquire hemagglutination inhibition (HI) reactivity. HI assays for the mouse and ferret sera were performed using turkey red blood cells (RBCs) with influenza viruses expressing mH5/B chimeric HA (FIG. 39A), mH8/B chimeric HA (FIG. 39B), mH11/B chimeric HA (FIG. 39C), or mH13/B chimeric HA (FIG. 39D) or wild type influenza B virus strain B/Yamagata/16/88 (FIGS. 39A-39D). B Yamagata 88 wild type refers to wild type influenza B/Yamagata/16/88 virus. mH5/B Mal refers to influenza B/Malaysia/2506/04 MA virus encoding the mH5/B chimeric HA described in FIGS. 29 and 30. mH8/B Mal refers to influenza B/Malaysia/2506/04 MA virus encoding the mH8/B chimeric HA described in FIGS. 31 and 32. mH11/B Mal refers to influenza B/Malaysia/2506/04 MA virus encoding the mH11/B chimeric HA described in FIGS. 33 and 34. mH13/B Mal refers to influenza B/Malaysia/2506/04 MA virus encoding the mH13/B chimeric HA described in FIGS. 35 and 36.

FIGS. 40A AND 40B depict weight loss (FIG. 40A) and survival (FIG. 40B) of chimeric HA-vaccinated mice after challenge with B/Malaysia/2506/04 (Victoria-like). Vaccination with the chimeric HA regiment resulted in complete protection from mortality with minimal weight loss. Circles: Group 1 (chimeric HA); squares: Group 2 (prime only); triangles pointing up: Group 3 (TIV); triangles pointing down: Group 4 (irrelevant protein); diamonds: naïve.

FIG. 41A: Nucleic acid sequence encoding influenza A/black headed gull/Sweden/1/99 virus HA (SEQ ID NO: 71). FIG. 41B: Amino acid sequence (SEQ ID NO: 72) of the nucleic acid of FIG. 41A.

### 5. DETAILED DESCRIPTION

### 5.1 Chimeric Influenza Virus Hemagglutinin Polypeptides

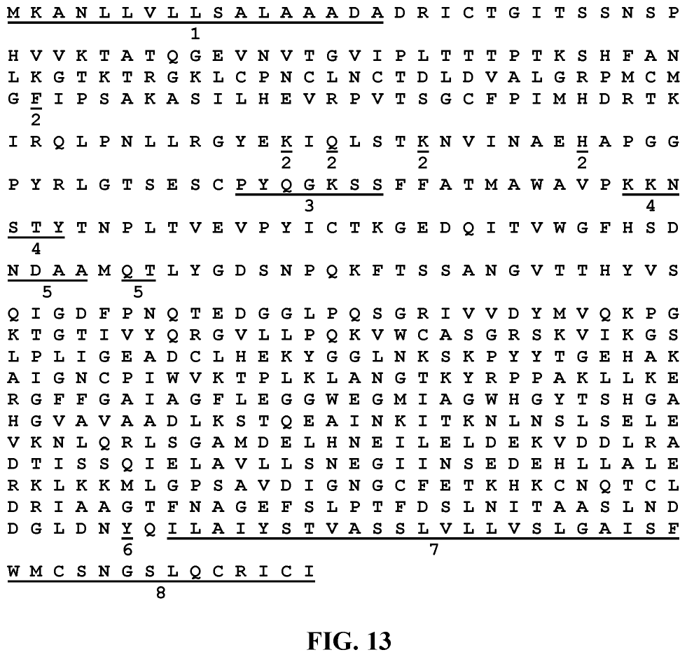

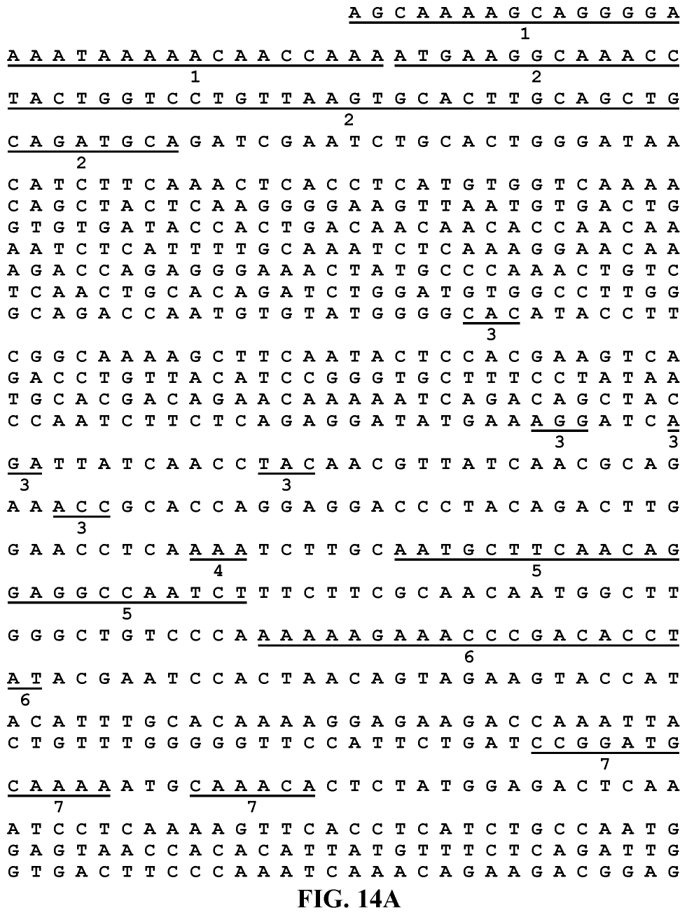

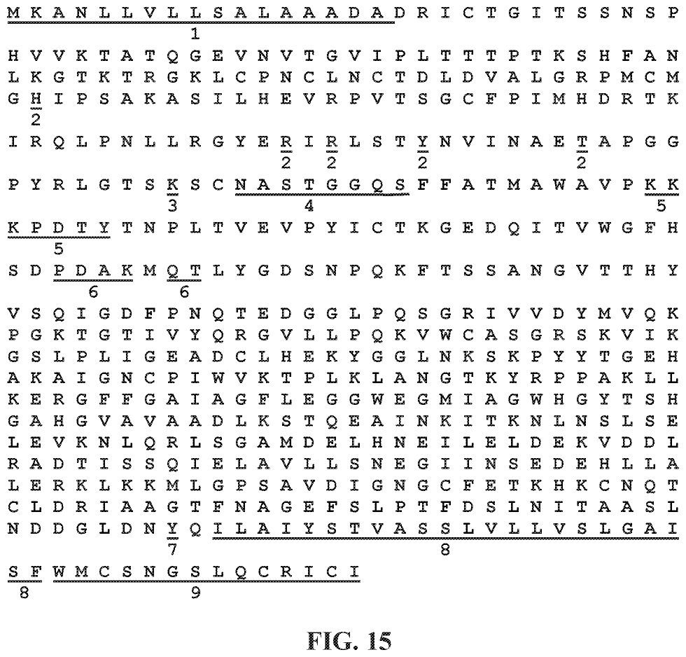

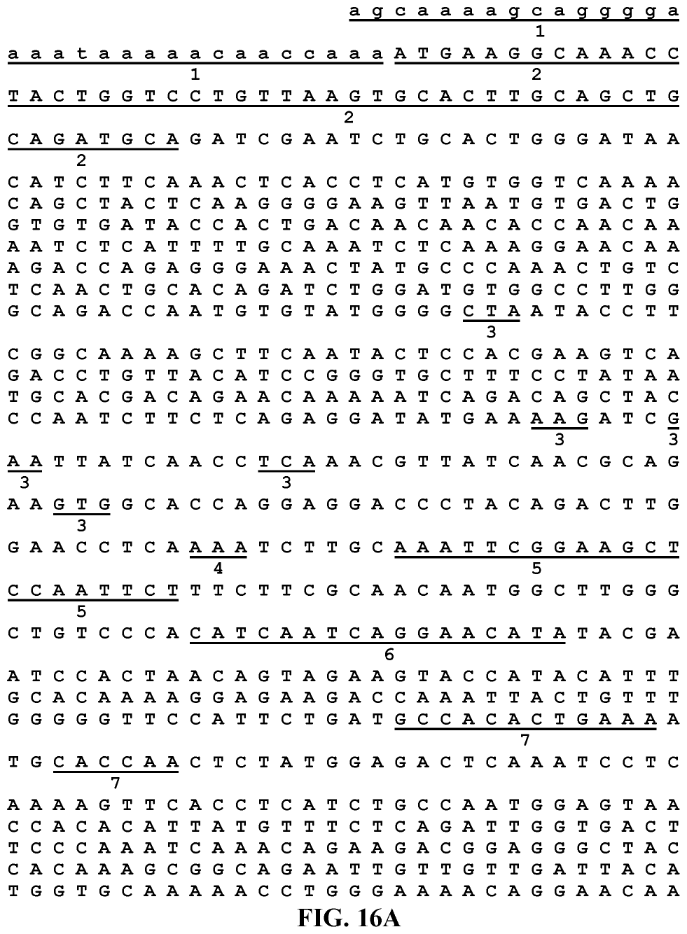

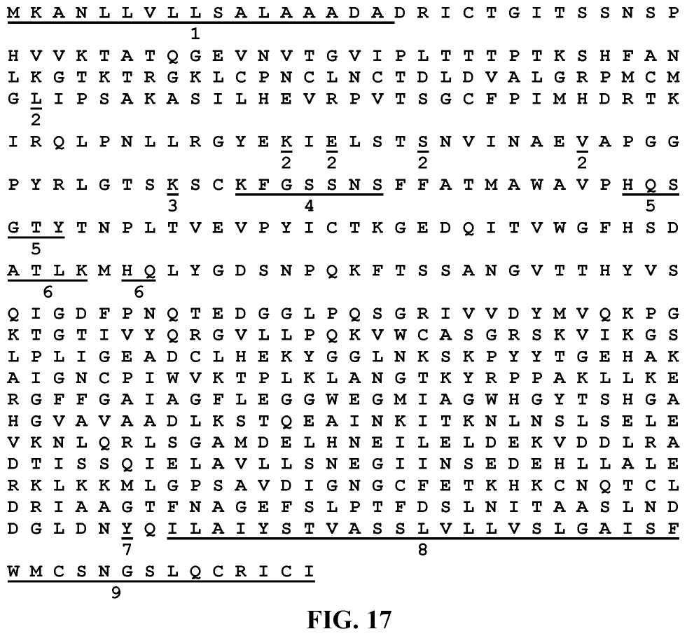

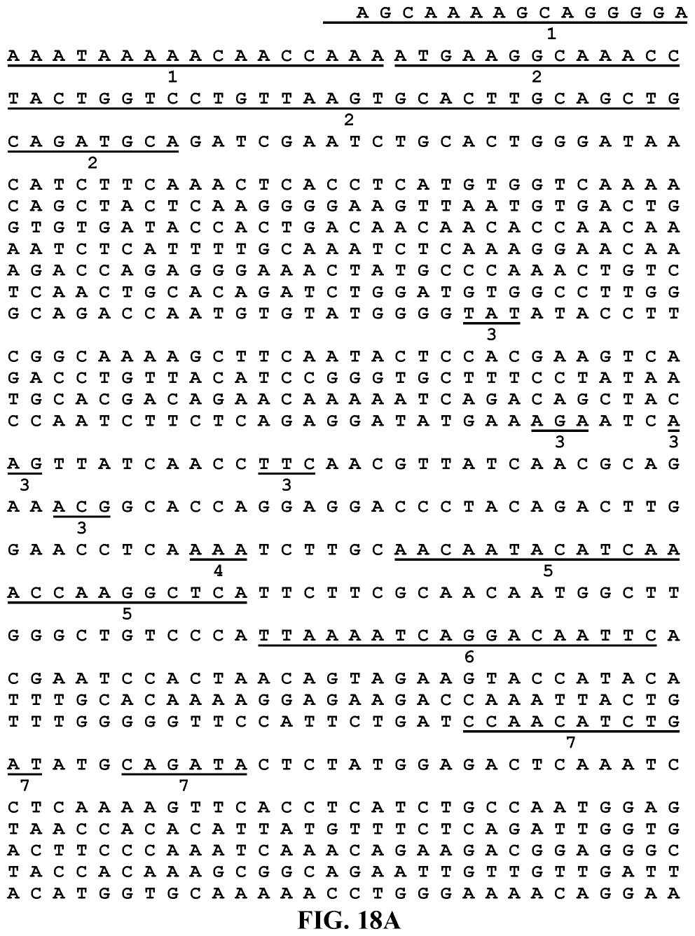

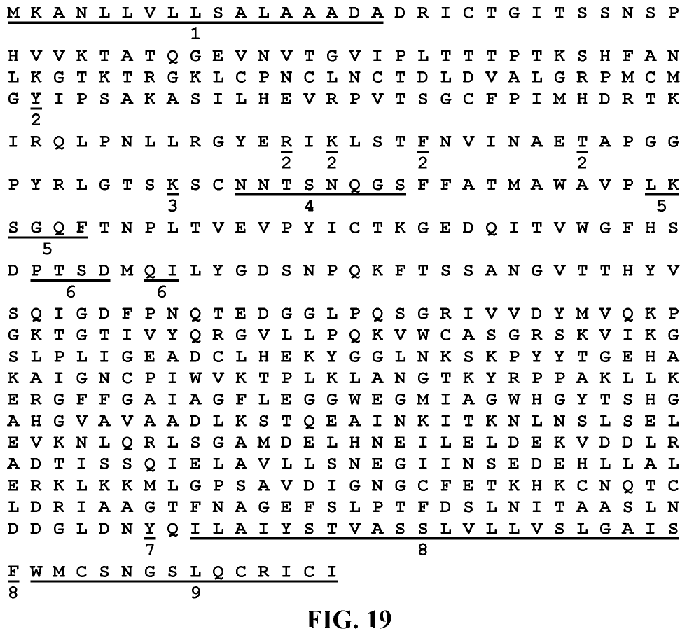

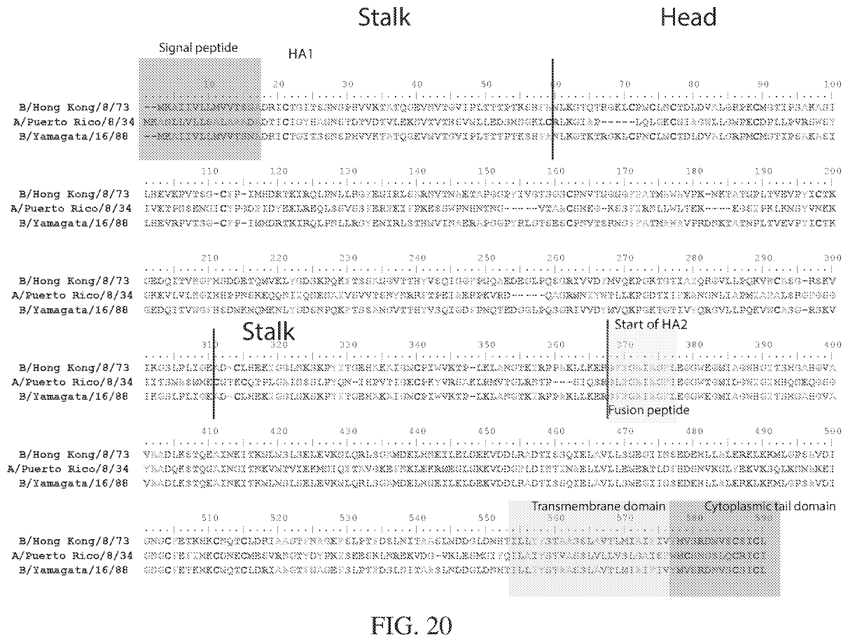

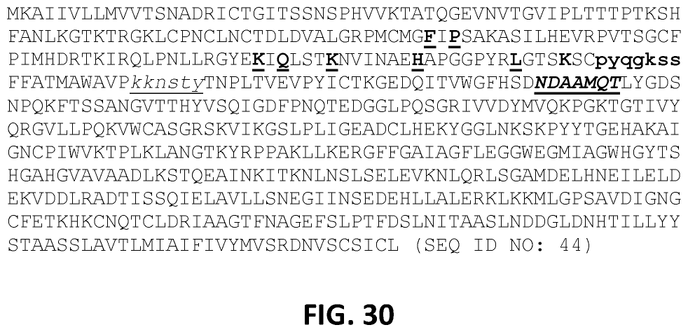

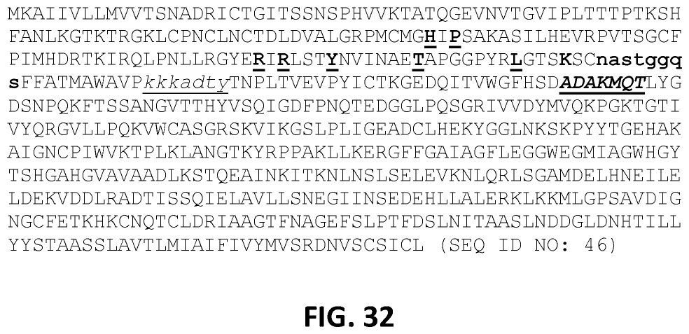

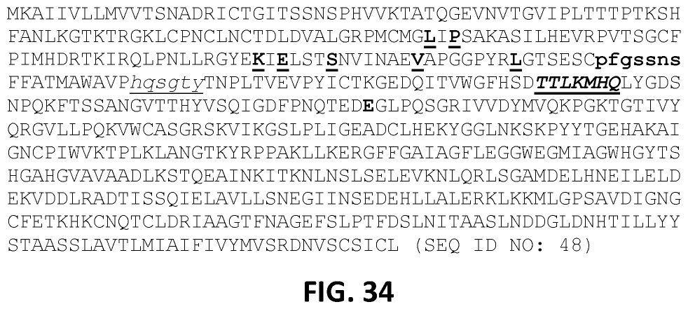

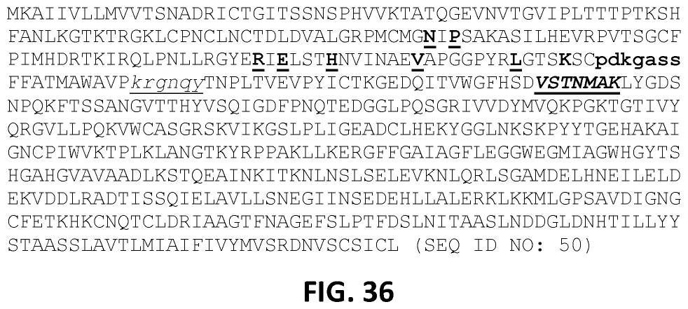

In another aspect, provided herein are chimeric hemagglutinin (HA) polypeptides comprising an HA ectodomain of an influenza B virus comprising 2, 3, 4, 5, 6, 7, 8, 9, 10, 11, 12 or more amino acid substitutions within an antigenic loop of the globular head domain of the influenza B virus HA, wherein the amino acid substitutions substitute 2, 3, 4, 5, 6, 7, 8, 9, 10, 11, 12 or more amino acid residues in the loop of the globular head of the influenza B virus HA with amino acid residues found in a corresponding region of the globular domain of an influenza A virus HA. In another aspect, provided herein are chimeric hemagglutinin (HA) polypeptides comprising an HA ectodomain of an influenza B virus comprising 2, 3, 4, 5, 6, 7, 8, 9, 10, 11, 12 or more amino acid substitutions within the 120 loop, 150 loop, 160 loop or 190 helix of the globular head domain of the influenza B virus HA, wherein the amino acid substitutions substitute 2, 3, 4, 5, 6, 7, 8, 9, 10, 11, 12 or more amino acid residues in the 120 loop, 150 loop, 160 loop or 190 helix of the globular head of the influenza B virus HA with amino acid residues found in a corresponding region of the globular domain of an influenza A virus HA. In one embodiment, provided herein are chimeric hemagglutinin (HA) polypeptides comprising an HA ectodomain of an influenza B virus comprising 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA. In another embodiment, provided herein are chimeric hemagglutinin (HA) polypeptides comprising an HA ectodomain of an influenza B virus comprising 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA. In another embodiment, provided herein are chimeric hemagglutinin (HA) polypeptides comprising an HA ectodomain of an influenza B virus comprising 2, 3, 4, 5 or more amino acid substitutions within 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA. In another embodiment, provided herein are chimeric hemagglutinin (HA) polypeptides comprising an HA ectodomain of an influenza B virus comprising 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMQT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In some embodiments, the chimeric HA polypeptide may also comprise the signal peptide, transmembrane domain, and cytoplasmic tail domain from the influenza B virus HA. In other embodiments, the chimeric HA polypeptide comprises the signal peptide from the influenza B virus HA but lacks the transmembrane and cytoplasmic tail domains. In some embodiments, the chimeric HA polypeptide may also comprise the signal peptide, transmembrane domain, and cytoplasmic tail domain from an influenza A virus HA. In other embodiments, the chimeric HA polypeptide comprises the signal peptide from an influenza A virus HA but lacks the transmembrane and cytoplasmic tail domains. In some embodiments, the chimeric HA comprises the signal peptide of the HA of the influenza virus backbone of the chimeric HA. For example, if the chimeric HA is engineered for an influenza A virus backbone (e.g., the influenza virus comprising or engineered to express the chimeric HA is an influenza A virus), then the chimeric HA comprises the signal peptide of the influenza A virus. In some embodiments, the chimeric HA comprises the signal peptide, transmembrane domain, and cytoplasmic domain of the HA of the influenza virus backbone of the chimeric HA. In some embodiments, the chimeric HA polypeptide may also comprise the signal peptide from the HA of the influenza virus that is engineered to express the chimeric HA. In some embodiments, the chimeric HA polypeptide may also comprise the signal peptide, transmembrane domain, and cytoplasmic tail domain from the HA of the influenza virus that is engineered to express the chimeric HA. Also provided herein are nucleic acids comprising nucleotide sequences encoding such a chimeric HA. In some embodiments, the nucleic acids comprise nucleotide sequences encoding such a chimeric HA and the 5′ and 3′ non-coding regions from the influenza B virus HA. In some embodiments, the nucleic acids comprise nucleotide sequences encoding such a chimeric HA and the 5′ and 3′ non-coding regions from an influenza A virus HA. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA. In specific embodiments, the chimeric HA polypeptide is soluble. In certain embodiments, the chimeric HA polypeptides comprise 1, 2, 3, 4, 5 or more amino acid substitutions in the globular head domain of the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid).

In another aspect, provided herein are chimeric hemagglutinin (HA) polypeptides comprising an HA ectodomain of an influenza B virus comprising one, two, three or all of the following: (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNOMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In some embodiments, the chimeric HA polypeptide may also comprise the signal peptide, transmembrane domain, and cytoplasmic tail domain from the influenza B virus HA. In other embodiments, the chimeric HA polypeptide comprises the signal peptide from the influenza B virus HA but lacks the transmembrane and cytoplasmic tail domains. In some embodiments, the chimeric HA polypeptide may also comprise the signal peptide, transmembrane domain, and cytoplasmic tail domain from an influenza A virus HA. In other embodiments, the chimeric HA polypeptide comprises the signal peptide from an influenza A virus HA but lacks the transmembrane and cytoplasmic tail domains. In some embodiments, the chimeric HA comprises the signal peptide of the HA of the influenza virus backbone of the chimeric HA. For example, if the chimeric HA is engineered for an influenza A virus backbone (e.g., the influenza virus comprising or engineered to express the chimeric HA is an influenza A virus), then the chimeric HA comprises the signal peptide of the influenza A virus. In some embodiments, the chimeric HA comprises the signal peptide, transmembrane domain, and cytoplasmic domain of the HA of the influenza virus backbone of the chimeric HA. In some embodiments, the chimeric HA polypeptide may also comprise the signal peptide from the HA of the influenza virus that is engineered to express the chimeric HA. In some embodiments, the chimeric HA polypeptide may also comprise the signal peptide, transmembrane domain, and cytoplasmic tail domain from the HA of the influenza virus that is engineered to express the chimeric HA. Also provided herein are nucleic acids comprising nucleotide sequences encoding such a chimeric HA. In some embodiments, the nucleic acids comprise nucleotide sequences encoding such a chimeric HA and the 5′ and 3′ non-coding regions from the influenza B virus HA. In some embodiments, the nucleic acids comprise nucleotide sequences encoding such a chimeric HA and the 5′ and 3′ non-coding regions from an influenza A virus HA. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA. In specific embodiments, the chimeric HA polypeptide is soluble. In certain embodiments, the chimeric HA polypeptides comprise 1, 2, 3, 4, 5 or more amino acid substitutions in the globular head domain of the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid).

In another aspect, provided herein are chimeric hemagglutinin (HA) polypeptides comprising (i) a hemagglutinin ectodomain from an influenza B virus with 2, 3, 4, 5, 6, 7, 8, 9, 10, 11, 12 or more amino acid substitutions within the 120 loop, 150 loop, 160 loop or 190 helix of the globular head domain of the influenza B virus HA and (ii) a signal peptide, a transmembrane domain and a cytoplasmic tail domain from an influenza A virus, wherein the amino acid substitutions substitute 2, 3, 4, 5, 6, 7, 8, 9, 10, 11, 12 or more amino acid residues in the 120 loop, 150 loop, 160 loop or 190 helix of the globular head of the influenza B virus HA with amino acid residues found in a corresponding region of the globular domain of an influenza A virus HA. In one embodiment, provided herein are chimeric hemagglutinin (HA) polypeptides comprising (i) a hemagglutinin ectodomain from an influenza B virus with 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA and (ii) a signal peptide, a transmembrane domain and a cytoplasmic tail domain from an influenza A virus, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA. In another embodiment, provided herein are chimeric hemagglutinin (HA) polypeptides comprising (i) a hemagglutinin ectodomain from an influenza B virus with 2, 3, 4, 5, 6, 7, 8, 9 or more amino acid substitutions within the 150 loop of the globular head domain of the influenza B virus HA and (ii) a signal peptide, a transmembrane domain and a cytoplasmic tail domain from an influenza A virus, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA. In another embodiment, provided herein are chimeric hemagglutinin (HA) polypeptides comprising (i) a hemagglutinin ectodomain from an influenza B virus with 2, 3, 4, 5 or more amino acid substitutions within the 160 loop of the globular head domain of the influenza B virus HA and (ii) a signal peptide, a transmembrane domain and a cytoplasmic tail domain from an influenza A virus, wherein the amino acid substitutions substitute 2, 3, 4, 5 or more amino acid residues in the 160 loop of the globular head of the influenza B virus HA with amino acid residues found in a corresponding region of the globular domain of an influenza A virus HA. In another embodiment, provided herein are chimeric hemagglutinin (HA) polypeptides comprising (i) a hemagglutinin ectodomain from an influenza B virus with 2, 3, 4, 5, 6, 7, 8 or more amino acid substitutions within the 190 helix of the globular head domain of the influenza B virus HA and (ii) a signal peptide, a transmembrane domain and a cytoplasmic tail domain from an influenza A virus, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the globular head domain of the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the influenza B virus is from the Yamagata lineage. In other embodiments, the influenza B virus is from the Victoria lineage. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99). Also provided herein are nucleic acids comprising nucleotide sequences encoding said chimeric HA polypeptides. In some embodiments, the nucleic acids comprise nucleotide sequences encoding such a chimeric HA and the 5′ non-coding region and 3′ non-coding region from an influenza A virus.

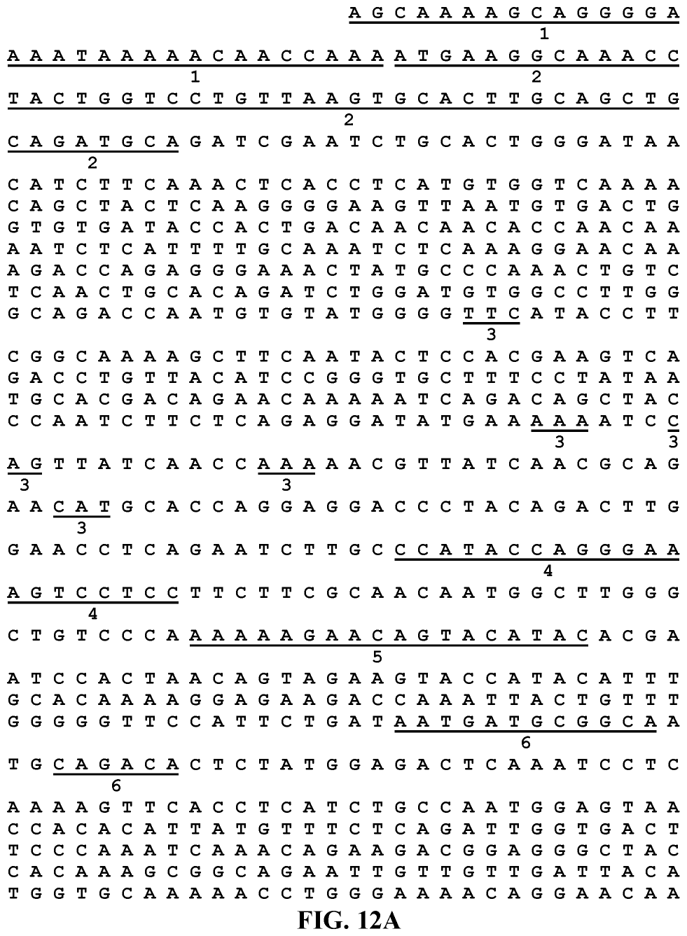

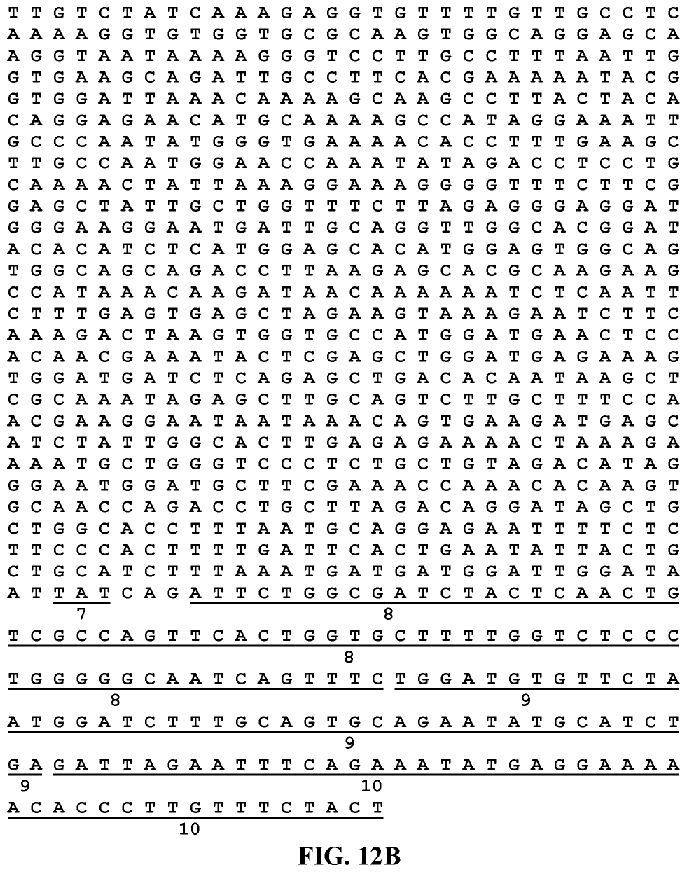

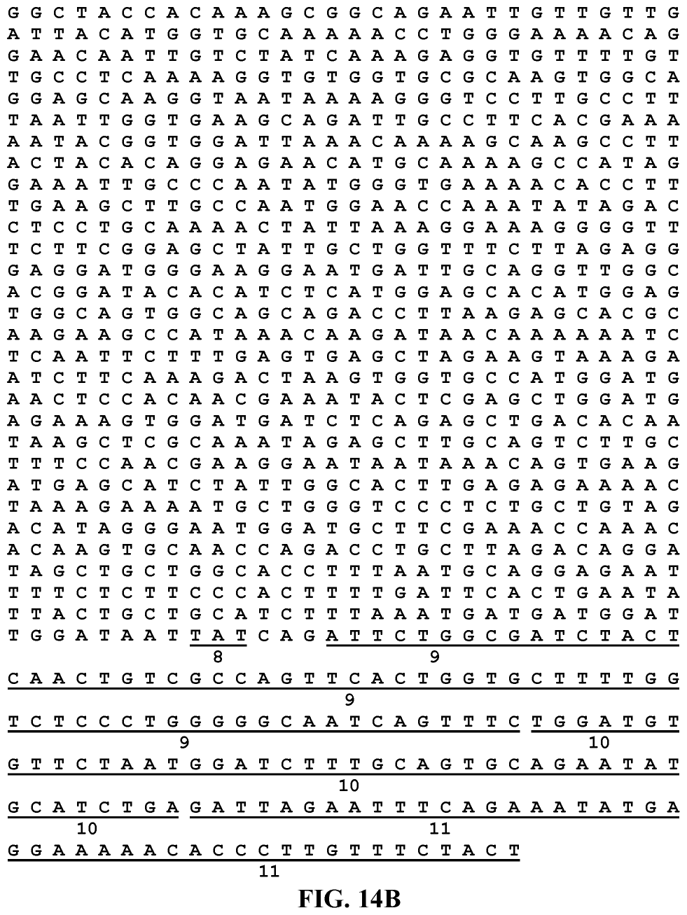

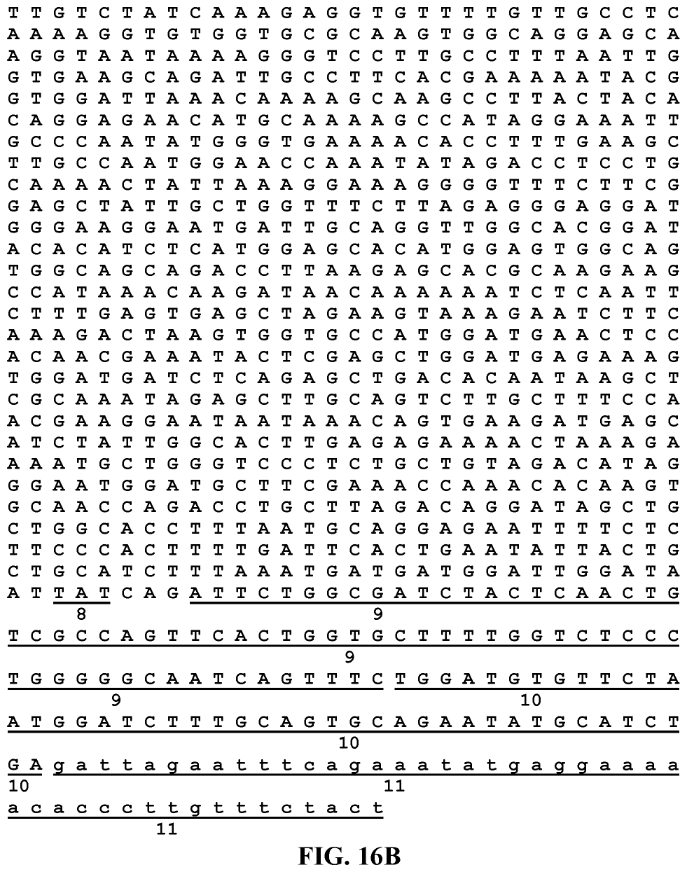

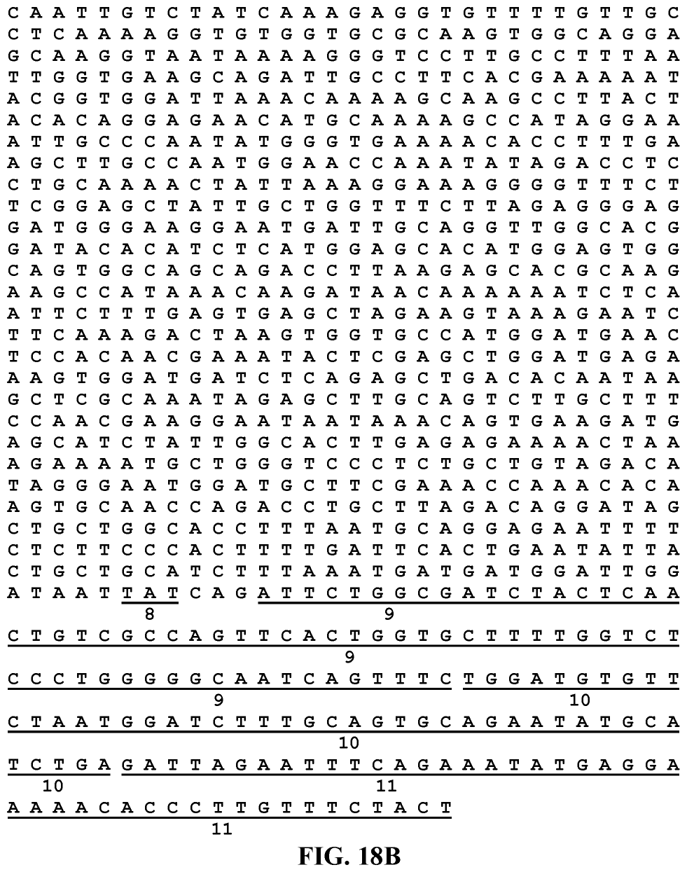

In another aspect, provided herein are chimeric hemagglutinin (HA) polypeptides comprising: (a) a hemagglutinin ectodomain from an influenza B virus with one, two, three or all of the following (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA; and (b) a signal peptide, a transmembrane domain and a cytoplasmic tail domain from an influenza A virus. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO: 64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the globular head domain of the influenza B virus HA which are outside of the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the influenza B virus is from the Yamagata lineage. In other embodiments, the influenza B virus is from the Victoria lineage. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99). Also provided herein are nucleic acids comprising nucleotide sequences encoding said chimeric HA polypeptides. In some embodiments, the nucleic acids comprise nucleotide sequences encoding a chimeric HA polypeptide and the 5′ non-coding region and 3′ non-coding region from an influenza A virus. In some embodiments, the nucleic acids comprise nucleotide sequences encoding a chimeric HA polypeptide and the 5′ non-coding region and 3′ non-coding region from an influenza B virus. In some embodiments, the nucleic acids comprise nucleotide sequences encoding such a chimeric HA and the 5′ and 3′ non-coding regions of the HA of the influenza virus backbone of the chimeric HA. For example, if the chimeric HA is engineered for an influenza A virus backbone (e.g., the influenza virus comprising or engineered to express the chimeric HA is an influenza A virus), then the nucleic acids comprise nucleotide sequences encoding such a chimeric HA and the 5′ and 3′ non-coding regions of the influenza A virus. In some embodiments, the nucleic acids comprise nucleotide sequences encoding such a chimeric HA and the 5′ and 3′ non-coding regions from the influenza virus HA of the influenza virus that is engineered to express the chimeric HA. In a specific embodiment, the nucleic acid comprises the nucleotide sequence set forth in FIG. 12A and FIG. 12B or the complement thereof. In a specific embodiment, the nucleic acid comprises the nucleotide sequence set forth in FIG. 14A and FIG. 14B or the complement thereof. In a specific embodiment, the nucleic acid comprises the nucleotide sequence set forth in FIG. 16A and FIG. 16B or the complement thereof. In a specific embodiment, the nucleic acid comprises the nucleotide sequence set forth in FIG. 18A and FIG. 18B or the complement thereof.

In another aspect, provided herein are chimeric hemagglutinin (HA) polypeptides comprising (i) a hemagglutinin ectodomain from a first influenza B virus strain with 2, 3, 4, 5, 6, 7, 8, 9, 10, 11, 12 or more amino acid substitutions within the 120 loop, 150 loop, 160 loop or 190 helix of the globular head domain of the influenza B virus HA and (ii) a signal peptide, a transmembrane domain and a cytoplasmic tail domain from a second influenza B virus strain, wherein the amino acid substitutions substitute 2, 3, 4, 5, 6, 7, 8, 9, 10, 11, 12 or more amino acid residues in the 120 loop, 150 loop, 160 loop or 190 helix of the globular head of the first influenza B virus strain HA with amino acid residues found in a corresponding region of the globular domain of an influenza A virus HA. In one embodiment, provided herein are chimeric hemagglutinin (HA) polypeptides comprising (i) a hemagglutinin ectodomain from an first influenza B virus strain with 2, 3, 4, 5, 6, 7, 8, 9, 10, 11, 12, 13, 14, 15, 16, 17, 18, 19, 20 or more amino acid substitutions within the 120 loop of the globular head domain of the first influenza B virus strain and (ii) a signal peptide, a transmembrane domain and a cytoplasmic tail domain from a second influenza B virus strain, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the first influenza B virus strain HA with amino acid residues found in a corresponding region of the globular domain of an influenza A virus HA. In another embodiment, provided herein are chimeric hemagglutinin (HA) polypeptides comprising (i) a hemagglutinin ectodomain from an first influenza B virus strain with 2, 3, 4, 5, 6, 7, 8, 9 or more amino acid substitutions within the 150 loop of the globular head domain of the first influenza B virus strain HA and (ii) a signal peptide, a transmembrane domain and a cytoplasmic tail domain from a second influenza B virus strain, wherein the amino acid substitutions substitute 2, 3, 4, 5, 6, 7, 8, 9 or more amino acid residues in the 150 loop of the globular head of the in first influenza B virus strain HA with amino acid residues found in a corresponding region of the globular domain of a second influenza B virus strain HA. In another embodiment, provided herein are chimeric hemagglutinin (HA) polypeptides comprising (i) a hemagglutinin ectodomain from a first influenza B virus strain with 2, 3, 4, 5 or more amino acid substitutions within the 160 loop of the globular head domain of the first influenza B virus strain HA and (ii) a signal peptide, a transmembrane domain and a cytoplasmic tail domain from a second influenza B virus strain, wherein the amino acid substitutions substitute 2, 3, 4, 5 or more amino acid residues in the 160 loop of the globular head of the first influenza B virus strain HA with amino acid residues found in a corresponding region of the globular domain of an influenza A virus HA. In another embodiment, provided herein are chimeric hemagglutinin (HA) polypeptides comprising (i) a hemagglutinin ectodomain from a first influenza B virus strain with 2, 3, 4, 5, 6, 7, 8 or more amino acid substitutions within the 190 helix of the globular head domain of the influenza B virus HA and (ii) a signal peptide, a transmembrane domain and a cytoplasmic tail domain from a second influenza B virus strain, wherein the amino acid substitutions substitute 2, 3, 4, 5, 6, 7, 8 or more amino acid residues in the 190 helix of the globular head of the first influenza B virus strain HA with amino acid residues found in a corresponding region of the globular domain of an influenza A virus HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and/or 190 helix, respectively, set forth in FIG. 13. In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and/or 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the following underlined and bold amino acid residues ADAKMOT (SEQ ID NO: 54) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and/or 190 helix, respectively, set forth in FIG. 15. In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and/or 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO: 56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNOMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and/or 190 helix, respectively, set forth in FIG. 17. In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and/or 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTFNVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises a 120 loop, a 150 loop, a 160 loop, and/or a 190 helix with the amino acid sequences of the 120 loop, 150 loop, 160 loop, and/or 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO: 60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNQMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and/or 190 helix, respectively, set forth in FIG. 36. In certain embodiments, the chimeric HA polypeptides comprise 1, 2, 3, 4, 5 or more amino acid substitutions in the globular head domain of the influenza B virus HA which are outside of one, two, three, or all of the following: the 120 loop, 150 loop, 160 loop and/or 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the first influenza B virus strain and the second influenza B virus strain are from the same lineage. In some embodiments, the first influenza B virus strain and the second influenza B virus strain are from the same lineage but are different strains. In a specific embodiment, the first influenza B virus strain is the same strain as the second influenza B virus strain. In another embodiment, the first influenza B virus strain is a different strain than the second influenza B virus strain. In some embodiments, the first influenza B virus strain and the second influenza B virus strain are from different lineages. In some embodiments, the first influenza B virus strain is from the Yamagata lineage. In other embodiments, the first influenza B virus is from the Victoria lineage. In some embodiments, the second influenza B virus strain is from the Yamagata lineage. In other embodiments, the second influenza B virus is from the Victoria lineage. In a specific embodiment, the second influenza B virus strain is the same strain as the influenza virus backbone of an influenza virus either comprising, containing, or both the chimeric HA. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99). Also provided herein are nucleic acids comprising nucleotide sequences encoding said chimeric HA polypeptides. In some embodiments, the nucleic acids comprise nucleotide sequences encoding a chimeric HA polypeptide and the 5′ non-coding region and 3′ non-coding region from the second influenza B virus. In some embodiments, the nucleic acids comprise nucleotide sequences encoding such a chimeric HA and the 5′ and 3′ non-coding regions of the HA of the influenza virus backbone of an influenza virus either comprising, containing, or both the chimeric HA.

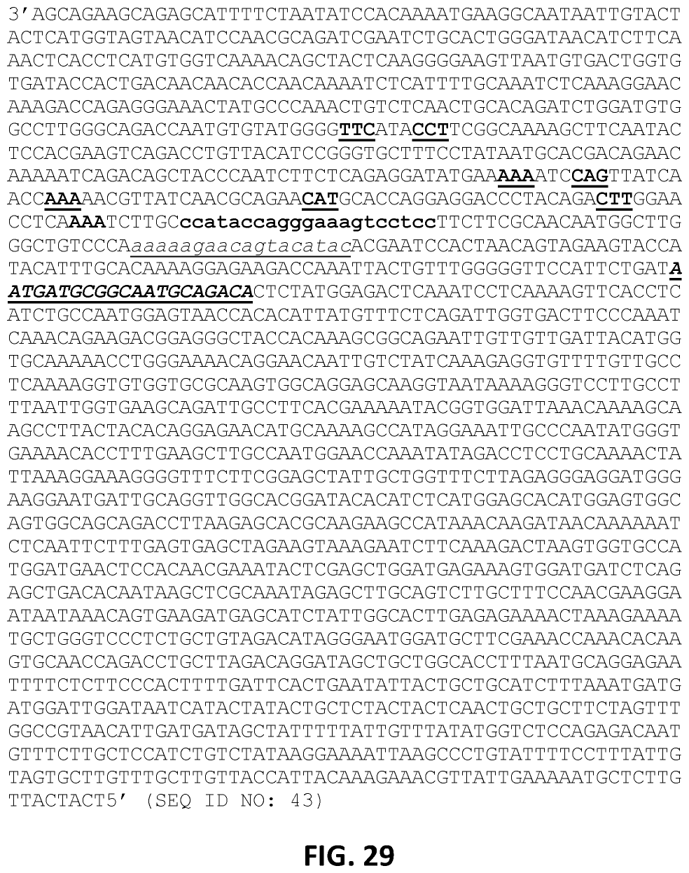

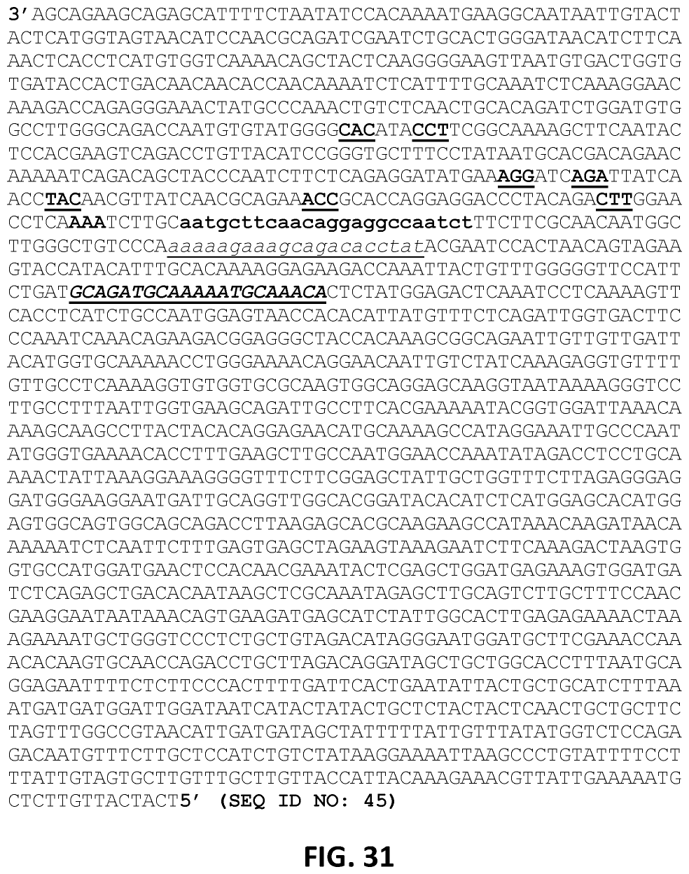

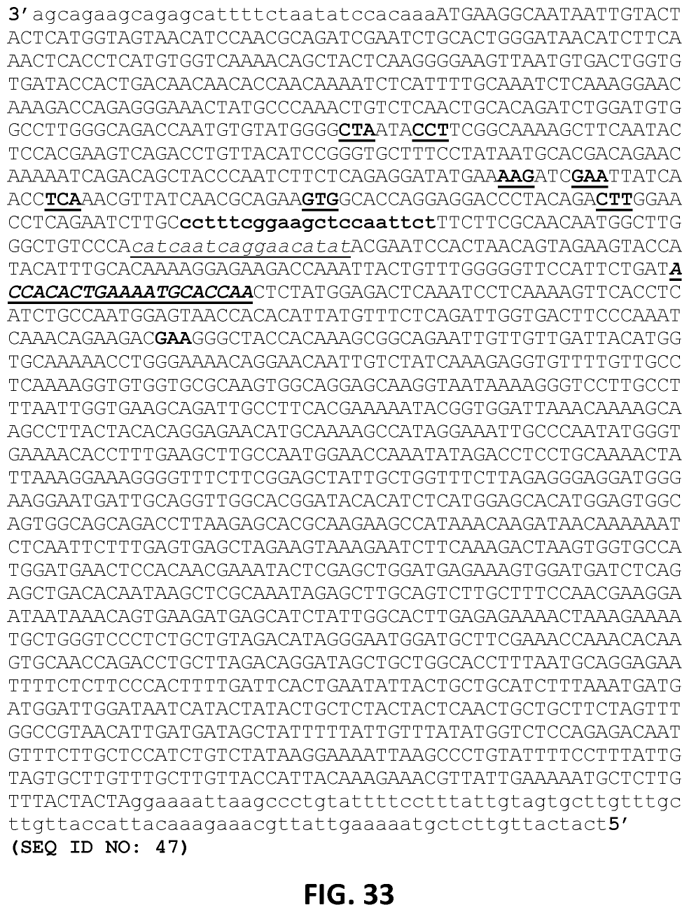

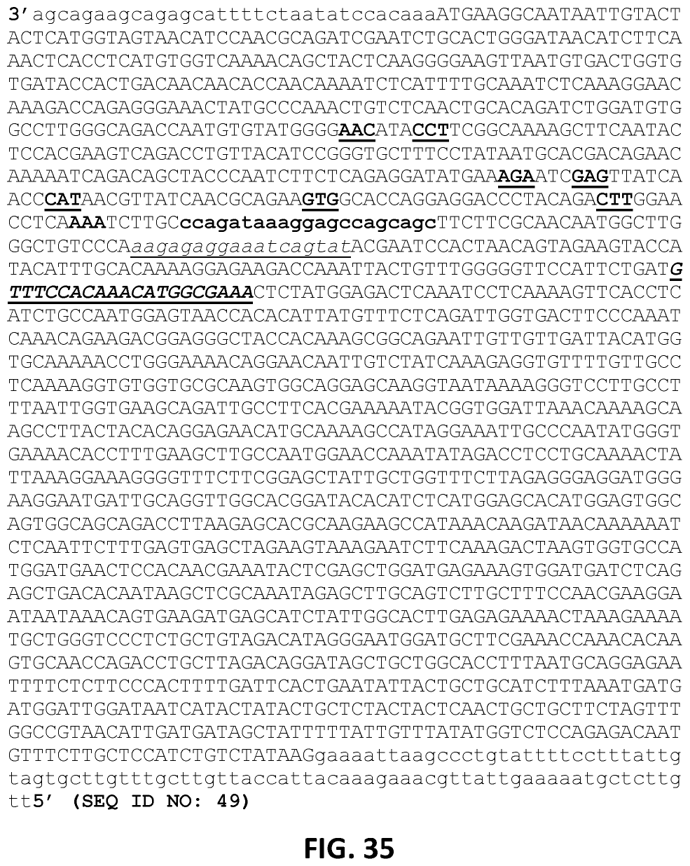

In another aspect, provided herein are chimeric hemagglutinin (HA) polypeptides comprising: (a) a hemagglutinin ectodomain from a first influenza B virus strain with one, two, three or all of the following (i) 2, 3, 4, 5, 6, 7, 8, 9, 10, 11, 12, 13, 14, 15, 16, 17, 18, 19, 20 or more amino acid substitutions within the 120 loop of the globular head domain of the first influenza B virus strain HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the first influenza B virus strain HA with amino acid residues found in a corresponding region of the globular domain of an influenza A virus HA; (ii) 2, 3, 4, 5, 6, 7, 8, 9 or more amino acid substitutions within the 150 loop of the globular head domain of the first influenza B virus strain HA, wherein the amino acid substitutions substitute 2, 3, 4, 5, 6, 7, 8, 9 or more amino acid residues in the 150 loop of the globular head of the first influenza B virus strain HA with amino acid residues found in a corresponding region of the globular domain of an influenza A virus HA; (iii) 2, 3, 4, 5 or more amino acid substitutions within the 160 loop of the globular head domain of the first influenza B virus strain HA, wherein the amino acid substitutions substitute 2, 3, 4, 5 or more amino acid residues in the 160 loop of the globular head of the first influenza B virus strain HA with amino acid residues found in a corresponding region of the globular domain of an influenza A virus HA; and (iv) 2, 3, 4, 5, 6, 7, 8 or more amino acid substitutions within the 190 helix of the globular head domain of the first influenza B virus strain HA, wherein the amino acid substitutions substitute 2, 3, 4, 5, 6, 7, 8 or more amino acid residues in the 190 helix of the globular head of the first influenza B virus strain HA with amino acid residues found in a corresponding region of the globular domain of an influenza A virus HA; and (b) a signal peptide, a transmembrane domain and a cytoplasmic tail domain from a second influenza B virus strain. In a specific embodiment, the first influenza B virus strain is the same strain as the second influenza B virus strain. In another embodiment, the first influenza B virus strain is a different strain than the second influenza B virus strain. In a specific embodiment, the second influenza B virus strain is the same strain as the influenza virus backbone of an influenza virus either comprising, containing, or both the chimeric HA. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues FIP and KIQLSTKNVINAEHAPGGPYRL (SEQ ID NO: 2) from influenza A virus A/Vietnam/1203/04 (HALo). In specific embodiments, the following amino acid residues in influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4) from influenza A virus A/Vietnam/1203/04 (HALo). In specific embodiments, the following amino acid residues in the 150 loop of influenza B virus B/Yamagata/16/88 NVTSRNG (SEQ ID NO: 3) are substituted with amino acid residues YQGKSS (SEQ ID NO:4). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19) from influenza A virus A/Vietnam/1203/04 (HALo). In specific embodiments, the following amino acid residues in the 150 loop of influenza B virus B/Yamagata/16/88 PNVTSRNG (SEQ ID NO: 18) are substituted with amino acid residues PYQGKSS (SEQ ID NO:19). In specific embodiments, the following underlined and bold amino acid residues in the 160 loop of influenza B virus B/Yamagata/16/88 RDNKTA (SEQ ID NO: 5) are substituted with the following bold and underlined amino acid residues KKNSTY (SEQ ID NO: 6) from influenza A virus A/Vietnam/1203/04 (HALo). In specific embodiments, the following acid residues in the 160 loop of influenza B virus B/Yamagata/16/88 RDNKTA (SEQ ID NO: 5) are substituted with amino acid residues KKNSTY (SEQ ID NO: 6). In specific embodiments, the following underlined and bold amino acid residues in the 190 helix of influenza B virus B/Yamagata/16/88 NKNQMKN (SEQ ID NO: 7) are substituted with the following underlined and bold amino acid residues NDAAMQT (SEQ ID NO: 8) from influenza A virus A/Vietnam/1203/04 (HALo). In specific embodiments, the following amino acid residues in the 190 helix of influenza B virus B/Yamagata/16/88 NKNQMKN (SEQ ID NO: 7) are substituted with amino acid residues NDAAMQT (SEQ ID NO: 8). In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and 190 helix, respectively, set forth in FIG. 13. In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and 190 helix, respectively, set forth in FIG. 30. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues HIP and RIRLSTYNVINAETAPGGPYRL (SEQ ID NO: 51) from influenza A virus A/Mallard/Sweden/24/2002 virus. In specific embodiments, the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51). In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52) from influenza A virus A/Mallard/Sweden/24/2002 virus. In specific embodiments, the following amino acid residues in the 150 loop of influenza B virus B/Yamagata/16/88 PNVTSRNG (SEQ ID NO: 18) are substituted with amino acid residues NASTGGQS (SEQ ID NO: 52). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKADTY (SEQ ID NO: 53) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKADTY (SEQ ID NO: 53). In specific embodiments, the following underlined and bold amino acid residues in the 160 loop of influenza B virus B/Yamagata/16/88 RDNKTA (SEQ ID NO: 5) are substituted with the following bold and underlined amino acid residues KKKPDTY (SEQ ID NO: 68) from influenza A virus A/Mallard/Sweden/24/2002 virus. In specific embodiments, the following amino acid residues in the 160 loop of influenza B virus B/Yamagata/16/88 RDNKTA (SEQ ID NO: 5) are substituted with amino acid residues KKKPDTY (SEQ ID NO: 68). In specific embodiments, the following underlined and bold amino acid residues in the 190 helix of influenza B virus B/Yamagata/16/88 NKNQMKN (SEQ ID NO: 7) are substituted with the influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues ADAKMQT (SEQ ID NO: 54). In specific embodiments, the following underlined and bold amino acid residues in the 190 helix of influenza B virus B/Yamagata/16/88 NKNQMKN (SEQ ID NO: 7) are substituted with the following underlined and bold amino acid residues PDAKMQT (SEQ ID NO: 69) from influenza A virus A/Mallard/Sweden/24/2002 virus. In specific embodiments, the following amino acid residues in the 190 helix of influenza B virus B/Yamagata/16/88 NKNQMKN (SEQ ID NO: 7) are substituted with amino acid residues PDAKMQT (SEQ ID NO: 69). In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and 190 helix, respectively, set forth in FIG. 15. In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and 190 helix, respectively, set forth in FIG. 32. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues LIP and KIELSTSNVINAEVAPGGPYRL (SEQ ID NO: 55) from influenza A virus A/northern shoveler/Netherlands/18/99. In specific embodiments, the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55). In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues PFGSSNS (SEQ ID NO:56). In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67) from influenza A virus A/northern shoveler/Netherlands/18/99. In specific embodiments, the following amino acid residues in the 150 loop of influenza B virus B/Yamagata/16/88 PNVTSRNG (SEQ ID NO: 18) are substituted with amino acid residues KFGSSNS (SEQ ID NO:67). In specific embodiments, the following underlined and bold amino acid residues in the 160 loop of influenza B virus B/Yamagata/16/88 RDNKTA (SEQ ID NO: 5) are substituted with the following bold and underlined amino acid residues HQSGTY (SEQ ID NO: 57) from influenza A virus A/northern shoveler/Netherlands/18/99. In specific embodiments, the following amino acid residues in the 160 loop of influenza B virus B/Yamagata/16/88 RDNKTA (SEQ ID NO: 5) are substituted with amino acid residues HQSGTY (SEQ ID NO: 57). In specific embodiments, the following underlined and bold amino acid residues in the 190 helix of influenza B virus B/Yamagata/16/88 NKNQMKN (SEQ ID NO: 7) are substituted with the following underlined and bold amino acid residues TTLKMHQ (SEQ ID NO: 58) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues TTLKMHQ (SEQ ID NO: 58). In specific embodiments, the following underlined and bold amino acid residues in the 190 helix of influenza B virus B/Yamagata/16/88 NKNQMKN (SEQ ID NO: 7) are substituted with the following underlined and bold amino acid residues ATLKMHQ (SEQ ID NO: 70) from influenza A virus A/northern shoveler/Netherlands/18/99. In specific embodiments, the following amino acid residues in the 190 helix of influenza B virus B/Yamagata/16/88 NKNQMKN (SEQ ID NO: 7) are substituted with amino acid residues ATLKMHQ (SEQ ID NO: 70). In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and 190 helix, respectively, set forth in FIG. 17. In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and 190 helix, respectively, set forth in FIG. 34. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues YIP and RIKLSTENVINAETAPGGPYRL (SEQ ID NO: 63) from influenza A virus A/mallard/interior Alaska/7MP0167/2007. In specific embodiments, the following amino acid residues in influenza B virus B/Yamagata/16/88 TIP and NIRLSTHNVINAERAPGGPYRL (SEQ ID NO: 1) are substituted with amino acid residues YIP and RIKLSTFNVINAETAPGGPYRL (SEQ ID NO: 63). In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64) from influenza A virus A/mallard/interior Alaska/7MP0167/2007. In specific embodiments, the following amino acid residues in the 150 loop of influenza B virus B/Yamagata/16/88 PNVTSRNG (SEQ ID NO: 18) are substituted with amino acid residues NNTSNQGS (SEQ ID NO:64). In specific embodiments, the following underlined and bold amino acid residues in the 160 loop of influenza B virus B/Yamagata/16/88 RDNKTA (SEQ ID NO: 5) are substituted with amino acid residues LKSGQF (SEQ ID NO: 65) from influenza A virus A/mallard/interior Alaska/7MP0167/2007. In specific embodiments, the following amino acid residues in the 160 loop of influenza B virus B/Yamagata/16/88 RDNKTA (SEQ ID NO: 5) are substituted with amino acid residues LKSGQF (SEQ ID NO: 65). In specific embodiments, the following underlined and bold amino acid residues in the 190 helix of influenza B virus B/Yamagata/16/88 NKNQMKN (SEQ ID NO: 7) are substituted with the following underlined and bold amino acid residues PTSDMQI (SEQ ID NO: 66) from influenza A virus A/mallard/interior Alaska/7MP0167/2007. In specific embodiments, the following amino acid residues in the 190 helix of influenza B virus B/Yamagata/16/88 NKNQMKN (SEQ ID NO: 7) are substituted with amino acid residues PTSDMQI (SEQ ID NO: 66). In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and 190 helix, respectively, set forth in FIG. 19. In specific embodiments, the following underlined and bold amino acid residues in the 120 loop of influenza B virus B/Yamagata/16/88 TIP and NIRLSTHNVINAERAPGGPYRL (SEQ ID NO: 1) are substituted with the following underlined and bold amino acid residues NIP and RIELSTHNVINAEVAPGGPYRL (SEQ ID NO: 59) from influenza A virus A/black headed gull/Sweden/1/99. In specific embodiments, the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59). In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60) from influenza A virus A/black headed gull/Sweden/1/99. In specific embodiments, the following amino acid residues in the 150 loop of influenza B virus B/Yamagata/16/88 PNVTSRNG (SEQ ID NO: 18) are substituted with amino acid residues PDKGASS (SEQ ID NO:60). In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61) from influenza A virus A/black headed gull/Sweden/1/99. In specific embodiments, the following amino acid residues in the 160 loop of influenza B virus B/Yamagata/16/88 RDNKTA (SEQ ID NO: 5) are substituted with amino acid residues KRGNQY (SEQ ID NO: 61). In specific embodiments, the following underlined and bold amino acid residues in the 190 helix of influenza B virus B/Yamagata/16/88 NKNOMKN (SEQ ID NO: 7) are substituted with the following underlined and bold amino acid residues VSTNMAK (SEQ ID NO: 62) from influenza A virus A/black headed gull/Sweden/1/99. In specific embodiments, the following amino acid residues in the 190 helix of influenza B virus B/Yamagata/16/88 NKNQMKN (SEQ ID NO: 7) are substituted with amino acid residues VSTNMAK (SEQ ID NO: 62). In specific embodiments, a chimeric HA polypeptide described herein comprises one, two, three, or all of the following: a 120 loop, a 150 loop, a 160 loop, and/or a 190 helix with the amino acid sequences of the 120 loop, 150 loop, 160 loop, and 190 helix, respectively, set forth in FIG. 36. In certain embodiments, the chimeric HA polypeptides comprise 1, 2, 3, 4, 5 or more amino acid substitutions in the globular head domain of the influenza B virus HA which are outside of one, two, three, or all of the following: the 120 loop, 150 loop, 160 loop and 190 helix. For example, the last amino acid of the ectodomain of an influenza B virus HA may be substituted with another amino acid and amino acid 147 of influenza B virus HA (including the signal peptide) may be substituted with another amino acid. As another example, amino acid position 156 (glutamic acid) of the immature influenza B/Yamagata/16/88 virus HA may be substituted with another amino acid (for example, lysine). As another example, amino acid position 250 (glycine) of the immature influenza B/Yamagata/16/88 virus HA may be substituted with another amino acid (for example, glutamic acid). In some embodiments, the first influenza B virus strain and the second influenza B virus strain are from the same lineage. In some embodiments, the first influenza B virus strain and the second influenza B virus strain are from the same lineage but are different strains. In a specific embodiment, the first influenza B virus strain is the same strain as the second influenza B virus strain. In another embodiment, the first influenza B virus strain is a different strain than the second influenza B virus strain. In some embodiments, the first influenza B virus strain and the second influenza B virus strain are from different lineages. In some embodiments, the first influenza B virus strain is from the Yamagata lineage. In other embodiments, the first influenza B virus is from the Victoria lineage. In some embodiments, the second influenza B virus strain is from the Yamagata lineage. In other embodiments, the second influenza B virus is from the Victoria lineage. In a specific embodiment, the second influenza B virus strain is the same strain as the influenza virus backbone of an influenza virus either comprising, containing, or both the chimeric HA. In specific embodiments, the influenza A virus from which the amino acid residues are derived for the amino acid substitutions in one, two, three or more of the loops is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska 7MP0167_2007), or H13 strain (e.g., A/black headed gull/Sweden/1/99). Also provided herein are nucleic acids comprising nucleotide sequences encoding said chimeric HA polypeptides. In some embodiments, the nucleic acids comprise nucleotide sequences encoding a chimeric HA polypeptide and the 5′ non-coding region and 3′ non-coding region from the second influenza B virus strain. In some embodiments, the nucleic acids comprise nucleotide sequences encoding such a chimeric HA and the 5′ and 3′ non-coding regions of the HA of the influenza virus backbone of an influenza virus either comprising, containing, or both the chimeric HA. In a specific embodiment, the nucleic acid comprises the nucleotide sequence set forth in FIG. 29 or the complement thereof. In a specific embodiment, the nucleic acid comprises the nucleotide sequence set forth in FIG. 31 or the complement thereof. In a specific embodiment, the nucleic acid comprises the nucleotide sequence set forth in FIG. 33 or the complement thereof. In a specific embodiment, the nucleic acid comprises the nucleotide sequence set forth in FIG. 35 or the complement thereof.

In another aspect, provided herein are chimeric hemagglutinin (HA) polypeptides comprising an HA ectodomain of an influenza B virus comprising 2, 3, 4, 5, 6, 7, 8, 9, 10, 11, 12 or more amino acid substitutions within an antigenic loop of the globular head domain of the influenza B virus HA (e.g., 120 loop, 150 loop, 160 loop and/or 190 helix), wherein the amino acid substitutions substitute 2, 3, 4, 5, 6, 7, 8, 9, 10, 11, 12 or more amino acid residues in the loop of the globular head of the influenza B virus HA with random amino acid residues that do not affect the conformation/structure of the HA.

The amino acid residues in the globular head domain of an influenza A virus HA in a region corresponding to an antigenic loop (e.g., 120 loop, 150 loop, 160 loop and/or 190 helix) in the globular head domain of an influenza B virus HA may be identified using techniques known to one skilled in the art. In specific embodiments, the amino acid residues in the globular head domain of an influenza A virus HA in a region corresponding to an antigenic loop (e.g., 120 loop, 150 loop, 160 loop and/or 190 helix) in the globular head domain of an influenza B virus HA are identified by comparing the amino acid sequences and/or structural information (e.g., crystal structures) of influenza A viruses and influenza B viruses. In particular embodiments, alignments of the amino acid sequences of HA of influenza A viruses and influenza B viruses as well as assessing the viruses for structural similarity enables the skilled person in the art to select the amino acid residues in the influenza B virus HA antigenic loop to substitute with amino acid residues from a corresponding region in the globular head domain of an influenza A virus HA. For example, one might want to refrain from substituting amino acid residues, such as cysteine, proline or both, in the influenza B virus HA antigenic loop that may impact the folding of the chimeric HA with amino acid residues from a corresponding region in the globular head domain of an influenza A virus HA. In addition, one might want to refrain from substituting amino acid residues in the influenza B virus HA antigenic loop that impact the coding for N-linked glycosylation sites (N—X—S/T). In selecting the amino acid residues to substitute, care should be taken to maintain the conformation/structure of the HA. In some embodiments, amino acid residues that are highly conserved in an antigenic loop of the globular head domain of influenza B virus HAs, one might want to refrain from substituting with amino acid residues from a corresponding region in the globular head domain of an influenza A virus HA. For example, those amino acid residues identified by Wang et al., 2008, Journal of Virology 82:3011-3020 as being variant among influenza B viruses may be selected as amino acid residues within an antigenic loop of the globular head domain of an influenza B virus to substitute with other amino acid residues (e.g., other amino acid residues from a corresponding region of the globular head domain of an influenza A virus HA), while those amino acid residues within the antigenic loop of the globular head domain of an influenza B virus HA may not be substituted. In a specific embodiment, when amino acid residues that are highly conserved in an antigenic loop of the globular head domain of influenza B virus HAs and amino acid residues in a corresponding region of the globular head domain of influenza A virus HAs, one might want to refrain from substituting with amino acid residues in the antigenic loop of the globular head domain of an influenza B virus HA with amino acid residues from a corresponding region in the globular head domain of an influenza A virus HA. For example, one of skill in the art may not want to substitute the methionine in the 190 helix of an influenza B virus with another amino acid residue. See, e.g., Section 6, infra. In certain embodiments, with respect to amino acid residues such as proline found in an antigenic loop of the globular head domain of an influenza B virus HA, one might want to refrain from substituting with amino acid residues from a corresponding region in the globular head domain of an influenza A virus HA. In some embodiments, with respect to amino acid residues such as cysteine, proline or both found in an antigenic loop of the globular head domain of an influenza B virus HA, one might want to refrain from substituting with amino acid residues from a corresponding region in the globular head domain of an influenza A virus HA. In certain embodiments, one might want to refrain from substituting amino acid residues such as proline found in an antigenic loop of the globular head domain of an influenza B virus HA with amino acid residues from a corresponding region in the globular head domain of an influenza A virus HA. In specific embodiments, the amino acid residues substituted in an antigenic loop of the globular head domain of an influenza B virus are not consecutive amino acid residues. For example, amino acid residues that are found conformationally close to one another may be substituted for other amino acid residues. In other embodiments, the amino acid residues substituted in an antigenic loop of the globular head domain of an influenza B virus are consecutive amino acid residues. In certain embodiments, an amino acid residue found in the antigenic loop of an influenza B virus is substituted with a conservative amino acid residue (i.e., a conservative substitution). The effect of amino acid substitutions on the conformation/structure may be determined by assays known to one of skill in the art, e.g., structure programs, crystallography, or functional assays. See, e.g., Section 5.11, infra, and Section 6, infra. In a particular embodiment, the chimeric HA polypeptides may be evaluated for antigenic conservation using a panel of monoclonal antibodies that bind to conserved epitopes in the globular head domain of HA and the stem domain of HA. In a specific embodiment, the methods described in Section 6, infra, are used to evaluate antigenic conservation of the chimeric HA. In addition, the chimeric HA polypeptides described herein may be evaluated to determine whether the antigenic loops of the influenza B virus HA were mutated using techniques known to one of skill in the art or described herein (see, e.g., Section 6, infra, including the HI assay described therein). In particular, the chimeric HA polypeptides described herein may be evaluated to determine if the amino acid substitutions in the antigenic loop(s) of the influenza B virus HA result in loss of a variable region(s) of the influenza B virus HA using techniques known to one of skill in the art or described herein (see, e.g., Section 6, infra, including the HI assay described therein). In a specific embodiment, the chimeric HA polypeptides described herein may be evaluated to determine if the amino acid substitutions in the antigenic loop(s) of the influenza B virus HA reduce or eliminate the immunodominant epitopes of the influenza B virus HA using techniques known to one of skill in the art or described herein (see, e.g., Section 6, infra, including the HI assay described therein). In a specific embodiment, a chimeric HA polypeptide described herein is assessed in an HI assay, such as described in Section 6, infra, to evaluate the replacement of the antigenic loop(s) in the influenza B virus HA.

In some embodiments, 1, 2, 3, 4, 5, 6, 7, 8, 9, 10, 11, 12, 13, 14, 15, 16, 17, 18, 19, 20 or more amino acid residues in 120 loop of an influenza B virus HA are substituted with 1, 2, 3, 4, 5, 6, 7, 8, 9, 10, 11, 12, 13, 14, 15, 16, 17, 18, 19, 20 or more amino acid residues in site E of the globular head domain of an influenza A virus H3 HA. In some embodiments, 1, 2, 3, 4, 5, 6, 7, 8, 9, 10, 11, 12, 13, 14, 15, 16, 17, 18, 19, 20 or more amino acid residues in 120 loop of an influenza B virus HA are substituted with 1, 2, 3, 4, 5, 6, 7, 8, 9, 10, 11, 12, 13, 14, 15, 16, 17, 18, 19, 20 or more amino acid residues in site Sa of the globular head domain of an influenza A virus H1 HA. In certain embodiments, 1, 2, 3, 4, 5, 6, 7, 8, 9, 10, 11, 12, 13, 14, 15, 16, 17, 18, 19, 20 or more amino acid residues in 120 loop of an influenza B virus HA are substituted with 1, 2, 3, 4, 5, 6, 7, 8, 9, 10, 11, 12, 13, 14, 15, 16, 17, 18, 19, 20 or more amino acid residues in site Cb of the globular head domain of an influenza A virus H1 HA. In some embodiments, 1, 2, 3, 4, 5, 6, 7, 8, 9, 10, 11, 12, 13, 14, 15, 16, 17, 18, 19, 20 or more amino acid residues in 120 loop of an influenza B virus HA are substituted with 1, 2, 3, 4, 5, 6, 7, 8, 9, 10, 11, 12, 13, 14, 15, 16, 17, 18, 19, 20 or more amino acid residues in sites Sa and/or Cb of the globular head domain of an influenza A virus H1 HA.

In certain embodiments, 1, 2, 3, 4, 5, 6, 7, 8, 9 or more amino acid residues in 150 loop of an influenza B virus HA are substituted with 1, 2, 3, 4, 5, 6, 7, 8, 9 or more amino acid residues in site A of the globular head domain of an influenza A virus H3 HA. In some embodiments, 1, 2, 3, 4, 5, 6, 7, 8, 9 or more amino acid residues in 150 loop of an influenza B virus HA are substituted with 1, 2, 3, 4, 5, 6, 7, 8, 9 or more amino acid residues in site Ca of the globular head domain of an influenza A virus H1 HA.

In certain embodiments, 1, 2, 3, 4, 5 or more amino acid residues in 160 loop of an influenza B virus HA are substituted with 1, 2, 3, 4, 5 or more amino acid residues in site B of the globular head domain of an influenza A virus H3 HA. In some embodiments, 1, 2, 3, 4, 5 or more amino acid residues in 160 loop of an influenza B virus HA are substituted with 1, 2, 3, 4, 5 or more amino acid residues in site Sa of the globular head domain of an influenza A virus H1 HA.

In certain embodiments, 1, 2, 3, 4, 5, 6, 7, 8 or more amino acid residues in 190 helix of an influenza B virus HA are substituted with 1, 2, 3, 4, 5, 6, 7, 8 or more amino acid residues in site B of the globular head domain of an influenza A virus H3 HA. In some embodiments, 1, 2, 3, 4, 5, 6, 7, 8 or more amino acid residues in 190 helix of an influenza B virus HA are substituted with 1, 2, 3, 4, 5, 6, 7, 8 or more amino acid residues in site Sb of the globular head domain of an influenza A virus H1 HA.

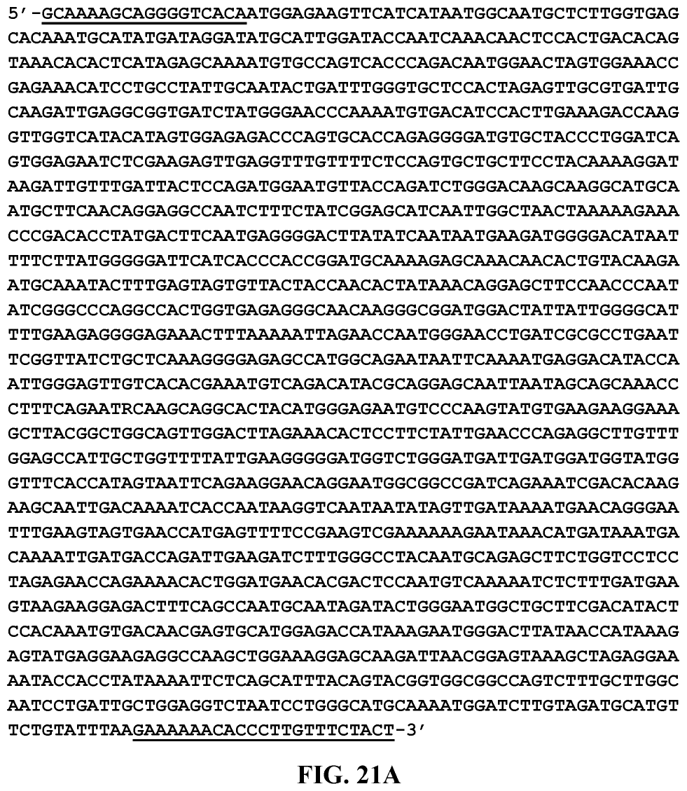

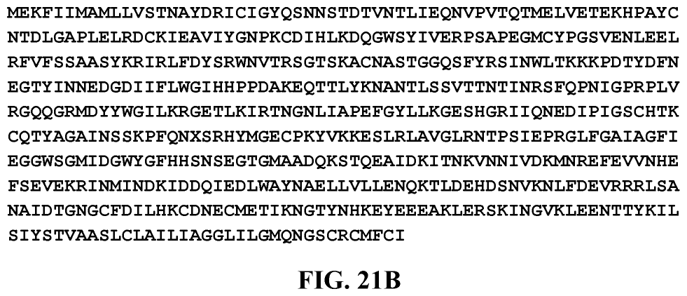

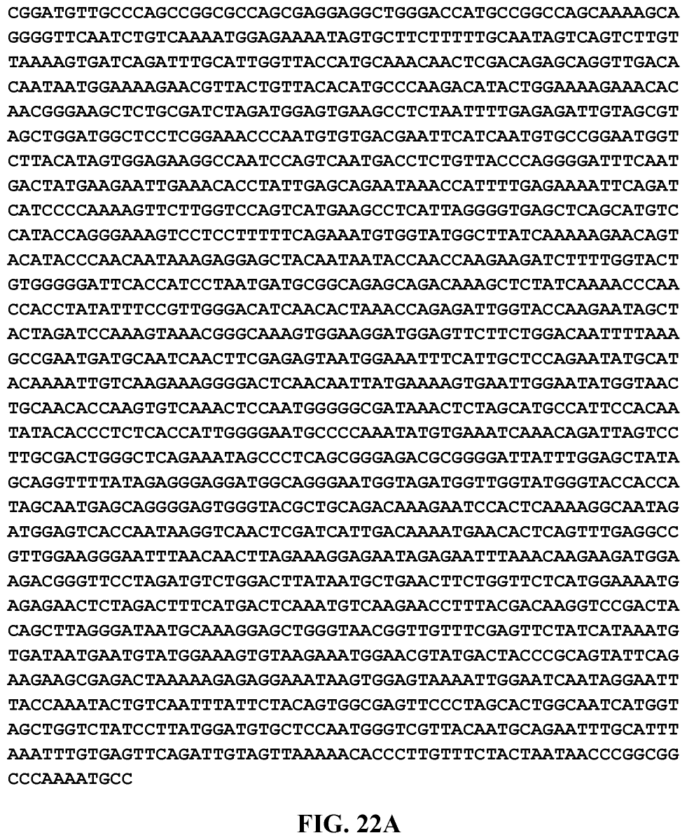

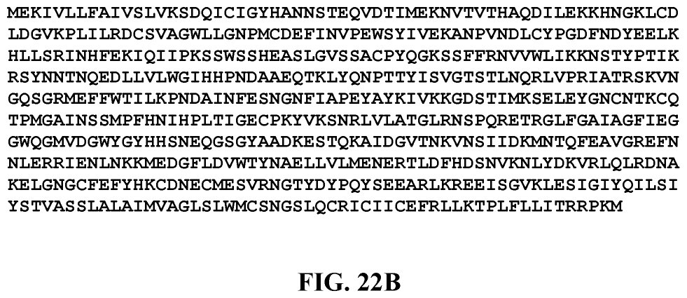

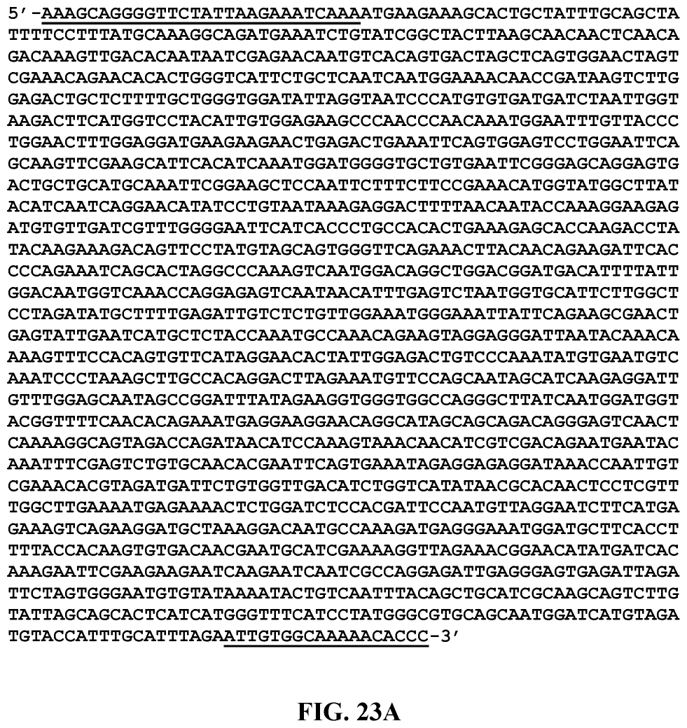

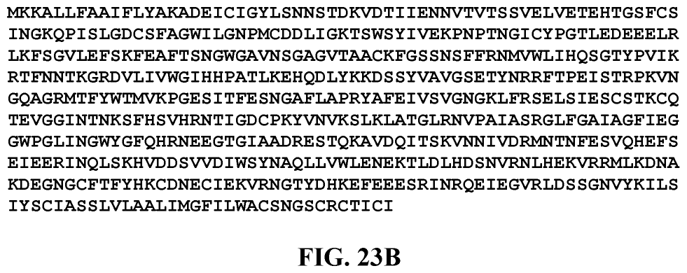

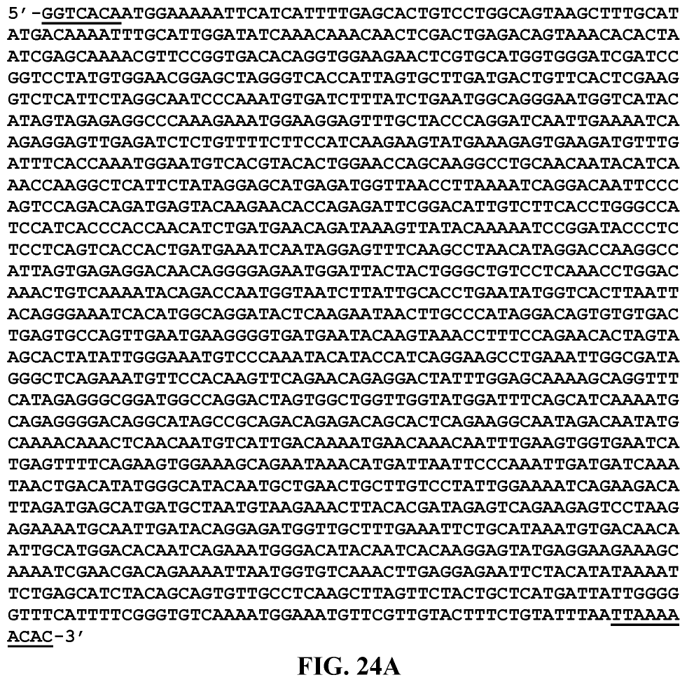

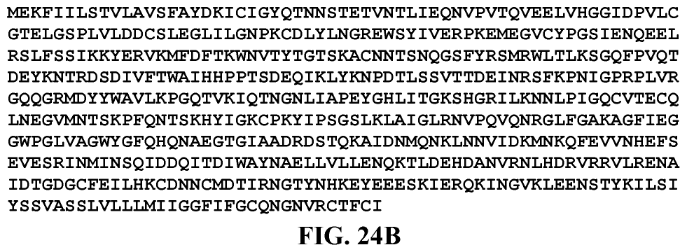

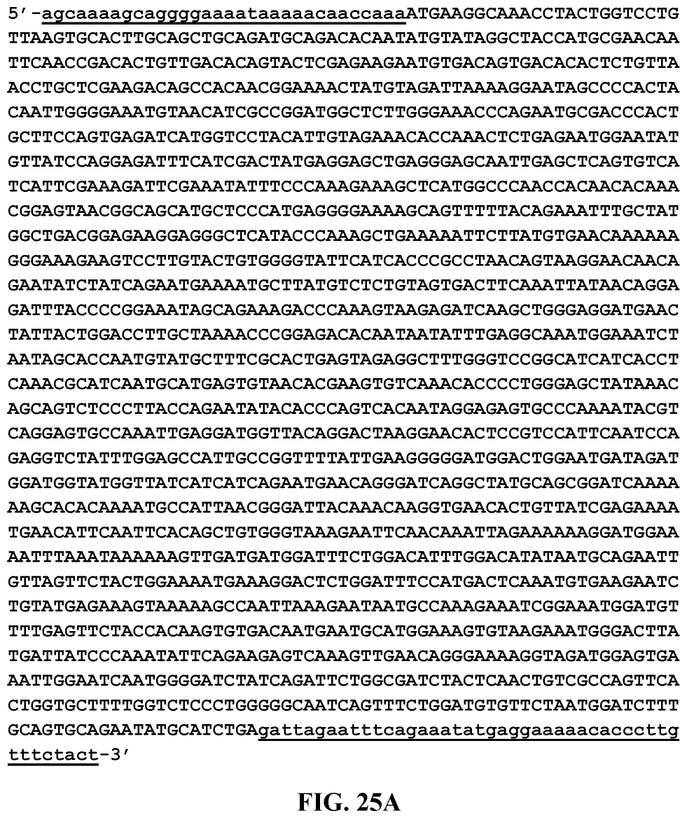

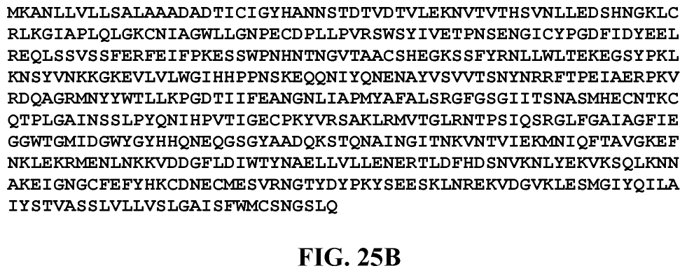

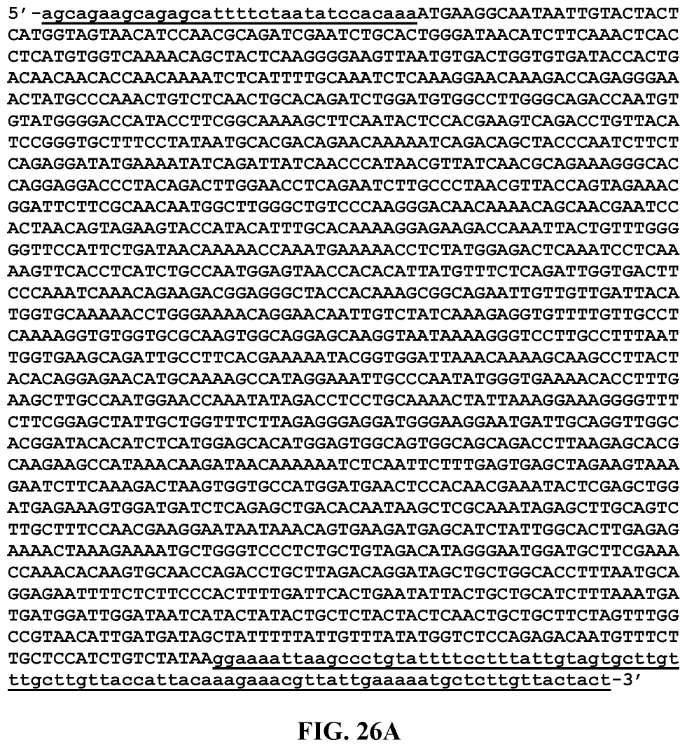

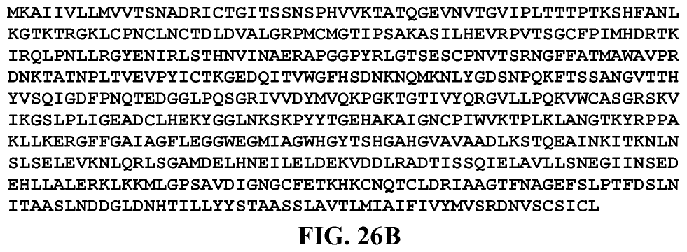

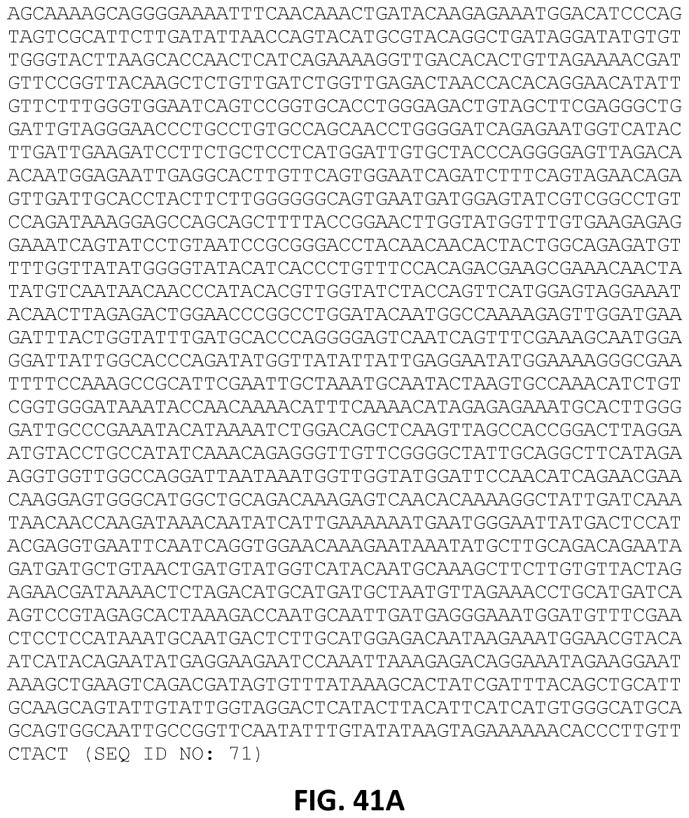

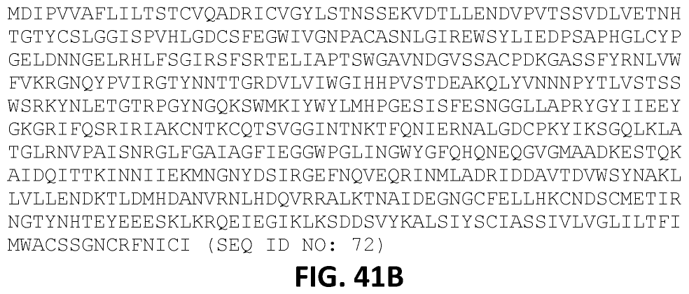

In a specific embodiment, the influenza A virus HA utilized in the generation of a chimeric HA polypeptide described herein is the HA from an influenza virus of the H1, H2, H3, H4, H5, H6, H7, H8, H9, H10, H11, H12, H13, H14, H15, H16, H17, or H18 subtype. In a specific embodiment, the influenza A virus HA utilized in the generation of a chimeric HA polypeptide described herein is the HA from an influenza virus of the H2, H3, H4, H5, H6, H7, H8, H9, H10, H11, H12, H13, H14, H15, H16, H17, or H18 subtype. In a specific embodiment, the influenza A virus HA utilized in the generation of a chimeric HA polypeptide described herein is the HA from an influenza virus of the H5, H8, H11, H12, or H13 subtype. In a specific embodiment, the influenza A virus HA utilized in the generation of a chimeric HA polypeptide described herein is the HA from an influenza virus of the H5 subtype. In a specific embodiment, the influenza A virus HA utilized in the generation of a chimeric HA polypeptide described herein is the HA from an influenza virus of the H8 subtype. In a specific embodiment, the influenza A virus HA utilized in the generation of a chimeric HA polypeptide described herein is the HA from an influenza virus of the H11 subtype. In a specific embodiment, the influenza A virus HA utilized in the generation of a chimeric HA polypeptide described herein is the HA from an influenza virus of the H12 subtype. In a specific embodiment, the influenza A virus HA utilized in the generation of a chimeric HA polypeptide described herein is the HA from an influenza virus of the H13 subtype. In a specific embodiment, the influenza A virus HA utilized in the generation of a chimeric HA polypeptide described herein is the HA from an avian influenza virus. In a specific embodiment, the influenza A virus HA utilized in the generation of a chimeric HA polypeptide described herein is the HA from influenza A/mallard/Sweden/24/2002 virus (GenBank Accession No. CY060249.1; GenBank GI No. 294441479; see, also, FIG. 21A and FIG. 21B). In a specific embodiment, the influenza A virus HA utilized in the generation of a chimeric HA polypeptide described herein is the HA from influenza A/Vietnam/1203/04 virus (GenBank Accession No. EF541403.1; GenBank GI No. 145284465; see, also, FIG. 22A and FIG. 22B and Steel et al., 2009, Journal of Virology, 83 (4): 1742-1753 for the HA of influenza A/Vietnam/1203/04 (HALo) virus). In a specific embodiment, the influenza A virus HA utilized in the generation of a chimeric HA polypeptide described herein is the HA from influenza A/northern shoveler/Netherlands/18/99 virus (GenBank Accession No. CY060417.1; GenBank GI No. 294441876; see, also, FIG. 23A and FIG. 23B). In a specific embodiment, the influenza A virus HA utilized in the generation of a chimeric HA polypeptide described herein is the HA from influenza A_mallard_interior Alaska 7MP0167_2007 virus (GenBank Accession No. CY077198.1; GenBank GI No. 312652817; see, also, FIG. 24A and FIG. 24B). In a specific embodiment, the influenza A virus HA utilized in the generation of a chimeric HA polypeptide described herein is the HA from influenza A/Puerto Rico/8/34 virus (GenBank Accession No. AF389118.1; GenBank GI No. 21693168; see, also, FIG. 25A and FIG. 25B). In a specific embodiment, the influenza A virus HA utilized in the generation of a chimeric HA polypeptide described herein is the HA from influenza A/black headed gull/Sweden/1/99 (GenBank Accession No. AY684887.1; see, also, FIGS. 41A and 41B). In a specific embodiment, the influenza B virus HA utilized in the generation of a chimeric HA polypeptide described herein is an HA from an influenza B virus of the Yamagata lineage. In a specific embodiment, the influenza B virus HA utilized in the generation of a chimeric HA polypeptide described herein is an HA from an influenza B virus of the Victoria lineage. In a specific embodiment, the influenza B virus HA utilized in the generation of a chimeric HA polypeptide described herein is the HA from influenza B/Yamagata/16/88 virus (see, FIG. 26A and FIG. 26B). In a specific embodiment, the influenza B virus HA utilized in the generation of a chimeric HA polypeptide described herein is the HA from an influenza B/Malaysia/2506/04 mouse adapted (MA) virus (see, e.g., SEQ ID NO:73 and 83). In a specific embodiment, the influenza B virus HA utilized in the generation of a chimeric HA polypeptide described herein is the HA from influenza B/Malaysia/2506/04 virus (see, e.g., GenBank Accession No. CY040449.1).

In a specific embodiment, a chimeric HA polypeptide is a chimeric HA polypeptide described in Section 6, infra. In a specific embodiment, a chimeric HA polypeptide comprises the amino acid sequence of the chimeric HA polypeptide in FIG. 13, 15, 17, 19, 30, 32, 34, or 36. In another specific embodiment, a chimeric HA polypeptide comprises the amino acid sequence of the chimeric HA polypeptide in FIG. 13, 15, 17, 19, 30, 32, 24 or 36 without the signal peptide. In another specific embodiment, a chimeric HA polypeptide comprises the amino acid sequences of the ectodomain of the chimeric HA polypeptide in FIG. 13, 15, 17, 19, 30, 32, 34 or 36.

In a specific embodiment, the influenza B virus HA sequence utilized to generate a chimeric HA polypeptide described herein is the HA sequence from an influenza B virus described in Section 5.4, infra. In a specific embodiment, the influenza B virus HA sequence utilized to generate a chimeric HA polypeptide described herein is the HA sequence from an influenza B virus described in Section 6, infra. In a specific embodiment, the influenza A virus HA sequence utilized to generate a chimeric HA polypeptide described herein is the HA sequence from an influenza A virus described in Section 5.4, infra. In a specific embodiment, the influenza A virus HA sequence utilized to generate a chimeric HA polypeptide described herein is the HA sequence from an influenza A virus described in Section 6, infra. For example, the influenza A virus HA may be from a group 1 or a group 2 virus. In specific embodiments, the influenza A virus HA is from an H1, H2, H3, H4, H5, H6, H7, H8, H9, H10, H11, H12, H13, H14, H15, H16, or H17 influenza A virus.

In certain embodiments, the chimeric influenza virus hemagglutinin polypeptides provided herein are capable of forming a three dimensional structure that is similar to the three dimensional structure of a native influenza hemagglutinin. Structural similarity might be evaluated based on any technique deemed suitable by those of skill in the art. For instance, reaction, e.g. under non-denaturing conditions, of a chimeric influenza virus hemagglutinin polypeptide with a neutralizing antibody or antiserum that recognizes a native influenza hemagglutinin might indicate structural similarity. Useful neutralizing antibodies or antisera are described in, e.g. Sui, et al., 2009, Nat. Struct. Mol. Biol. 16 (3): 265-273, Ekiert et al., Feb. 26, 2009, Science [DOI: 10.1126/science.1171491], and Kashyap et al., 2008, Proc. Natl. Acad. Sci. USA 105 (16): 5986-5991, the contents of which are hereby incorporated by reference in their entireties. In certain embodiments, the antibody or antiserum is an antibody or antiserum that reacts with a non-contiguous epitope (i.e., not contiguous in primary sequence) that is formed by the tertiary or quaternary structure of a hemagglutinin.

In certain embodiments, a chimeric influenza hemagglutinin (HA) polypeptide described herein retains one, two, or more, or all of the functions of a wild-type influenza HA. Nonlimiting examples of functions of a wild-type influenza HA include fusogenic activity, receptor binding activity, budding, and particle formation. In a specific embodiment, a chimeric influenza hemagglutinin (HA) polypeptide described herein has fusogenic activity. Assays known to one skilled in the art can be utilized the assess the fusogenic activity of a chimeric influenza hemagglutinin (HA) polypeptide described herein, such as, for example, immunofluorescence assays and pseudotyped virus-like-particle assays.

### 5.2 Nucleic Acids Encoding Chimeric Hemagglutinin (HA) Polypeptide

Provided herein are nucleic acids comprising nucleotide sequences that encode the chimeric influenza virus hemagglutinin polypeptides described herein. Due to the degeneracy of the genetic code, any nucleic acid that encodes a chimeric hemagglutinin (HA) polypeptide described herein is encompassed herein. In certain embodiments, nucleic acids corresponding to naturally occurring influenza virus nucleic acids encoding an HA1 domain (e.g., an HA1 stem segment (such as, an HA1N-terminal stem segment and an HA1C-terminal stem segment)), HA2 domain, HA luminal domain, HA transmembrane domain, and/or HA cytoplasmic domain are used to produce a chimeric influenza virus hemagglutinin polypeptide. In certain embodiments, the nucleic acid comprises one, two, or three of the following: nucleotide sequences encoding an influenza virus HA signal peptide, nucleotide sequences encoding an influenza virus HA transmembrane domain, and nucleotide sequences encoding an influenza virus HA cytoplasmic domain. In specific embodiments, the nucleic acid comprises nucleotide sequences encoding said chimeric HA polypeptide and preferably comprises the 5′ non-coding region and 3′ non-coding region from the HA of the same influenza virus as the influenza virus engineered to express the chimeric HA polypeptide. In specific embodiments, the nucleic acid comprises nucleotide sequences encoding said chimeric HA polypeptide, the 5′ 5′ non-coding region and 3′ non-coding region from the HA of the same influenza virus as the influenza virus engineered to express the chimeric HA polypeptide, and nucleotide sequences encoding the influenza virus HA signal peptide from the HA of the same influenza virus as the influenza virus engineered to express the chimeric HA polypeptide. In specific embodiments, the nucleic acid comprises nucleotide sequences encoding said chimeric HA polypeptide, the 5′ 5′ non-coding region and 3′ non-coding region from the HA of the same influenza virus as the influenza virus engineered to express the chimeric HA polypeptide, and nucleotide sequences encoding one, two, or three of the following: the influenza virus HA signal peptide, the influenza virus HA transmembrane domain, and the influenza virus HA cytoplasmic domain from the HA of the same influenza virus as the influenza virus engineered to express the chimeric HA polypeptide.

Also provided herein are nucleic acids capable of hybridizing to a nucleic acid encoding a chimeric influenza virus hemagglutinin polypeptide. In certain embodiments, provided herein are nucleic acids capable of hybridizing to a fragment of a nucleic acid encoding a chimeric influenza virus hemagglutinin polypeptide. In other embodiments, provided herein are nucleic acids capable of hybridizing to the full length of a nucleic acid encoding a chimeric influenza virus hemagglutinin polypeptide. General parameters for hybridization conditions for nucleic acids are described in Sambrook et al., Molecular Cloning-A Laboratory Manual (2nd Ed.), Vols. 1-3, Cold Spring Harbor Laboratory, Cold Spring Harbor, New York (1989), and in Ausubel et al., Current Protocols in Molecular Biology, vol. 2, Current Protocols Publishing, New York (1994). Hybridization may be performed under high stringency conditions, medium stringency conditions, or low stringency conditions. Those of skill in the art will understand that low, medium and high stringency conditions are contingent upon multiple factors all of which interact and are also dependent upon the nucleic acids in question. For example, high stringency conditions may include temperatures within 5° C. melting temperature of the nucleic acid(s), a low salt concentration (e.g., less than 250 mM), and a high co-solvent concentration (e.g., 1-20% of co-solvent, e.g., DMSO). Low stringency conditions, on the other hand, may include temperatures greater than 10° C. below the melting temperature of the nucleic acid(s), a high salt concentration (e.g., greater than 1000 mM) and the absence of co-solvents. In specific embodiments, the nucleic acid is capable of hybridizing under high stringency conditions to the full length of the nucleotide sequence set forth in SEQ ID NO: 20. In specific embodiments, the nucleic acid is capable of hybridizing under high stringency conditions to the full length of the coding sequence of the nucleotide sequence set forth in SEQ ID NO: 20. In specific embodiments, the nucleic acid is capable of hybridizing under high stringency conditions to the full length of the nucleotide sequence set forth in SEQ ID NO: 22. In specific embodiments, the nucleic acid is capable of hybridizing under high stringency conditions to the full length of the coding sequence of the nucleotide sequence set forth in SEQ ID NO: 22. In specific embodiments, the nucleic acid is capable of hybridizing under high stringency conditions to the full length of the nucleotide sequence set forth in SEQ ID NO: 24. In specific embodiments, the nucleic acid is capable of hybridizing under high stringency conditions to the full length of the coding sequence of the nucleotide sequence set forth in SEQ ID NO: 24. In specific embodiments, the nucleic acid is capable of hybridizing under high stringency conditions to the full length of the nucleotide sequence set forth in SEQ ID NO: 26. In specific embodiments, the nucleic acid is capable of hybridizing under high stringency conditions to the full length of the coding sequence of the nucleotide sequence set forth in SEQ ID NO: 26. In specific embodiments, the nucleic acid is capable of hybridizing under high stringency conditions to the full length of the nucleotide sequence set forth in SEQ ID NO: 43. In specific embodiments, the nucleic acid is capable of hybridizing under high stringency conditions to the full length of the coding sequence of the nucleotide sequence set forth in SEQ ID NO: 43. In specific embodiments, the nucleic acid is capable of hybridizing under high stringency conditions to the full length of the nucleotide sequence set forth in SEQ ID NO: 45. In specific embodiments, the nucleic acid is capable of hybridizing under high stringency conditions to the full length of the coding sequence of the nucleotide sequence set forth in SEQ ID NO: 45. In specific embodiments, the nucleic acid is capable of hybridizing under high stringency conditions to the full length of the nucleotide sequence set forth in SEQ ID NO: 47. In specific embodiments, the nucleic acid is capable of hybridizing under high stringency conditions to the full length of the coding sequence of the nucleotide sequence set forth in SEQ ID NO: 47. In specific embodiments, the nucleic acid is capable of hybridizing under high stringency conditions to the full length of the nucleotide sequence set forth in SEQ ID NO: 49. In specific embodiments, the nucleic acid is capable of hybridizing under high stringency conditions to the full length of the coding sequence of the nucleotide sequence set forth in SEQ ID NO: 49. In a specific embodiment, a chimeric HA is encoded by the nucleic acid sequence set forth in FIG. 12A and FIG. 12B, or the complement thereof. In a specific embodiment, a chimeric HA is encoded by a nucleic acid sequence comprising the nucleotide sequence encoding the ectodomain set forth in FIG. 12A and FIG. 12B (i.e., excluding the 5′ and 3′ noncoding regions and nucleic acid sequences encoding the signal peptide, transmembrane domain, and cytoplasmic domain set forth in FIG. 12A and FIG. 12B), or the complement thereof. In a specific embodiment, a chimeric HA is encoded by the nucleic acid sequence set forth in FIG. 14A and FIG. 14B, or the complement thereof. In a specific embodiment, a chimeric HA is encoded by a nucleic acid sequence comprising the nucleotide sequence encoding the ectodomain set forth in FIG. 14A and FIG. 14B (i.e., excluding the 5′ and 3′ noncoding regions and nucleic acid sequences encoding the signal peptide, transmembrane domain, and cytoplasmic domain set forth in FIG. 14A and FIG. 14B), or the complement thereof. In a specific embodiment, a chimeric HA is encoded by the nucleic acid sequence set forth in FIG. 16A and FIG. 16B, or the complement thereof. In a specific embodiment, a chimeric HA is encoded by a nucleic acid sequence comprising a nucleotide sequence encoding the ectodomain set forth in FIG. 16A and FIG. 16B (i.e., excluding the 5′ and 3′ noncoding regions and nucleic acid sequences encoding the signal peptide, transmembrane domain, and cytoplasmic domain set forth in FIG. 16A and FIG. 16B), or the complement thereof. In a specific embodiment, a chimeric HA is encoded by the nucleic acid sequence set forth in FIG. 18A and FIG. 18B, or the complement thereof. In a specific embodiment, a chimeric HA is encoded by a nucleic acid sequence comprising the nucleotide sequence encoding the ectodomain set forth in FIG. 18A and FIG. 18B (i.e., excluding the 5′ and 3′ noncoding regions and nucleic acid sequences encoding the signal peptide, transmembrane domain, and cytoplasmic domain set forth in FIG. 18A and FIG. 18B), or the complement thereof. In a specific embodiment, a chimeric HA is encoded by the nucleic acid sequence set forth in FIG. 29, or the complement thereof. In a specific embodiment, a chimeric HA is encoded by a nucleic acid sequence comprising the nucleotide sequence encoding the ectodomain set forth in FIG. 29 (i.e., excluding the 5′ and 3′ noncoding regions and nucleic acid sequences encoding the signal peptide, transmembrane domain, and cytoplasmic domain set forth in FIG. 29), or the complement thereof. In a specific embodiment, a chimeric HA is encoded by the nucleic acid sequence set forth in FIG. 31, or the complement thereof. In a specific embodiment, a chimeric HA is encoded by a nucleic acid sequence comprising the nucleotide sequence encoding the ectodomain set forth in FIG. 31 (i.e., excluding the 5′ and 3′ noncoding regions and nucleic acid sequences encoding the signal peptide, transmembrane domain, and cytoplasmic domain set forth in FIG. 31), or the complement thereof. In a specific embodiment, a chimeric HA is encoded by the nucleic acid sequence set forth in FIG. 33, or the complement thereof. In a specific embodiment, a chimeric HA is encoded by a nucleic acid sequence comprising the nucleotide sequence encoding the ectodomain set forth in FIG. 33 (i.e., excluding the 5′ and 3′ noncoding regions and nucleic acid sequences encoding the signal peptide, transmembrane domain, and cytoplasmic domain set forth in FIG. 33), or the complement thereof. In a specific embodiment, a chimeric HA is encoded by the nucleic acid sequence set forth in FIG. 35, or the complement thereof. In a specific embodiment, a chimeric HA is encoded by a nucleic acid sequence comprising the nucleotide sequence encoding the ectodomain set forth in FIG. 35 (i.e., excluding the 5′ and 3′ noncoding regions and nucleic acid sequences encoding the signal peptide, transmembrane domain, and cytoplasmic domain set forth in FIG. 35), or the complement thereof.

In a specific embodiment, a chimeric HA polypeptide is encoded by a nucleic acid sequence comprising the nucleotide sequence encoding the ectodomain set forth in FIG. 12 or a complement thereof, and one, two or all of the following: (1) the nucleotide sequence encoding the signal peptide set forth in FIG. 12, or a complement thereof; (2) the nucleotide sequence encoding the transmembrane domain set forth in FIG. 12, or a complement thereof; and (3) the nucleotide sequence encoding the cytoplasmic domain set forth in FIG. 12, or a complement thereof. In a specific embodiment, a chimeric HA polypeptide is encoded by a nucleic acid sequence comprising the nucleotide sequence encoding the ectodomain set forth in FIG. 14 or a complement thereof, and one, two or all of the following: (1) the nucleotide sequence encoding the signal peptide set forth in FIG. 14, or a complement thereof; (2) the nucleotide sequence encoding the transmembrane domain set forth in FIG. 14, or a complement thereof; and (3) the nucleotide sequence encoding the cytoplasmic domain set forth in FIG. 14, or a complement thereof. In a specific embodiment, a chimeric HA polypeptide is encoded by a nucleic acid sequence comprising the nucleotide sequence encoding the ectodomain set forth in FIG. 16 or a complement thereof, and one, two or all of the following: (1) the nucleotide sequence encoding the signal peptide set forth in FIG. 16, or a complement thereof; (2) the nucleotide sequence encoding the transmembrane domain set forth in FIG. 16, or a complement thereof; and (3) the nucleotide sequence encoding the cytoplasmic domain set forth in FIG. 16, or a complement thereof. In a specific embodiment, a chimeric HA polypeptide is encoded by a nucleic acid sequence comprising the nucleotide sequence encoding the ectodomain set forth in FIG. 18 or a complement thereof, and one, two or all of the following: (1) the nucleotide sequence encoding the signal peptide set forth in FIG. 18, or a complement thereof; (2) the nucleotide sequence encoding the transmembrane domain set forth in FIG. 18, or a complement thereof; and (3) the nucleotide sequence encoding the cytoplasmic domain set forth in FIG. 18, or a complement thereof. In a specific embodiment, a chimeric HA polypeptide is encoded by a nucleic acid sequence comprising the nucleotide sequence encoding the ectodomain set forth in FIG. 29 or a complement thereof, and one, two or all of the following: (1) the nucleotide sequence encoding the signal peptide set forth in FIG. 29, or a complement thereof; (2) the nucleotide sequence encoding the transmembrane domain set forth in FIG. 29, or a complement thereof; and (3) the nucleotide sequence encoding the cytoplasmic domain set forth in FIG. 29, or a complement thereof. In a specific embodiment, a chimeric HA polypeptide is encoded by a nucleic acid sequence comprising the nucleotide sequence encoding the ectodomain set forth in FIG. 31 or a complement thereof, and one, two or all of the following: (1) the nucleotide sequence encoding the signal peptide set forth in FIG. 31, or a complement thereof; (2) the nucleotide sequence encoding the transmembrane domain set forth in FIG. 31, or a complement thereof; and (3) the nucleotide sequence encoding the cytoplasmic domain set forth in FIG. 31, or a complement thereof. In a specific embodiment, a chimeric HA polypeptide is encoded by a nucleic acid sequence comprising the nucleotide sequence encoding the ectodomain set forth in FIG. 33 or a complement thereof, and one, two or all of the following: (1) the nucleotide sequence encoding the signal peptide set forth in FIG. 33, or a complement thereof; (2) the nucleotide sequence encoding the transmembrane domain set forth in FIG. 33, or a complement thereof; and (3) the nucleotide sequence encoding the cytoplasmic domain set forth in FIG. 33, or a complement thereof. In a specific embodiment, a chimeric HA polypeptide is encoded by a nucleic acid sequence comprising the nucleotide sequence encoding the ectodomain set forth in FIG. 35 or a complement thereof, and one, two or all of the following: (1) the nucleotide sequence encoding the signal peptide set forth in FIG. 35, or a complement thereof; (2) the nucleotide sequence encoding the transmembrane domain set forth in FIG. 35, or a complement thereof; and (3) the nucleotide sequence encoding the cytoplasmic domain set forth in FIG. 35, or a complement thereof.

In a specific embodiment, a nucleic acid sequence encoding a chimeric HA polypeptide comprises the nucleotide sequence set forth in FIG. 12, 14, 16, 18, 29, 31, 33, or 35, or a complement thereof. In another specific embodiment, a nucleic acid sequence encoding a chimeric HA polypeptide comprises the nucleotide sequence set forth in FIG. 12, 14, 16, 18, 29, 31, 33, or 35, or a complement thereof, without the signal peptide. In another specific embodiment, a nucleic acid sequence encoding a chimeric HA polypeptide comprises the nucleotide sequence set forth in FIG. 12, 14, 16, 18, 29, 31, 33, or 35, or a complement thereof, without the 5′ non-coding region, 3′ non-coding region or both. In another specific embodiment, a nucleic acid sequence encoding a chimeric HA polypeptide comprises the nucleotide sequence set forth in FIG. 12, 14, 16, 18, 29, 31, 33, or 35, or a complement thereof, without the signal peptide and without the 5′ non-coding region, 3′ non-coding region or both.

In some embodiments, a nucleic acid encoding a chimeric influenza virus hemagglutinin polypeptide is isolated. In certain embodiments, an “isolated” nucleic acid refers to a nucleic acid molecule which is separated from other nucleic acid molecules which are present in the natural source of the nucleic acid. In other words, the isolated nucleic acid can comprise heterologous nucleic acids that are not associated with it in nature. In other embodiments, an “isolated” nucleic acid, such as a cDNA molecule, can be substantially free of other cellular material, or culture medium when produced by recombinant techniques, or substantially free of chemical precursors or other chemicals when chemically synthesized. The term “substantially free of cellular material” includes preparations of nucleic acid in which the nucleic acid is separated from cellular components of the cells from which it is isolated or recombinantly produced. Thus, nucleic acid that is substantially free of cellular material includes preparations of nucleic acid having less than about 30%, 20%, 10%, or 5% (by dry weight) of other nucleic acids. The term “substantially free of culture medium” includes preparations of nucleic acid in which the culture medium represents less than about 50%, 20%, 10%, or 5% of the volume of the preparation. The term “substantially free of chemical precursors or other chemicals” includes preparations in which the nucleic acid is separated from chemical precursors or other chemicals which are involved in the synthesis of the nucleic acid. In specific embodiments, such preparations of the nucleic acid have less than about 50%, 30%, 20%, 10%, 5% (by dry weight) of chemical precursors or compounds other than the nucleic acid of interest.

In addition, provided herein are nucleic acids encoding the individual components of a chimeric influenza virus hemagglutinin polypeptide. In specific embodiments, nucleic acids encoding the globular head domain and/or the stem domain of the chimeric influenza virus hemagglutinin polypeptide are provided. Nucleic acids encoding components of a chimeric influenza virus hemagglutinin polypeptide may be assembled using standard molecular biology techniques known to one of skill in the art. In specific embodiments, the individual components of a chimeric influenza virus hemagglutinin polypeptide can be expressed by the same or different vector.

### 5.3 Expression of Chimeric Hemagglutinin (HA) Polypeptide

Provided herein are vectors, including expression vectors, containing a nucleic acid encoding a chimeric influenza virus hemagglutinin polypeptide described herein. In a specific embodiment, the vector is an expression vector that is capable of directing the expression of a nucleic acid encoding a chimeric influenza virus hemagglutinin polypeptide. Non-limiting examples of expression vectors include, but are not limited to, plasmids and viral vectors, such as replication defective retroviruses, adenoviruses, adeno-associated viruses and baculoviruses. Expression vectors also may include, without limitation, transgenic animals and non-mammalian cells/organisms, e.g., mammalian cells/organisms that have been engineered to perform mammalian N-linked glycosylation.

In some embodiments, provided herein are expression vectors encoding components of a chimeric hemagglutinin (HA) polypeptide (e.g., the stem domain and the head domain, or portions of either domain). Such vectors may be used to express the components in one or more host cells and the components may be isolated and conjugated together with a linker using techniques known to one of skill in the art.

An expression vector comprises a nucleic acid encoding a chimeric hemagglutinin (HA) polypeptide described herein and in a form suitable for expression of the nucleic acid in a host cell. In a specific embodiment, an expression vector includes one or more regulatory sequences, selected on the basis of the host cells to be used for expression, which is operably linked to the nucleic acid to be expressed. Within an expression vector, “operably linked” is intended to mean that a nucleic acid of interest is linked to the regulatory sequence(s) in a manner which allows for expression of the nucleic acid (e.g., in an in vitro transcription/translation system or in a host cell when the vector is introduced into the host cell). Regulatory sequences include promoters, enhancers and other expression control elements (e.g., polyadenylation signals). Regulatory sequences include those which direct constitutive expression of a nucleic acid in many types of host cells, those which direct expression of the nucleic acid only in certain host cells (e.g., tissue-specific regulatory sequences), and those which direct the expression of the nucleic acid upon stimulation with a particular agent (e.g., inducible regulatory sequences). It will be appreciated by those skilled in the art that the design of the expression vector can depend on such factors as the choice of the host cell to be transformed, the level of expression of protein desired, etc. The term “host cell” is intended to include a particular subject cell transformed or transfected with a nucleic acid and the progeny or potential progeny of such a cell. Progeny of such a cell may not be identical to the parent cell transformed or transfected with the nucleic acid due to mutations or environmental influences that may occur in succeeding generations or integration of the nucleic acid into the host cell genome. In specific embodiments, the host cell is a cell line.

Expression vectors can be designed for expression of a chimeric hemagglutinin (HA) polypeptide described herein using prokaryotic (e.g., E. coli) or eukaryotic cells (e.g., insect cells (using baculovirus expression vectors, see, e.g., Treanor et al., 2007, JAMA, 297 (14): 1577-1582 incorporated by reference herein in its entirety), yeast cells, plant cells, algae, avian, or mammalian cells). Examples of yeast host cells include, but are not limited to S. pombe and S. cerevisiae and examples, infra. An example of avian cells includes, but is not limited to EB66 cells. Examples of mammalian host cells include, but are not limited to, Crucell Per.C6 cells, Vero cells, CHO cells, VERO cells, BHK cells, HeLa cells, COS cells, MDCK cells, 293 cells, 3T3 cells or WI38 cells. In certain embodiments, the hosts cells are myeloma cells, e.g., NS0 cells, 45.6 TG1.7 cells, AF-2 clone 9B5 cells, AF-2 clone 9B5 cells, J558L cells, MOPC 315 cells, MPC-11 cells, NCI-H929 cells, NP cells, NS0/1 cells, P3 NS1 Ag4 cells, P3/NS1/1-Ag4-1 cells, P3U1 cells, P3X63Ag8 cells, P3X63Ag8.653 cells, P3X63Ag8U.1 cells, RPMI 8226 cells, Sp 20-Ag14 cells, U266B1 cells, X63AG8.653 cells, Y3.Ag.1.2.3 cells, and YO cells. Non-limiting examples of insect cells include Sf9, Sf21, Trichoplusia ni, Spodoptera frugiperda and Bombyx mori. In a particular embodiment, a mammalian cell culture system (e.g. Chinese hamster ovary or baby hamster kidney cells) is used for expression of a chimeric hemagglutinin (HA) polypeptide. In another embodiment, a plant cell culture system is used for expression of a chimeric hemagglutinin (HA) polypeptide. See, e.g., U.S. Pat. Nos. 7,504,560; 6,770,799; 6,551,820; 6,136,320; 6,034,298; 5,914,935; 5,612,487; and 5,484,719, and U.S. patent application publication Nos. 2009/0208477, 2009/0082548, 2009/0053762, 2008/0038232, 2007/0275014 and 2006/0204487 for plant cells and methods for the production of proteins utilizing plant cell culture systems. In specific embodiments, plant cell culture systems are not used for expression of a chimeric hemagglutinin (HA) polypeptide. The host cells comprising the nucleic acids that encode the chimeric hemagglutinin (HA) polypeptides described herein can be isolated, i.e., the cells are outside of the body of a subject. In certain embodiments, the cells are engineered to express nucleic acids that encode the chimeric influenza virus hemagglutinin polypeptides described herein. In specific embodiments, the host cells are cells from a cell line.

An expression vector can be introduced into host cells via conventional transformation or transfection techniques. Such techniques include, but are not limited to, calcium phosphate or calcium chloride co-precipitation, DEAE-dextran-mediated transfection, lipofection, and electroporation. Suitable methods for transforming or transfecting host cells can be found in Sambrook et al., 1989, Molecular Cloning-A Laboratory Manual, 2nd Edition, Cold Spring Harbor Press, New York, and other laboratory manuals. In certain embodiments, a host cell is transiently transfected with an expression vector containing a nucleic acid encoding a chimeric hemagglutinin (HA) polypeptide. In other embodiments, a host cell is stably transfected with an expression vector containing a nucleic acid encoding a chimeric hemagglutinin (HA) polypeptide.

For stable transfection of mammalian cells, it is known that, depending upon the expression vector and transfection technique used, only a small fraction of cells may integrate the foreign DNA into their genome. In order to identify and select these integrants, a nucleic acid that encodes a selectable marker (e.g., for resistance to antibiotics) is generally introduced into the host cells along with the nucleic acid of interest. Examples of selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

As an alternative to recombinant expression of a chimeric hemagglutinin (HA) polypeptide using a host cell, an expression vector containing a nucleic acid encoding a chimeric hemagglutinin (HA) polypeptide can be transcribed and translated in vitro using, e.g., T7 promoter regulatory sequences and T7 polymerase. In a specific embodiment, a coupled transcription/translation system, such as Promega TNT®, or a cell lysate or cell extract comprising the components necessary for transcription and translation may be used to produce a chimeric hemagglutinin (HA) polypeptide.

Once a chimeric hemagglutinin (HA) polypeptide has been produced, it may be isolated or purified by any method known in the art for isolation or purification of a protein, for example, by chromatography (e.g., ion exchange, affinity, particularly by affinity for the specific antigen, by Protein A, and sizing column chromatography), centrifugation, differential solubility, or by any other standard technique for the isolation or purification of proteins.

Accordingly, provided herein are methods for producing a chimeric hemagglutinin (HA) polypeptide. In one embodiment, the method comprises culturing a host cell containing a nucleic acid encoding the polypeptide in a suitable medium such that the polypeptide is produced. In some embodiments, the method further comprises isolating the polypeptide from the medium or the host cell.

Also provided herein are methods for producing a virus comprising a chimeric HA described herein, comprising propagating the virus in any substrate that allows the virus to grow to titers that permit their use in accordance with the methods described herein. In one embodiment, the substrate allows the viruses to grow to titers comparable to those determined for the corresponding wild-type viruses. In a specific embodiment, the virus is propagated in embryonated eggs (e.g., chicken eggs). In a specific embodiment, the virus is propagated in 8 day old, 9-day old, 8-10 day old, 10 day old, 11-day old, 10-12 day old, or 12-day old embryonated eggs (e.g., chicken eggs). In certain embodiments, the virus is propagated in MDCK cells, Vero cells, 293T cells, or other cell lines known in the art. In certain embodiments, the virus is propagated in cells derived from embryonated eggs.

### 5.4 Influenza Virus Vectors

In one aspect, provided herein are influenza viruses containing a chimeric influenza virus hemagglutinin polypeptide described herein. In a specific embodiment, the chimeric hemagglutinin (HA) polypeptide is incorporated into the virions of the influenza virus. The influenza viruses may be conjugated to moieties that target the viruses to particular cell types, such as immune cells. In some embodiments, the virions of the influenza virus have incorporated into them or express a heterologous polypeptide in addition to a chimeric hemagglutinin (HA) polypeptide. The heterologous polypeptide may be a polypeptide that has immunopotentiating activity, or that targets the influenza virus to a particular cell type, such as an antibody that binds to an antigen on a specific cell type or a ligand that binds a specific receptor on a specific cell type.

Influenza viruses containing a chimeric hemagglutinin (HA) polypeptide may be produced by supplying in trans the chimeric hemagglutinin (HA) polypeptide during production of virions using techniques known to one skilled in the art, such as reverse genetics and helper-free plasmid rescue. Alternatively, the replication of a parental influenza virus comprising a genome engineered to express a chimeric hemagglutinin (HA) polypeptide in cells susceptible to infection with the virus wherein hemagglutinin function is provided in trans will produce progeny influenza viruses containing the chimeric hemagglutinin (HA) polypeptide.

In another aspect, provided herein are influenza viruses comprising a genome engineered to express a chimeric hemagglutinin (HA) polypeptide. In a specific embodiment, the genome of a parental influenza virus is engineered to encode a chimeric hemagglutinin (HA) polypeptide, which is expressed by progeny influenza virus. In another specific embodiment, the genome of a parental influenza virus is engineered to encode a chimeric hemagglutinin (HA) polypeptide, which is expressed and incorporated into the virions of progeny influenza virus. Thus, the progeny influenza virus resulting from the replication of the parental influenza virus contain a chimeric hemagglutinin (HA) polypeptide. The virions of the parental influenza virus may have incorporated into them a chimeric hemagglutinin (HA) polypeptide that contains a stem and/or head domain from the same or a different lineage or strain of influenza B virus. Alternatively, the virions of the parental influenza virus may have incorporated into them a moiety that is capable of functionally replacing one or more of the activities of influenza virus hemagglutinin polypeptide (e.g., the receptor binding and/or fusogenic activities of influenza virus hemagglutinin). In specific embodiments, the parental influenza virus is an influenza A virus. In specific embodiments, the parental influenza virus is an influenza B virus.

In some embodiments, the virions of the parental influenza virus have incorporated into them a heterologous polypeptide. In certain embodiments, the genome of a parental influenza virus is engineered to encode a heterologous polypeptide and a chimeric hemagglutinin (HA) polypeptide, which are expressed by progeny influenza virus. In specific embodiments, the chimeric hemagglutinin (HA) polypeptide, the heterologous polypeptide or both are incorporated into virions of the progeny influenza virus.

Since the genome of influenza A and B viruses consist of eight (8) single-stranded, negative sense segments (influenza C viruses consist of seven (7) single-stranded, negative sense segments), the genome of a parental influenza virus may be engineered to express a chimeric hemagglutinin (HA) polypeptide (and any other polypeptide, such as a heterologous polypeptide) using a recombinant segment and techniques known to one skilled in the art, such a reverse genetics and helper-free plasmid rescue. In one embodiment, the recombinant segment comprises a nucleic acid encoding the chimeric hemagglutinin (HA) polypeptide as well as the 3′ and 5′ incorporation signals which are required for proper replication, transcription and packaging of the vRNAs (Fujii et al., 2003, Proc. Natl. Acad. Sci. USA 100:2002-2007; Zheng, et al., 1996, Virology 217:242-251, International Publication No. WO 2011/014645, all of which are incorporated by reference herein in their entireties). In a specific embodiment, the recombinant segment uses the 3′ and 5′ noncoding and/or nontranslated sequences of segments of influenza viruses that are from a different or the same lineage or strain as the parental influenza virus. In some embodiments, the recombinant segment comprises the 3′ noncoding region of an influenza virus hemagglutinin polypeptide, the untranslated regions of an influenza virus hemagglutinin polypeptide, and the 5′ non-coding region of an influenza virus hemagglutinin polypeptide. In specific embodiments, the recombinant segment comprises the 3′ and 5′ noncoding and/or nontranslated sequences of the HA segment of an influenza virus that is the same lineage or strain as the influenza virus lineage or strain as the HA1 stem segment, the globular head domain, and/or the HA2 of a chimeric hemagglutinin (HA) polypeptide. In specific embodiments, the recombinant segment comprises packaging signals, such as the 5′ and 3′ non-coding regions and signal peptide of the HA segment of an influenza virus, from the same type, lineage, or strain as the influenza virus backbone. For example, if the chimeric HA is engineered to be expressed from an influenza A virus, then the nucleotide sequence encoding chimeric HA comprises the 5′ and 3′ non-coding regions and the nucleotide sequence encoding the signal peptide of the HA segment of the influenza A virus. In another example, if the chimeric HA is engineered to be expressed from an influenza B virus, then the nucleotide sequence encoding chimeric HA comprises the 5′ and 3′ non-coding regions and the nucleotide sequence encoding the signal peptide of the HA segment of the influenza B virus. In certain embodiments, the recombinant segment encoding the chimeric hemagglutinin (HA) polypeptide may replace the HA segment of a parental influenza virus.

In some embodiments, a chimeric hemagglutinin gene segment encodes a chimeric hemagglutinin (HA) polypeptide. In specific embodiments, the chimeric hemagglutinin (HA) gene segment and at least one other influenza virus gene segment comprise packaging signals that enable the chimeric hemagglutinin (HA) gene segment and the at least one other gene segment to segregate together during replication of a recombinant influenza virus (see, Gao & Palese 2009, PNAS 106:15891-15896; and International Application Publication No. WO11/014645).

In some embodiments, the genome of a parental influenza virus may be engineered to express a chimeric hemagglutinin (HA) polypeptide using a recombinant segment that is bicistronic. Bicistronic techniques allow the engineering of coding sequences of multiple proteins into a single mRNA through the use of internal ribosome entry site (IRES) sequences. IRES sequences direct the internal recruitment of ribosomes to the RNA molecule and allow downstream translation in a cap independent manner. Briefly, a coding region of one protein is inserted into the open reading frame (ORF) of a second protein. The insertion is flanked by an IRES and any untranslated signal sequences necessary for proper expression and/or function. The insertion must not disrupt the ORF, polyadenylation or transcriptional promoters of the second protein (see, e.g., García-Sastre et al., 1994, J. Virol. 68:6254-6261 and García-Sastre et al., 1994 Dev. Biol. Stand. 82:237-246, each of which is hereby incorporated by reference in its entirety). See also, e.g., U.S. Pat. Nos. 6,887,699, 6,001,634, 5,854,037 and 5,820,871, each of which is incorporated herein by reference in its entirety. Any IRES known in the art or described herein may be used in accordance with the invention (e.g., the IRES of BiP gene, nucleotides 372 to 592 of GenBank database entry HUMGRP78; or the IRES of encephalomyocarditis virus (EMCV), nucleotides 1430-2115 of GenBank database entry CQ867238). Thus, in certain embodiments, a parental influenza virus is engineered to contain a bicistronic RNA segment that expresses the chimeric hemagglutinin (HA) polypeptide and another polypeptide, such as a gene expressed by the parental influenza virus. In some embodiments, the parental influenza virus gene is the HA gene.

Techniques known to one skilled in the art may be used to produce an influenza virus containing a chimeric hemagglutinin (HA) polypeptide and an influenza virus comprising a genome engineered to express a chimeric hemagglutinin (HA) polypeptide. For example, reverse genetics techniques may be used to generate such an influenza virus. Briefly, reverse genetics techniques generally involve the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in European Patent Publication EP 0702085A1, published Feb. 20, 1996; in U.S. patent application Ser. No. 09/152,845; in International Patent Publications PCT WO 97/12032 published Apr. 3, 1997; WO 96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

Alternatively, helper-free plasmid technology may be used to produce an influenza virus containing a chimeric hemagglutinin (HA) polypeptide and an influenza virus comprising a genome engineered to express a chimeric hemagglutinin (HA) polypeptide. Briefly, full length cDNAs of viral segments are amplified using PCR with primers that include unique restriction sites, which allow the insertion of the PCR product into the plasmid vector (Flandorfer et al., 2003, J. Virol. 77:9116-9123; Nakaya et al., 2001, J. Virol. 75:11868-11873; both of which are incorporated herein by reference in their entireties). The plasmid vector is designed so that an exact negative (vRNA sense) transcript is expressed. For example, the plasmid vector may be designed to position the PCR product between a truncated human RNA polymerase I promoter and a hepatitis delta virus ribozyme sequence such that an exact negative (vRNA sense) transcript is produced from the polymerase I promoter. Separate plasmid vectors comprising each viral segment as well as expression vectors comprising necessary viral proteins may be transfected into cells leading to production of recombinant viral particles. In another example, plasmid vectors from which both the viral genomic RNA and mRNA encoding the necessary viral proteins are expressed may be used. For a detailed description of helper-free plasmid technology see, e.g., International Publication No. WO 01/04333; U.S. Pat. Nos. 6,951,754, 7,384,774, 6,649,372, and 7,312,064; Fodor et al., 1999, J. Virol. 73:9679-9682; Quinlivan et al., 2005, J. Virol. 79:8431-8439; Hoffmann et al., 2000, Proc. Natl. Acad. Sci. USA 97:6108-6113; and Neumann et al., 1999, Proc. Natl. Acad. Sci. USA 96:9345-9350, which are incorporated herein by reference in their entireties.

The influenza viruses described herein may be propagated in any substrate that allows the virus to grow to titers that permit their use in accordance with the methods described herein. Thus, in certain embodiments, provided herein is a method for producing a virus described herein comprising propagating the virus in a substrate. In one embodiment, the substrate allows the viruses to grow to titers comparable to those determined for the corresponding wild-type viruses. In certain embodiments, the substrate is one which is biologically relevant to the influenza virus or to the virus from which the HA function is derived. In a specific embodiment, an attenuated influenza virus by virtue of, e.g., a mutation in the NS1 gene, may be propagated in an IFN-deficient substrate. For example, a suitable IFN-deficient substrate may be one that is defective in its ability to produce or respond to interferon, or is one which an IFN-deficient substrate may be used for the growth of any number of viruses which may require interferon-deficient growth environment. See, for example, U.S. Pat. Nos. 6,573,079, issued Jun. 3, 2003, U.S. Pat. No. 6,852,522, issued Feb. 8, 2005, and U.S. Pat. No. 7,494,808, issued Feb. 24, 2009, the entire contents of each of which is incorporated herein by reference in its entirety. In a specific embodiment, the virus is propagated in embryonated eggs (e.g., chicken eggs). In a specific embodiment, the virus is propagated in 8 day old, 9-day old, 8-10 day old, 10 day old, 11-day old, 10-12 day old, or 12-day old embryonated eggs (e.g., chicken eggs). In certain embodiments, the virus is propagated in a cell line susceptible to influenza virus infection. In certain embodiments, the virus is propagated in MDCK cells, Vero cells, 293T cells, or other cell lines known in the art. In certain embodiments, the virus is propagated in cells derived from embryonated eggs.

The influenza viruses described herein may be isolated and purified by any method known to those of skill in the art. In one embodiment, the virus is removed from cell culture and separated from cellular components, typically by well known clarification procedures, e.g., such as gradient centrifugation and column chromatography, and may be further purified as desired using procedures well known to those skilled in the art, e.g., plaque assays.

In certain embodiments, the influenza viruses, or influenza virus polypeptides, genes or genome segments for use as described herein are obtained or derived from an influenza A virus. In certain embodiments, the influenza viruses, or influenza virus polypeptides, genes or genome segments for use as described herein are obtained or derived from a single influenza A virus subtype or strain. In other embodiments, the influenza viruses, or influenza virus polypeptides, genes or genome segments for use as described herein are obtained or derived from two or more influenza A virus subtypes or strains. In a specific embodiment, the influenza A virus is an influenza virus of the H1, H2, H3, H4, H5, H6, H7, H8, H9, H10, H11, H12, H13, H14, H15, H16, H17, or H18 subtype. In a specific embodiment, the influenza A virus is an influenza virus of the H2, H4, H5, H6, H7, H8, H9, H10, H11, H12, H13, H14, H15, H16, H17, or H18 subtype. In a specific embodiment, the influenza A virus is an influenza virus of the H5, H8, H11, H12, or H13 subtype. In a specific embodiment, the influenza A virus is an influenza virus of the H5 subtype. In a specific embodiment, the influenza A virus is an influenza virus of the H8 subtype. In a specific embodiment, the influenza A virus is an influenza virus of the H11 subtype. In a specific embodiment, the influenza A virus is an influenza virus of the H12 subtype. In a specific embodiment, the influenza A virus is an influenza virus of the H13 subtype. In a specific embodiment, the influenza A virus is an avian influenza virus.

In some embodiments, the influenza viruses, or influenza virus polypeptides, genes or genome segments for use as described herein are obtained or derived from an influenza B virus. In certain embodiments, the influenza viruses, or influenza virus polypeptides, genes or genome segments for use as described herein are obtained or derived from a single influenza B virus lineage or strain. In other embodiments, the influenza viruses, or influenza virus polypeptides, genes or genome segments for use as described herein are obtained or derived from two or more influenza B virus lineages or strains. In other embodiments, the influenza viruses, or influenza virus polypeptides, genes or genome segments for use as described herein are obtained or derived from a combination of influenza A and influenza B virus subtypes, lineages, or strains.

In some embodiments, the influenza viruses, or influenza virus polypeptides, genes or genome segments for use as described herein are obtained or derived from an influenza C virus. In certain embodiments, the influenza viruses, or influenza virus polypeptides, genes or genome segments for use as described herein are obtained or derived from a single influenza C virus subtype or strain. In other embodiments, the influenza viruses, or influenza virus polypeptides, genes or genome segments for use as described herein are obtained or derived from two or more influenza C virus subtypes or strains. In other embodiments, the influenza viruses, or influenza virus polypeptides, genes or genome segments for use as described herein are obtained or derived from a combination of influenza C virus and influenza A virus and/or influenza B virus subtypes or strains.

Non-limiting examples of influenza A viruses include subtype H10N4, subtype H10N5, subtype H10N7, subtype H10N8, subtype H10N9, subtype H11N1, subtype H11N13, subtype H11N2, subtype H11N4, subtype H11N6, subtype H11N8, subtype H11N9, subtype H12N1, subtype H12N4, subtype H12N5, subtype H12N8, subtype H13N2, subtype H13N3, subtype H13N6, subtype H13N7, subtype H14N5, subtype H14N6, subtype H15N8, subtype H15N9, subtype H16N3, subtype H1N1, subtype H1N2, subtype H1N3, subtype H1N6, subtype H1N9, subtype H2N1, subtype H2N2, subtype H2N3, subtype H2N5, subtype H2N7, subtype H2N8, subtype H2N9, subtype H3N1, subtype H3N2, subtype H3N3, subtype H3N4, subtype H3N5, subtype H3N6, subtype H3N8, subtype H3N9, subtype H4N1, subtype H4N2, subtype H4N3, subtype H4N4, subtype H4N5, subtype H4N6, subtype H4N8, subtype H4N9, subtype H5N1, subtype H5N2, subtype H5N3, subtype H5N4, subtype H5N6, subtype H5N7, subtype H5N8, subtype H5N9, subtype H6N1, subtype H6N2, subtype H6N3, subtype H6N4, subtype H6N5, subtype H6N6, subtype H6N7, subtype H6N8, subtype H6N9, subtype H7N1, subtype H7N2, subtype H7N3, subtype H7N4, subtype H7N5, subtype H7N7, subtype H7N8, subtype H7N9, subtype H8N4, subtype H8N5, subtype H9N1, subtype H9N2, subtype H9N3, subtype H9N5, subtype H9N6, subtype H9N7, subtype H9N8, and subtype H9N9.

Specific examples of strains of influenza A virus include, but are not limited to: A/Victoria/361/2011 (H3N2); A/California/4/2009 (H1N1); A/California/7/2009 (H1N1); A/Perth/16/2009 (H3N2); A/Brisbane/59/2007 (H1N1); A/Brisbane/10/2007 ((H3N2); A/sw/Iowa/15/30 (H1N1); A/WSN/33 (H1N1); A/eq/Prague/1/56 (H7N7); A/PR/8/34; A/mallard/Potsdam/178-4/83 (H2N2); A/herring gull/DE/712/88 (H16N3); A/sw/Hong Kong/168/1993 (H1N1); A/mallard/Alberta/211/98 (H1N1); A/shorebird/Delaware/168/06 (H16N3); A/sw/Netherlands/25/80 (H1N1); A/sw/Germany/2/81 (H1N1); A/sw/Hannover/1/81 (H1N1); A/sw/Potsdam/1/81 (H1N1); A/sw/Potsdam/15/81 (H1N1); A/sw/Potsdam/268/81 (H1N1); A/sw/Finistere/2899/82 (H1N1); A/sw/Potsdam/35/82 (H3N2); A/sw/Cote d′Armor/3633/84 (H3N2); A/sw/Gent/1/84 (H3N2); A/sw/Netherlands/12/85 (H1N1); A/sw/Karrenzien/2/87 (H3N2); A/sw/Schwerin/103/89 (H1N1); A/turkey/Germany/3/91 (H1N1); A/sw/Germany/8533/91 (H1N1); A/sw/Belgium/220/92 (H3N2); A/sw/Gent/V230/92 (H1N1); A/sw/Leipzig/145/92 (H3N2); A/sw/Re220/92 hp (H3N2); A/sw/Bakum/909/93 (H3N2); A/sw/Schleswig-Holstein/1/93 (H1N1); A/sw/Scotland/419440/94 (H1N2); A/sw/Bakum/5/95 (H1N1); A/sw/Best/5C/96 (H1N1); A/sw/England/17394/96 (H1N2); A/sw/Jena/5/96 (H3N2); A/sw/Oedenrode/7C/96 (H3N2); A/sw/Lohne/1/97 (H3N2); A/sw/Cote d′Armor/790/97 (H1N2); A/sw/Bakum/1362/98 (H3N2); A/sw/Italy/1521/98 (H1N2); A/sw/Italy/1553-2/98 (H3N2); A/sw/Italy/1566/98 (H1N1); A/sw/Italy/1589/98 (H1N1); A/sw/Bakum/8602/99 (H3N2); A/sw/Cotes d′Armor/604/99 (H1N2); A/sw/Cote d′Armor/1482/99 (H1N1); A/sw/Gent/7625/99 (H1N2); A/Hong Kong/1774/99 (H3N2); A/sw/Hong Kong/5190/99 (H3N2); A/sw/Hong Kong/5200/99 (H3N2); A/sw/Hong Kong/5212/99 (H3N2); A/sw/Ille et Villaine/1455/99 (H1N1); A/sw/Italy/1654-1/99 (H1N2); A/sw/Italy/2034/99 (H1N1); A/sw/Italy/2064/99 (H1N2); A/sw/Berlin/1578/00 (H3N2); A/sw/Bakum/1832/00 (H1N2); A/sw/Bakum/1833/00 (H1N2); A/sw/Cote d′Armor/800/00 (H1N2); A/sw/Hong Kong/7982/00 (H3N2); A/sw/Italy/1081/00 (H1N2); A/sw/Belzig/2/01 (H1N1); A/sw/Belzig/54/01 (H3N2); A/sw/Hong Kong/9296/01 (H3N2); A/sw/Hong Kong/9745/01 (H3N2); A/sw/Spain/33601/01 (H3N2); A/sw/Hong Kong/1144/02 (H3N2); A/sw/Hong Kong/1197/02 (H3N2); A/sw/Spain/39139/02 (H3N2); A/sw/Spain/42386/02 (H3N2); A/Switzerland/8808/2002 (H1N1); A/sw/Bakum/1769/03 (H3N2); A/sw/Bissendorf/IDT1864/03 (H3N2); A/sw/Ehren/IDT2570/03 (H1N2); A/sw/Gescher/IDT2702/03 (H1N2); A/sw/Haselünne/2617/03 hp (H1N1); A/sw/Löningen/IDT2530/03 (H1N2); A/sw/IVD/IDT2674/03 (H1N2); A/sw/Nordkirchen/IDT1993/03 (H3N2); A/sw/Nordwalde/IDT2197/03 (H1N2); A/sw/Norden/IDT2308/03 (H1N2); A/sw/Spain/50047/03 (H1N1); A/sw/Spain/51915/03 (H1N1); A/sw/Vechta/2623/03 (H1N1); A/sw/Visbek/IDT2869/03 (H1N2); A/sw/Waltersdorf/IDT2527/03 (H1N2); A/sw/Damme/IDT2890/04 (H3N2); A/sw/Geldern/IDT2888/04 (H1N1); A/sw/Granstedt/IDT3475/04 (H1N2); A/sw/Greven/IDT2889/04 (H1N1); A/sw/Gudensberg/IDT2930/04 (H1N2); A/sw/Gudensberg/IDT2931/04 (H1N2); A/sw/Lohne/IDT3357/04 (H3N2); A/sw/Nortrup/IDT3685/04 (H1N2); A/sw/Seesen/IDT3055/04 (H3N2); A/sw/Spain/53207/04 (H1N1); A/sw/Spain/54008/04 (H3N2); A/sw/Stolzenau/IDT3296/04 (H1N2); A/sw/Wedel/IDT2965/04 (H1N1); A/sw/Bad Griesbach/IDT4191/05 (H3N2); A/sw/Cloppenburg/IDT4777/05 (H1N2); A/sw/Dötlingen/IDT3780/05 (H1N2); A/sw/Dötlingen/IDT4735/05 (H1N2); A/sw/Egglham/IDT5250/05 (H3N2); A/sw/Harkenblek/IDT4097/05 (H3N2); A/sw/Hertzen/IDT4317/05 (H3N2); A/sw/Krogel/IDT4192/05 (H1N1); A/sw/Laer/IDT3893/05 (H1N1); A/sw/Laer/IDT4126/05 (H3N2); A/sw/Merzen/IDT4114/05 (H3N2); A/sw/Muesleringen-S/IDT4263/05 (H3N2); A/sw/Osterhofen/IDT4004/05 (H3N2); A/sw/Sprenge/IDT3805/05 (H1N2); A/sw/Stadtlohn/IDT3853/05 (H1N2); A/sw/Voglarn/IDT4096/05 (H1N1); A/sw/Wohlerst/IDT4093/05 (H1N1); A/sw/Bad Griesbach/IDT5604/06 (H1N1); A/sw/Herzlake/IDT5335/06 (H3N2); A/sw/Herzlake/IDT5336/06 (H3N2); A/sw/Herzlake/IDT5337/06 (H3N2); and A/wild boar/Germany/R169/2006 (H3N2).

Other specific examples of strains of influenza A virus include, but are not limited to: A/Toronto/3141/2009 (H1N1); A/Regensburg/D6/2009 (H1N1); A/Bayern/62/2009 (H1N1); A/Bayern/62/2009 (H1N1); A/Bradenburg/19/2009 (H1N1); A/Bradenburg/20/2009 (H1N1); A/Distrito Federal/2611/2009 (H1N1); A/Mato Grosso/2329/2009 (H1N1); A/Sao Paulo/1454/2009 (H1N1); A/Sao Paulo/2233/2009 (H1N1); A/Stockholm/37/2009 (H1N1); A/Stockholm/41/2009 (H1N1); A/Stockholm/45/2009 (H1N1); A/swine/Alberta/OTH-33-1/2009 (H1N1); A/swine/Alberta/OTH-33-14/2009 (H1N1); A/swine/Alberta/OTH-33-2/2009 (H1N1); A/swine/Alberta/OTH-33-21/2009 (H1N1); A/swine/Alberta/OTH-33-22/2009 (H1N1); A/swine/Alberta/OTH-33-23/2009 (H1N1); A/swine/Alberta/OTH-33-24/2009 (H1N1); A/swine/Alberta/OTH-33-25/2009 (H1N1); A/swine/Alberta/OTH-33-3/2009 (H1N1); A/swine/Alberta/OTH-33-7/2009 (H1N1); A/Beijing/502/2009 (H1N1); A/Firenze/10/2009 (H1N1); A/Hong Kong/2369/2009 (H1N1); A/Italy/85/2009 (H1N1); A/Santo Domingo/572N/2009 (H1N1); A/Catalonia/385/2009 (H1N1); A/Catalonia/386/2009 (H1N1); A/Catalonia/387/2009 (H1N1); A/Catalonia/390/2009 (H1N1); A/Catalonia/394/2009 (H1N1); A/Catalonia/397/2009 (H1N1); A/Catalonia/398/2009 (H1N1); A/Catalonia/399/2009 (H1N1); A/Sao Paulo/2303/2009 (H1N1); A/Akita/1/2009 (H1N1); A/Castro/JXP/2009 (H1N1); A/Fukushima/1/2009 (H1N1); A/Israel/276/2009 (H1N1); A/Israel/277/2009 (H1N1); A/Israel/70/2009 (H1N1); A/Iwate/1/2009 (H1N1); A/Iwate/2/2009 (H1N1); A/Kagoshima/1/2009 (H1N1); A/Osaka/180/2009 (H1N1); A/Puerto Montt/Bio87/2009 (H1N1); A/Sao Paulo/2303/2009 (H1N1); A/Sapporo/1/2009 (H1N1); A/Stockholm/30/2009 (H1N1); A/Stockholm/31/2009 (H1N1); A/Stockholm/32/2009 (H1N1); A/Stockholm/33/2009 (H1N1); A/Stockholm/34/2009 (H1N1); A/Stockholm/35/2009 (H1N1); A/Stockholm/36/2009 (H1N1); A/Stockholm/38/2009 (H1N1); A/Stockholm/39/2009 (H1N1); A/Stockholm/40/2009 (H1N1) A/Stockholm/42/2009 (H1N1); A/Stockholm/43/2009 (H1N1); A/Stockholm/44/2009 (H1N1); A/Utsunomiya/2/2009 (H1N1); A/WRAIR/0573N/2009 (H1N1); and A/Zhejiang/DTID-ZJU01/2009 (H1N1).

Non-limiting examples of influenza B viruses include strain Aichi/5/88, strain B/Brisbane/60/2008; Akita/27/2001, strain Akita/5/2001, strain Alaska/16/2000, strain Alaska/1777/2005, strain Argentina/69/2001, strain Arizona/146/2005, strain Arizona/148/2005, strain Bangkok/163/90, strain Bangkok/34/99, strain Bangkok/460/03, strain Bangkok/54/99, strain Barcelona/215/03, strain Beijing/15/84, strain Beijing/184/93, strain Beijing/243/97, strain Beijing/43/75, strain Beijing/5/76, strain Beijing/76/98, strain Belgium/WV106/2002, strain Belgium/WV107/2002, strain Belgium/WV109/2002, strain Belgium/WV114/2002, strain Belgium/WV122/2002, strain Bonn/43, strain Brazil/952/2001, strain Bucharest/795/03, strain Buenos Aires/161/00), strain Buenos Aires/9/95, strain Buenos Aires/SW16/97, strain Buenos Aires/VL518/99, strain Canada/464/2001, strain Canada/464/2002, strain Chaco/366/00, strain Chaco/R113/00, strain Cheju/303/03, strain Chiba/447/98, strain Chongqing/3/2000, strain clinical isolate SA1 Thailand/2002, strain clinical isolate SA10 Thailand/2002, strain clinical isolate SA100 Philippines/2002, strain clinical isolate SA101 Philippines/2002, strain clinical isolate SA110 Philippines/2002), strain clinical isolate SA112 Philippines/2002, strain clinical isolate SA113 Philippines/2002, strain clinical isolate SA114 Philippines/2002, strain clinical isolate SA2 Thailand/2002, strain clinical isolate SA20 Thailand/2002, strain clinical isolate SA38 Philippines/2002, strain clinical isolate SA39 Thailand/2002, strain clinical isolate SA99 Philippines/2002, strain CNIC/27/2001, strain Colorado/2597/2004, strain Cordoba/VA418/99, strain Czechoslovakia/16/89, strain Czechoslovakia/69/90, strain Daeku/10/97, strain Daeku/45/97, strain Daeku/47/97, strain Daeku/9/97, strain B/Du/4/78, strain B/Durban/39/98, strain Durban/43/98, strain Durban/44/98, strain B/Durban/52/98, strain Durban/55/98, strain Durban/56/98, strain England/1716/2005, strain England/2054/2005), strain England/23/04, strain Finland/154/2002, strain Finland/159/2002, strain Finland/160/2002, strain Finland/161/2002, strain Finland/162/03, strain Finland/162/2002, strain Finland/162/91, strain Finland/164/2003, strain Finland/172/91, strain Finland/173/2003, strain Finland/176/2003, strain Finland/184/91, strain Finland/188/2003, strain Finland/190/2003, strain Finland/220/2003, strain Finland/WV5/2002, strain Fujian/36/82, strain Geneva/5079/03, strain Genoa/11/02, strain Genoa/2/02, strain Genoa/21/02, strain Genova/54/02, strain Genova/55/02, strain Guangdong/05/94, strain Guangdong/08/93, strain Guangdong/5/94, strain Guangdong/55/89, strain Guangdong/8/93, strain Guangzhou/7/97, strain Guangzhou/86/92, strain Guangzhou/87/92, strain Gyeonggi/592/2005, strain Hannover/2/90, strain Harbin/07/94, strain Hawaii/10/2001, strain Hawaii/1990/2004, strain Hawaii/38/2001, strain Hawaii/9/2001, strain Hebei/19/94, strain Hebei/3/94), strain Henan/22/97, strain Hiroshima/23/2001, strain Hong Kong/110/99, strain Hong Kong/1115/2002, strain Hong Kong/112/2001, strain Hong Kong/123/2001, strain Hong Kong/1351/2002, strain Hong Kong/1434/2002, strain Hong Kong/147/99, strain Hong Kong/156/99, strain Hong Kong/157/99, strain Hong Kong/22/2001, strain Hong Kong/22/89, strain Hong Kong/336/2001, strain Hong Kong/666/2001, strain Hong Kong/9/89, strain Houston/1/91, strain Houston/1/96, strain Houston/2/96, strain Hunan/4/72, strain Ibaraki/2/85, strain ncheon/297/2005, strain India/3/89, strain India/77276/2001, strain Israel/95/03, strain Israel/WV187/2002, strain Japan/1224/2005, strain Jiangsu/10/03, strain Johannesburg/1/99, strain Johannesburg/96/01, strain Kadoma/1076/99, strain Kadoma/122/99, strain Kagoshima/15/94, strain Kansas/22992/99, strain Khazkov/224/91, strain Kobe/1/2002, strain, strain Kouchi/193/99, strain Lazio/1/02, strain Lee/40, strain Leningrad/129/91, strain Lissabon/2/90), strain Los Angeles/1/02, strain Lusaka/270/99, strain Lyon/1271/96, strain Malaysia/83077/2001, strain Maputo/1/99, strain Mar del Plata/595/99, strain Maryland/1/01, strain Memphis/1/01, strain Memphis/12/97-MA, strain Michigan/22572/99, strain Mie/1/93, strain Milano/1/01, strain Minsk/318/90, strain Moscow/3/03, strain Nagoya/20/99, strain Nanchang/1/00, strain Nashville/107/93, strain Nashville/45/91, strain Nebraska/2/01, strain Netherland/801/90, strain Netherlands/429/98, strain New York/1/2002, strain NIB/48/90, strain Ningxia/45/83, strain Norway/1/84, strain Oman/16299/2001, strain Osaka/1059/97, strain Osaka/983/97-V2, strain Oslo/1329/2002, strain Oslo/1846/2002, strain Panama/45/90, strain Paris/329/90, strain Parma/23/02, strain Perth/211/2001, strain Peru/1364/2004, strain Philippines/5072/2001, strain Pusan/270/99, strain Quebec/173/98, strain Quebec/465/98, strain Quebec/7/01, strain Roma/1/03, strain Saga/S172/99, strain Seoul/13/95, strain Seoul/37/91, strain Shangdong/7/97, strain Shanghai/361/2002), strain Shiga/T30/98, strain Sichuan/379/99, strain Singapore/222/79, strain Spain/WV27/2002, strain Stockholm/10/90, strain Switzerland/5441/90, strain Taiwan/0409/00, strain Taiwan/0722/02, strain Taiwan/97271/2001, strain Tehran/80/02, strain Tokyo/6/98, strain Trieste/28/02, strain Ulan Ude/4/02, strain United Kingdom/34304/99, strain USSR/100/83, strain Victoria/103/89, strain Vienna/1/99, strain Wuhan/356/2000, strain WV194/2002, strain Xuanwu/23/82, strain Yamagata/1311/2003, strain Yamagata/K500/2001, strain Alaska/12/96, strain GA/86, strain NAGASAKI/1/87, strain Tokyo/942/96, strain B/Wisconsin/1/2010; and strain Rochester/02/2001. In a specific embodiment, the influenza B virus is B/Malaysia/2506/04.

Non-limiting examples of influenza C viruses include strain Aichi/1/81, strain Ann Arbor/1/50, strain Aomori/74, strain California/78, strain England/83, strain Greece/79, strain Hiroshima/246/2000, strain Hiroshima/252/2000, strain Hyogo/1/83, strain Johannesburg/66, strain Kanagawa/1/76, strain Kyoto/1/79, strain Mississippi/80, strain Miyagi/1/97, strain Miyagi/5/2000, strain Miyagi/9/96, strain Nara/2/85, strain NewJersey/76, strain pig/Beijing/115/81, strain Saitama/3/2000), strain Shizuoka/79, strain Yamagata/2/98, strain Yamagata/6/2000, strain Yamagata/9/96, strain BERLIN/1/85, strain ENGLAND/892/8, strain GREAT LAKES/1167/54, strain JJ/50, strain PIG/BEIJING/10/81, strain PIG/BEIJING/439/82), strain TAYLOR/1233/47, and strain C/YAMAGATA/10/81.

In certain embodiments, the influenza viruses provided herein have an attenuated phenotype. In specific embodiments, the attenuated influenza virus is based on influenza A virus. In other embodiments, the attenuated influenza virus is based on influenza B virus. In yet other embodiments, the attenuated influenza virus is based on influenza C virus. In other embodiments, the attenuated influenza virus may comprise genes or genome segments from one or more strains or subtypes of influenza A, influenza B, and/or influenza C virus. In some embodiments, the attenuated backbone virus comprises genes from an influenza A virus and an influenza B virus. In specific embodiments, the attenuated influenza virus comprises, encodes, or both, a chimeric HA and has a backbone of an influenza A virus. In specific embodiments, the attenuated influenza virus comprises, encodes, or both, a chimeric HA and has a backbone of an influenza B virus.

In specific embodiments, attenuation of influenza virus is desired such that the virus remains, at least partially, infectious and can replicate in vivo, but only generate low titers resulting in subclinical levels of infection that are non-pathogenic. Such attenuated viruses are especially suited for embodiments described herein wherein the virus or an immunogenic composition thereof is administered to a subject to induce an immune response. Attenuation of the influenza virus can be accomplished according to any method known in the art, such as, e.g., selecting viral mutants generated by chemical mutagenesis, mutation of the genome by genetic engineering, selecting reassortant viruses that contain segments with attenuated function (e.g., truncated NS1 protein (see, e.g., Hai et al., 2008, Journal of Virology 82 (21): 10580-10590, which is incorporated by reference herein in its entirety) or NS1 deletion (see, e.g., Wressnigg et al., 2009, Vaccine 27:2851-2857, which is incorporated by reference herein in its entirety)), or selecting for conditional virus mutants (e.g., cold-adapted viruses, see, e.g., Alexandrova et al., 1990, Vaccine, 8:61-64, which is incorporated by reference herein in its entirety). Alternatively, naturally occurring attenuated influenza viruses may be used as influenza virus backbones for the influenza virus vectors.

In certain embodiments, an influenza virus comprising a chimeric HA described herein has one, two, or more of the functions of an influenza virus comprising a wild-type influenza virus HA. Nonlimiting examples of functions of a wild-type influenza virus HA include fusogenic activity, receptor binding activity, budding, and particle formation. In a specific embodiment, an influenza virus comprising a chimeric influenza HA polypeptide described herein has fusogenic activity. Assays known to one skilled in the art can be utilized to assess the fusogenic activity of an influenza virus comprising a chimeric influenza HA polypeptide described herein, such as, for example, immunofluorescence assays and pseudotyped virus-like-particle assays. In a specific embodiment, an influenza virus comprising a chimeric influenza HA polypeptide described herein has replication activity. Assays known to one skilled in the art can be utilized the assess the replication activity of an influenza virus comprising a chimeric influenza HA polypeptide described herein, such as, for example, plaque assay and western blot analyses.

### 5.5 Virus-Like Particles and Virosomes

The chimeric influenza virus hemagglutinin polypeptides described herein can be incorporated into virus-like particle (VLP) vectors, e.g., purified/isolated VLPs. VLPs generally comprise a viral polypeptide(s) typically derived from a structural protein(s) of a virus. In some embodiments, the VLPs are not capable of replicating. In certain embodiments, the VLPs may lack the complete genome of a virus or comprise a portion of the genome of a virus. In some embodiments, the VLPs are not capable of infecting a cell. In some embodiments, the VLPs express on their surface one or more of viral (e.g., virus surface glycoprotein) or non-viral (e.g., antibody or protein) targeting moieties known to one skilled in the art or described herein. In some embodiments, the VLPs comprise a chimeric hemagglutinin (HA) polypeptide and a viral structural protein, such as HIV gag. In a specific embodiment, the VLPs comprise a chimeric hemagglutinin (HA) polypeptide and an HIV gag polypeptide. In another specific embodiment, the VLPs comprise a chimeric hemagglutinin (HA) polypeptide and influenza virus neuraminidase polypeptide. In another specific embodiment, the VLPs comprise a chimeric hemagglutinin (HA) polypeptide, influenza virus neuraminidase polypeptide, and influenza virus Ml polypeptide.

Methods for producing and characterizing recombinantly produced VLPs have been described based on several viruses, including influenza virus (Bright et al. (2007) Vaccine. 25:3871), human papilloma virus type 1 (Hagnesee et al. (1991) J. Virol. 67:315), human papilloma virus type 16 (Kirnbauer et al. Proc. Natl. Acad. Sci. (1992) 89:12180), HIV-1 (Haffer et al., (1990) J. Virol. 64:2653), and hepatitis A (Winokur (1991) 65:5029), each of which is incorporated herein in its entirety. Methods for expressing VLPs that contain NDV proteins are provided by Pantua et al. (2006) J. Virol. 80:11062-11073, and in United States patent application Publication No. 20090068221, published Mar. 12, 2009, each of which is incorporated in its entirety herein. In a specific embodiment, the VLPs comprising chimeric hemagglutinin (HA) polypeptide described herein are generated using baculovirus, as described in the Examples section below. In other embodiments, the VLPs comprising chimeric hemagglutinin (HA) polypeptides described herein are generated using 293T cells.

In specific embodiments, VLPs, e.g., VLPs comprising a chimeric hemagglutinin (HA) polypeptide are expressed in cells (such as, e.g., mammalian cells (e.g., 293T cells) and insect cells (e.g., High Five cells and Sf9 cells). In certain embodiments, the VLPs are expressed in cells that express surface glycoproteins that comprise sialic acid. In certain embodiments, VLPs, e.g., VLPs comprising a chimeric hemagglutinin (HA) polypeptide, are expressed in cells that do not express surface glycoproteins that comprise sialic acid.

In a specific embodiment, a chimeric hemagglutinin (HA) polypeptide may be incorporated into a virosome. A virosome containing a chimeric hemagglutinin (HA) polypeptide may be produced using techniques known to those skilled in the art. For example, a virosome may be produced by disrupting a purified virus, extracting the genome, and reassembling particles with the viral proteins (e.g., a chimeric hemagglutinin (HA) polypeptide) and lipids to form lipid particles containing viral proteins.

Also provided herein are methods for producing and characterizing recombinantly produced VLPs comprising a chimeric HA described herein. Methods for producing and characterizing recombinantly produced VLPs have been described based on several viruses, including influenza virus (Bright et al. (2007) Vaccine. 25:3871), human papilloma virus type 1 (Hagnesee et al. (1991) J. Virol. 67:315), human papilloma virus type 16 (Kirnbauer et al. Proc. Natl. Acad. Sci. (1992) 89:12180), HIV-1 (Haffer et al., (1990) J. Virol. 64:2653), and hepatitis A (Winokur (1991) 65:5029), each of which is incorporated herein in its entirety. Methods for expressing VLPs that contain NDV proteins are provided by Pantua et al. (2006) J. Virol. 80:11062-11073, and in United States patent application Publication No. 20090068221, published Mar. 12, 2009, each of which is incorporated in its entirety herein. In a specific embodiment, the VLPs comprising chimeric HA polypeptides described herein are generated using baculovirus. In other embodiments, the VLPs comprising chimeric HA polypeptides described herein are generated using 293T cells.

In specific embodiments, VLPs, e.g., VLPs comprising a chimeric HA polypeptide, are expressed in cells (e.g., 293T cells). In certain embodiments, the VLPs are expressed in cells that express surface glycoproteins that comprise sialic acid. In accordance with such embodiments, the cells are cultured in the presence of neuraminidase (e.g., viral of bacterial neuraminidase). In certain embodiments, VLPs, e.g., VLPs comprising a chimeric HA polypeptide, are expressed in cells that do not express surface glycoproteins that comprise sialic acid.

In a specific embodiment, a chimeric HA polypeptide may be incorporated into a virosome. A virosome containing a chimeric HA polypeptide may be produced using techniques known to those skilled in the art. For example, a virosome may be produced by disrupting a purified virus, extracting the genome, and reassembling particles with the viral proteins (e.g., a chimeric HA polypeptide) and lipids to form lipid particles containing viral proteins.

### 5.6 Generation of Antibodies Against Chimeric Hemagglutinin (HA) Polypeptides

The chimeric hemagglutinin (HA) polypeptides, nucleic acids encoding such polypeptides, or vectors comprising such nucleic acids or polypeptides described herein may be used to elicit antibodies (e.g., neutralizing antibodies) against influenza, for example, against the stalk region of an influenza virus hemagglutinin polypeptide (e.g., subdominant epitopes of the stalk region of the influenza virus hemagglutinin polypeptide). In specific embodiments, the chimeric HA polypeptides, nucleic acids encoding such polypeptides, or vectors comprising such nucleic acids or polypeptides described herein may be used to elicit antibodies against conserved epitopes in the globular head domain of the chimeric HA. In specific embodiments, the chimeric HA polypeptides, nucleic acids encoding such polypeptides, or vectors comprising such nucleic acids or polypeptides described herein may be used to elicit antibodies against conserved epitopes in the stalk domain of the chimeric HA. In specific embodiments, the chimeric HA polypeptides, nucleic acids encoding such polypeptides, or vectors comprising such nucleic acids or polypeptides described herein may be used to elicit antibodies against conserved epitopes in the globular head domain and the stalk domain of the chimeric HA. In a specific embodiment, the chimeric hemagglutinin (HA) polypeptide, nucleic acids encoding such polypeptides, or vectors comprising such nucleic acids or polypeptides described herein, or immunogenic compositions described herein may be administered to a non-human subject (e.g., a mouse, rabbit, rat, guinea pig, etc.) to induce an immune response that includes the production of antibodies which may be isolated using techniques known to one of skill in the art (e.g., immunoaffinity chromatography, centrifugation, precipitation, etc.).

In certain embodiments, the non-human subjects administered a chimeric HA polypeptide(s), nucleic acid(s) encoding such polypeptide(s), or vector(s) comprising such nucleic acid(s) or polypeptide(s) described herein, or an immunogenic composition(s) described herein are transgenic animals (e.g., transgenic mice) that are capable of producing human antibodies. Human antibodies can be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. Companies such as Abgenix, Inc. (Freemont, Calif.), Genpharm (San Jose, Calif.), Regeneron (Tarryton, N.J.), and Medarex, Inc. (Princeton, N.J.) can be engaged to provide human antibodies directed against a selected antigen.

Alternatively, the chimeric hemagglutinin (HA) polypeptide described herein may be used to screen for antibodies from antibody libraries. For example, an isolated chimeric hemagglutinin (HA) polypeptide may be immobilized to a solid support (e.g., a silica gel, a resin, a derivatized plastic film, a glass bead, cotton, a plastic bead, a polystyrene bead, an alumina gel, or a polysaccharide, a magnetic bead), and screened for binding to antibodies. As an alternative, the antibodies may be immobilized to a solid support and screened for binding to the isolated chimeric hemagglutinin (HA) polypeptides. Any screening assay, such as a panning assay, ELISA, surface plasmon resonance, or other antibody screening assay known in the art may be used to screen for antibodies that bind to the chimeric hemagglutinin (HA) polypeptide. The antibody library screened may be a commercially available antibody library, an in vitro generated library, or a library obtained by identifying and cloning or isolating antibodies from an individual infected with influenza. In particular embodiments, the antibody library is generated from a survivor of an influenza virus outbreak. Antibody libraries may be generated in accordance with methods known in the art. In a particular embodiment, the antibody library is generated by cloning the antibodies and using them in phage display libraries or a phagemid display library.

Antibodies identified in the methods described herein may be tested for neutralizing activity and lack of autoreactivity using the biological assays known in the art or described herein. In one embodiment, an antibody isolated from a non-human animal or an antibody library neutralizes a hemagglutinin polypeptide from more than one influenza strain of a particular lineage or lineages. In some embodiments, an antibody elicited or identified using a chimeric hemagglutinin (HA) polypeptide, a nucleic acid encoding such a polypeptide(s), or a vector encoding such a nucleic acid or polypeptide neutralizes an influenza B virus of the Victoria lineage and/or an influenza B virus of the Yamagata lineage. In one embodiment, the neutralizing antibody neutralizes one or more influenza A viruses and one or more influenza B viruses.

Antibodies identified or elicited using a chimeric hemagglutinin (HA) polypeptide, a nucleic acid encoding such a polypeptide(s), or a vector comprising such a nucleic acid or polypeptide include immunoglobulin molecules and immunologically active portions of immunoglobulin molecules, i.e., molecules that contain an antigen binding site that specifically binds to a hemagglutinin polypeptide. The immunoglobulin molecules may be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. Antibodies include, but are not limited to, monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies elicited or identified using a method described herein), and epitope-binding fragments of any of the above.

Antibodies elicited or identified using a chimeric hemagglutinin (HA) polypeptide, nucleic acids encoding such a polypeptide(s) or a vector comprising such a nucleic acid or polypeptide may be used in diagnostic immunoassays, passive immunotherapy, and generation of antiidiotypic antibodies. The antibodies before being used in passive immunotherapy may be modified, e.g., the antibodies may be chimerized or humanized. See, e.g., U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741, each of which is incorporated herein by reference in its entirety, for reviews on the generation of chimeric and humanized antibodies. In addition, the ability of the antibodies to neutralize hemagglutinin polypeptides and the specificity of the antibodies for the polypeptides may be tested prior to using the antibodies in passive immunotherapy.

Antibodies elicited or identified using a chimeric hemagglutinin (HA) polypeptide, a nucleic acid encoding such a polypeptide(s), or a vector comprising such a nucleic acid or polypeptide may be used to monitor the efficacy of a therapy and/or disease progression. Without being bound by any particular theory, the level of antibodies elicited or identified using a chimeric hemagglutinin (HA) polypeptide may be indicative of the degree of protection against influenza virus disease: for example, a low level of influenza-specific antibodies may indicate that revaccination, or booster vaccination(s), are required. Any immunoassay system known in the art may be used for this purpose including, but not limited to, competitive and noncompetitive assay systems using techniques such as radioimmunoassays, ELISA (enzyme linked immunosorbent assays), “sandwich” immunoassays, precipitin reactions, gel diffusion precipitin reactions, immunodiffusion assays, agglutination assays, complement fixation assays, immunoradiometric assays, fluorescent immunoassays, protein A immunoassays and immunoelectrophoresis assays, to name but a few. Further, without being bound by any particular theory, elicited or identified can be utilized in an assay to determine the anti-influenza properties of the antibody (ies), which may be indicative of the level of protected provided by vaccination with the chimeric hemagglutinin (HA) polypeptide, the nucleic acid encoding such a polypeptide(s), or the vector comprising such a nucleic acid or polypeptide. Any assay known in the art for evaluating anti-influenza properties may be used for this purpose including, but not limited to, hemagglutinin inhibition assays, influenza virus growth curves, and plaque reduction assays, to name but a few.

Antibodies elicited or identified using a chimeric hemagglutinin (HA) polypeptide, a nucleic acid encoding such a polypeptide(s), or a vector comprising such a nucleic acid or polypeptide may be used in the production of antiidiotypic antibody. The antiidiotypic antibody can then in turn be used for immunization, in order to produce a subpopulation of antibodies that bind a particular antigen of influenza, e.g., a neutralizing epitope of a hemagglutinin polypeptide (Jerne, 1974, Ann. Immunol. (Paris) 125c: 373; Jerne et al., 1982, EMBO J. 1:234, incorporated herein by reference in its entirety).

### 5.7 Compositions

The nucleic acids, vectors, polypeptides, antibodies, or cells described herein (sometimes referred to herein as “active compounds”) may be incorporated into compositions. In specific embodiments, an active compound described herein is a chimeric hemagglutinin (HA) polypeptide described herein, a nucleic acid encoding such a polypeptide(s), a vector (e.g., a viral vector) either containing, expressing, or both such a polypeptide(s), or cells described herein. In a specific embodiment, the compositions are pharmaceutical compositions, such as immunogenic compositions (e.g., vaccine formulations). The pharmaceutical compositions provided herein can be in any form that allows for the composition to be administered to a subject. In a specific embodiment, the pharmaceutical compositions are suitable for veterinary and/or human administration. The compositions may be used in methods of preventing or treating an influenza virus disease.

In one embodiment, a pharmaceutical composition comprises a chimeric hemagglutinin (HA) polypeptide, in an admixture with a pharmaceutically acceptable carrier. In another embodiment, a pharmaceutical composition (e.g., an immunogenic composition) comprises a nucleic acid encoding a chimeric hemagglutinin (HA) polypeptide described herein, in an admixture with a pharmaceutically acceptable carrier. In another embodiment, a pharmaceutical composition (e.g., an immunogenic composition) comprises an expression vector comprising a nucleic acid encoding a chimeric hemagglutinin (HA) polypeptide, in an admixture with a pharmaceutically acceptable carrier. In another embodiment, a pharmaceutical composition (e.g., an immunogenic composition) comprises an influenza virus or non-influenza virus containing a chimeric hemagglutinin (HA) polypeptide, in an admixture with a pharmaceutically acceptable carrier. In another embodiment, a pharmaceutical composition (e.g., an immunogenic composition) comprises an influenza virus or non-influenza virus having a genome engineered to express a chimeric hemagglutinin (HA) polypeptide, in admixture with a pharmaceutically acceptable carrier. In another embodiment, a pharmaceutical composition (e.g., an immunogenic composition) comprises a virus-like particle or virosome containing a chimeric hemagglutinin (HA) polypeptide, in an admixture with a pharmaceutically acceptable carrier. In certain embodiments, the composition further comprises one or more adjuvants (see, e.g., Section 5.7.5, infra, such as AS03 or MF59).

In some embodiments, a pharmaceutical composition may comprise one or more other therapies in addition to a therapy that utilizes a chimeric hemagglutinin (HA) polypeptide described herein.

As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, pharmaceutical compositions are formulated to be suitable for the intended route of administration to a subject. For example, the pharmaceutical composition may be formulated to be suitable for parenteral, oral, intradermal, transdermal, colorectal, intraperitoneal, and rectal administration. In a specific embodiment, the pharmaceutical composition may be formulated for intravenous, oral, intraperitoneal, intranasal, intratracheal, subcutaneous, intramuscular, topical, intradermal, transdermal or pulmonary administration.

In certain embodiments, biodegradable polymers, such as ethylene vinyl acetate, polyanhydrides, polyethylene glycol (PEGylation), polymethyl methacrylate polymers, polylactides, poly(lactide-co-glycolides), polyglycolic acid, collagen, polyorthoesters, and polylactic acid, may be used as carriers. In some embodiments, the active compounds are prepared with carriers that increase the protection of the compound against rapid elimination from the body, such as a controlled release formulation, including implants and microencapsulated delivery systems. Methods for preparation of such formulations will be apparent to those skilled in the art. Liposomes or micelles can also be used as pharmaceutically acceptable carriers. These can be prepared according to methods known to those skilled in the art, for example, as described in U.S. Pat. No. 4,522,811. In certain embodiments, the pharmaceutical compositions comprise one or more adjuvants (see, e.g., Section 5.7, infra, for adjuvants, such as AS03 or MF49).

In specific embodiments, immunogenic compositions described herein are monovalent formulations. In other embodiments, immunogenic compositions described herein are multivalent formulations. In one example, a multivalent formulation comprises more than one vector expressing a chimeric hemagglutinin (HA) polypeptide. In another example, a multivalent formulation comprises more than one virus containing a chimeric hemagglutinin (HA) polypeptide. In certain embodiments, a multivalent formulation may comprise one or more different chimeric hemagglutinin (HA) polypeptides expressed using a single vector. In certain embodiments, immunogenic compositions described herein are trivalent vaccines which comprise one chimeric hemagglutinin (HA) polypeptide. In some embodiments, immunogenic compositions described herein are trivalent vaccines which comprise three different influenza viruses, each influenza virus comprising a different chimeric HA. In some embodiments, immunogenic compositions described herein are quadrivalent vaccines which comprise at least two different chimeric hemagglutinin (HA) polypeptides described herein. In some embodiments, immunogenic compositions described herein are quadrivalent vaccines which comprise four different influenza viruses, each influenza virus comprising a different chimeric HA. In certain embodiments, a composition described herein comprises one or more of the chimeric hemagglutinin (HA) polypeptides described in International Publication Nos. WO 2014/099931 and WO 2013/043729 and one or more of the chimeric HA polypeptides described herein. In certain embodiments, a compositions described herein may comprise a chimeric HA described herein and a recombinant HA, wherein the recombinant HA comprises the globular head domain of an HA of one subtype of an influenza A virus and the stem domain of an HA of a different subtype of an influenza A virus. For example, the recombinant HA may comprise the globular head domain of an HA of an influenza A virus of the H5 subtype and the stem domain of an HA of an influenza A virus of the H1 or H3 subtype. In another example, the recombinant HA may comprise the globular head domain of an HA of an influenza A virus of the H7 subtype and the stem domain of an influenza A virus of the H1 or H3 subtype.

In certain embodiments, the pharmaceutical compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative. In a specific embodiment, thimerosal is used during the manufacture of a pharmaceutical composition described herein and the thimerosal is removed via purification steps following production of the pharmaceutical composition, i.e., the pharmaceutical composition contains trace amounts of thimerosal (<0.3 μg of mercury per dose after purification; such pharmaceutical compositions are considered thimerosal-free products).

In certain embodiments, the pharmaceutical compositions described herein additionally comprise egg protein (e.g., ovalbumin or other egg proteins). The amount of egg protein in the pharmaceutical compositions described herein may range from about 0.0005 to about 1.2 μg of egg protein to 1 ml of pharmaceutical composition. In other embodiments, the pharmaceutical compositions described herein do not comprise egg protein.

In certain embodiments, the pharmaceutical compositions described herein additionally comprise one or more antimicrobial agents (e.g., antibiotics) including, but not limited to gentamicin, neomycin, polymyxin (e.g., polymyxin B), and kanamycin, streptomycin. In other embodiments, the pharmaceutical compositions described herein do not comprise any antibiotics.

In certain embodiments, the pharmaceutical compositions described herein additionally comprise one or more components used to inactivate a virus, e.g., formalin or formaldehyde or a detergent such as sodium deoxycholate, octoxynol 9 (Triton X-100), and octoxynol 10. In other embodiments, the pharmaceutical compositions described herein do not comprise any components used to inactivate a virus.

In certain embodiments, the pharmaceutical compositions described herein additionally comprise gelatin. In other embodiments, the pharmaceutical compositions described herein do not comprise gelatin.

In certain embodiments, the pharmaceutical compositions described herein additionally comprise one or more buffers, e.g., phosphate buffer and sucrose phosphate glutamate buffer. In other embodiments, the pharmaceutical compositions described herein do not comprise buffers.

In certain embodiments, the pharmaceutical compositions described herein additionally comprise one or more salts, e.g., sodium chloride, calcium chloride, sodium phosphate, monosodium glutamate, and aluminum salts (e.g., aluminum hydroxide, aluminum phosphate, alum (potassium aluminum sulfate), or a mixture of such aluminum salts). In other embodiments, the pharmaceutical compositions described herein do not comprise salts.

In specific embodiments, the pharmaceutical compositions described herein are low-additive influenza virus vaccines, i.e., the pharmaceutical compositions do not comprise one or more additives commonly found in influenza virus vaccines. Low-additive influenza vaccines have been described (see, e.g., International Application No. PCT/IB2008/002238 published as International Publication No. WO 09/001217, which is herein incorporated by reference in its entirety).

The pharmaceutical compositions described herein can be included in a container, pack, or dispenser together with instructions for administration.

The pharmaceutical compositions described herein can be stored before use, e.g., the pharmaceutical compositions can be stored frozen (e.g., at about −20° C. or at about −70° C.); stored in refrigerated conditions (e.g., at about 4° C.); or stored at room temperature (see International Application No. PCT/IB2007/001149 published as International Publication No. WO 07/110776, which is herein incorporated by reference in its entirety, for methods of storing compositions comprising influenza vaccines without refrigeration).

In certain embodiments, when the active compound in a pharmaceutical composition described herein is a cell engineered to express a chimeric hemagglutinin (HA) polypeptide, the cell is not a mammalian cell (e.g., the cell is a CB-1 cell).

### 5.7.1 Subunit Vaccines

In a specific embodiment, provided herein are subunit vaccines comprising a chimeric hemagglutinin (HA) polypeptide described herein. In some embodiments, a subunit vaccine comprises a chimeric hemagglutinin (HA) polypeptide, and one or more surface glycoproteins (e.g., influenza virus neuraminidase), other targeting moieties, or adjuvants. In a specific embodiment, the adjuvant is a type of, or a specific, adjuvant described in Section 5.7.5, infra. In specific embodiments, a subunit vaccine comprises a single chimeric hemagglutinin (HA) polypeptide. In other embodiments, a subunit vaccine comprises two, three, four or more chimeric hemagglutinin (HA) polypeptides. In specific embodiments, the chimeric hemagglutinin (HA) polypeptide(s) used in a subunit vaccine are not membrane-bound, i.e., are soluble. In specific embodiments, the polypeptide components of the subunit vaccine are generated in a baculovirus expression system. In a particular embodiment, a subunit vaccine comprises a purified chimeric HA polypeptide described herein which is produced in a continuous insect cell line, such as one derived from the fall armyworm Spodoptera frugiperda using a baculovirus vector (e.g., Autographa californica nuclear polyhedrosis virus). The chimeric HA polypeptide may be extracted from the cells and further purified by column chromatography. In some embodiments, a subunit vaccine comprises more than one chimeric HA polypeptide described herein.

In a specific embodiment, the subunit vaccine is prepared using influenza virus that was propagated in embryonated chicken eggs (i.e., the components of the subunit vaccine (e.g., a chimeric HA polypeptide) are isolated from virus that was propagated in embryonated chicken eggs). In another specific embodiment, the subunit vaccine is prepared using influenza virus that was not propagated in embryonated chicken eggs (i.e., the components of the subunit vaccine (e.g., a chimeric polypeptide) are isolated from virus that was not propagated in embryonated chicken eggs). In another specific embodiment, the subunit vaccine is prepared using influenza virus that was propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) (i.e., the components of the subunit vaccine (e.g., a chimeric HA polypeptide) are isolated from virus that was propagated in mammalian cells). In another specific embodiment, the chimeric HA polypeptide(s) in a subunit vaccine are prepared using an expression vector, e.g., a viral vector, plant vector, baculovirus vector, or a bacterial vector (i.e., the chimeric HA polypeptide(s) in the subunit vaccine are obtained/isolated from an expression vector).

### 5.7.2 Live Virus Vaccines

In one embodiment, provided herein are immunogenic compositions (e.g., vaccines) comprising live influenza virus containing a chimeric hemagglutinin (HA) polypeptide. In another embodiment, provided herein are immunogenic compositions (e.g., vaccines) comprising live influenza virus that is engineered to encode a chimeric hemagglutinin (HA) polypeptide, which is expressed by progeny virus produced in the subjects administered the compositions. In specific embodiments, the chimeric hemagglutinin (HA) polypeptide is membrane-bound. In other specific embodiments, the chimeric hemagglutinin (HA) polypeptide is not membrane-bound, i.e., it is soluble. In some embodiments, the live influenza virus is attenuated. In some embodiments, an immunogenic composition comprises two, three, four or more live influenza viruses containing or engineered to express two, three, four or more different chimeric hemagglutinin (HA) polypeptides.

An immunogenic composition comprising a live influenza virus for administration to a subject may be preferred because multiplication of the virus in the subject may lead to a prolonged stimulus of similar kind and magnitude to that occurring in natural infections, and therefore, confer substantial, long lasting immunity.

In a specific embodiment, the live virus that contains a chimeric HA polypeptide is propagated in embryonated chicken eggs before its use in an immunogenic composition described herein. In another specific embodiment, the live virus that contains a chimeric HA polypeptide is not propagated in embryonated chicken eggs before its use in an immunogenic composition described herein. In another specific embodiment, the live virus that contains a chimeric HA polypeptide is propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) before its use in an immunogenic composition described herein.

### 5.7.3 Inactivated Virus Vaccines

In one embodiment, provided herein are immunogenic compositions (e.g., vaccines) comprising an inactivated influenza virus containing a chimeric hemagglutinin (HA) polypeptide. In specific embodiments, the chimeric hemagglutinin (HA) polypeptide is membrane-bound. In some embodiments, an immunogenic composition comprises two, three, four or more inactivated influenza viruses containing two, three, four or more different chimeric hemagglutinin (HA) polypeptides. In certain embodiments, the inactivated influenza virus immunogenic compositions comprise one or more adjuvants. In a specific embodiment, the adjuvant is a type of, or a specific, adjuvant described in Section 5.7.5, infra.

Techniques known to one of skill in the art may be used to inactivate viruses containing a chimeric hemagglutinin (HA) polypeptide. Common methods use formalin, heat, or detergent for inactivation. See, e.g., U.S. Pat. No. 6,635,246, which is herein incorporated by reference in its entirety. Other methods include those described in U.S. Pat. Nos. 5,891,705; 5,106,619 and 4,693,981, which are incorporated herein by reference in their entireties.

### 5.7.4 Split Virus Vaccines

In one embodiment, an immunogenic composition comprising a chimeric hemagglutinin (HA) polypeptide is a split virus vaccine. In some embodiments, split virus vaccine contains two, three, four or more different chimeric hemagglutinin (HA) polypeptides. In certain embodiments, the chimeric hemagglutinin (HA) polypeptide is/was membrane-bound. In certain embodiments, the split virus vaccines comprise one or more adjuvants.

Techniques for producing split virus vaccines are known to those skilled in the art. By way of non-limiting example, an influenza virus split vaccine may be prepared using inactivated particles disrupted with detergents. One example of a split virus vaccine that can be adapted for use in accordance with the methods described herein is the Fluzone®, Influenza Virus Vaccine (Zonal Purified, Subvirion) for intramuscular use, which is formulated as a sterile suspension prepared from influenza viruses propagated in embryonated chicken eggs. The virus-containing fluids are harvested and inactivated with formaldehyde. Influenza virus is concentrated and purified in a linear sucrose density gradient solution using a continuous flow centrifuge. The virus is then chemically disrupted using a nonionic surfactant, octoxinol-9, (Triton® X-100-A registered trademark of Union Carbide, Co.) producing a “split virus.” The split virus is then further purified by chemical means and suspended in sodium phosphate-buffered isotonic sodium chloride solution.

### 5.7.5 Adjuvants

In certain embodiments, the compositions described herein comprise, or are administered in combination with, an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to a chimeric hemagglutinin (HA) polypeptide, but when the compound is administered alone does not generate an immune response to the polypeptide. In some embodiments, the adjuvant generates an immune response to the polypeptide and does not produce an allergy or other adverse reaction. Adjuvants can enhance an immune response by several mechanisms including, e.g., lymphocyte recruitment, stimulation of B and/or T cells, and stimulation of macrophages.

In certain embodiments, an adjuvant augments the intrinsic response to the chimeric hemagglutinin (HA) polypeptide without causing conformational changes in the polypeptide that affect the qualitative form of the response. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In specific embodiments, the adjuvant is AS03 (GlaxoSmithKline). In specific embodiments, the adjuvant is MF59 (Novartis). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as polyglutamic acid or polylysine. It should be understood that different formulations of chimeric HA polypeptides may comprise different adjuvants or may comprise the same adjuvant.

### 5.8 Prophylactic and Therapeutic Uses

In one aspect, provided herein are methods for inducing an immune response in a subject utilizing an active compound (e.g., a chimeric hemagglutinin (HA) polypeptide described herein, a nucleic acid encoding such a polypeptide(s), a vector (e.g., a viral vector) either containing, expressing, or both, such a polypeptide(s), cells described herein) or a composition described herein. In a specific embodiment, a method for inducing an immune response to an influenza virus hemagglutinin polypeptide in a subject comprises administering to a subject in need thereof an effective amount of a chimeric hemagglutinin (HA) polypeptide described herein or an immunogenic composition thereof. In another embodiment, a method for inducing an immune response to an influenza virus hemagglutinin polypeptide in a subject comprises administering to a subject in need thereof an effective amount of a nucleic acid encoding a chimeric hemagglutinin (HA) polypeptide described herein or an immunogenic composition thereof. In another embodiment, a method for inducing an immune response to an influenza virus hemagglutinin polypeptide in a subject comprises administering to a subject in need thereof an effective amount of a viral vector either containing, expressing, or both a chimeric hemagglutinin (HA) polypeptide described herein or an immunogenic composition thereof. In certain embodiments, a chimeric hemagglutinin (HA) polypeptide described herein used in the method is a purified chimeric hemagglutinin (HA) polypeptide described herein derived from a mammalian cell, a plant cell, or an insect cell.

In a specific embodiment, a method for inducing an immune response to an influenza virus hemagglutinin polypeptide in a subject comprises administering to a subject in need thereof a subunit vaccine described herein. In another embodiment, a method for inducing an immune response to an influenza virus hemagglutinin polypeptide in a subject comprises administering to a subject in need thereof a live influenza virus vaccine described herein. In particular embodiments, the live influenza virus vaccine comprises an attenuated influenza virus. In another embodiment, a method for inducing an immune response to an influenza virus hemagglutinin polypeptide in a subject comprises administering to a subject in need thereof an inactivated influenza virus vaccine described herein. In another embodiment, a method for inducing an immune response to an influenza virus hemagglutinin polypeptide in a subject comprises administering to a subject in need thereof a split virus vaccine described herein. In another embodiment, a method for inducing an immune response to an influenza virus hemagglutinin polypeptide in a subject comprises administering to a subject in need thereof a virus-like particle vaccine described herein. In another embodiment, a method for inducing an immune response to an influenza hemagglutinin polypeptide comprises administering to a subject in need thereof a virosome described herein. In certain embodiments, a chimeric hemagglutinin (HA) polypeptide described herein used in the method is a purified chimeric hemagglutinin (HA) polypeptide described herein derived from a mammalian cell, a plant cell, or an insect cell.

In a specific embodiment, a method for inducing an immune response to an influenza virus hemagglutinin polypeptide in a subject comprises: (a) administering to a subject in need thereof a first immunogenic composition comprising a chimeric HA described herein; and (b) a certain period of time after the administration of the first immunogenic composition, administering to the subject a second immunogenic composition comprising a second chimeric HA described herein. In specific embodiments, the first and second chimeric HA polypeptides comprise an ectodomain with the same stem domains but have one, two, three or all of the following: different 120 loops, different 150 loops, different 160 loops, and different 190 helices. In specific embodiments, the method further comprises administering to the subject a third immunogenic composition comprising a third chimeric HA described herein. In specific embodiments, the first, second, and third chimeric HA polypeptides comprise an ectodomain with the same stem domains but have one, two, three or all of the following: different 120 loops, different 150 loops, different 160 loops, and different 190 helices. In specific embodiments, the second immunogenic composition is administered about 6 weeks, about 12 weeks, about 4 months, about 6 months, or about 9 months after the administration of the first immunogenic composition. In another specific embodiment, the second immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the first immunogenic composition. In specific embodiments, the third immunogenic composition is administered about 6 weeks, about 12 weeks, about 4 months, about 6 months, or about 9 months after the administration of the second immunogenic composition. In another specific embodiment, the third immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the second immunogenic composition.

In another embodiment, provided herein is a method of immunizing a subject against an influenza virus disease or infection (e.g., an influenza B virus disease or infection) comprising (i) a first administration of a first chimeric HA polypeptide to the subject, a first nucleic acid encoding such a polypeptide(s), a first vector either containing, expressing, or both such a polypeptide(s), or cells described herein; and (ii) a second administration of a second chimeric HA polypeptide, a second nucleic acid encoding such a polypeptide(s), a second vector either containing, expressing, or both such a polypeptide(s), or cells described herein to the subject, wherein the first and second chimeric HA polypeptides comprise an ectodomain with the same stem domains but have one, two, three or all of the following: (i) different 120 loops, (ii) different 150 loops, (iii) different 160 loops, and (iv) different 190 helices. The first and second administrations may be separated by at least 1 day, 2 days, 3 days, 5 days, 10 days, 15 days, 30 days, 45 days, 2 months, 75 days, 3 months, or at least 6 months. In certain embodiments, the first and second administrations may be separated by 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months. In certain embodiments, booster inoculations may be administered to the subject at 6 to 12 month intervals following the second inoculation. In certain embodiments, the booster inoculation may be administered to the subject at 1 week to 9 month, 3 week to 8 month, 6 week to 12 week, 4 week to 6 month, 5 week to 5 month, 6 week to 4 month, 7 week to 4 month, 8 week to 4 month, 8 week to 3 month, 3 month to 6 month, 3 month to 9 month, or 6 month to 9 month intervals following the second inoculation. In certain embodiments, the booster inoculation comprises a third chimeric HA polypeptide, a third nucleic acid encoding such a polypeptide(s), a third vector either containing, expressing, or both such a polypeptide(s), or cells described herein, wherein the first, second, and third chimeric HA polypeptides comprise an ectodomain with the same stem domains but have either one, two, three, or all of the following: (i) different 120 loops, (ii) different 150 loops, (iii) different 160 loops, and (iv) different 190 helices.

In another embodiment, provided herein is a method of immunizing a subject against an influenza virus disease or infection (e.g., an influenza B virus infection or disease) comprising (i) a first administration of a first immunogenic composition to the subject, wherein the first immunogenic composition comprises a live attenuated influenza virus either containing, expressing, or both, a first chimeric HA polypeptide described herein; and (ii) a second administration of a second immunogenic composition to the subject, wherein the second immunogenic composition is an inactivated influenza virus vaccine comprising a second chimeric HA polypeptide described herein and an adjuvant (see, e.g., Section 5.75 for adjuvants), wherein the first and second chimeric HA polypeptides comprise an ectodomain with the same stem domains but have one, two, three or all of the following: (i) different 120 loops, (ii) different 150 loops, (iii) different 160 loops, and (iv) different 190 helices. The first and second administrations may be separated by at least 1 day, 2 days, 3 days, 5 days, 10 days, 15 days, 30 days, 45 days, 2 months, 75 days, 3 months, or at least 6 months. In certain embodiments, the first and second administrations may be separated by 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months. In certain embodiments, booster inoculations may be administered to the subject at 6 to 12 month intervals following the second inoculation. In certain embodiments, the booster inoculation may be administered to the subject at 1 week to 9 month, 3 week to 8 month, 6 week to 12 week, 4 week to 6 month, 5 week to 5 month, 6 week to 4 month, 7 week to 4 month, 8 week to 4 month, 8 week to 3 month, 3 month to 6 month, 3 month to 9 month, or 6 month to 9 month intervals following the second inoculation. In certain embodiments, the booster inoculation comprises a third chimeric HA polypeptide, a third nucleic acid encoding such a polypeptide(s), a third vector either containing, expressing, or both such a polypeptide(s), or cells described herein, wherein the first, second, and third chimeric HA polypeptides comprise an ectodomain with the same stem domains but have either one, two, three, or all of the following: (i) different 120 loops, (ii) different 150 loops, (iii) different 160 loops, and (iv) different 190 helices.

In another embodiment, provided herein is a method of immunizing a subject against an influenza virus disease or infection (e.g., an influenza B virus disease or infection) comprising (i) a first administration of a first immunogenic composition to the subject, wherein the first immunogenic composition comprises a live attenuated influenza virus either containing, expressing, or both, a first chimeric HA polypeptide described herein; and (ii) a second administration of a second immunogenic composition to the subject, wherein the second immunogenic composition is a subunit influenza virus vaccine comprising a second chimeric HA polypeptide described herein and an adjuvant (see, e.g., Section 5.75 for adjuvants, such as AS03 or MF59), wherein the first and second chimeric HA polypeptides comprise an ectodomain with the same stem domains but have one, two, three or all of the following: (i) different 120 loops, (ii) different 150 loops, (iii) different 160 loops, and (iv) different 190 helices. The first and second administrations may be separated by at least 1 day, 2 days, 3 days, 5 days, 10 days, 15 days, 30 days, 45 days, 2 months, 75 days, 3 months, or at least 6 months. In certain embodiments, the first and second administrations may be separated by 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months. In certain embodiments, booster inoculations may be administered to the subject at 6 to 12 month intervals following the second inoculation. In certain embodiments, the booster inoculation may be administered to the subject at 1 week to 9 month, 3 week to 8 month, 6 week to 12 week, 4 week to 6 month, 5 week to 5 month, 6 week to 4 month, 7 week to 4 month, 8 week to 4 month, 8 week to 3 month, 3 month to 6 month, 3 month to 9 month, or 6 month to 9 month intervals following the second inoculation. In certain embodiments, the booster inoculation comprises a third chimeric HA polypeptide, a third nucleic acid encoding such a polypeptide(s), a third vector either containing, expressing, or both such a polypeptide(s), or cells described herein, wherein the first, second, and third chimeric HA polypeptides comprise an ectodomain with the same stem domains but have either one, two, three, or all of the following: (i) different 120 loops, (ii) different 150 loops, (iii) different 160 loops, and (iv) different 190 helices.

In another embodiment, provided herein is a method of immunizing a subject against an influenza virus disease or infection (e.g., influenza B virus disease or infection) comprising (i) a first administration of a first immunogenic composition to the subject, wherein the first immunogenic composition comprises a live attenuated influenza virus either containing, expressing, or both, a first chimeric HA polypeptide described herein; and (ii) a second administration of a second immunogenic composition to the subject, wherein the second immunogenic composition is a split influenza virus vaccine comprising a second chimeric HA polypeptide described herein and an adjuvant (see, e.g., Section 5.75 for adjuvants, such as AS03 or MF59), wherein the first and second chimeric HA polypeptides comprise an ectodomain with the same stem domains but have one, two, three or all of the following: (i) different 120 loops, (ii) different 150 loops, (iii) different 160 loops, and (iv) different 190 helices. The first and second administrations may be separated by at least 1 day, 2 days, 3 days, 5 days, 10 days, 15 days, 30 days, 45 days, 2 months, 75 days, 3 months, or at least 6 months. In certain embodiments, the first and second administrations may be separated by 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months. In certain embodiments, booster inoculations may be administered to the subject at 6 to 12 month intervals following the second inoculation. In certain embodiments, the booster inoculation may be administered to the subject at 1 week to 9 month, 3 week to 8 month, 6 week to 12 week, 4 week to 6 month, 5 week to 5 month, 6 week to 4 month, 7 week to 4 month, 8 week to 4 month, 8 week to 3 month, 3 month to 6 month, 3 month to 9 month, or 6 month to 9 month intervals following the second inoculation. In certain embodiments, the booster inoculation comprises a third chimeric HA polypeptide, a third nucleic acid encoding such a polypeptide(s), a third vector either containing, expressing, or both such a polypeptide(s), or cells described herein, wherein the first, second, and third chimeric HA polypeptides comprise an ectodomain with the same stem domains but have either one, two, three, or all of the following: (i) different 120 loops, (ii) different 150 loops, (iii) different 160 loops, and (iv) different 190 helices.

In a specific embodiment, provided herein is a method for immunizing a subject against an influenza virus disease or infection (e.g., influenza B virus disease or infection), the method comprising: (a) a first administration of a nucleic acid encoding a first chimeric HA polypeptide, wherein the first chimeric HA polypeptide comprises a first ectodomain of a first influenza B virus comprising: (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a first subtype; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the first subtype; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the first subtype; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the first subtype; and (b) a second administration of a second chimeric HA polypeptide to the subject a first period of time after the first administration, wherein the second chimeric HA polypeptide comprises a second ectodomain of a second influenza B virus comprising: (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a second subtype; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the second subtype; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the second subtype; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the second subtype; and (c) a booster inoculation comprising a third chimeric HA polypeptide to the subject a second period of time after the second administration, wherein the third chimeric HA polypeptide comprises a third ectodomain of a third influenza B virus comprising (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a third subtype; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the third subtype; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the third subtype; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the third subtype. In a specific embodiment, the first, second, and third ectodomains comprise the same stem domain. In a particular embodiment, the stem domain is the stem domain of influenza B/Yamagata/16/88. In specific embodiment, the first, second, and third subtypes are different from each other. In a specific embodiment, the first subtype is the H13 subtype, the second subtype is the H5 subtype, and the third subtype is the H8 subtype. In some embodiments, the first, second, and third subtypes are different and the subtypes are selected from H5, H8, H11, H12, and H13. In a specific embodiment, the first chimeric HA polypeptide comprises the 120 loop, the 150 loop, the 160 loop, and the 190 helix of the chimeric HA of FIG. 36. In a specific embodiment, the second chimeric HA polypeptide comprises the 120 loop, the 150 loop, the 160 loop, and the 190 helix of the chimeric HA of FIG. 30. In a specific embodiment, the third chimeric HA polypeptide comprises the 120 loop, the 150 loop, the 160 loop, and the 190 helix of the chimeric HA of FIG. 32. In specific embodiments, the first period of time after the first administration is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months. In specific embodiments, the second period of time after the second administration is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months.

In a specific embodiment, a method for immunizing a subject against an influenza virus disease or infection (e.g., influenza B virus disease or infection), the method comprising: (a) a first administration of a first immunogenic composition comprising a first chimeric HA polypeptide, wherein the first chimeric HA polypeptide comprises a first ectodomain of a first influenza B virus comprising: (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of the H13 subtype; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the first subtype; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the first subtype; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the first subtype; and (b) a second administration of a second immunogenic composition comprising a second chimeric HA polypeptide to the subject a first period of time after the first administration, wherein the second chimeric HA polypeptide comprises a second ectodomain of a second influenza B virus comprising: (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a second subtype; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the second subtype; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the second subtype; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the second subtype; and (c) an third immunogenic composition comprising a third chimeric HA polypeptide to the subject a second period of time after the second administration, wherein the third chimeric HA polypeptide comprises a third ectodomain of a third influenza B virus comprising (i) 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a third subtype; (ii) 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the third subtype; (iii) 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the third subtype; and (iv) 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the third subtype. In a specific embodiment, the first, second, and third ectodomains comprise the same stem domain. In a particular embodiment, the stem domain is the stem domain of influenza B/Yamagata/16/88. In specific embodiment, the first, second, and third subtypes are different from each other. In a specific embodiment, the first subtype is the H13 subtype, the second subtype is the H5 subtype, and the third subtype is the H8 subtype. In some embodiments, the first, second, and third subtypes are different and the subtypes are selected from H5, H8, H11, H12, and H13. In a specific embodiment, the first chimeric HA polypeptide comprises the 120 loop, the 150 loop, the 160 loop, and the 190 helix of the chimeric HA of FIG. 36. In a specific embodiment, the second chimeric HA polypeptide comprises the 120 loop, the 150 loop, the 160 loop, and the 190 helix of the chimeric HA of FIG. 30. In a specific embodiment, the third chimeric HA polypeptide comprises the 120 loop, the 150 loop, the 160 loop, and the 190 helix of the chimeric HA of FIG. 32. In specific embodiments, either first, second or third chimeric HA polypeptides, or two or three of the first, second and third chimeric HA polypeptides may be administered to the subject as protein, in an influenza virus or other vector, or as a nucleic acid sequence. In specific embodiments, the first period of time after the first administration is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months. In specific embodiments, the second period of time after the second administration is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months.

In some embodiments, the immune response induced by an active compound or a composition described herein is effective to prevent and/or treat an influenza virus infection caused by one or both lineages of influenza B virus. In certain embodiments, the immune response induced by an active compound or a composition described herein is effective to prevent and/or treat an influenza virus infection caused by one or more strains within the same lineage of influenza B virus. In a specific embodiment, the immune response induced by an active compound or a composition described herein induces antibodies to the stem domain of an influenza B virus HA. In a specific embodiment, the immune response induced by an active compound or a composition described herein induces antibodies to subdominant epitopes in the globular head domain and the stem domain of an influenza B virus HA.

In some embodiments, the immune response induced by an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or a composition described herein is effective to reduce symptoms resulting from an influenza virus disease/infection. Symptoms of influenza virus disease/infection include, but are not limited to, body aches (especially joints and throat), fever, nausea, headaches, irritated eyes, fatigue, sore throat, reddened eyes or skin, and abdominal pain.

In some embodiments, the immune response induced by an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or a composition described herein is effective to reduce the hospitalization of a subject suffering from an influenza virus disease/infection. In some embodiments, the immune response induced by an active compound or a composition described herein is effective to reduce the duration of hospitalization of a subject suffering from an influenza virus disease/infection.

In another aspect, provided herein are methods for preventing and/or treating an influenza virus infection in a subject utilizing an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or a composition described herein. In one embodiment, a method for preventing or treating an influenza virus infection in a subject comprises administering to a subject in need thereof a chimeric hemagglutinin (HA) polypeptide, a nucleic acid encoding such a polypeptide(s), a vector either containing, expressing, or both such a polypeptide(s), or a composition of any one of the foregoing. In a specific embodiment, a method for preventing or treating an influenza virus infection in a subject comprises administering to a subject in need thereof a subunit vaccine, a live influenza virus vaccine, an inactivated influenza virus vaccine, a split virus vaccine, a virus-like particle vaccine, or virosome.

In another aspect, provided herein are methods for preventing and/or treating an influenza virus disease in a subject utilizing a chimeric hemagglutinin (HA) polypeptide described herein, a nucleic acid encoding such a polypeptide(s), a vector either containing, expressing, or both such a polypeptide(s), or cells described herein. In a specific embodiment, a method for preventing or treating an influenza virus disease in a subject comprises administering to a subject in need thereof an effective amount of a chimeric hemagglutinin (HA) polypeptide or an immunogenic composition thereof. In another embodiment, a method for preventing or treating an influenza virus disease in a subject comprises administering to a subject in need thereof an effective amount of a nucleic acid encoding a chimeric hemagglutinin (HA) polypeptide or an immunogenic composition thereof. In another embodiment, a method for preventing or treating an influenza virus disease in a subject comprises administering to a subject in need thereof an effective amount of a viral vector either containing, expressing, or both a chimeric hemagglutinin (HA) polypeptide or an immunogenic composition thereof.

In a specific embodiment, a method for preventing or treating an influenza virus disease in a subject comprises administering to a subject in need thereof a subunit vaccine described herein. In another embodiment, a method for preventing or treating an influenza virus disease in a subject comprises administering to a subject in need thereof a live influenza virus vaccine described herein. In particular embodiments, the live influenza virus vaccine comprises an attenuated virus. In another embodiment, a method for preventing or treating an influenza virus disease in a subject comprises administering to a subject in need thereof an inactivated influenza virus vaccine described herein. In another embodiment, a method for preventing or treating an influenza virus disease in a subject comprises administering to a subject in need thereof a split virus vaccine described herein. In another embodiment, a method for preventing or treating an influenza virus disease comprises administering to a subject in need thereof a virus-like particle vaccine described herein. In another embodiment, a method for preventing or treating an influenza virus disease in a subject, comprising administering to a subject in need thereof a virosome described herein.

In another aspect, provided herein are methods of preventing and/or treating an influenza virus disease in a subject by administering antibodies (e.g., neutralizing antibodies) described herein. In a specific embodiment, a method for preventing or treating an influenza virus disease in a subject comprises administering to a subject in need thereof an effective amount of an antibody described herein, or a pharmaceutical composition thereof. In particular embodiments, the antibody is a monoclonal antibody. In particular embodiments, the antibody is a human antibody. In particular embodiments, the antibody is a humanized antibody.

In certain embodiments, the methods for preventing or treating an influenza virus disease or infection in a subject (e.g., a human or non-human animal) provided herein result in a reduction in the replication of the influenza virus in the subject as measured by in vivo and in vitro assays known to those of skill in the art and described herein. In some embodiments, the replication of the influenza virus is reduced by approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs.

### 5.8.1 Combination Therapies

In various embodiments, a chimeric hemagglutinin (HA) polypeptide described herein, a nucleic acid encoding such a polypeptide(s), a vector (e.g., a viral vector) either containing, expressing, or both such a polypeptide(s), cells described herein, or a neutralizing antibody may be administered to a subject in combination with one or more other therapies (e.g., an antiviral, antibacterial, or immunomodulatory therapies). In some embodiments, a pharmaceutical composition (e.g., an immunogenic composition) described herein may be administered to a subject in combination with one or more therapies. The one or more other therapies may be beneficial in the treatment or prevention of an influenza virus disease or may ameliorate a symptom or condition associated with an influenza virus disease. In some embodiments, the one or more other therapies are pain relievers, anti-fever medications, or therapies that alleviate or assist with breathing. In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In specific embodiments, two or more therapies are administered within the same patent visit.

### 5.8.2 Patient Populations

In certain embodiments, an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or composition described herein may be administered to a naïve subject, i.e., a subject that does not have a disease caused by influenza virus infection or has not been and is not currently infected with an influenza virus infection. In one embodiment, an active compound or composition described herein is administered to a naïve subject that is at risk of acquiring an influenza virus infection. In one embodiment, an active compound or composition described herein is administered to a subject that does not have a disease caused by the specific influenza virus, or has not been and is not infected with the specific influenza virus to which the chimeric hemagglutinin (HA) polypeptide. An active compound or composition described herein may also be administered to a subject that is and/or has been infected with the influenza virus or another lineage, type, subtype or strain of the influenza virus to which the chimeric hemagglutinin (HA) polypeptide induces an immune response.

In certain embodiments, an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or composition described herein is administered to a patient who has been diagnosed with an influenza virus infection. In some embodiments, an active compound or composition described herein is administered to a patient infected with an influenza virus before symptoms manifest or symptoms become severe (e.g., before the patient requires hospitalization). In some embodiments, an active compound or composition described herein is administered to a patient that is infected with or has been diagnosed with a different lineage or strain of influenza B virus than that of the influenza B virus from which the chimeric hemagglutinin (HA) polypeptide of the active compound or composition was derived.

In some embodiments, a subject to be administered an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or composition described herein is an animal. In certain embodiments, the animal is a bird. In certain embodiments, the animal is a canine. In certain embodiments, the animal is a feline. In certain embodiments, the animal is a horse. In certain embodiments, the animal is a cow. In certain embodiments, the animal is a mammal, e.g., a horse, swine, mouse, or primate, preferably a human.

In specific embodiments, a subject administered an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or composition described herein is a human infant. As used herein, the term “human infant” refers to a newborn to 1 year old human. In specific embodiments, a subject administered an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or composition described herein is a human child. As used herein, the term “human child” refers to a human that is 1 year to 18 years old. In specific embodiments, a subject administered an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or composition described herein is a human adult. As used herein, the term “human adult” refers to a human that is 18 years or older. In specific embodiments, a subject administered an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or composition described herein is an elderly human. As used herein, the term “elderly human” refers to a human 65 years or older.

In certain embodiments, an immunogenic formulation comprising a live influenza virus vector is not given concurrently with other live-virus vaccines.

### 5.9 MODES OF ADMINISTRATION

### 5.9.1 Routes of Delivery

An active compound (e.g., a chimeric hemagglutinin (HA) polypeptide described herein (e.g., a nucleic acid encoding such a polypeptide(s), a vector (e.g., a viral vector) either containing, expressing, or both such a polypeptide(s), cells described herein) or composition described herein may be delivered to a subject by a variety of routes. These include, but are not limited to, intranasal, intratracheal, oral, intradermal, intramuscular, intraperitoneal, transdermal, intravenous, conjunctival and subcutaneous routes. In some embodiments, a composition is formulated for topical administration, for example, for application to the skin. In specific embodiments, the route of administration is nasal, e.g., as part of a nasal spray. In certain embodiments, a composition is formulated for intramuscular administration. In some embodiments, a composition is formulated for subcutaneous administration. In certain embodiments, a composition is not formulated for administration by injection. In specific embodiments for live virus vaccines, the vaccine is formulated for administration by a route other than injection.

In cases where the antigen is a viral vector or a virus-like particle vector, for example, it may be preferable to introduce an immunogenic composition via the natural route of infection of the backbone virus from which the vector was derived. Alternatively, it may be preferable to introduce a chimeric hemagglutinin (HA) polypeptide via the natural route of infection of the influenza virus from which polypeptide is derived. The ability of an antigen, particularly a viral vector, to induce a vigorous secretory and cellular immune response can be used advantageously. For example, infection of the respiratory tract by a viral vector may induce a strong secretory immune response, for example in the urogenital system, with concomitant protection against an influenza virus. In addition, in a preferred embodiment it may be desirable to introduce the pharmaceutical compositions into the lungs by any suitable route. Pulmonary administration can also be employed, e.g., by use of an inhaler or nebulizer, and formulation with an aerosolizing agent for use as a spray.

In a specific embodiment, a subunit vaccine is administered intramuscularly. In another embodiment, a live influenza virus vaccine is administered intranasally. In another embodiment, an inactivated influenza virus vaccine, or a split influenza virus vaccine is administered intramuscularly. In another embodiment, a virus-like particle or composition thereof is administered intramuscularly.

### 5.9.2 Dosage and Frequency of Administration

The amount of an active compound (e.g., a chimeric hemagglutinin (HA) polypeptide described herein, a nucleic acid encoding such a polypeptide(s), a vector (e.g., a viral vector) either containing, expressing, or both such a polypeptide(s), cells described herein) or composition which will be effective in the treatment and/or prevention of an influenza virus infection or an influenza virus disease will depend on the nature of the disease, and can be determined by standard clinical techniques.

The precise dose to be employed in the formulation will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, health), whether the patient is human or an animal, other medications administered, and whether treatment is prophylactic or therapeutic. Usually, the patient is a human but nonhuman mammals including transgenic mammals can also be treated. Treatment dosages are optimally titrated to optimize safety and efficacy.

In certain embodiments, the dose of a viral vector (e.g., an influenza virus) described herein may be 10⁴ plaque forming units (PFU) to 10⁸ PFU. In certain embodiments, the dose of a chimeric hemagglutinin (HA) polypeptide described herein (e.g., as provided in split virus vaccines and subunit vaccines) may range from about 1 μg to 150 μg. In certain embodiments, the dose for VLPs may range from about 1 μg to about 150 μg of a chimeric HA polypeptide. In some embodiments, an inactivated vaccine is formulated such that it may contain about 1 μg to about 150 μg of a chimeric hemagglutinin (HA) polypeptide described herein.

In certain embodiments, an in vitro assay is employed to help identify optimal dosage ranges. Effective doses may be extrapolated from dose response curves derived from in vitro or animal model test systems.

### 5.10 Assessment of Antibodies in a Subject

In another aspect, chimeric HA described herein, or virus containing, or expressing, or both a chimeric HA described herein, can be used to assess the antibody response of a subject (e.g., a naive subject or an immunized/vaccinated subject) or a population of subjects to an influenza virus hemagglutinin polypeptide (e.g., a chimeric HA). In specific embodiments, a chimeric HA or a virus containing, expressing, or both a chimeric HA can be used to assess the presence of stem-specific antibodies in the subject or population of subjects.

In a specific embodiment, the antibody response of a subject or a population of subjects that has been an immunized/vaccinated with a chimeric HA described herein, or a virus containing, or expressing, or both a chimeric HA described herein, is assessed to identify the types of stalk-specific antibodies in the subject or population of subjects. Such an assessment may allow for the identification surrogate markers/endpoints important in determining the clinical response to administration of chimeric HA described herein, or a virus containing, expressing, or both a chimeric HA described herein. In such an approach, a biological sample, e.g., blood, from the subject or population of subjects may be isolated and tested directly for the presence of antibodies, or may be processed (e.g., to obtain sera) and subsequently tested for the presence of antibodies.

In another specific embodiment, the antibody profile of a naive subject (i.e., a subject that has not been immunized/vaccinated with a chimeric HA described herein, or a virus containing or expressing or both a chimeric HA) or a population of naive subjects is assessed to determine whether said subject or population of subjects possesses globular head-specific and/or stem specific antibodies against various influenza virus strains or subtypes. Such an assessment may allow for the generation of a chimeric HA, or viruses containing, expressing, or both chimeric HA, that are suitable for administration to said subject or population of subjects, e.g., chimeric HAs, comprising a head domain to which said subject or population of subjects is naive (does not have antibodies against). Such an assessment may determine an immunization strategy for the patient.

In another specific embodiment, provided herein is a method of assessing/detecting the presence of antibodies in a subject that are specific for a stem domain of a particular influenza virus strain or lineage comprising contacting in vitro a biological sample (e.g., blood, sera) from said subject with a chimeric HA described herein, wherein said chimeric HA comprises a stem domain from the strain or lineage of interest. In another specific embodiment, provided herein is a method of assessing/detecting the presence of antibodies in a subject that are specific for a stem domain of a particular influenza virus strain or lineage comprising contacting in vitro a biological sample (e.g., blood, sera) from said subject with a virus expressing/containing a chimeric HA described herein, wherein said chimeric HA comprises a stem domain from the strain or lineage of interest.

### 5.11 Biological Assays

Also provided herein are biological assays that may be used to characterize chimeric HA, nucleic acid encoding such chimeric HA, and viruses containing, expressing, or both such chimeric HA. See, also, Section 6, infra.

### 5.11.1 Assays for Testing Activity of Chimeric Influenza Virus Hemagglutinin Polypeptides

Assays for testing the expression of a chimeric hemagglutinin (HA) polypeptide in a vector disclosed herein may be conducted using any assay known in the art. For example, an assay for incorporation into a viral vector comprises growing the virus as described in this section or Sections 5.4 or 5.5, purifying the viral particles by centrifugation through a sucrose cushion, and subsequent analysis for chimeric hemagglutinin (HA) polypeptide expression by an immunoassay, such as Western blotting, using methods well known in the art. Methods for determining whether a hemagglutinin polypeptide is chimeric are known to those of skill in the art (see, e.g., the Examples 3 and 4 of International Publication No. WO 2013/043729, which is incorporated herein by reference in its entirety).

In one embodiment, a chimeric hemagglutinin (HA) polypeptide disclosed herein is assayed for proper folding and functionality by testing its ability to bind specifically to a neutralizing antibody directed to an influenza virus hemagglutinin polypeptide, such as the stalk region of the chimeric hemagglutinin (HA) polypeptide, respectively, using any assay for antibody-antigen interaction known in the art. Neutralizing antibodies for use in such assays include, for example, the neutralizing antibodies described in Ekiert et al., 2009, Science Express, 26 Feb. 2009; Kashyap et al., 2008, Proc Natl Acad Sci USA 105:5986-5991; Sui et al. 2009, Nature Structural and Molecular Biology, 16:265-273; Wang et al., 2010, PLOS Pathogens 6(2):1-9; U.S. Pat. Nos. 5,589,174, 5,631,350, 6,337,070, and 6,720,409; International Application No. PCT/US2007/068983 published as International Publication No. WO 2007/134237; International Application No. PCT/US2008/075998 published as International Publication No. WO 2009/036157; International Application No. PCT/EP2007/059356 published as International Publication No. WO 2008/028946; and International Application No. PCT/US2008/085876 published as International Publication No. WO 2009/079259. These antibodies include CR6261, CR6325, CR6329, CR6307, CR6323, 2A, D7, D8, F10, G17, H40, A66, D80, E88, E90, H98, C179 (FERM BP-4517), AI3C (FERM BP-4516), among others.

In another embodiment, a chimeric hemagglutinin (HA) polypeptide disclosed herein is assayed for proper folding by determination of the structure or conformation of the chimeric hemagglutinin (HA) polypeptide using any method known in the art such as, e.g., NMR, X-ray crystallographic methods, or secondary structure prediction methods, e.g., circular dichroism.

In another embodiment, a chimeric HA disclosed herein is assayed for retention of one, two, or more, or all of the functions of a wild-type influenza HA. Nonlimiting examples of functions of a wild-type influenza HA include fusogenic activity, receptor binding activity, budding, and particle formation. In a specific embodiment, a chimeric HA disclosed herein is assayed for fusogenic activity. Assays known to one skilled in the art can be utilized the assess the fusogenic activity of a chimeric influenza hemagglutinin (HA) polypeptide described herein, such as, for example, immunofluorescence assays and pseudotyped virus-like-particle assays. In certain embodiments, the activity of a chimeric HA polypeptide described herein is assessed in one or more of the following assays: hemagglutination assay(s), fusion assay(s) or budding assay(s).

### 5.11.2 Assays for Testing Activity of Antibodies Generated Using Chimeric Influenza Virus Hemagglutinin Polypeptides

Antibodies described herein may be characterized in a variety of ways known to one of skill in the art (e.g. ELISA, Surface Plasmon resonance display (BIAcore), Western blot, immunofluorescence, immunostaining and/or microneutralization assays). Such an assay may be performed in solution (e.g., Houghten, 1992, Bio/Techniques 13:412 421), on beads (Lam, 1991, Nature 354:82 84), on chips (Fodor, 1993, Nature 364:555 556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865 1869) or on phage (Scott and Smith, 1990, Science 249:386 390; Cwirla et al., 1990, Proc. Natl. Acad. Sci. USA 87:6378 6382; and Felici, 1991, J. Mol. Biol. 222:301 310) (each of these references is incorporated herein in its entirety by reference).

Specific binding of an antibody to a chimeric hemagglutinin (HA) polypeptide or a domain thereof and cross-reactivity with other antigens can be assessed by any method known in the art. Immunoassays which can be used to analyze specific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety).

The binding affinity of an antibody to a chimeric hemagglutinin (HA) polypeptide or a domain thereof and the off-rate of an antibody-antigen interaction can be determined by competitive binding assays. One example of a competitive binding assay is a radioimmunoassay comprising the incubation of labeled antigen (e.g., 3H or 1251) with the antibody of interest in the presence of increasing amounts of unlabeled antigen, and the detection of the antibody bound to the labeled antigen. The affinity of the antibody for a chimeric hemagglutinin (HA) polypeptide and the binding off-rates can be determined from the data by Scatchard plot analysis. Competition with a second antibody can also be determined using radioimmunoassays. In this case, a chimeric hemagglutinin (HA) polypeptide is incubated with the test antibody conjugated to a labeled compound (e.g., 3H or 1251) in the presence of increasing amounts of an unlabeled second antibody.

In a specific embodiment, the binding affinity of an antibody to a chimeric HA polypeptide or a domain thereof is determined using an assay described in Nachbagauer et al., mBio. 2016 January-February; 7(1): e01996-15.

In certain embodiments, antibody binding affinity and rate constants are measured using the KinExA 3000 System (Sapidyne Instruments, Boise, ID). In some embodiments, surface plasmon resonance (e.g., BIAcore kinetic) analysis is used to determine the binding on and off rates of the antibodies to an influenza virus hemagglutinin polypeptide. In specific embodiments, an assay described in Tan et al., PLOS Pathog. 2016 April; 12(4): e1005578 is used to determine the binding on and off rates of antibodies to a chimeric HA polypeptide.

The neutralizing activity of an antibody can be determined utilizing any assay known to one skilled in the art. Antibodies described herein can be assayed for their ability to inhibit the binding of an influenza virus, or any other composition comprising a chimeric hemagglutinin (HA) polypeptide to its host cell receptor (i.e., sialic acid) using techniques known to those of skill in the art. In a specific embodiment, an assay described in one of the following articles is used to determine the neutralizing activity of an antibody Tan et al., PLoS Pathog. 2016 April; 12(4): e1005578; Pica et al., Proc Natl Acad Sci USA. 2012 Feb. 14; 109(7): 2573-2578; and Nachbagauer et al., mBio. 2016 January-February; 7(1): e01996-15.

In other embodiments, an antibody suitable for use in the methods described herein does not inhibit influenza virus receptor binding, yet is still found to be neutralizing in an assay described herein. In some embodiments, an antibody suitable for use in accordance with the methods described herein reduces or inhibits virus-host membrane fusion in an assay known in the art or described herein.

In one embodiment, virus-host membrane fusion is assayed in an in vitro assay using an influenza virus containing a reporter and a host cell capable of being infected with the virus. An antibody inhibits fusion if reporter activity is inhibited or reduced compared to a negative control (e.g., reporter activity in the presence of a control antibody or in the absence of antibody). In a specific embodiment, a reporter assay described in Heaton et al., J Virol. 2013 August; 87 (15): 8272-81 is used.

### 5.11.3 Antiviral Activity Assays

Antibodies described herein or compositions thereof can be assessed in vitro for antiviral activity. In one embodiment, the antibodies or compositions thereof are tested in vitro for their effect on growth of an influenza virus. Growth of influenza virus can be assessed by any method known in the art or described herein (e.g. in cell culture). In a specific embodiment, cells are infected at a MOI of 0.0005 and 0.001, 0.001 and 0.01, 0.01 and 0.1, 0.1 and 1, or 1 and 10, or a MOI of 0.0005, 0.001, 0.005, 0.01, 0.05, 0.1, 0.5, 1, 5 or 10 and incubated with serum free media supplemented. Viral titers are determined in the supernatant by hemagglutinin plaques or any other viral assay described herein. Cells in which viral titers can be assessed include, but are not limited to, EFK-2 cells, Vero cells, MDCK cells, primary human umbilical vein endothelial cells (HUVEC), H292 human epithelial cell line and HeLa cells. In vitro assays include those that measure altered viral replication (as determined, e.g., by plaque formation) or the production of viral proteins (as determined, e.g., by Western blot analysis) or viral RNAs (as determined, e.g., by RT-PCR or northern blot analysis) in cultured cells in vitro using methods which are well known in the art or described herein.

In one non-limiting example, a monolayer of the target mammalian cell line is infected with different amounts (e.g., multiplicity of 3 plaque forming units (pfu) or 5 pfu) of virus (e.g., influenza) and subsequently cultured in the presence or absence of various dilutions of antibodies (e.g., 0.1 μg/ml, 1 μg/ml, 5 μg/ml, or 10 μg/ml). Infected cultures are harvested 48 hours or 72 hours post infection and titered by standard plaque assays known in the art on the appropriate target cell line (e.g., Vero cells).

In a non-limiting example of a hemagglutination assay, cells are contacted with an antibody and are concurrently or subsequently infected with the virus (e.g., at an MOI of 1) and the virus is incubated under conditions to permit virus replication (e.g., 20-24 hours). The antibodies are preferably present throughout the course of infection. Viral replication and release of viral particles is then determined by hemagglutination assays using 0.5% chicken red blood cells. See, e.g., Kashyap et al., PNAS USA 105:5986-5991. In some embodiments, a compound is considered an inhibitor of viral replication if it reduces viral replication by at least 2 wells of HA, which equals approximately a 75% reduction in viral titer. In specific embodiments, an inhibitor reduces viral titer in this assay by 50% or more, by 55% or more, by 60% or more, by 65% or more, by 70% or more, by 75% or more, by 80% or more, by 85% or more, by 90% or more, or by 95% or more. In other specific embodiments an inhibitor results in a reduction of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs in influenza virus titer in the subject. The log-reduction in Influenza virus titer may be as compared to a negative control, as compared to another treatment, or as compared to the titer in the patient prior to antibody administration.

In a non-limiting example of a hemagglutination assay, cells are contacted with an antibody and are concurrently or subsequently infected with the virus (e.g., at an MOI of 1) and the virus is incubated under conditions to permit virus replication (e.g., 20-24 hours). The antibodies are preferably present throughout the course of infection. Viral replication and release of viral particles is then determined by hemagglutination assays using 0.5% chicken red blood cells. See, e.g., Kashyap et al., PNAS USA 105:5986-5991. In some embodiments, a compound is considered an inhibitor of viral replication if it reduces viral replication by at least 2 wells of HA, which equals approximately a 75% reduction in viral titer. In specific embodiments, an inhibitor reduces viral titer in this assay by 50% or more, by 55% or more, by 60% or more, by 65% or more, by 70% or more, by 75% or more, by 80% or more, by 85% or more, by 90% or more, or by 95% or more. In other specific embodiments an inhibitor results in a reduction of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs in influenza virus titer in the subject. The log-reduction in Influenza virus titer may be as compared to a negative control, as compared to another treatment, or as compared to the titer in the patient prior to antibody administration.

### 5.11.4 Cytotoxicity Assays

Many assays well-known in the art can be used to assess viability of cells (infected or uninfected) or cell lines following exposure to an active compound or a composition thereof and, thus, determine the cytotoxicity of the compound or composition. For example, cell proliferation can be assayed by measuring Bromodeoxyuridine (BrdU) incorporation (See, e.g., Hoshino et al., 1986, Int. J. Cancer 38, 369; Campana et al., 1988, J. Immunol. Meth. 107:79), (3H) thymidine incorporation (See, e.g., Chen, J., 1996, Oncogene 13:1395-403; Jeoung, J., 1995, J. Biol. Chem. 270:18367 73), by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability.

In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes. These changes are given a designation of T (100% toxic), PVH (partially toxic-very heavy-80%), PH (partially toxic-heavy-60%), P (partially toxic-40%), Ps (partially toxic-slight-20%), or 0 (no toxicity-0%), conforming to the degree of cytotoxicity seen. A 50% cell inhibitory (cytotoxic) concentration (IC₅₀) is determined by regression analysis of these data.

In a specific embodiment, the cells used in the cytotoxicity assay are animal cells, including primary cells and cell lines. In some embodiments, the cells are human cells. In certain embodiments, cytotoxicity is assessed in one or more of the following cell lines: U937, a human monocyte cell line; primary peripheral blood mononuclear cells (PBMC); Huh7, a human hepatoblastoma cell line; 293T, a human embryonic kidney cell line; and THP-1, monocytic cells. In certain embodiments, cytotoxicity is assessed in one or more of the following cell lines: MDCK, MEF, Huh 7.5, Detroit, or human tracheobronchial epithelial (HTBE) cells.

Active compounds or compositions thereof can be tested for in vivo toxicity in animal models. For example, animal models, described herein and/or others known in the art, used to test the activities of active compounds can also be used to determine the in vivo toxicity of these compounds. For example, animals are administered a range of concentrations of active compounds. Subsequently, the animals are monitored over time for lethality, weight loss or failure to gain weight, and/or levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and/or regimen in addition to dosages.

The toxicity and/or efficacy of an active compound can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. An active compound that exhibits large therapeutic indices is preferred. While an active compound that exhibits toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of an active compound for use in humans. The dosage of such agents lies preferably within a range of circulating concentrations that include the ED₅₀ with little or no toxicity. The dosage may vary within this range depending upon the dosage form employed and the route of administration utilized. For any active compound used in a method described herein, the effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high-performance liquid chromatography. Additional information concerning dosage determination is provided herein.

Further, any assays known to those skilled in the art can be used to evaluate the prophylactic and/or therapeutic utility of the active compounds and compositions described herein, for example, by measuring viral infection or a condition or symptoms associated therewith.

The cytotoxicity assays described herein and known to those skilled in the art are particularly useful for live attenuated influenza viruses.

### 5.11.5 In Vivo Antiviral Activity

Active compounds and compositions thereof are preferably assayed in vivo for the desired therapeutic or prophylactic activity prior to use in humans. For example, in vivo assays can be used to determine whether it is preferable to administer an active compound or composition thereof and/or another therapy. For example, to assess the use of an active compound or composition thereof to prevent an influenza virus disease, the composition can be administered before the animal is infected with influenza virus. Alternatively, or in addition, an active compound or composition thereof can be administered to the animal at the same time that the animal is infected with influenza virus. To assess the use of an active compound or composition thereof to treat an influenza virus infection or disease associated therewith, the compound or composition may be administered after infecting the animal with influenza virus. In a specific embodiment, an active compound or composition thereof is administered to the animal more than one time.

Active compounds and compositions thereof can be tested for antiviral activity in animal model systems including, but are not limited to, rats, mice, chicken, cows, monkeys, pigs, ferrets, goats, sheep, dogs, rabbits, guinea pigs, etc. In a specific embodiment, active compounds and compositions thereof are tested in a mouse model system. Such model systems are widely used and well-known to the skilled artisan. In a specific embodiment, active compounds and compositions thereof are tested in a mouse model system. Non-limiting examples of animal models for influenza virus are provided in this section.

In general, animals are infected with influenza virus and concurrently or subsequently treated with an active compound or composition thereof, or placebo. Alternatively, animals are treated with an active compound or composition thereof or placebo and subsequently infected with influenza virus. Samples obtained from these animals (e.g., serum, urine, sputum, semen, saliva, plasma, or tissue sample) can be tested for viral replication via well known methods in the art, e.g., those that measure altered viral titers (as determined, e.g., by plaque formation), the production of viral proteins (as determined, e.g., by Western blot, ELISA, or flow cytometry analysis) or the production of viral nucleic acids (as determined, e.g., by RT-PCR or northern blot analysis). For quantitation of virus in tissue samples, tissue samples are homogenized in phosphate-buffered saline (PBS), and dilutions of clarified homogenates are adsorbed for 1 hour at 37° C. onto monolayers of cells (e.g., Vero, CEF or MDCK cells). In other assays, histopathologic evaluations are performed after infection, preferably evaluations of the organ(s) the virus is known to target for infection. Virus immunohistochemistry can be performed using a viral-specific monoclonal antibody.

The effect of an active compound or composition thereof on the virulence of a virus can also be determined using in vivo assays in which the titer of the virus in an infected subject administered an active compound or composition thereof, the length of survival of an infected subject administered an active compound or composition thereof, the immune response in an infected subject administered an active compound or composition thereof, the number, duration and/or severity of the symptoms in an infected subject administered an active compound or composition thereof, and/or the time period before onset of one or more symptoms in an infected subject administered an active compound or composition thereof, is assessed. Techniques known to one of skill in the art can be used to measure such effects. In certain embodiments, an active compound or composition thereof results in a 0.5 fold, 1 fold, 2 fold, 4 fold, 6 fold, 8 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titer of influenza virus relative to an untreated subject. In some embodiments, an active compound or composition thereof results in a reduction in titer of influenza virus relative to an untreated subject of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs.

Influenza virus animal models, such as ferret, mouse, guinea pig, squirrel monkey, macaque, and chicken, developed for use to test antiviral agents against influenza virus have been described. See, e.g., Sidwell et al., Antiviral Res., 2000, 48:1-16; Lowen A. C. et al. PNAS, 2006, 103:9988-92; and McCauley et al., Antiviral Res., 1995, 27:179-186 and Rimmelzwann et al., Avian Diseases, 2003, 47:931-933. For mouse models of influenza, non-limiting examples of parameters that can be used to assay antiviral activity of active compounds administered to the influenza-infected mice include pneumonia-associated death, serum al-acid glycoprotein increase, animal weight, lung virus assayed by hemagglutinin, lung virus assayed by plaque assays, and histopathological change in the lung. Statistical analysis is carried out to calculate significance (e.g., a P value of 0.05 or less).

In other assays, histopathologic evaluations are performed after infection of an animal model subject. Nasal turbinates and trachea may be examined for epithelial changes and subepithelial inflammation. The lungs may be examined for bronchiolar epithelial changes and peribronchiolar inflammation in large, medium, and small or terminal bronchioles. The alveoli are also evaluated for inflammatory changes. The medium bronchioles are graded on a scale of 0 to 3+ as follows: 0 (normal: lined by medium to tall columnar epithelial cells with ciliated apical borders and basal pseudostratified nuclei; minimal inflammation); 1+ (epithelial layer columnar and even in outline with only slightly increased proliferation; cilia still visible on many cells); 2+ (prominent changes in the epithelial layer ranging from attenuation to marked proliferation; cells disorganized and layer outline irregular at the luminal border); 3+ (epithelial layer markedly disrupted and disorganized with necrotic cells visible in the lumen; some bronchioles attenuated and others in marked reactive proliferation).

The trachea is graded on a scale of 0 to 2.5+ as follows: 0 (normal: Lined by medium to tall columnar epithelial cells with ciliated apical border, nuclei basal and pseudostratified. Cytoplasm evident between apical border and nucleus. Occasional small focus with squamous cells); 1+ (focal squamous metaplasia of the epithelial layer); 2+ (diffuse squamous metaplasia of much of the epithelial layer, cilia may be evident focally); 2.5+ (diffuse squamous metaplasia with very few cilia evident).

Virus immunohistochemistry is performed using a viral-specific monoclonal antibody (e.g. NP-, N- or HN-specific monoclonal antibodies). Staining is graded 0 to 3+ as follows: 0 (no infected cells); 0.5+ (few infected cells); 1+ (few infected cells, as widely separated individual cells); 1.5+ (few infected cells, as widely separated singles and in small clusters); 2+ (moderate numbers of infected cells, usually affecting clusters of adjacent cells in portions of the epithelial layer lining bronchioles, or in small sublobular foci in alveoli); 3+ (numerous infected cells, affecting most of the epithelial layer in bronchioles, or widespread in large sublobular foci in alveoli).

In one example, the ability to induce lung lesions and cause infection in an animal model of virus infection is compared using wild-type virus and mock virus. Lung lesions can be assessed as a percentage of lung lobes that are healthy by visual inspection. Animals are euthanized 5 days p.i. by intravenous administration of pentobarbital, and their lungs are removed in toto. The percentage of the surface of each pulmonary lobe that is affected by macroscopic lesions is estimated visually. The percentages are averaged to obtain a mean value for the 7 pulmonary lobes of each animal. In other assays, nasal swabs can be tested to determine virus burden or titer. Nasal swabs can be taken during necropsy to determine viral burden post-infection.

In one embodiment, virus is quantified in tissue samples. For example, tissue samples are homogenized in phosphate-buffered saline (PBS), and dilutions of clarified homogenates adsorbed for 1 h at 37° C. onto monolayers of cells (e.g., MDCK cells). Infected monolayers are then overlaid with a solution of minimal essential medium containing 0.1% bovine serum albumin (BSA), 0.01% DEAE-dextran, 0.1% NaHCO₃, and 1% agar. Plates are incubated 2 to 3 days until plaques could be visualized. Tissue culture infectious dose (TCID) assays to titrate virus from PR8-infected samples are carried out as follows. Confluent monolayers of cells (e.g., MDCK cells) in 96-well plates are incubated with log dilutions of clarified tissue homogenates in media. Two to three days after inoculation, 0.05-ml aliquots from each well are assessed for viral growth by hemagglutination assay (HA assay).

### 5.11.5.1.1 Assays in Humans

In one embodiment, an active compound or composition thereof that modulates replication of an influenza virus are assessed in infected human subjects. In accordance with this embodiment, an active compound or composition thereof is administered to the human subject, and the effect of the active compound or composition on viral replication is determined by, e.g., analyzing the level of the virus or viral nucleic acids in a biological sample (e.g., serum or plasma). An active compound or composition thereof that alters virus replication can be identified by comparing the level of virus replication in a subject or group of subjects treated with a control to that in a subject or group of subjects treated with an active compound or composition thereof. Alternatively, alterations in viral replication can be identified by comparing the level of the virus replication in a subject or group of subjects before and after the administration of an active compound or composition thereof. Techniques known to those of skill in the art can be used to obtain the biological sample and analyze the mRNA or protein expression.

In another embodiment, the effect of an active compound or composition thereof on the severity of one or more symptoms associated with an influenza virus infection/disease are assessed in an infected subject. In accordance with this embodiment, an active compound or composition thereof or a control is administered to a human subject suffering from influenza virus infection and the effect of the active compound or composition on one or more symptoms of the virus infection is determined. An active compound or composition thereof that reduces one or more symptoms can be identified by comparing the subjects treated with a control to the subjects treated with the active compound or composition. In a specific embodiment, administration of an active compound (e.g., a chimeric hemagglutinin (HA) polypeptide described herein an) or composition thereof results in a decrease in hospitalization of a human or population of humans caused by influenza virus disease or infection. In another specific embodiment, administration of an active compound (e.g., a chimeric hemagglutinin (HA) polypeptide described herein) or composition thereof results in a reduced need for respiratory/breathing assistance in a human or population of humans with an influenza virus disease or infection. In another specific embodiment, administration of an active compound (e.g., a chimeric hemagglutinin (HA) polypeptide described herein) or composition thereof results in a reduced length of illness of a human or population of humans with an influenza virus disease or infection. In another specific embodiment, administration of an active compound (e.g., a chimeric hemagglutinin (HA) polypeptide described herein) or composition thereof results in improvement (e.g., an increase) in lung volume as assessed by, e.g., whole body or lung plethysmography. In another embodiment, an active compound or composition thereof is administered to a healthy human subject and monitored for efficacy as a vaccine (e.g., the subject is monitored for the onset of symptoms of influenza virus infection; the ability of influenza virus to infect the subject; and/or a reduction in/absence of one or more symptoms associated with influenza virus infection). Techniques known to physicians familiar with infectious diseases can be used to determine whether an active compound or composition thereof reduces one or more symptoms associated with the influenza virus disease.

### 5.12 Kits

Provided herein is a pharmaceutical pack or kit comprising one or more containers filled with one or more of the ingredients of the pharmaceutical/immunogenic compositions described herein, such as one or more active compounds provided herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in accordance with the methods described herein. In one embodiment, a kit comprises an active compound described herein, preferably one or more chimeric hemagglutinin (HA) polypeptides, in one or more containers. In certain embodiments, a kit comprises a vaccine described herein, e.g., a split virus vaccine, a subunit vaccine, an inactivated influenza virus vaccine, or a live influenza virus vaccine, wherein said vaccine comprises one or more chimeric hemagglutinin (HA) polypeptides described herein.

### 6. EXAMPLES

### 6.1 Example 1: Design and Incorporation of Chimeric Hemagglutinins as a Universal Vaccination Strategy Against Influenza B Viruses

While much of the focus for universal vaccination strategies has been targeted against influenza A virus strains, influenza B viruses remain an important component of the annual vaccine. One strategy to universally target influenza B virus strains is to replace dominant antigenic sites on the head of the hemagglutinin (HA) with sequences from exotic influenza A virus strains. Through replacement of these antigenic loops, chimeric HAs have been generated that are easily incorporated into functional viruses through reverse genetics. Initial in vitro characterization of the chimeric HA and viruses expressing these HA, including growth analysis and surface expression were performed. Additionally, through replacement of antigenic sites, a decrease in HI endpoint titer in viruses with chimeric HA compared to wild type HA was apparent, emphasizing that antigenic sites have functionally been changed. These chimeric viruses will be further assessed for their ability to provide protection in animals during challenge in future studies.

### 6.1.1 Materials and Methods

Structural Overlays: Structural overlays were performed using Pymol software.

Sequence Alignments: Sequence alignments of HAs to determine corresponding loop/helix sequences were performed using BioEdit software.

Plaque Assay: Step 1: 2× media was prepared for agarose overlay (50 mL total volume: 25 mL 2×MEM, 8 ML WFI water, 660 μL sodium bicarbonate, 500 μL dextrin) and stored in water bath at 37° C. Step 2: Viral dilutions (dilutions used depend on sample and expected titer) were performed as follows: (a) PBS/1% BSA+1% penicillin/streptomycin was combined with Ca²⁺/Mg²⁺, and 450 μL was added to each tube for dilutions; (b) 50 μL of virus was added to the first tube, the tube was mixed, and then 50 μL from the tube was added to the next tube, etc. Step 3: Pre-plated cells were washed in 6-well plates with 1 mL PBS. Step 4:300 μL virus dilution (from step 2) was added to each well; most dilute to least dilute to avoid concentration variance. Step 5: Adsorption: cells were incubated at 33° C. or 37° C. (depending on the virus) for 1 hour; and were rocked every 10-15 minutes. Step 6: When adsorption was almost finished, 2% agarose was heated in microwave and 15 mL was added to media from step 1. Step 7: TPCK (N-tosyl-L-phenylalanyl chloromethyl ketone) treated trypsin (1 mg/mL) at 1/1000 dilution was added to media from step 1 (resulting concentrating is 1 μg/mL). Step 8: agarose/media/trypsin mixture was poured evenly on 6 well plates (2 mL/well) and allowed to harden without disturbing overlay. Plates were stored at 33° C. for 72 hours or 37° C. for 48 hours depending on the virus.

Immunostaining: Plaque assays were fixed using 4% formaldehyde in PBS. Cells were blocked with PBS/nonfat dried milk solution and antibodies were also prepared in this mixture. Polyclonal mouse serum raised against A/Puerto Rico/8/34 (PR8) virus or PR8 virus containing a luciferase gene was used as a primary antibody and was incubated for at least 2 hours. Wells were washed with PBS prior to application of the secondary antibody. Secondary anti-mouse HRP antibody was added in the PBS/nonfat dried milk blocking solution and was incubated for at least 1 hour. Staining was performed using TrueBlue stain that was removed with a water wash.

Growth curves in MDCK cells: Cells were plated to form a confluent monolayer in 6 well tissue culture treated plates. Viruses to be characterized were diluted in PBS/BSA. Prior to addition of virus sample to each well of cells, the growth media was removed and cells were washed one time with PBS. 300 microliters of each virus sample was added per well, with each virus with its respective time point having a separate well. Plates with inoculum added were incubated for 1 hour at 33 degrees Celsius. At each designated time point (8, 24, 48, 72 hours) supernatant was removed from the well and stored. Plaque assays were performed with a range of dilutions for each sample in order to titrate the amount of virus present in the sample obtained from the time course. Data was plotted using GraphPad Prism software.

Surface staining: 293T cells were plated the day prior to transfection. Cells were transfected with 500 ng of empty vector plasmid (pDZ), or the desired HA construct using Lipofectamine 2000. 1-2 days later, cells were fixed and stained using the appropriate antibody (polyclonal serum or monoclonal antibody) for that assay. Anti-mouse Alexa488 antibody was used as a secondary antibody for visualization. Stained cells were visualized on a fluorescent microscope.

HI assay: Prior to performing HI assay, any serum that was to be used as antibody was pretreated with Receptor-Destroying Enzyme (BioWhittaker, Walkersville, MD), 100 units/mL, prepared according to manufacturer's instructions. This resulted in serum being at a 1:10 dilution starting concentration (at the highest) for the HI assay. The HA assay was performed on test virus and positive control virus and the dilution that yielded 8 HAU (4 positive wells) was determined. This standardized virus was used for the assay. To find the dilution that would yield 4 wells, the last positive well was divided by 8. This provided the dilution factor, i.e., if the last dilution to have positive agglutination was 128 HAU, then to obtain 8 HAU the following calculation was performed: 128/8=16. To make 8 HAU, a 1:16 dilution was performed. In order to make sure the titer of the new dilution was 8 HAU, back titration was performed.

The following lanes of controls were included on the plate: (A) lane with PBS+virus (no antibody); (B) lane with 50 μL PBS (no antibody) and no virus. To plate, 50 μL antibody was added to column 1 and 25 μL PBS was added to other wells. 25 μL from the first column to the second column (2 fold dilutions of antibody) was transferred until the last columns with PBS alone and no antibody. Each well had 25 μL of the dilution. 25 μL of the appropriate virus was added to all other wells (either test virus, or positive control virus). PBS was added for “no virus” control. Virus and antibody were pre-incubated at room temperature for 30 minutes. 50 μL of 0.5% RBCs (chicken or turkey) in PBS was added as usual for hemagglutination assay. The plates were kept at 4° C. for 30 minutes. Pictures of the results were captured with a scanner.

Generation of chimeric HAs: Design of the HA constructs was based on determining corresponding residues for each loop or helix that was described by Wang et al., 2008, Journal of Virology, 82 (6): 3011-3020. Corresponding residues were determined by protein structural alignments as well as sequence alignments. The non-coding regions, signal peptide, transmembrane domain, and cytoplasmic tail domain were derived from influenza A/Puerto Rico/8/34 virus. In some instances, the non-coding regions, signal peptide, transmembrane domain, and cytoplasmic tail domain will be derived from other influenza A virus HA sequences or from influenza B HA sequences from viruses of Victoria, Yamagata, or potentially more historic lineages.

For generation of chimeric HA constructs, HAs were synthesized. Alternatively, the HA segment was generated by performing PCR on previously constructed plasmids and by using these PCR products (with at least 15 nucleotides overlap between each product) during the In-Fusion® (Clontech) cloning reaction. To insert the HA segment into its vector, plasmid pDZ was linearized with SAPI restriction enzyme. Each HA fragment contained 15 nucleotide overlap with the pDZ linearized vector. This overlap region was included during design for synthesis of the HA, or was artificially added on by adding this sequence to the primers amplifying from the non-coding regions of the HA during PCR.

In-Fusion® cloning reactions were performed with the generated HA products and linearized pDZ by using the reaction buffer as described by the manufacturer Clontech. Amounts of PCR fragments of HA, synthesized HA, or linearized pDZ vector were adjusted to have equal molar ratios (calculated based on fragment length). The In-Fusion® reaction incubated in a PCR machine at 50 degrees Celsius for 15 minutes then was removed. The resulting cloning product was transformed into Stellar cells and was plated in LB plates containing ampicillin. Resulting colonies were screened for presence of the correct HA insertion sequence and could be grown up and purified as mini-, midi-, or maxi-preps of the DNA. This resulted in the final plasmid product. The concentration of the final product was determined by methods such as using a Nanodrop.

### 6.1.2 Results

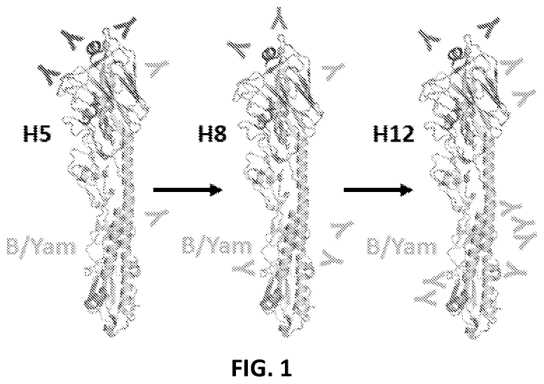

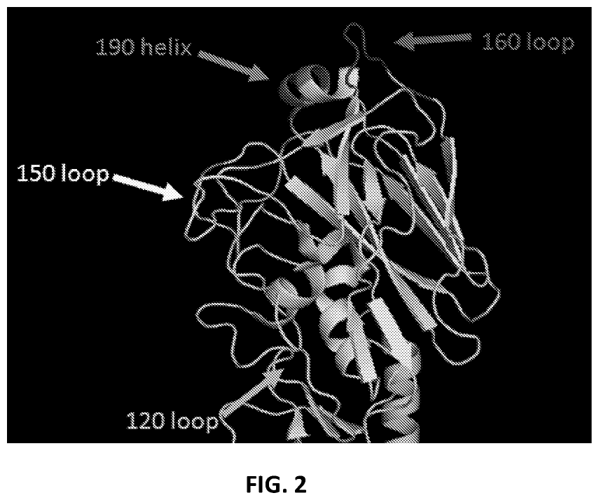

To generate influenza B virus chimeric HAs that could serve as universal vaccine candidates, key antigenic sites in the influenza B virus globular head domain of an influenza B/Yamagata/16/88 virus were modified based on antigenic sites from the globular head domain of influenza A viruses of the H5, H8, or H12 subtypes (influenza A/Vietnam/1203/04 virus (HALo), influenza A/mallard/Sweden/24/2002 virus, and influenza A_mallard_interior Alaska 7MP0167_2007 virus, respectively) (referred to as “chimeric HAs”) (FIG. 1). Constructs were also generated in which key antigenic sites in the influenza B virus globular head domain of an influenza B/Yamagata/16/88 virus were modified based on antigenic sites from the globular head domain of influenza A virus of the H11 subtype (influenza A/northern shoveler/Netherlands/18/99 virus). In particular, the antigenic sites of the influenza B virus globular head domain that were modified based on antigenic sites from the globular head domain of influenza A viruses were the 120 loop, the 150 loop, the 160 loop, and/or the 190 helix (see Wang et al., 2008, Journal of Virology, 82 (6): 3011-3020 for a description of the 120 loop, 150 loop, 160 loop, and 190 helix) (FIG. 2). Viruses expressing these chimeric HAs were rescued.

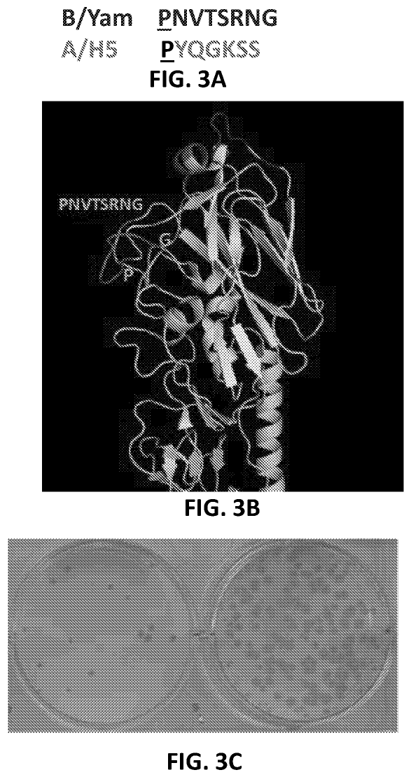

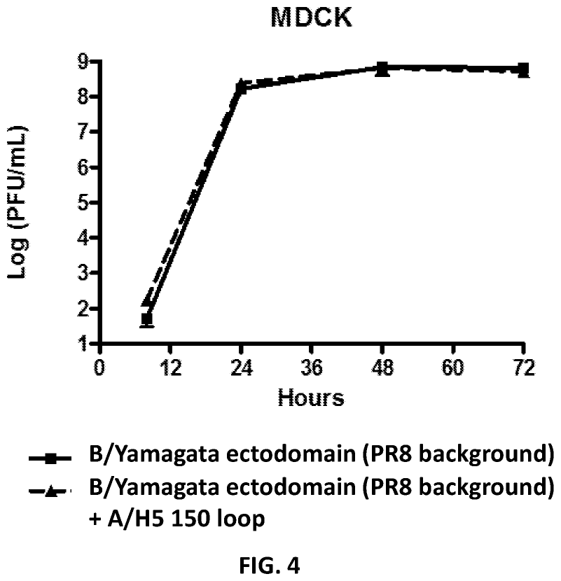

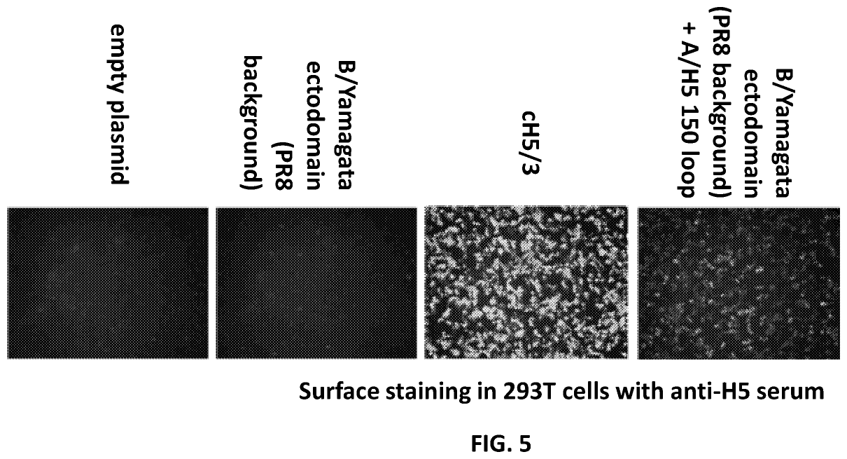

Amino acid residues relating to the 150 loop of the influenza B/Yamagata/16/88 virus HA globular head were modified based on amino acids from the influenza A/Vietnam/1203/04 (HALo) virus globular head domain (FIG. 3A, FIG. 3B). In particular, the amino acid sequence PNVTSRNG (SEQ ID NO:18) of the influenza B/Yamagata/16/88 virus HA 150 loop was replaced with the amino acid sequence PYQGKSS (SEQ ID NO: 19) from influenza A/Vietnam/1203/04 (HALo) virus globular head domain (FIG. 3A). Influenza A/Puerto Rico/8/34 virus (“PR8”) was used as the backbone (i.e., the PB2, PB1, PA, NP, NA, M, and NS are from PR8) to rescue virus expressing this chimeric HA construct. In addition, the non-coding regions, signal peptide, transmembrane domain, and cytoplasmic tail of the chimeric HA construct were from PR8. This virus was successfully rescued, having a stock titer of 5.0×10° plaque forming units (PFU)/mL (FIG. 3C). Furthermore, this virus was not attenuated when grown in MDCK cells grown at 33 degrees Celsius, as compared to a control PR8 virus expressing an HA comprising the PR8 the non-coding regions, signal peptide, transmembrane domain, and cytoplasmic tail and the ectodomain of the influenza B/Yamagata/16/88 virus hemagglutinin (FIG. 4). This virus with 150 loop amino acid residues from the HA polypeptide of B/Yamagata/16/88 replaced with amino acid residues from the A/Vietnam/1203/04 (HALo) HA polypeptide had similar growth kinetics and grew to a similar peak titer in MDCK cells compared to its viral counterpart in which the 150 loop residues remained from B/Yamagata/16/88. This demonstrated that by replacing the 150 loop antigenic site that growth was not impaired for this virus. This chimeric HA polypeptide retained its ability to be recognized by anti-influenza A virus H5 serum, as determined by immunofluorescent staining of 293T cells transfected with plasmid expressing this chimeric HA polypeptide with anti-influenza A virus H5 serum (FIG. 5). FIG. 5 assessed whether the H5 150 loop epitope that was transplanted into a B/Yamagata/16/88 HA ectodomain remained conformationally intact. An intact conformation was investigated using mouse polyclonal serum with anti-H5 activity. As expected, cells transfected with empty vector (pDZ) were not stained by the anti-H5 serum. Also as expected, anti-H5 serum did not bind the second negative control in which the transfected HA had a B/Yamagata/16/88 ectodomain and A/Puerto Rico/8/34 packaging signals (non-coding region (nucleotides), signal peptide, transmembrane domain, and cytoplasmic tail domain). This was expected since no H5 regions were present in the ectodomain. Staining was seen with the anti-H5 serum against the positive control cH5/3 HA. This construct expresses an HA protein that has an H5 head domain and a Perth (H3) stalk domain. Only the HA head would be stained in this case (not the stalk). Finally, the construct with an H5 150 loop, B/Yamagata/16/88 ectodomain, and A/Puerto Rico/8/34 packaging signals (non-coding region (nucleotides), signal peptide, transmembrane domain, and cytoplasmic tail domain) was able to be stained with anti-H5 serum. The staining that was observed occurred to a lesser degree than that observed with CH5/3 HA. Without being bound by any particular theory, the decreased staining may be because the anti-H5 serum would only recognize the transplanted 150 loop in comparison to the entire HA head. Similar chimeric HAs were also constructed by replacing the 150 loop of influenza B/Yamagata/16/88 virus HA with amino acids from influenza A/mallard Sweden/24/2002 virus (H8), or influenza A/northern shoveler/Netherlands/18/99 virus (H11) and viruses were rescued in a similar manner. A similar chimeric HA was also constructed by replacing the 150 loop of influenza B/Yamagata/16/88 virus HA with amino acids from influenza A_mallard_interior Alaska_7MP0167_2007 virus (H12).

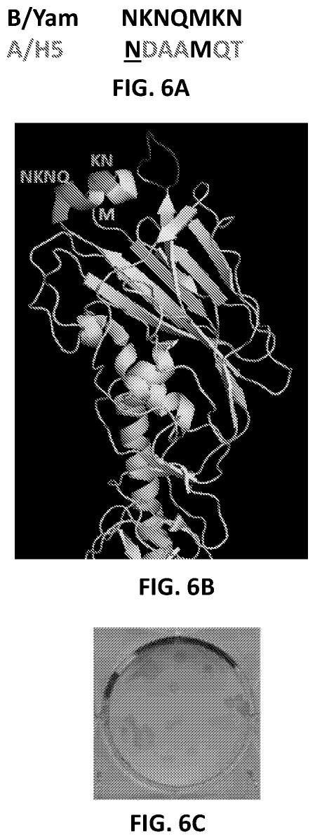

An additional chimeric HA construct was generated by modifying the NKNQMKN (SEQ ID NO: 7) sequence of the 190 helix of the influenza B/Yamagata/16/88 virus HA globular head with amino acid residues from the influenza A/Vietnam/1203/04 (HALo) virus globular head domain. In particular, it was sought to replace the amino acid sequence NKNQMKN (SEQ ID NO: 7) sequence of the 190 helix of the influenza B/Yamagata/16/88 virus with the amino acid sequence NDAAEQT from the influenza A/Vietnam/1203/04 (HALo) virus globular head domain. However, virus expressing this construct could not be rescued thus far. Thus, the M amino acid residue of the amino acid sequence NKNQMKN (SEQ ID NO: 7) from the 190 helix of the influenza B/Yamagata/16/88 virus HA globular head domain, which is a conserved residue, was retained. Accordingly, the NKNQMKN (SEQ ID NO: 7) sequence of the 190 helix of the influenza B/Yamagata/16/88 virus HA globular head domain with the amino acid sequence NDAAMQT (SEQ ID NO: 8) (FIG. 6A and FIG. 6B). As above, PR8 was used as the backbone (i.e., the PB2, PB1, PA, NP, NA, M, and NS are from PR8) to rescue virus expressing this chimeric HA construct. In addition, the non-coding regions, signal peptide, transmembrane domain, and cytoplasmic tail of this chimeric HA construct were from PR8. This virus was successfully rescued, having a stock titer of 5.3×10⁸ plaque forming units (PFU)/mL (FIG. 6C).

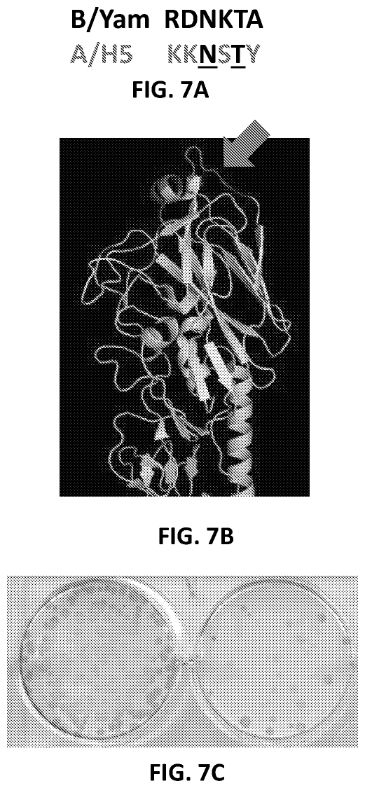

An additional chimeric HA construct was generated by modification of the RDNKTA (SEQ ID NO: 5) sequence of the 160 loop of the influenza B/Yamagata/16/88 virus with amino acid residues from the influenza A/Vietnam/1203/04 (HALo) virus globular head domain. In particular, the amino acid sequence RDNKTA (SEQ ID NO: 5) sequence of the 160 loop of the influenza B/Yamagata/16/88 virus were replaced with the amino acid sequence KKNSTY (SEQ ID NO: 6) (the N and T amino acids, which are not present in the influenza A/Vietnam/1203/04 (HALo) virus globular head domain, had to be retained from the influenza B/Yamagata/16/88 virus HA globular head domain in order to rescue the virus) (FIG. 7A and FIG. 7B). As above, PR8 was used as the backbone (i.e., the PB2, PB1, PA, NP, NA, M, and NS are from PR8) to rescue virus expressing this chimeric HA construct. In addition, the non-coding regions, signal peptide, transmembrane domain, and cytoplasmic tail of this chimeric HA construct were from PR8. This virus was successfully rescued, having a stock titer of 7.3×10⁸ plaque forming units (PFU)/mL (FIG. 7C).

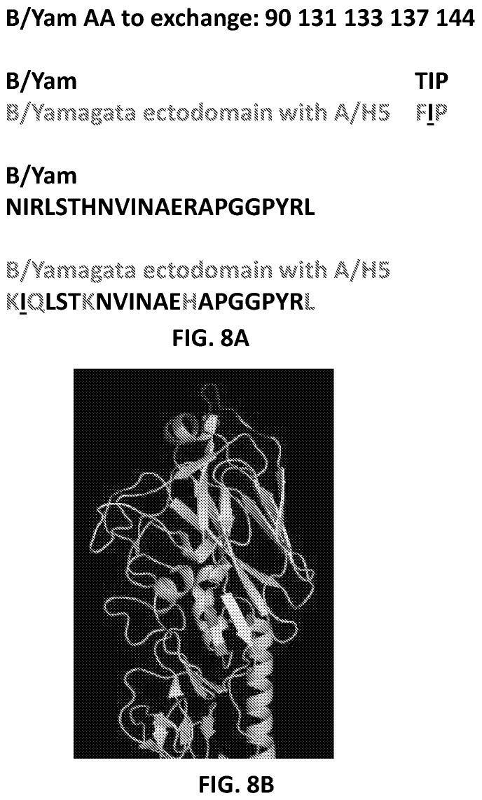

An additional chimeric HA construct was generated by modifying amino acid residues of the 120 loop of the influenza B/Yamagata/16/88 virus globular head domain with amino acid residues from the influenza A/Vietnam/1203/04 (HALo) virus globular head domain. The amino acid sequence of the 120 loop is NIRLSTHNVINAERAPGGPYRL (SEQ ID NO: 1) and the amino acid sequence at positions 90-92 (corresponding to the numbering of the immature HA, i.e., including the signal sequence) of the globular head is TIP (FIG. 8A). Amino acid substitutions were made based on the amino acid sequence of the globular head domain of the influenza A/Vietnam/1203/04 (HALo) virus. The resulting chimeric HA polypeptide comprised the sequences: FIP and KIQLSTKNVINAEHAPGGPYRL (SEQ ID NO: 2), in place of the TIP and NIRLSTHNVINAERAPGGPYRL (SEQ ID NO: 1) sequences, respectively (FIG. 8A and FIG. 8B) and TIP at positions 90-92.

The ability of these chimeric HAs to bind cross-protective antibodies that bind the influenza virus stalk and/or head domains was evaluated. To this end, antibodies CR9114, 5A7, CR8059, CR8033, and Antibody X were utilized. A summary of these antibodies is provided in Table 2, below.

TABLE 2
Cross-protective antibodies utilized

| | Head or stalk | |
|---|---|---|
| Antibody | reactive? | Lineage specificity? |
| CR9114 | Stalk | Binds influenza A virus and influenza B |
| | | virus (both lineages). Neutralizes |
| | | influenza A but not B by micro- |
| | | neutralization. Is protective against |
| | | influenza A and B in vivo |
| 5A7 | Stalk. Near C- | Broadly neutralizes viruses (in vitro) |
| | terminal of HA1 | from Victoria and Yamagata lineages |
| | (near head/stalk | |
| | interface) | |
| CR8059 | Head | Binds and neutralizes Yamagata and |
| (CR8071 is | | Victoria lineages (microneutralization); |
| stable variant) | | no HI reactivity against either) |
| CR8033 | Head | Binds and neutralizes Yamagata and |
| | | Victoria lineages (microneutralization); |
| | | HI activity against Yamagata lineage |
| | | only. |
| Antibody X | Head | Binds and neutralizes Yamagata and |
| | | Victoria lineages (plaque reduction, |
| | | microneutralization); no HI activity. |

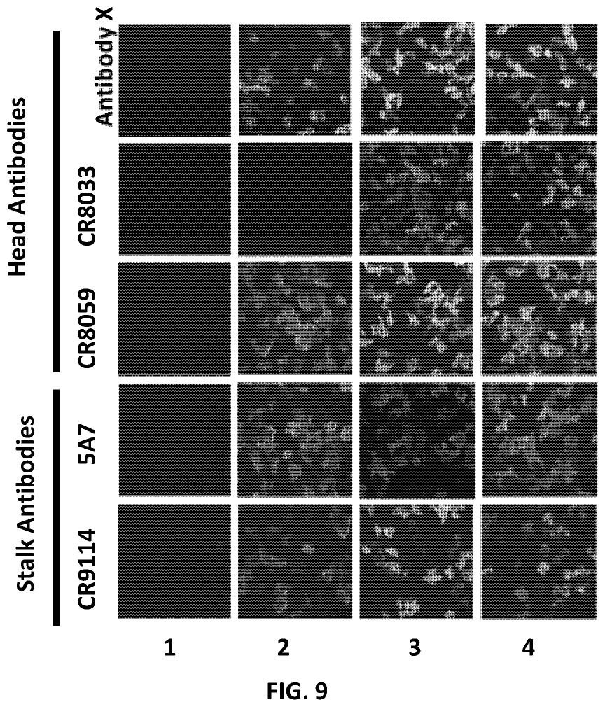

The tested chimeric HA polypeptides retained their abilities to be recognized by the tested anti-stalk and anti-head antibodies (FIG. 9). Surface staining was performed on transfected 293T cells with monoclonal antibodies known to bind influenza B HA head or stalk domains (FIG. 9).

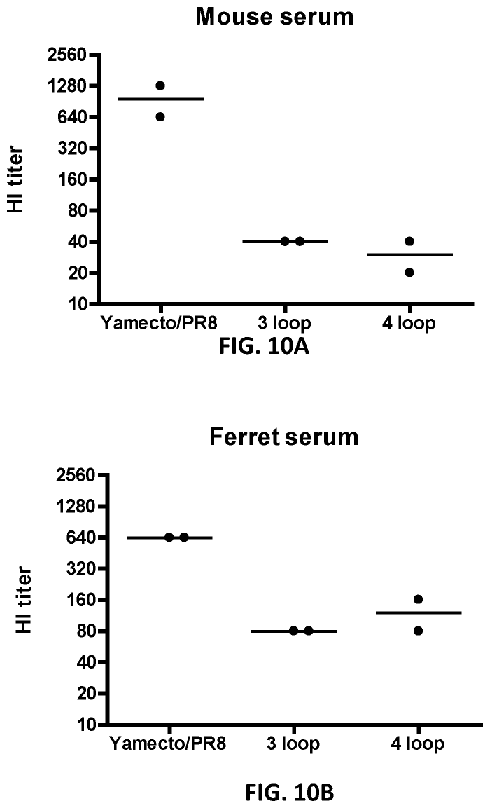

Finally, the HI activity of the chimeric HAs were tested to determine whether the antigenic loops of the influenza B virus were successfully replaced. Specifically, HI assays were performed with mouse (FIG. 10A) or ferret (FIG. 10B) serum. The chimeric HA polypeptides displayed drastically reduced strain specific HI activity.

In FIG. 10A, a hemagglutination inhibition (HI) assay was performed with mouse serum and viruses with either an influenza B/Yamagata/16/88 HA ectodomain and A/Puerto Rico/8/34 HA packaging signals (non-coding region (nucleotides), signal peptide, transmembrane domain, and cytoplasmic tail domain) or viruses within that context and either 3 loops (150 loop, 160 loop, 190 helix) or 4 loops (120 loop, 150 loop, 160 loop, 190 helix) were replaced with A/Vietnam/1203/04 H5 HA amino acid sequences. Viruses with 3 of 4 loops had a lower HI titer compared to those with a complete influenza B virus HA ectodomain. This was an indirect method for evaluating that antigenic sites within the influenza B virus HA had either been replaced, or in the least ablated, by swapping in exotic influenza A virus HA sequences (such as those from H5).

In FIG. 10B, a hemagglutination inhibition (HI) assay was performed with ferret serum and viruses with either an influenza B/Yamagata/16/88 HA ectodomain and A/Puerto Rico/8/34 HA packaging signals (non-coding region (nucleotides), signal peptide, transmembrane domain, and cytoplasmic tail domain) or viruses within that context and either 3 loops (150 loop, 160 loop, 190 helix) or 4 loops (120 loop, 150 loop, 160 loop, 190 helix) were replaced with A/Vietnam/1203/04 H5 HA amino acid sequences. Viruses with 3 of 4 loops had a lower HI titer compared to those with a complete influenza B virus HA ectodomain. This was an indirect method for evaluating that antigenic sites within the influenza B virus HA had either been replaced, or in the least ablated, by swapping in exotic influenza A virus HA sequences (such as those from H5).

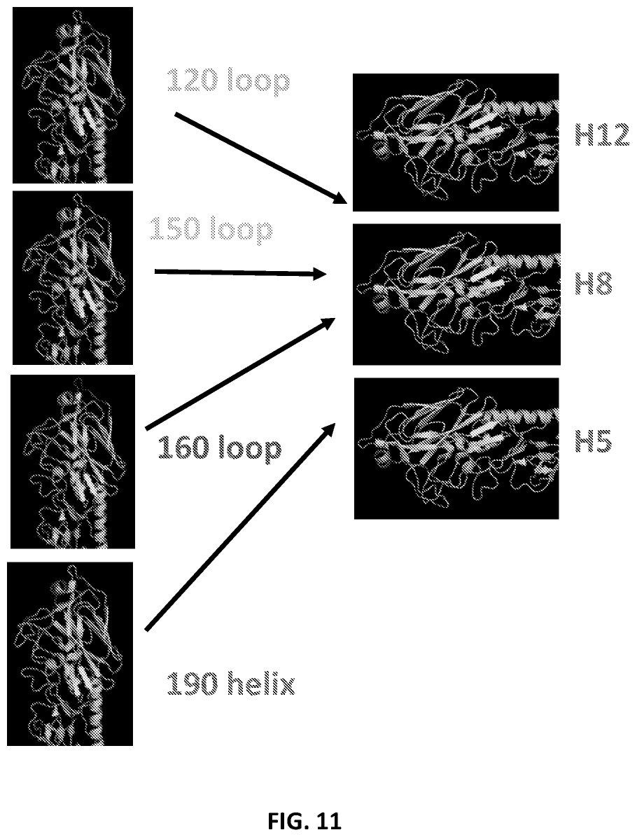

Chimeric HAs may also be generated by modifying each of the 120 loop, the 150 loop, the 160 loop, and the 190 helix of an influenza B virus HA with amino acid residues from influenza A viruses (e.g., of the H5, H8, H11, or H12 subtypes) (see, e.g., FIG. 11). Alternatively, the 120 loop, the 150 loop, the 160 loop, and/or the 190 helix may be modified with randomized amino acid residues or amino acid residues from an influenza A virus, e.g., an influenza A virus of an H11 subtype.

Table 3 provides a summary of the chimeric HA constructs cloned, expressed, and rescued in the influenza A virus backbone thus far. Such chimeric HA constructs may also be rescued in an influenza B virus backbone. All chimeric HA constructs were generated using the HA sequence from influenza B/Yamagata/16/88 virus, and the HA sequence from one or more of the following: influenza A/Vietnam/1203/04 (HALo) (H5) virus, influenza A/mallard/Sweden/24/2002 virus (H8), influenza A/northern shoveler/Netherlands/18/99 virus (H11), and/or influenza A_mallard_interior Alaska_7MP0167_2007 virus (H12). 3 loop design refers to a chimeric HA construct in which the 150 loop, 160 loop, and the 190 helix have been modified with the corresponding loops from the indicated influenza virus HA subtype (H5, H8, H11, or H12). 4 loop design refers to a chimeric HA construct in which the 120 loop, 150 loop, 160 loop, and the 190 helix have been modified with the corresponding regions from the indicated influenza virus HA subtype (H5, H8, H11, or H12).

TABLE 3
The influenza B virus HA sequence is from influenza B/Yamagata/16/88 virus.
The influenza A virus H5 HA sequence is from influenza A/Vietnam/1203/04(HALo)
virus. The influenza A virus H8 HA sequence is from influenza
A/mallard/Sweden/24/2002 virus. The influenza A virus H11 HA sequence is from
influenza A/northem shoveler/Netherlands/18/99 virus. The influenza A virus H12 HA
sequence is from influenza A_mallard_interior Alaska_7MP0167_2007 virus.

| | | | Rescued in A |
|---|---|---|---|
| | Cloned | Expressed | backbone |
| H5 - 3 loop design (150, 160, 190) | Yes | Yes | Yes |
| H5 - 4 loop design (120, 150, 160, | Yes (see, FIGS. 12A, | Yes | Yes |
| 190) | 12B, and 13) | | |
| H8 - 3 loop design (150, 160, 190) | Yes | Not tested | Not tested |
| H8 - 4 loop design (120, 150, 160, | Yes (see, FIGS. 14A, | Not tested | Not tested |
| 190) | 14B, and 15) | | |
| H11 - 3 loop design (150, 160, | Yes | Yes | Yes |
| 190) | | | |
| H11 -4 loop design (120, 150, | Yes (see, FIGS. 16A, | Not tested | Not tested |
| 160, 190) | 16B, and 17) | | |
| H12-3 loop design (150, 160, | Yes | Not tested | Not tested |
| 190) | | | |
| H12 - 4 loop design (120, 150, | Yes (see, FIGS. 18A, | Not tested | Not tested |
| 160, 190) | 18B, and 19) | | |

In conclusion, chimeric HA constructs can be incorporated into rescued influenza A viruses that grow efficiently in eggs. The chimeric HA constructs retain conserved epitopes in both the stalk and the head domain. Finally, the chimeric HA constructs display drastically decreased strain specific HI activity as compared to control HA constructs.

### 6.2 Example 2: Design and Characterization of Chimeric HA

This example provides and analyzes chimeric HA constructs which were prepared according to similar methods as described in Example 1 (Section 6.1, supra).

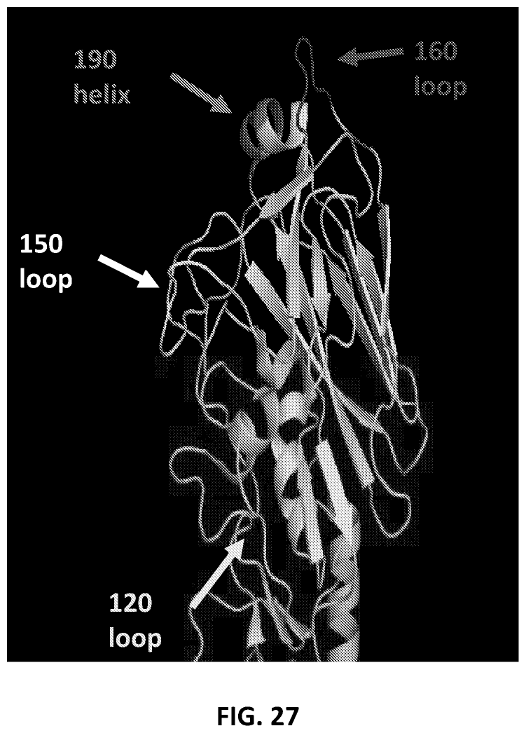

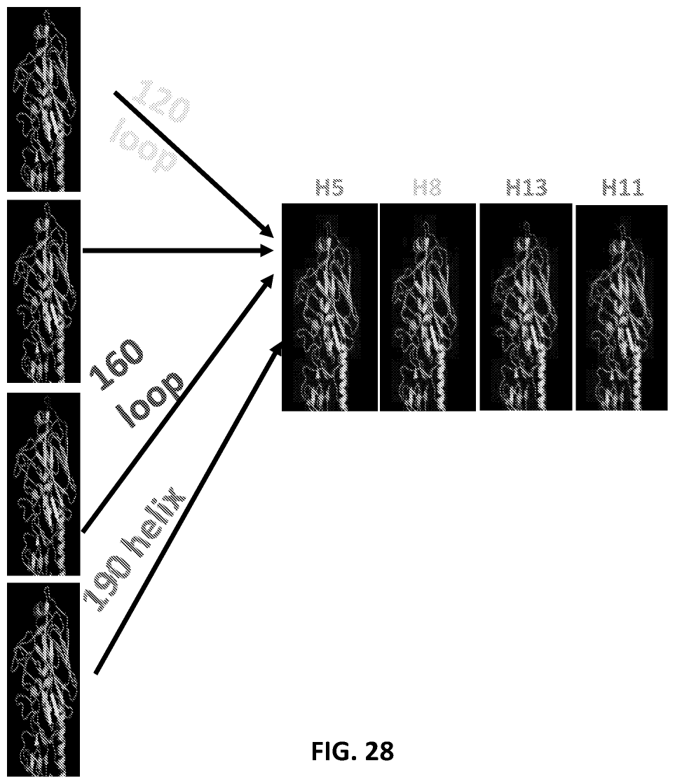

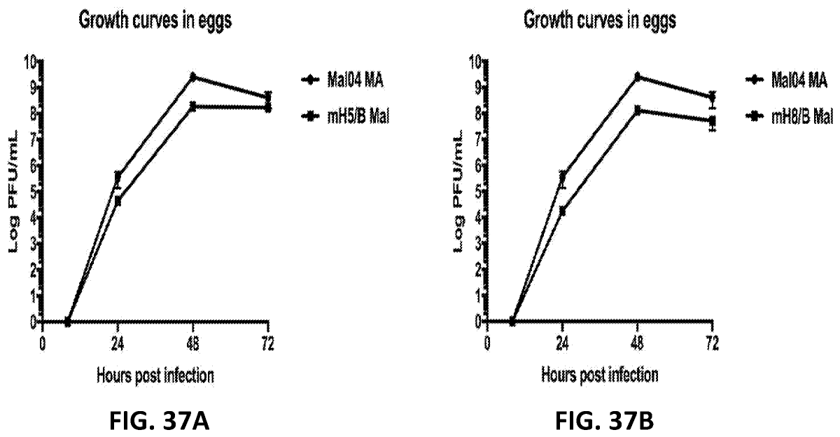

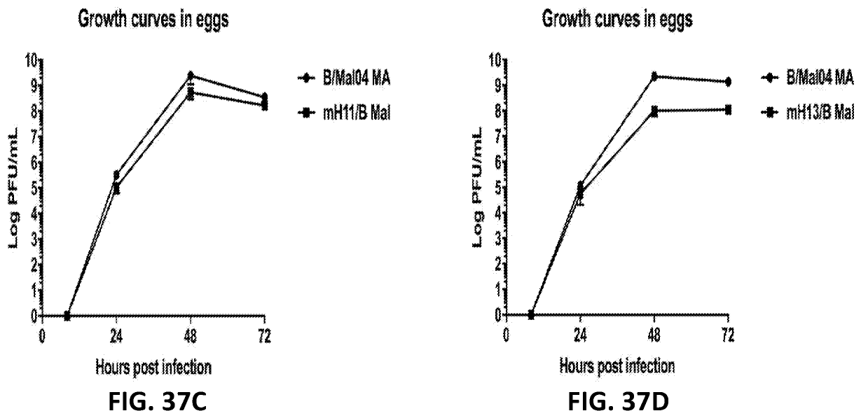

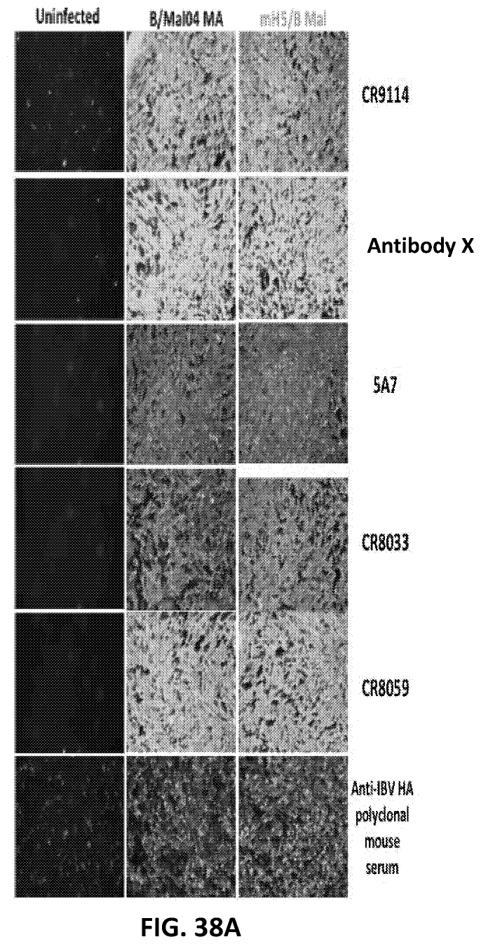

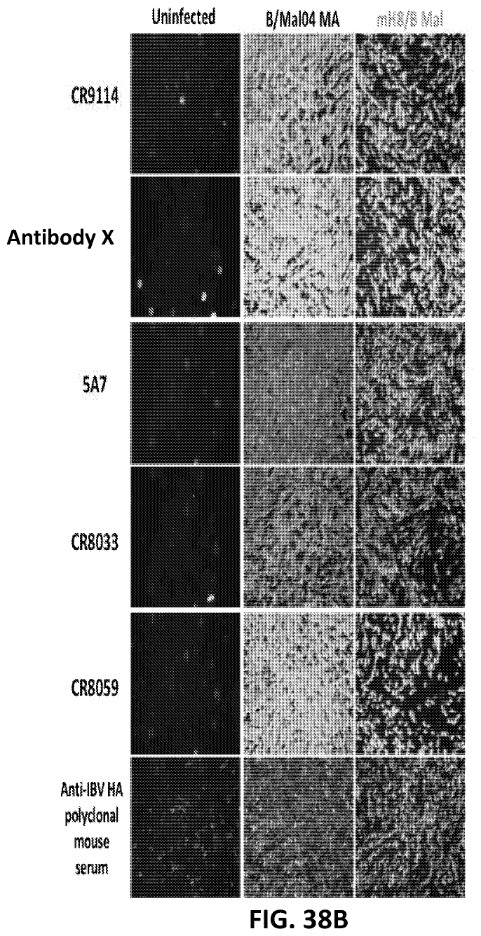

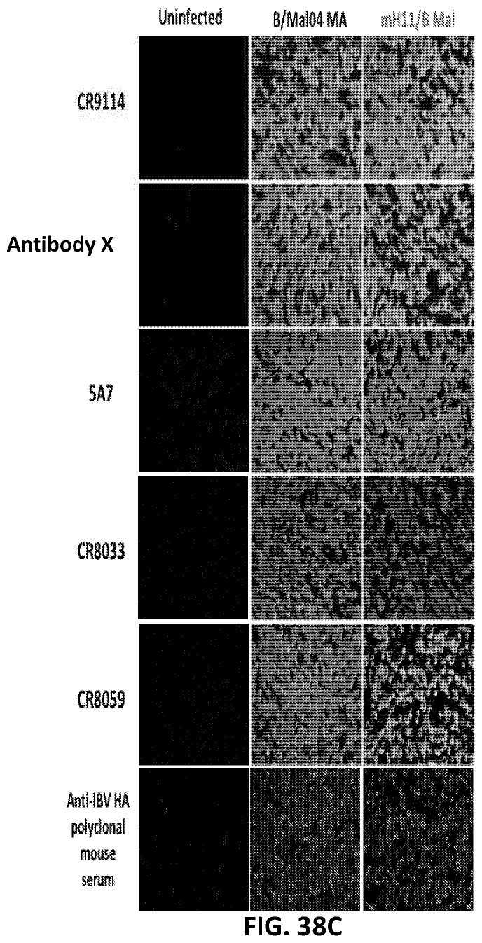

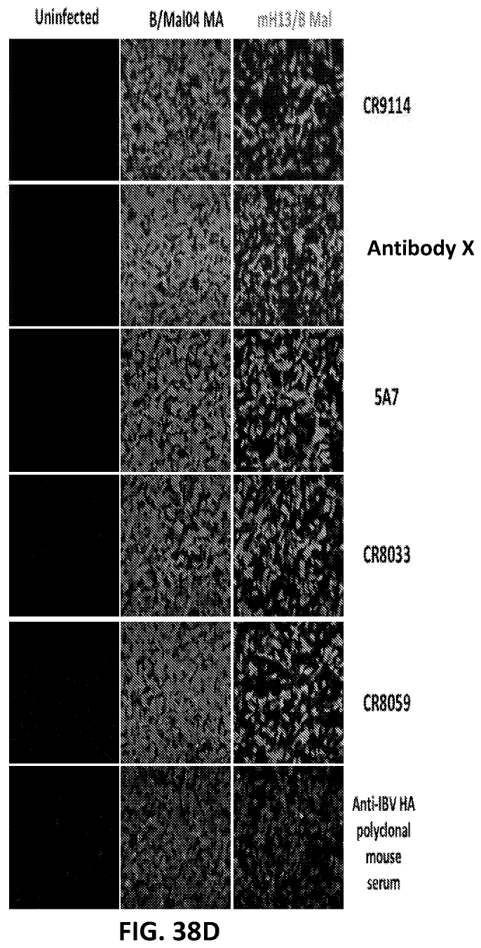

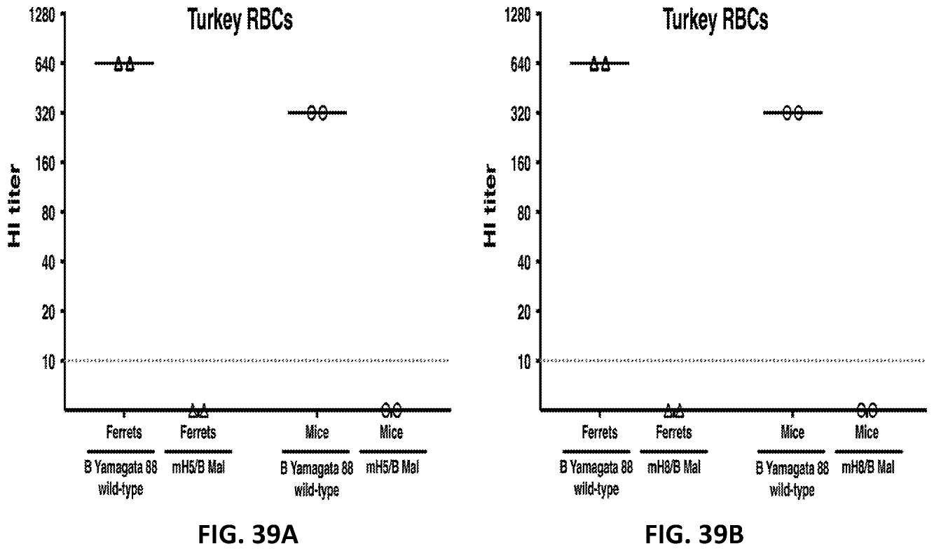

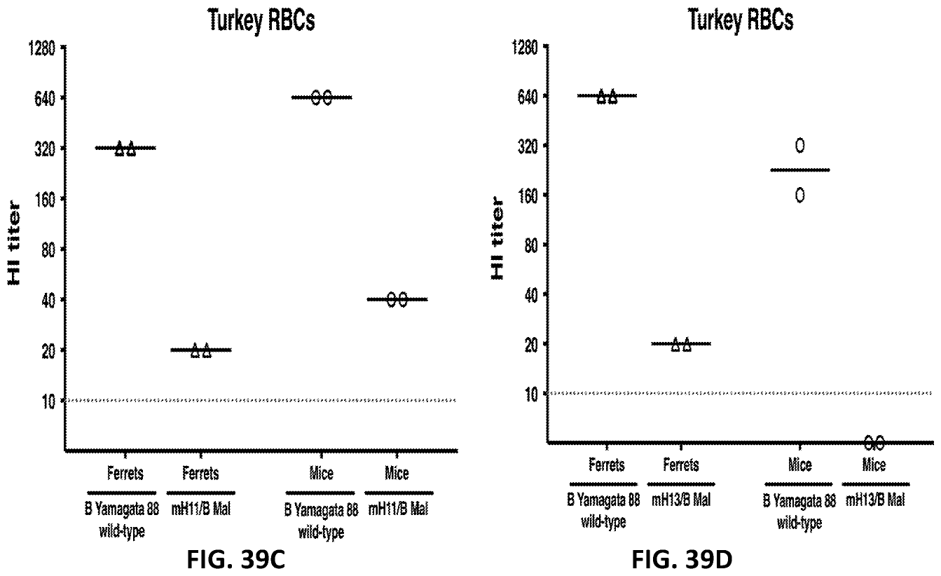

Through replacement of dominant antigenic sites on the head of the influenza B virus hemagglutinin (HA) with sequences from exotic influenza A virus strains (FIGS. 27 and 28), chimeric HAs have been generated that are easily incorporated into functional viruses through reverse genetics. Initial in vitro characterization of the chimeric HA and viruses expressing these HA, including growth analysis (FIG. 37) and surface expression (FIG. 38) were performed. Additionally, through replacement of antigenic sites, a decrease in HI endpoint titer in viruses with chimeric HA compared to wild type HA was apparent (FIG. 39), emphasizing that antigenic sites have functionally been changed.

### 6.2.1 Materials and Methods

Structural Overlays: Structural overlays were performed using Pymol software.

Sequence Alignments: Sequence alignments of HAs to determine corresponding loop/helix sequences were performed using BioEdit software.

Plaque Assay: Step 1: 2× media was prepared for agarose overlay (50 mL total volume: 25 mL 2×MEM, 9 ML WFI water, 660 μL 7.5% sodium bicarbonate, 500 μL 1% dextran) and stored in water bath at 37° C. Step 2: Viral dilutions (dilutions used depend on sample and expected titer) were performed as follows: (a) PBS/0.3% BSA+1% penicillin/streptomycin was combined with Ca²⁺/Mg²⁺, and 450 μL was added to each tube for dilutions; (b) 50 μL of virus was added to the first tube, the tube was mixed, and then 50 μL from the tube was added to the next tube, etc. Step 3: Pre-plated cells were washed in 6-well plates with 1 mL PBS. Step 4:300 μL virus dilution (from step 2) was added to each well; most dilute to least dilute to avoid concentration variance. Step 5: Adsorption: cells were incubated at 33° C. or 37° C. (depending on the virus) for 1 hour; and were rocked every 10-15 minutes. Step 6: When adsorption was almost finished, 2% agarose was heated in microwave and 15 mL was added to media from step 1. Step 7: TPCK (N-tosyl-L-phenylalanyl chloromethyl ketone) treated trypsin (1 mg/mL) at 1/1000 dilution was added to media from step 1 (resulting concentrating is 1 μg/mL). Step 8: agarose/media/trypsin mixture was poured evenly on 6 well plates (2 mL/well) and allowed to harden without disturbing overlay. Plates were stored at 33° C. for 72 hours or 37° C. for 48 hours depending on the virus.

Surface staining: MDCK cells were plated the day prior to infection. Cells were infected with indicated viruses at a multiplicity of infection (MOI) of 5 without TPCK-trypsin and incubated at 33 degrees Celsius. 17 hours post-infection, cells were fixed with methanol-free 4% PFA for immunofluorescence surface staining using the indicated anti-influenza B HA cross-protective human/mouse monoclonal antibodies and anti-influenza B virus HA polyclonal mouse serum. Secondary Alexa-Fluor 488 anti-human or anti-mouse antibody was used. Images were taken using Zeiss LSM 880 confocal microscope.

Growth curves in eggs: 10-day old embryonated chicken eggs were infected with 500 plaque forming units/egg of wild type influenza B/Malaysia/2506/04 MA virus or influenza B/Malaysia/2506/04 MA virus expressing chimeric HA. Growth curves were performed in triplicate. Allantoic fluids were harvested at 8 hours, 24 hours, 48 hours, and 72 hours post infection. Plaque assays were performed on MDCK cells as described above to determine virus titers.

HI assay: Mouse and ferret sera were raised against wild type influenza B virus strain B/Yamagata/16/88 to acquire hemagglutination inhibition (HI) reactivity. Prior to performing HI assay, any serum that was to be used as antibody for the HI assay was pretreated with Receptor-Destroying Enzyme (BioWhittaker, Walkersville, MD), 100 units/mL, prepared according to manufacturer's instructions. This resulted in serum being at a 1:10 dilution starting concentration (at the highest) for the HI assay. The HA assay was performed on test virus and positive control virus and the dilution that yielded 8 HAU (4 positive wells) was determined. This standardized virus was used for the assay. To find the dilution that would yield 4 wells, the last positive well was divided by 8. This provided the dilution factor, i.e., if the last dilution to have positive agglutination was 128 HAU, then to obtain 8 HAU the following calculation was performed: 128/8=16. To make 8 HAU, a 1:16 dilution was performed. In order to make sure the titer of the new dilution was 8 HAU, back titration was performed.

The following lanes of controls were included on the plate: (A) lane with PBS+virus (no antibody); (B) lane with 50 μL PBS (no antibody) and no virus. To plate, 50 μL antibody was added to column 1 and 25 μL PBS was added to other wells. 25 μL from the first column to the second column (2 fold dilutions of antibody) was transferred until the last columns with PBS alone and no antibody. Each well had 25 μL of the dilution. 25 μL of the appropriate virus was added to all other wells (either test virus, or positive control virus). PBS was added for “no virus” control. Virus and antibody were pre-incubated at room temperature for 30 minutes. 50 μL of 0.5% RBCs (turkey) in PBS was added as usual for hemagglutination assay. The plates were kept at 4° C. for 30 minutes. Pictures of the results were captured with a scanner.

Generation of chimeric HAs: Design of the HA constructs was based on determining corresponding residues for each loop or helix that was described by Wang et al., 2008, Journal of Virology, 82 (6): 3011-3020. Corresponding residues were determined by protein structural alignments as well as sequence alignments.

For generation of chimeric HA constructs, HAs were synthesized. Alternatively, the HA segment was generated by performing PCR on previously constructed plasmids and by using these PCR products (with at least 15 nucleotides overlap between each product) during the In-Fusion® (Clontech) cloning reaction. To insert the HA segment into its vector, plasmid pDZ was linearized with SAPI restriction enzyme. Each HA fragment contained 15 nucleotide overlap with the pDZ linearized vector. This overlap region was included during design for synthesis of the HA, or was artificially added on by adding this sequence to the primers amplifying from the non-coding regions of the HA during PCR.

In-Fusion® cloning reactions were performed with the generated HA products and linearized pDZ by using the reaction buffer as described by the manufacturer Clontech. Amounts of PCR fragments of HA, synthesized HA, or linearized pDZ vector were adjusted to have equal molar ratios (calculated based on fragment length). The In-Fusion® reaction incubated in a PCR machine at 50 degrees Celsius for 15 minutes then was removed. The resulting cloning product was transformed into Stellar cells and was plated in LB plates containing ampicillin. Resulting colonies were screened for presence of the correct HA insertion sequence and could be grown up and purified as mini-, midi-, or maxi-preps of the DNA. This resulted in the final plasmid product. The concentration of the final product was determined by methods such as using a Nanodrop.

### 6.2.2 Results

To generate influenza B virus chimeric HAs that could serve as universal vaccine candidates, key antigenic sites (the 120 loop, the 150 loop, the 160 loop, and/or the 190 helix (see Wang et al., 2008, Journal of Virology, 82 (6): 3011-3020 for a description of the 120 loop, 150 loop, 160 loop, and 190 helix)) in the influenza B virus globular head domain of influenza B/Yamagata/16/88 virus HA were modified based on antigenic sites from the globular head domain of influenza A viruses of the H5, H8, H11, or H13 subtypes (influenza A/Vietnam/1203/04 virus (HALo) (H5), influenza A/Mallard/Sweden/24/2002 virus (H8), influenza A/northern shoveler/Netherlands/18/99 virus (H11)), or A/black headed gull/Sweden/1/99 (H13), respectively) (referred to as mH5/B, mH8/B, mH11/B, and mH13/B, respectively, chimeric HAs) (FIG. 27 and FIG. 28).

To generate the mH5/B chimeric HA for rescue in an influenza B/Malaysia/2506/04 MA virus backbone, amino acid residues relating to the 120 loop, 150 loop, 160 loop, and 190 helix of the influenza B/Yamagata/16/88 virus HA globular head were modified based on amino acids from the influenza A/Vietnam/1203/04 (HALo) (H5) virus globular head domain (FIGS. 29 and 30). In particular, the amino acid sequences from of the influenza B/Yamagata/16/88 virus HA 120 loop (TIP and NIRLSTHNVINAERAPGGPYRL (SEQ ID NO: 1)), 150 loop (PNVTSRNG (SEQ ID NO: 18)), 160 loop (RDNKTA (SEQ ID NO: 5)), and 190 helix (NKNQMKN (SEQ ID NO: 7)) were replaced with amino acid sequences FIP, KIQLSTKNVINAEHAPGGPYRL (SEQ ID NO: 2), PYQGKSS (SEQ ID NO: 19), KKNSTY (SEQ ID NO: 6), and NDAAMQT (SEQ ID NO: 8), respectively, based on amino acid sequences of the influenza A/Vietnam/1203/04 (HALo) (H5) virus globular head domain. Influenza B/Malaysia/2506/04 MA virus was used as the backbone (i.e., the PB2, PB1, PA, NP, NA, M, and NS are from influenza B/Malaysia/2506/04 MA virus) to rescue virus expressing this chimeric HA construct. Additionally, the glutamic acid (E) amino acid at position 156 of the immature influenza B/Yamagata/16/88 virus HA was substituted with a lysine (K) (i.e., an E156K mutation) in the chimeric HA. This virus was successfully rescued, having a stock titer of 6.95×10⁸ PFU/mL. This virus was slightly attenuated when grown in 10-day old embryonated eggs (500 PFU/egg inoculant) at 33 degrees Celsius, as compared to a control influenza B/Malaysia/2506/04 MA virus (FIG. 37A). The peak titer p value was 0.0005.

To generate the mH8/B chimeric HA for rescue in an influenza B/Malaysia/2506/04 MA virus backbone, amino acid residues relating to the 120 loop, 150 loop, 160 loop, and 190 helix of the influenza B/Yamagata/16/88 virus HA globular head were modified based on amino acids from the influenza A/Mallard/Sweden/24/2002 virus (H8) globular head domain (FIGS. 31 and 32). In particular, the amino acid sequences of the influenza B/Yamagata/16/88 virus HA 120 loop (TIP and NIRLSTHNVINAERAPGGPYRL (SEQ ID NO: 1)), 150 loop (PNVTSRNG (SEQ ID NO: 18)), 160 loop (RDNKTA (SEQ ID NO: 5)), and 190 helix (NKNQMKN (SEQ ID NO: 7)) were replaced with amino acid sequences HIP, RIRLSTYNVINAETAPGGPYRL (SEQ ID NO: 51), NASTGGQS (SEQ ID NO: 52), KKKADTY (SEQ ID NO: 53), and ADAKMQT (SEQ ID NO: 54), respectively, based on amino acid sequences of the influenza A/Mallard/Sweden/24/2002 virus (H8) globular head domain. Influenza B/Malaysia/2506/04 MA virus was used as the backbone (i.e., the PB2, PB1, PA, NP, NA, M, and NS are from influenza B/Malaysia/2506/04 MA virus) to rescue virus expressing this chimeric HA construct. Additionally, the glutamic acid (E) amino acid at position 156 of the immature influenza B/Yamagata/16/88 virus HA was substituted with a lysine (K) (i.e., an E156K mutation) in the chimeric HA. This virus was successfully rescued, having a stock titer of 1.16×10⁹ PFU/mL. This virus was slightly attenuated when grown in 10-day old embryonated eggs (500 PFU/egg inoculant) at 33 degrees Celsius, as compared to a control influenza B/Malaysia/2506/04 MA virus expressing an HA comprising the influenza B/Malaysia/2506/04 MA virus (FIG. 37B). The peak titer p value was 0.0004.

To generate the mH11/B chimeric HA for rescue in an influenza B/Malaysia/2506/04 MA virus backbone, amino acid residues relating to the 120 loop, 150 loop, 160 loop, and 190 helix of the influenza B/Yamagata/16/88 virus HA globular head were modified based on amino acids from the influenza A/northern shoveler/Netherlands/18/99 virus (H11) globular head domain (FIGS. 33 and 34). In particular, the amino acid sequences of the influenza B/Yamagata/16/88 virus HA 120 loop (TIP and NIRLSTHNVINAERAPGGPYRL (SEQ ID NO: 1)), 150 loop (PNVTSRNG (SEQ ID NO: 18)), 160 loop (RDNKTA (SEQ ID NO: 5)), and 190 helix (NKNQMKN (SEQ ID NO: 7)) were replaced with amino acid sequences LIP, KIELSTSNVINAEVAPGGPYRL (SEQ ID NO: 55), PFGSSNS (SEQ ID NO: 56), HQSGTY (SEQ ID NO: 57), and TTLKMHQ (SEQ ID NO: 58), respectively, based on amino acid sequences of the influenza A/northern shoveler/Netherlands/18/99 virus (H11) globular head domain. Influenza B/Malaysia/2506/04 MA virus was used as the backbone (i.e., the PB2, PB1, PA, NP, NA, M, and NS are from influenza B/Malaysia/2506/04 MA virus; see SEQ ID NOs: 80, 81, 82, 84, 85, 86, and 87 for the nucleotide sequences) to rescue virus expressing this chimeric HA construct. Additionally, the glycine (G) amino acid at position 250 of the immature influenza B/Yamagata/16/88 virus HA was substituted with a glutamic acid (E) (i.e., a G250E mutation) in the chimeric HA. This virus was successfully rescued, having a stock titer of 1.42×10⁸ PFU/mL. This virus was slightly attenuated when grown in 10-day old embryonated eggs (500 PFU/egg inoculant) at 33 degrees Celsius, as compared to a control influenza B/Malaysia/2506/04 MA virus (FIG. 37C). The peak titer p value was 0.2310.

To generate the mH13/B chimeric HA for rescue in an influenza B/Malaysia/2506/04 MA virus backbone, amino acid residues relating to the 120 loop, 150 loop, 160 loop, and 190 helix of the influenza B/Yamagata/16/88 virus HA globular head were modified based on amino acids from the influenza A/black headed gull/Sweden/1/99 virus globular head domain (FIGS. 35 and 36). In particular, the amino acid sequences of the influenza B/Yamagata/16/88 virus HA 120 loop (TIP and NIRLSTHNVINAERAPGGPYRL (SEQ ID NO: 1)), 150 loop (PNVTSRNG (SEQ ID NO: 18)), 160 loop (RDNKTA (SEQ ID NO: 5)), and 190 helix (NKNQMKN (SEQ ID NO: 7)) were replaced with amino acid sequences NIP, RIELSTHNVINAEVAPGGPYRL (SEQ ID NO: 59), PDKGASS (SEQ ID NO: 60), KRGNQY (SEQ ID NO: 61), and VSTNMAK (SEQ ID NO: 62), respectively, based on amino acid sequences of the influenza A/black headed gull/Sweden/1/99 virus globular head domain. Influenza B/Malaysia/2506/04 MA virus was used as the backbone (i.e., the PB2, PB1, PA, NP, NA, M, and NS are from influenza B/Malaysia/2506/04 MA virus) to rescue virus expressing this chimeric HA construct. Additionally, the glutamic acid (E) amino acid at position 156 of the immature influenza B/Yamagata/16/88 virus HA was substituted with a lysine (K) (i.e., an E156K mutation) in the chimeric HA. This virus was successfully rescued, having a stock titer of 2.19×10⁸ PFU/mL. This virus was slightly attenuated when grown in 10-day old embryonated eggs (500 PFU/egg inoculant) at 33 degrees Celsius, as compared to a control influenza B/Malaysia/2506/04 MA virus (FIG. 37D). The peak titer p value was 0.0072.

The ability of these chimeric HAs to bind cross-protective antibodies that bind the influenza virus stalk and/or head domains was evaluated. To this end, antibodies CR9114, 5A7, CR8059, CR8033, and Antibody X (sometimes referred to as IIC7) were utilized. A summary of these antibodies is provided in Table 2, above. The tested chimeric HA polypeptides retained their abilities to be recognized by the tested anti-stalk and anti-head antibodies (FIGS. 38A-38D). Surface staining was performed on MDCK cells infected with the indicated viruses at a multiplicity of infection of 5 without TPCK-trypsin at 17 hours post-infection with monoclonal antibodies known to bind influenza B HA head or stalk domains (FIGS. 38A-38D).

The HI activity of the chimeric HAs was tested to determine whether the antigenic loops of the influenza B virus were successfully replaced. Specifically, HI assays were performed with mouse or ferret serum raised against wild type influenza B/Yamagata/16/88 virus (FIGS. 39A-39D). The chimeric HA polypeptides displayed drastically reduced strain specific HI activity (FIGS. 39A-39D). This was an indirect method for evaluating that antigenic sites within the influenza B virus HA had either been replaced, or in the least ablated, by swapping in exotic influenza A virus HA sequences (such as those from H5, H8, H11, or H13).

In conclusion, chimeric HA constructs can be incorporated into rescued influenza B viruses that grow to robust titers in eggs, despite being attenuated compared to the wild type virus. The chimeric HAs appeared to lose their B HA immunodominant head epitopes, while sub-dominant conserved, cross-protective epitopes on B HA were preserved.

### 6.3 Example 3: Vaccination Regimens Comprising Chimeric HA

Generation of Chimeric HA Constructs: mH5/B, mH8/B, and mH13/B chimeric HA constructs were generated as described in Section 6.2, supra. pDZ plasmid (see Martínez-Sobrido L, García-Sastre A. Generation of Recombinant Influenza Virus from Plasmid DNA. Journal of Visualized Experiments: JoVE. 2010; (42): 2057. doi: 10.3791/2057, which is incorporated by reference herein in its entirety) encoding the mH13/B chimeric HA was giga-prepped (service provided by Genewiz) to prepare the DNA used for the prime (see Table 4, infra). mH5/B and mH8/B chimeric HA recombinant proteins were synthesized using baculovirus expression system as previously described in Margine I, Palese P, Krammer F. Expression of Functional Recombinant Hemagglutinin and Neuraminidase Proteins from the Novel H7N9 Influenza Virus Using the Baculovirus Expression System. Journal of Visualized Experiments: JoVE. 2013; (81): 51112. doi: 10.3791/51112, which is incorporated by reference herein in its entirety . . .

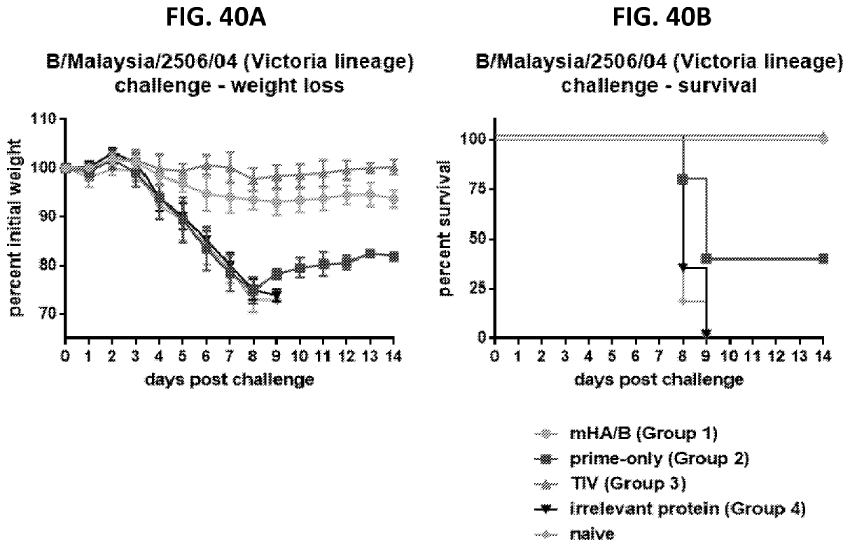

Mouse Models and In Vivo Analyses: Five 6 to 8 weeks old female BALB/c mice/group (ten groups in total) were used in this example (see Table 4, infra). Mice are vaccinated as indicated in Table 4, below. Specifically, mice are primed with either: (i) 80 μg of pDZ plasmid encoding mH13/B administered by electroporation (Groups 1, 2, 5-7, and 10); (ii) 1 μg of Fluzone (2006-2007 season, comprising an influenza B virus of the Victoria lineage; Sanofi Pasteur) administered intramuscularly (Group 3); (iii) 1 μg of Flulaval (2008-2009 season, comprising an influenza B virus of the Yamagata lineage; GlaxoSmithKline) administered intramuscularly (Group 8); or (iv) mock primed (Groups 4 and 9). Three weeks post-prime, mice in Groups 1, 2, 4-7, 9, and 10 are boosted with: (i) intramuscular administration of 5 μg of mH5/B protein adjuvanted with 5 μg of polyI: C and intranasal administration of 5 μg of mH5/B protein adjuvanted with 5 μg of polyI: C (Groups 1, 5, 6, and 10); or (ii) intramuscular administration of 5 μg of bovine serum albumin (BSA) adjuvanted with 5 μg of polyI: C and intranasal administration of 5 μg of BSA adjuvanted with 5 μg of polyI: C (Groups 2, 4, 7, and 9). Mice in Groups 3 and 8 do not receive a boost three-weeks post-prime. Six weeks post-prime, mice are boosted with: (i) intramuscular administration of 5 μg of mH8/B protein adjuvanted with 5 μg of polyI: C and intranasal administration of 5 μg of mH8/B protein adjuvanted with 5 μg of polyI: C (Groups 1, 5, 6, and 10); (ii) intramuscular administration of 5 μg of BSA adjuvanted with 5 μg of polyI: C and intranasal administration of 5 μg of BSA adjuvanted with 5 μg of polyI: C (Groups 2, 4, 7, and 9); (iii) 1 μg of Fluzone administered intramuscularly (Group 3); or (iv) 1 μg of Flulaval administered intramuscularly (Group 8). Four weeks later (i.e., 10 weeks after the prime), mice in Groups 1˜4 were challenged with influenza B/Malaysia/2506/04 virus (Victoria lineage) at 5 mouse lethal dose 50 (“LD₅₀”), intranasally, and mice in Groups 1˜4 were monitored for weight loss (FIG. 40A) and survival (FIG. 40B) compared to naïve mice. Mice in Group 1 were completely protected from mortality with minimal weight loss.

Additionally, 10 weeks after the prime, mice in Groups 6-9 are challenged with influenza B/Florida/4/06 virus (Yamagata lineage) at 5 mouse LD₅₀, intranasally, and mice in Groups 5 and 10 are terminally bled (for, e.g., passive transfer studies). The challenged mice are monitored for weight loss and survival.

TABLE 4
Chimeric HA vaccination in mice. TIV = trivalent influenza vaccine;
BSA = bovine serum albumin; IM = intramuscular; IN = intranasally;
VIC indicates that the virus is from the influenza B virus Victoria lineage;
YAM indicates that the virus is from the influenza B virus Yamagata lineage.
mH5/B refers to mH5/B chimeric HA described in FIGS. 29 and 30. mH8/B refers
to the mH8/B chimeric HA described in FIGS. 31 and 32. mH13/B refers to
the mH13/B chimeric HA described in FIGS. 35 and 36.

| | | Boost 1 | Boost 2 | Challenge |
|---|---|---|---|---|
| Group | Prime | (3 weeks later) | (3 weeks later) | (4 weeks later) |
| 1 | mH13/B | mH5/B protein 10 μg | mH8/B protein 10 μg | B/Malaysia/2506/04 |
| | DNA | (5 μg IM, 5 μg IN) | (5 μg IM, 5 μg IN) | (Vic) |
| | 80 μg | 10 μg polyI:C | 10 μg polyI:C | |
| 2 | mH13/B | BSA 10 μg (5 μg IM, | BSA 10 μg (5 μg IM, | B/Malaysia/2506/04 |
| | DNA | 5 μg IN) | 5 μg IN) | (Vic) |
| | 80 μg | 10 μg polyI:C | 10 μg polyI:C | |
| 3 | Fluzone | None | Fluzone 1 μg (IM) | B/Malaysia/2506/04 |
| | 1 μg (IM) (TIV) | | (TIV) | (Vic) |
| 4 | Mock | BSA 10 μg (5 μg IM, | BSA 10 μg (5 μg IM, | B/Malaysia/2506/04 |
| | | 5 μg IN) | 5 μg IN) | (Vic) |
| | | 10 μg polyI:C | 10 μg polyI:C | |
| 5 | mH13/B | mH5/B protein 10 μg | mH8/B protein 10 μg | Terminal Bleed |
| | DNA | (5 μg IM, 5 μg IN) | (5 μg IM, 5 μg IN) | |
| | 80 μg | 10 μg polyI:C | 10 μg polyI:C | |
| 6 | mH13/B | mH5/B protein 10 μg | mH8/B protein 10 μg | B/Florida/4/06 (Yam) |
| | DNA | (5 μg IM, 5 μg IN) | (5 μg IM, 5 μg IN) | |
| | 80 μg | 10 μg polyI:C | 10 μg polyI:C | |
| 7 | mH13/B | BSA 10 μg (5 μg IM, | BSA 10 μg (5 μg IM, | B/Florida/4/06 (Yam) |
| | DNA | 5 μg IN) | 5 μg IN) | |
| | 80 μg | 10 μg polyI:C | 10 μg polyI:C | |
| 8 | Flulaval | None | Flulaval 1 μg (IM) | B/Florida/4/06 (Yam) |
| | 1 μg (IM) (TIV) | | (TIV) | |
| 9 | Mock | BSA 10 μg (5 μg IM, | BSA 10 μg (5 μg IM, | B/Florida/4/06 (Yam) |
| | | 5 μg IN) | 5 μg IN) | |
| | | 10 μg polyI:C | 10 μg polyI:C | |
| 10 | mH13/B | mH5/B protein 10 μg | mH8/B protein 10 μg | Terminal Bleed |
| | DNA | (5 μg IM, 5 μg IN) | (5 μg IM, 5 μg IN) | |
| | 80 μg | 10 μg polyI:C | 10 μg polyI:C | |

### 7. EMBODIMENTS

Provided herein are the following exemplary embodiments:

1. A chimeric hemagglutinin (HA) polypeptide comprising a hemagglutinin ectodomain from an influenza B virus comprising one, two, three or all of the following:
a. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA;
b. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA;
c. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA; and
d. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA.

2. A chimeric hemagglutinin (HA) polypeptide comprising a hemagglutinin ectodomain from an influenza B virus comprising one, two, three or all of the following:
a. 2, 3, 4, 5, 6, 7, 8, 9, 10, 11, 12, 13, 14, 15, 16, 17, 18, 19, 20 or more amino acid substitutions within the 120 loop of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the influenza B virus HA with amino acid residues found in a corresponding region of an influenza A virus HA;
b 2, 3, 4, 5, 6, 7, 8, 9 or more amino acid substitutions within the 150 loop of the influenza B virus HA, wherein the amino acid substitutions substitute 2, 3, 4, 5, 6, 7, 8, 9 or more amino acid residues in the 150 loop of the influenza B virus HA with amino acid residues found in a corresponding region of an influenza A virus HA;
c. 2, 3, 4, 5 or more amino acid substitutions within the 160 loop of the influenza B virus HA, wherein the amino acid substitutions substitute 2, 3, 4, 5 or more amino acid residues in the 160 loop of the influenza B virus HA with amino acid residues found in a corresponding region of an influenza A virus HA; and
d. 2, 3, 4, 5, 6, 7, 8 or more amino acid substitutions within the 190 helix of the influenza B virus HA, wherein the amino acid substitutions substitute 2, 3, 4, 5, 6, 7, 8 or more amino acid residues in the 190 helix of the influenza B virus HA with amino acid residues found in a corresponding region of an influenza A virus HA.

3. The chimeric HA polypeptide of embodiment 1 or 2 which further comprises the signal peptide of the influenza B virus HA.

4. The chimeric HA polypeptide of embodiment 1, 2, or 3 which further comprises the transmembrane domain and cytoplasmic tail domain of the influenza B virus HA.

5. The chimeric HA polypeptide of any one of embodiments 1 to 4, wherein the influenza B virus is of the Yamagata lineage or of the Victoria lineage.

6. The chimeric HA polypeptide of any one of embodiments 1 to 4, wherein the influenza B virus is influenza B/Yamagata/16/88.

7. The chimeric HA polypeptide of any one of embodiments 1 to 6, wherein the influenza A virus is an influenza A virus of an H4, H5, H6, H7, H8, H9, H10, H11, H12, H13, H14, H15, H16, H17, or H18.

8 The chimeric HA polypeptide of any one of embodiments 1 to 6, wherein the influenza A virus is an H5 HA subtype.

9. The chimeric HA polypeptide of embodiment 7, wherein
a. the following amino acid residues in the 120 loop of influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2);
b. the following amino acid residues in the 150 loop of influenza B virus B/Yamagata/16/88 PNVTSRNG (SEQ ID NO: 18) are substituted with amino acid residues PYQGKSS (SEQ ID NO:19);
c. the following acid residues in the 160 loop of influenza B virus B/Yamagata/16/88 RDNKTA (SEQ ID NO: 5) are substituted with amino acid residues KKNSTY (SEQ ID NO: 6); and/or
d. the following amino acid residues in the 190 helix of influenza B virus B/Yamagata/16/88 NKNQMKN (SEQ ID NO: 7) are substituted with amino acid residues NDAAMQT (SEQ ID NO: 8).

10. The chimeric HA polypeptide of embodiment 8 or 9, wherein the chimeric HA comprises one, two, or more amino acid substitutions outside of one, two, three, or all of the following: the 120 loop, the 150 loop, the 160 loop, and the 190 helix.

11. The chimeric HA polypeptide of any one of embodiments 8 to 10, wherein the H5 subtype is influenza A/Vietnam/1203/04 (HALo) virus.

12. The chimeric HA polypeptide of any one of embodiments 1 to 6, wherein the influenza A virus is an H8 HA subtype.

13. The chimeric HA polypeptide of embodiment 12, wherein
a. the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51);
b. the following amino acid residues in the 150 loop of influenza B virus B/Yamagata/16/88 PNVTSRNG (SEQ ID NO: 18) are substituted with amino acid residues NASTGGQS (SEQ ID NO: 52);
c. the following amino acid residues in the 160 loop of influenza B virus KKKADTY (SEQ ID NO: 53); and/or
d. the following amino acid residues in the 190 helix of influenza B virus B/Yamagata/16/88 NKNQMKN (SEQ ID NO: 7) are substituted with amino acid residues ADAKMQT (SEQ ID NO: 54).

14. The chimeric HA polypeptide of embodiment 12, wherein
a. the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51);
b. the following amino acid residues in the 150 loop of influenza B virus B/Yamagata/16/88 PNVTSRNG (SEQ ID NO: 18) are substituted with amino acid residues NASTGGQS (SEQ ID NO: 52);
c. the following amino acid residues in the 160 loop of influenza B virus B/Yamagata/16/88 RDNKTA (SEQ ID NO: 5) are substituted with amino acid residues KKKADTY (SEQ ID NO: 53) or KKKPDTY (SEQ ID NO: 68); and/or
d. the following amino acid residues in the 190 helix of influenza B virus B/Yamagata/16/88 NKNQMKN (SEQ ID NO: 7) are substituted with amino acid residues ADAKMQT (SEQ ID NO: 54) or PDAKMQT (SEQ ID NO: 69).

15. The chimeric HA polypeptide of embodiment 12 or 13, wherein the chimeric HA comprises one, two, or more amino acid substitutions outside of one, two, three, or all of the following: the 120 loop, the 150 loop, the 160 loop, and the 190 helix.

16. The chimeric HA polypeptide of embodiment 12, 13 or 15, wherein the H8 subtype is influenza A/Mallard/Sweden/24/2002 virus.

17. The chimeric HA polypeptide of any one of embodiments 1 to 6, wherein the influenza A virus is an H11 HA subtype.

18. The chimeric HA polypeptide of embodiment 17, wherein
a. the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55);
b. following amino acid residues in the 150 loop of influenza B virus B/Yamagata/16/88 PNVTSRNG (SEQ ID NO: 18) are substituted with amino acid residues PFGSSNS (SEQ ID NO:56);
c. the following amino acid residues in the 160 loop of influenza B virus B/Yamagata/16/88 RDNKTA (SEQ ID NO: 5) are substituted with amino acid residues HQSGTY (SEQ ID NO: 57); and/or
d. the following amino acid residues in the 190 helix of influenza B virus B/Yamagata/16/88 NKNQMKN (SEQ ID NO: 7) are substituted with amino acid residues TTLKMHQ (SEQ ID NO: 58) or ATLKMHQ (SEQ ID NO: 70).

19. The chimeric HA polypeptide of embodiment 17, wherein
a. the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55);
b. following amino acid residues in the 150 loop of influenza B virus B/Yamagata/16/88 PNVTSRNG (SEQ ID NO: 18) are substituted with amino acid residues PFGSSNS (SEQ ID NO:56) or KFGSSNS (SEQ ID NO:67);
c. the following amino acid residues in the 160 loop of influenza B virus B/Yamagata/16/88 RDNKTA (SEQ ID NO: 5) are substituted with amino acid residues HQSGTY (SEQ ID NO: 57); and/or
d. the following amino acid residues in the 190 helix of influenza B virus B/Yamagata/16/88 NKNQMKN (SEQ ID NO: 7) are substituted with amino acid residues TTLKMHQ (SEQ ID NO: 58) or ATLKMHQ (SEQ ID NO: 70).

20. The chimeric HA polypeptide of embodiment 17 or 18, wherein the chimeric HA comprises one, two, or more amino acid substitutions outside of one, two, three, or all of the following: the 120 loop, the 150 loop, the 160 loop, and the 190 helix.

21. The chimeric HA polypeptide of embodiment 17, 18 or 20, wherein the H11 subtype is influenza A/northern shoveler/Netherlands/18/99.

22. The chimeric HA polypeptide of any one of embodiments 1 to 6, wherein the influenza A virus is an H12 HA subtype.

23. The chimeric HA polypeptide of embodiment 22, wherein
a. the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63);
b. the following amino acid residues in the 150 loop of influenza B virus B/Yamagata/16/88 PNVTSRNG (SEQ ID NO: 18) are substituted with amino acid residues NNTSNQGS (SEQ ID NO:64);
c. the following amino acid residues in the 160 loop of influenza B virus B/Yamagata/16/88 RDNKTA (SEQ ID NO: 5) are substituted with amino acid residues LKSGQF (SEQ ID NO: 65); and/or
d. the following amino acid residues in the 190 helix of influenza B virus B/Yamagata/16/88 NKNQMKN (SEQ ID NO: 7) are substituted with amino acid residues PTSDMQI (SEQ ID NO: 66).

24. The chimeric HA polypeptide of embodiment 22 or 23, wherein the chimeric HA comprises one, two, or more amino acid substitutions outside of one, two, three, or all of the following: the 120 loop, the 150 loop, the 160 loop, and the 190 helix.

25. The chimeric HA polypeptide of any one of embodiments 22 to 24, wherein the H12 subtype is influenza A/mallard/interior Alaska/7MP0167/2007.

26. The chimeric HA polypeptide of any one of embodiments 1 to 6, wherein the influenza A virus is an H13 HA subtype.

27. The chimeric HA polypeptide of embodiment 26, wherein
a. the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59);
b. the following amino acid residues in the 150 loop of influenza B virus B/Yamagata/16/88 PNVTSRNG (SEQ ID NO: 18) are substituted with amino acid residues PDKGASS (SEQ ID NO:60);
c. the following amino acid residues in the 160 loop of influenza B virus B/Yamagata/16/88 RDNKTA (SEQ ID NO: 5) are substituted with amino acid residues KRGNQY (SEQ ID NO: 61); and/or
d. the following amino acid residues in the 190 helix of influenza B virus B/Yamagata/16/88 NKNQMKN (SEQ ID NO: 7) are substituted with amino acid residues VSTNMAK (SEQ ID NO: 62).

28. The chimeric HA polypeptide of embodiment 26 or 27, wherein the chimeric HA comprises one, two, or more amino acid substitutions outside of one, two, three, or all of the following: the 120 loop, the 150 loop, the 160 loop, and the 190 helix.

29. The chimeric HA polypeptide of any one of embodiments 26 to 28, wherein the H13 subtype is influenza A/black headed gull/Sweden/1/99.

30. A chimeric hemagglutinin (HA) polypeptide comprising:
a. a hemagglutinin ectodomain from an influenza B virus with one, two, three or all of the following
   i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA;
   ii 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA;
   iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA; and
   iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA; and
b. a signal peptide, a transmembrane domain and a cytoplasmic tail domain from an influenza A virus.

31. A chimeric hemagglutinin (HA) polypeptide comprising:
a. a hemagglutinin ectodomain from an influenza B virus with one, two, three or all of the following
   i. 2, 3, 4, 5, 6, 7, 8, 9, 10, 11, 12, 13, 14, 15, 16, 17, 18, 19, 20 or more amino acid substitutions within the 120 loop of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the influenza B virus HA with amino acid residues found in a corresponding region of an influenza A virus HA;
   ii. 2, 3, 4, 5, 6, 7, 8, 9 or more amino acid substitutions within the 150 loop of the influenza B virus HA, wherein the amino acid substitutions substitute 2, 3, 4, 5, 6, 7, 8, 9 or more amino acid residues in the 150 loop of the influenza B virus HA with amino acid residues found in a corresponding region of an influenza A virus HA;
   iii. 2, 3, 4, 5 or more amino acid substitutions within the 160 loop of the influenza B virus HA, wherein the amino acid substitutions substitute 2, 3, 4, 5 or more amino acid residues in the 160 loop of the influenza B virus HA with amino acid residues found in a corresponding region of an influenza A virus HA; and
   iv. 2, 3, 4, 5, 6, 7, 8 or more amino acid substitutions within the 190 helix of the influenza B virus HA, wherein the amino acid substitutions substitute 2, 3, 4, 5, 6, 7, 8 or more amino acid residues in the 190 helix of the influenza B virus HA with amino acid residues found in a corresponding region of an influenza A virus HA; and
   b. a signal peptide, a transmembrane domain and a cytoplasmic tail domain from an influenza A virus.

32. The chimeric HA polypeptide of embodiment 30 or 31, wherein the influenza B virus is of the Yamagata lineage or of the Victoria lineage.

33. The chimeric HA polypeptide of embodiment 32, wherein the influenza B virus is influenza B/Yamagata/16/88.

34. The chimeric HA polypeptide of any one of embodiments 30 to 33, wherein the influenza A virus is an influenza A virus of an H4, H5, H6, H7, H8, H9, H10, H11, H12, H13, H14, H15, H16, H17, or H18.

35. The chimeric HA polypeptide of any one of embodiments 30 to 33, wherein the influenza A virus HA is an H5 HA subtype.

36. The chimeric HA polypeptide of embodiment 35, wherein
a. the following amino acid residues in the 120 loop of influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2);
b. the following amino acid residues in the 150 loop of influenza B virus B/Yamagata/16/88 PNVTSRNG (SEQ ID NO: 18) are substituted with amino acid residues PYQGKSS (SEQ ID NO:19);
c. the following acid residues in the 160 loop of influenza B virus B/Yamagata/16/88 RDNKTA (SEQ ID NO: 5) are substituted with amino acid residues KKNSTY (SEQ ID NO: 6); and/or
d. the following amino acid residues in the 190 helix of influenza B virus B/Yamagata/16/88 NKNQMKN (SEQ ID NO: 7) are substituted with amino acid residues NDAAMQT (SEQ ID NO: 8).

37. The chimeric HA polypeptide of embodiment 35 or 36, wherein the chimeric HA comprises one, two, or more amino acid substitutions outside of one, two, three, or all of the following: the 120 loop, the 150 loop, the 160 loop, and the 190 helix.

38. The chimeric HA polypeptide of any one of embodiments 35 to 37, wherein the H5 subtype is influenza A/Vietnam/1203/04 (HALo) virus.

39. The chimeric HA polypeptide of any one of embodiments 30 to 33, wherein the influenza A virus HA is an H8 HA subtype.

40. The chimeric HA polypeptide of embodiment 39, wherein
a. the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51);
b. the following amino acid residues in the 150 loop of influenza B virus B/Yamagata/16/88 PNVTSRNG (SEQ ID NO: 18) are substituted with amino acid residues NASTGGQS (SEQ ID NO: 52);
c. the following amino acid residues in the 160 loop of influenza B virus B/Yamagata/16/88 RDNKTA (SEQ ID NO: 5) are substituted with amino acid residues KKKADTY (SEQ ID NO: 53); and/or
d. the following amino acid residues in the 190 helix of influenza B virus B/Yamagata/16/88 NKNQMKN (SEQ ID NO: 7) are substituted with amino acid residues ADAKMQT (SEQ ID NO: 54).

41. The chimeric HA polypeptide of embodiment 39 or 40, wherein the chimeric HA comprises one, two, or more amino acid substitutions outside of one, two, three, or all of the following: the 120 loop, the 150 loop, the 160 loop, and the 190 helix.

42. The chimeric HA polypeptide of any one of embodiments 39 to 41, wherein the H8 subtype is influenza A/Mallard/Sweden/24/2002 virus.

43. The chimeric HA polypeptide of any one of embodiments 30 to 33, wherein the influenza A virus HA is an H11 HA subtype.

44. The chimeric HA polypeptide of embodiment 43, wherein
a. the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55);
b. following amino acid residues in the 150 loop of influenza B virus B/Yamagata/16/88 PNVTSRNG (SEQ ID NO: 18) are substituted with amino acid residues PFGSSNS (SEQ ID NO:56);
c. the following amino acid residues in the 160 loop of influenza B virus B/Yamagata/16/88 RDNKTA (SEQ ID NO: 5) are substituted with amino acid residues HQSGTY (SEQ ID NO: 57); and/or
d. the following amino acid residues in the 190 helix of influenza B virus B/Yamagata/16/88 NKNQMKN (SEQ ID NO: 7) are substituted with amino acid residues TTLKMHQ (SEQ ID NO: 58) or ATLKMHQ (SEQ ID NO: 70).

45. The chimeric HA polypeptide of embodiment 43 or 44, wherein the chimeric HA comprises one, two, or more amino acid substitutions outside of one, two, three, or all of the following: the 120 loop, the 150 loop, the 160 loop, and the 190 helix.

46. The chimeric HA polypeptide of any one of embodiments 43 to 45, wherein the H11 subtype is influenza A/northern shoveler/Netherlands/18/99.

47. The chimeric HA polypeptide of any one of embodiments 30 to 33, wherein the influenza A virus HA is an H12 HA subtype.

48. The chimeric HA polypeptide of embodiment 47, wherein
a. the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63);
b. the following amino acid residues in the 150 loop of influenza B virus B/Yamagata/16/88 PNVTSRNG (SEQ ID NO: 18) are substituted with amino acid residues NNTSNQGS (SEQ ID NO:64);
c. the following amino acid residues in the 160 loop of influenza B virus B/Yamagata/16/88 RDNKTA (SEQ ID NO: 5) are substituted with amino acid residues LKSGQF (SEQ ID NO: 65); and/or
d. the following amino acid residues in the 190 helix of influenza B virus B/Yamagata/16/88 NKNQMKN (SEQ ID NO: 7) are substituted with amino acid residues PTSDMQI (SEQ ID NO: 66).

49. The chimeric HA polypeptide of embodiment 47 or 48, wherein the chimeric HA comprises one, two, or more amino acid substitutions outside of one, two, three, or all of the following: the 120 loop, the 150 loop, the 160 loop, and the 190 helix.

50. The chimeric HA polypeptide of any one of embodiments 47 to 49, wherein the H12 subtype is influenza A/mallard/interior Alaska/7MP0167/2007.

51. The chimeric HA polypeptide of any one of embodiments 30 to 33, wherein the influenza A virus HA is an H13 HA subtype.

52. The chimeric HA polypeptide of embodiment 51, wherein
a. the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59);
b. the following amino acid residues in the 150 loop of influenza B virus B/Yamagata/16/88 PNVTSRNG (SEQ ID NO: 18) are substituted with amino acid residues PDKGASS (SEQ ID NO:60);
c. the following amino acid residues in the 160 loop of influenza B virus KRGNQY (SEQ ID NO: 61); and/or
d. the following amino acid residues in the 190 helix of influenza B virus B/Yamagata/16/88 NKNQMKN (SEQ ID NO: 7) are substituted with amino acid residues VSTNMAK (SEQ ID NO: 62).

53. The chimeric HA polypeptide of embodiment 51 or 52, wherein the chimeric HA comprises one, two, or more amino acid substitutions outside of one, two, three, or all of the following: the 120 loop, the 150 loop, the 160 loop, and the 190 helix.

54. The chimeric HA polypeptide of any one of embodiments 51 to 53, wherein the H13 subtype is influenza A/black headed gull/Sweden/1/99.

55. A chimeric hemagglutinin (HA) polypeptide comprising the amino acid sequence set forth in SEQ ID NO: 21 or SEQ ID NO: 27, or the amino acid sequence set forth in SEQ ID NO: 21 or SEQ ID NO: 27 without the signal peptide.

56. A chimeric hemagglutinin (HA) polypeptide comprising the amino acid sequence set forth in SEQ ID NO: 23 or SEQ ID NO: 25, or the amino acid sequence set forth in SEQ ID NO: 23 or SEQ ID NO: 25 without the signal peptide.

57. A chimeric hemagglutinin (HA) polypeptide comprising the amino acid sequence of the influenza virus HA ectodomain forth in SEQ ID NO: 21 or SEQ ID NO: 27.

58. A chimeric hemagglutinin (HA) polypeptide comprising the amino acid sequence of the influenza virus HA ectodomain forth in SEQ ID NO: 23 or SEQ ID NO: 25.

59. A chimeric hemagglutinin (HA) polypeptide comprising the amino acid sequence set forth in SEQ ID NO: 44, SEQ ID NO: 46, SEQ ID NO: 48, or SEQ ID NO: 50, or the amino acid sequence set forth in SEQ ID NO: 44, SEQ ID NO: 46, SEQ ID NO: 48, or SEQ ID NO: 50 without the signal peptide.

60. A chimeric hemagglutinin (HA) polypeptide comprising the amino acid sequence of the influenza virus HA ectodomain forth in SEQ ID NO: 44, SEQ ID NO: 46, SEQ ID NO: 48, or SEQ ID NO: 50.

61. The chimeric HA polypeptide of any one of embodiments 1 to 60, which is isolated.

62. A nucleic acid sequence encoding the chimeric HA polypeptide of any one of embodiments 1 to 13, 15 to 18, 20 to 29, 59, or 60.

63. A nucleic acid sequence encoding the chimeric HA polypeptide of any one of embodiments 30 to 55, or 57.

64. A nucleic acid sequence encoding the chimeric HA polypeptide of any one of embodiments 14, 18, 56 or 58.

65. A nucleic acid sequence comprising the nucleotide sequence set forth in SEQ ID NO: 20 or SEQ ID NO: 26, or a complement thereof.

66. A nucleic acid sequence comprising the nucleotide sequence set forth in SEQ ID NO: 22 or SEQ ID NO: 24, or a complement thereof.

67. A nucleic acid sequence comprising the nucleotide sequence set forth in SEQ ID NO: 43, SEQ ID NO: 45, SEQ ID NO: 47, or SEQ ID NO: 49, or a complement thereof.

68. The nucleic acid sequence of embodiment 62 or 67, further comprising a nucleotide sequence comprising the 5′ and 3′ non-coding regions of an influenza B virus.

69. The nucleic acid sequence of any one of embodiments 62 to 68, wherein the nucleic acid sequence is cDNA.

70. The nucleic acid sequence of any one of embodiments 62 to 69, which is isolated.

71. An expression vector comprising the nucleic acid sequence of any one of embodiments 62 to 69.

72. A viral vector comprising the nucleic acid sequence of any one of embodiments 62 to 69.

73. An influenza B virus engineered to express the nucleic acid sequence of embodiment 62, 67 or 68.

74. An influenza A virus engineered to express the nucleic acid sequence of embodiment 63 or 65.

75. An influenza virus engineered to express the chimeric HA polypeptide of any one of embodiments 1 to 13, 15 to 18, 20 to 29, 59 or 60.

76. The influenza virus of embodiment 75, wherein the influenza virus is an influenza B virus.

77. The influenza virus of embodiment 76, wherein the chimeric HA polypeptide is encoded by a nucleotide sequence comprising 5′ and 3′ noncoding regions of an influenza B virus.

78. An influenza B virus comprising the chimeric HA polypeptide of any one of embodiments 1 to 13, 15 to 18, 20 to 29, 59 or 60.

79. An influenza A virus engineered to express the chimeric HA polypeptide of any one of embodiments 1, 2, 30 to 55, or 57.

80. An influenza A virus comprising the chimeric HA polypeptide of any one of embodiments 1, 2, 30 to 55, or 57.

81. The influenza virus of embodiment 74 or 79, wherein the chimeric HA polypeptide is encoded by a nucleotide sequence comprising 5′ and 3′ noncoding regions of an influenza A virus.

82. The influenza virus of any one of embodiments 73 to 81, which is attenuated.

83. The influenza virus of embodiment 78 or 80, which is inactivated.

84. A virus-like particle comprising the chimeric HA polypeptide of any one of embodiments 1 to 60.

85. A subunit vaccine comprising the chimeric HA polypeptide of any one of embodiments 1 to 61.

86. A split vaccine comprising the chimeric HA polypeptide of any one of embodiments 1 to 61.

87. An immunogenic composition comprising the chimeric HA polypeptide of any one of embodiments 1 to 61.

88. An immunogenic composition comprising the influenza virus of any one of embodiments 73 to 83.

89. An immunogenic composition comprising the virus-like particle of embodiment 84.

90. The immunogenic composition of any one of embodiments 87 to 89, comprising an adjuvant.

91. The subunit vaccine of embodiment 85, comprising an adjuvant.

92. The split vaccine of embodiment 86, comprising an adjuvant.

93. A method of inducing an immune response against influenza B virus in a subject, comprising administering to the subject the immunogenic composition of any one of embodiments 87 to 90.

94. A method of inducing an immune response against influenza B virus in a subject, comprising administering to the subject the subunit vaccine of embodiment 85 or 91.

95. A method of inducing an immune response against influenza B virus in a subject, comprising administering to the subject the split vaccine of embodiment 86 or 92.

96. A method of inducing an immune response against influenza B virus in a subject comprising
a. administering to the subject a first immunogenic composition comprising a first chimeric HA, which is the chimeric HA of any one of embodiments 1 to 61; and
b. a certain period of time after the administration of the first immunogenic composition, administering to the subject a second immunogenic composition comprising a second chimeric HA, which is the chimeric HA of any one of embodiments 1 to 61, wherein the first and second chimeric HAs are not the same.

97. A method of inducing an immune response against influenza B virus in a subject that has been administered a first immunogenic composition, comprising administering to the subject a second immunogenic composition comprising a second chimeric HA, which is the chimeric HA of any one of embodiments 1 to 61, wherein the first immunogenic composition comprising a first chimeric HA, which is the chimeric HA of any one of embodiments 1 to 61, and wherein the first and second chimeric HAs are not the same.

98. The method of embodiment 96 or 97, wherein the first and second chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

99. The method of embodiment 96 to 98, wherein the method further comprises administering to the subject a third immunogenic composition comprising a third chimeric HA, which is the chimeric HA of any one of embodiments 1 to 61, wherein the first, second, and third chimeric HAs are not the same.

100. The method of embodiment 99, wherein the first, second, and third chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

101. The method of embodiment 99 or 100, wherein the third immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the second immunogenic composition.

102. The method of any one of embodiments 96 to 101, wherein the second immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the first immunogenic composition.

103. A method of inducing an immune response against influenza B virus in a subject, comprising:
a. a first administration of a first immunogenic composition comprising a first chimeric HA polypeptide to the subject, wherein the first chimeric HA polypeptide comprises a first ectodomain of a first influenza B virus comprising:
   i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a first subtype;
   ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the first subtype;
   iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the first subtype; and
   iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the first subtype; and
b. a second administration of a second immunogenic composition comprising a second chimeric HA polypeptide to the subject a first period of time after the first administration, wherein the second chimeric HA polypeptide comprises a second ectodomain of a second influenza B virus comprising:
   i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a second subtype;
   ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the second subtype;
   iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the second subtype; and
   iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the second subtype; and
c. a third administration of a third immunogenic composition comprising a third chimeric HA polypeptide to the subject a second period of time after the second administration, wherein the third chimeric HA polypeptide comprises a third ectodomain of a third influenza B virus comprising:
   i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a third subtype;
   ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the third subtype;
   iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the third subtype; and
   iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the third subtype.

104. The method of embodiment 103, wherein first chimeric HA polypeptide is administered to the subject as a nucleic acid encoding the polypeptide.

105. The method of embodiment 103, wherein the first chimeric HA polypeptide is administered to the subject as part of an influenza virus.

106. The method of any one of embodiments 103 to 105, wherein the first, second and third subtypes are different.

107. The method of embodiment 106, wherein the subtypes are selected from the group consisting of H4, H5, H6, H7, H8, H9, H10, H11, H12, H13, H14, H15, H16, H17 and H18.

108. The method of embodiment 106, wherein the subtypes are selected from the group consisting of H5, H8, H11, H12, and H13.

109. The method of any one of embodiments 103 to 105, wherein the first period of time is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the first administration.

110. The method of any one of embodiments 103 to 105, wherein the second period of time is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the second administration.

111. The method of any one of embodiments 93 to 110, wherein the subject is human.

112. An immunogenic composition of any one of embodiments 87 to 90 for use in a method of inducing an immune response against influenza B virus in a subject, wherein the method comprises administering to the subject said immunogenic composition.

113. A subunit vaccine of embodiment 85 or 91 for use in a method of inducing an immune response against influenza B virus in a subject, wherein the method comprises administering to the subject said subunit vaccine.

114. A split vaccine of embodiment 86 or 92 for use in a method of inducing an immune response against influenza B virus in a subject, wherein the method comprises administering to the subject said split vaccine.

115. A first immunogenic composition for use in a method of inducing an immune response against influenza B virus in a subject, wherein the method comprises:
a. administering to the subject said first immunogenic composition, wherein said first immunogenic composition comprises a first chimeric HA, which is the chimeric HA of any one of embodiments 1 to 61; and
b. a certain period of time after the administration of the first immunogenic composition, administering to the subject a second immunogenic composition comprising a second chimeric HA, which is the chimeric HA of any one of embodiments 1 to 61, wherein the first and second chimeric HAs are not the same.

116. A second immunogenic composition for use in a method of inducing an immune response against influenza B virus in a subject that has been administered a first immunogenic composition, wherein the method comprises administering to the subject said second immunogenic composition, wherein said second immunogenic composition comprises a second chimeric HA, which is the chimeric HA of any one of embodiments 1 to 61, wherein the first immunogenic composition comprising a first chimeric HA, which is the chimeric HA of any one of embodiments 1 to 61, and wherein the first and second chimeric HAs are not the same.

117. The first immunogenic composition for use of embodiment 115, wherein the first and second chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

118. The second immunogenic composition for use of embodiment 116, wherein the first and second chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

119. The first immunogenic composition for use of embodiment 115 or 117, wherein the method further comprises administering to the subject a third immunogenic composition comprising a third chimeric HA, which is the chimeric HA of any one of embodiments 1 to 61, wherein the first, second, and third chimeric HAs are not the same.

120. The second immunogenic composition of embodiment 116 or 118, wherein the method further comprises administering to the subject a third immunogenic composition comprising a third chimeric HA, which is the chimeric HA of any one of embodiments 1 to 61, wherein the first, second, and third chimeric HAs are not the same.

121. The first immunogenic composition for use of embodiment 119, wherein the first, second, and third chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

122. The second immunogenic composition for use of embodiment 120, wherein the first, second, and third chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

123. The first immunogenic composition for use of embodiment 119 or 121, wherein the third immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the second immunogenic composition.

124. The second immunogenic composition for use of embodiment 120 or 122, wherein the third immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the second immunogenic composition.

125. The first immunogenic composition for use of embodiment 115, 117, 119 or 121, wherein the second immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the first immunogenic composition.

126. The second immunogenic composition for use of embodiment 116, 118, 120 or 122, wherein the second immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the first immunogenic composition.

127. A first immunogenic composition for use in a method of inducing an immune response against influenza B virus in a subject, wherein the method comprises:
a. a first administration of the first immunogenic composition, wherein the first immunogenic composition comprises a first chimeric HA polypeptide to the subject, and wherein the first chimeric HA polypeptide comprises a first ectodomain of a first influenza B virus comprising:
   i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a first subtype;
   ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the first subtype;
   iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the first subtype; and
   iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the first subtype; and
b. a second administration of a second immunogenic composition comprising a second chimeric HA polypeptide to the subject a first period of time after the first administration, wherein the second chimeric HA polypeptide comprises a second ectodomain of a second influenza B virus comprising:
   i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a second subtype;
   ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the second subtype;
   iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the second subtype; and
   iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the second subtype; and
c. a third administration of a third immunogenic composition comprising a third chimeric HA polypeptide to the subject a second period of time after the second administration, wherein the third chimeric HA polypeptide comprises a third ectodomain of a third influenza B virus comprising:
   i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a third subtype;
   ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the third subtype;
   iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the third subtype; and
   iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the third subtype.

128. The first immunogenic composition for use of embodiment 127, wherein first chimeric HA polypeptide is administered to the subject as a nucleic acid encoding the polypeptide.

129. The first immunogenic composition for use of embodiment 127, wherein the first chimeric HA polypeptide is administered to the subject as part of an influenza virus.

130. The first immunogenic composition for use of any one of embodiments 127 to 129, wherein the first, second and third subtypes are different.

131. The first immunogenic composition for use of embodiment 130, wherein the subtypes are selected from the group consisting of H4, H5, H6, H7, H8, H9, H10, H11, H12, H13, H14, H15, H16, H17 and H18.

132. The first immunogenic composition for use of embodiment 130, wherein the subtypes are selected from the group consisting of H5, H8, H11, H12, and H13.

133. The first immunogenic composition for use of any one of embodiments 127 to 132, wherein the first period of time is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the first administration.

134. The first immunogenic composition for use of any one of embodiments 127 to 133, wherein the second period of time is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the second administration.

135. The immunogenic composition for use of any one of embodiments 112 or 115 to 134, or the vaccine for use of 113 or 114, wherein the subject is human.

### 8. EQUIVALENTS

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A chimeric hemagglutinin (HA) polypeptide comprising a hemagglutinin ectodomain from an influenza B virus comprising one, two, three or all of the following:
a. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA;
b. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA;
c. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA; and
d. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA.

2. A chimeric hemagglutinin (HA) polypeptide comprising a hemagglutinin ectodomain from an influenza B virus comprising one, two, three or all of the following:
a. 2, 3, 4, 5, 6, 7, 8, 9, 10, 11, 12, 13, 14, 15, 16, 17, 18, 19, 20 or more amino acid substitutions within the 120 loop of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the influenza B virus HA with amino acid residues found in a corresponding region of an influenza A virus HA;
b. 2, 3, 4, 5, 6, 7, 8, 9 or more amino acid substitutions within the 150 loop of the influenza B virus HA, wherein the amino acid substitutions substitute 2, 3, 4, 5, 6, 7, 8, 9 or more amino acid residues in the 150 loop of the influenza B virus HA with amino acid residues found in a corresponding region of an influenza A virus HA;
c. 2, 3, 4, 5 or more amino acid substitutions within the 160 loop of the influenza B virus HA, wherein the amino acid substitutions substitute 2, 3, 4, 5 or more amino acid residues in the 160 loop of the influenza B virus HA with amino acid residues found in a corresponding region of an influenza A virus HA; and
d. 2, 3, 4, 5, 6, 7, 8 or more amino acid substitutions within the 190 helix of the influenza B virus HA, wherein the amino acid substitutions substitute 2, 3, 4, 5, 6, 7, 8 or more amino acid residues in the 190 helix of the influenza B virus HA with amino acid residues found in a corresponding region of an influenza A virus HA.

3. The chimeric HA polypeptide of claim 1 or 2 which further comprises the signal peptide of the influenza B virus HA.

4. The chimeric HA polypeptide of claim 1, 2, or 3 which further comprises the transmembrane domain and cytoplasmic tail domain of the influenza B virus HA.

5. The chimeric HA polypeptide of any one of claims 1 to 4, wherein the influenza B virus is of the Yamagata lineage or of the Victoria lineage.

6. The chimeric HA polypeptide of any one of claims 1 to 4, wherein the influenza B virus is influenza B/Yamagata/16/88.

7. The chimeric HA polypeptide of any one of claims 1 to 6, wherein the influenza A virus is an influenza A virus of an H4, H5, H6, H7, H8, H9, H10, H11, H12, H13, H14, H15, H16, H17, or H18.

8. The chimeric HA polypeptide of any one of claims 1 to 6, wherein the influenza A virus is an H5 HA subtype.

9. The chimeric HA polypeptide of claim 7, wherein
a. the following amino acid residues in the 120 loop of influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2);
b. the following amino acid residues in the 150 loop of influenza B virus B/Yamagata/16/88 PNVTSRNG (SEQ ID NO: 18) are substituted with amino acid residues PYQGKSS (SEQ ID NO:19);
c. the following acid residues in the 160 loop of influenza B virus B/Yamagata/16/88 RDNKTA (SEQ ID NO: 5) are substituted with amino acid residues KKNSTY (SEQ ID NO: 6); and/or
d. the following amino acid residues in the 190 helix of influenza B virus B/Yamagata/16/88 NKNQMKN (SEQ ID NO: 7) are substituted with amino acid residues NDAAMQT (SEQ ID NO: 8).

10. The chimeric HA polypeptide of claim 8 or 9, wherein the chimeric HA comprises one, two, or more amino acid substitutions outside of one, two, three, or all of the following: the 120 loop, the 150 loop, the 160 loop, and the 190 helix.

11. The chimeric HA polypeptide of any one of claims 8 to 10, wherein the H5 subtype is influenza A/Vietnam/1203/04 (HALo) virus.

12. The chimeric HA polypeptide of any one of claims 1 to 6, wherein the influenza A virus is an H8 HA subtype.

13. The chimeric HA polypeptide of claim 12, wherein
a. the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51);
b. the following amino acid residues in the 150 loop of influenza B virus B/Yamagata/16/88 PNVTSRNG (SEQ ID NO: 18) are substituted with amino acid residues NASTGGQS (SEQ ID NO: 52);
c. the following amino acid residues in the 160 loop of influenza B virus B/Yamagata/16/88 RDNKTA (SEQ ID NO: 5) are substituted with amino acid residues KKKADTY (SEQ ID NO: 53); and/or
d. the following amino acid residues in the 190 helix of influenza B virus B/Yamagata/16/88 NKNQMKN (SEQ ID NO: 7) are substituted with amino acid residues ADAKMQT (SEQ ID NO: 54).

14. The chimeric HA polypeptide of claim 12, wherein
a. the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51);
b. the following amino acid residues in the 150 loop of influenza B virus B/Yamagata/16/88 PNVTSRNG (SEQ ID NO: 18) are substituted with amino acid residues NASTGGQS (SEQ ID NO: 52);
c. the following amino acid residues in the 160 loop of influenza B virus B/Yamagata/16/88 RDNKTA (SEQ ID NO: 5) are substituted with amino acid residues KKKADTY (SEQ ID NO: 53) or KKKPDTY (SEQ ID NO: 68); and/or
d. the following amino acid residues in the 190 helix of influenza B virus B/Yamagata/16/88 NKNQMKN (SEQ ID NO: 7) are substituted with amino acid residues ADAKMQT (SEQ ID NO: 54) or PDAKMQT (SEQ ID NO: 69).

15. The chimeric HA polypeptide of claim 12 or 13, wherein the chimeric HA comprises one, two, or more amino acid substitutions outside of one, two, three, or all of the following: the 120 loop, the 150 loop, the 160 loop, and the 190 helix.

16. The chimeric HA polypeptide of claim 12, 13 or 15, wherein the H8 subtype is influenza A/Mallard/Sweden/24/2002 virus.

17. The chimeric HA polypeptide of any one of claims 1 to 6, wherein the influenza A virus is an H11 HA subtype.

18. The chimeric HA polypeptide of claim 17, wherein
a. the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55);
b. following amino acid residues in the 150 loop of influenza B virus B/Yamagata/16/88 PNVTSRNG (SEQ ID NO: 18) are substituted with amino acid residues PFGSSNS (SEQ ID NO:56);
c. the following amino acid residues in the 160 loop of influenza B virus B/Yamagata/16/88 RDNKTA (SEQ ID NO: 5) are substituted with amino acid residues HQSGTY (SEQ ID NO: 57); and/or
d. the following amino acid residues in the 190 helix of influenza B virus B/Yamagata/16/88 NKNQMKN (SEQ ID NO: 7) are substituted with amino acid residues TTLKMHQ (SEQ ID NO: 58) or ATLKMHQ (SEQ ID NO: 70).

19. The chimeric HA polypeptide of claim 17, wherein
a. the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55);
b. following amino acid residues in the 150 loop of influenza B virus B/Yamagata/16/88 PNVTSRNG (SEQ ID NO: 18) are substituted with amino acid residues PFGSSNS (SEQ ID NO:56) or KFGSSNS (SEQ ID NO:67);
c. the following amino acid residues in the 160 loop of influenza B virus B/Yamagata/16/88 RDNKTA (SEQ ID NO: 5) are substituted with amino acid residues HQSGTY (SEQ ID NO: 57); and/or
d. the following amino acid residues in the 190 helix of influenza B virus B/Yamagata/16/88 NKNQMKN (SEQ ID NO: 7) are substituted with amino acid residues TTLKMHQ (SEQ ID NO: 58) or ATLKMHQ (SEQ ID NO: 70).

20. The chimeric HA polypeptide of claim 17 or 18, wherein the chimeric HA comprises one, two, or more amino acid substitutions outside of one, two, three, or all of the following: the 120 loop, the 150 loop, the 160 loop, and the 190 helix.

21. The chimeric HA polypeptide of claim 17, 18 or 20, wherein the H11 subtype is influenza A/northern shoveler/Netherlands/18/99.

22. The chimeric HA polypeptide of any one of claims 1 to 6, wherein the influenza A virus is an H12 HA subtype.

23. The chimeric HA polypeptide of claim 22, wherein
a. the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63);
b the following amino acid residues in the 150 loop of influenza B virus B/Yamagata/16/88 PNVTSRNG (SEQ ID NO: 18) are substituted with amino acid residues NNTSNQGS (SEQ ID NO:64);
c. the following amino acid residues in the 160 loop of influenza B virus B/Yamagata/16/88 RDNKTA (SEQ ID NO: 5) are substituted with amino acid residues LKSGQF (SEQ ID NO: 65); and/or
d. the following amino acid residues in the 190 helix of influenza B virus B/Yamagata/16/88 NKNQMKN (SEQ ID NO: 7) are substituted with amino acid residues PTSDMQI (SEQ ID NO: 66).

24. The chimeric HA polypeptide of claim 22 or 23, wherein the chimeric HA comprises one, two, or more amino acid substitutions outside of one, two, three, or all of the following: the 120 loop, the 150 loop, the 160 loop, and the 190 helix.

25. The chimeric HA polypeptide of any one of claims 22 to 24, wherein the H12 subtype is influenza A/mallard/interior Alaska/7MP0167/2007.

26. The chimeric HA polypeptide of any one of claims 1 to 6, wherein the influenza A virus is an H13 HA subtype.

27. The chimeric HA polypeptide of claim 26, wherein
a. the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59);
b. the following amino acid residues in the 150 loop of influenza B virus B/Yamagata/16/88 PNVTSRNG (SEQ ID NO: 18) are substituted with amino acid residues PDKGASS (SEQ ID NO:60);
c. the following amino acid residues in the 160 loop of influenza B virus B/Yamagata/16/88 RDNKTA (SEQ ID NO: 5) are substituted with amino acid residues KRGNQY (SEQ ID NO: 61); and/or
d. the following amino acid residues in the 190 helix of influenza B virus B/Yamagata/16/88 NKNQMKN (SEQ ID NO: 7) are substituted with amino acid residues VSTNMAK (SEQ ID NO: 62).

28. The chimeric HA polypeptide of claim 26 or 27, wherein the chimeric HA comprises one, two, or more amino acid substitutions outside of one, two, three, or all of the following: the 120 loop, the 150 loop, the 160 loop, and the 190 helix.

29. The chimeric HA polypeptide of any one of claims 26 to 28, wherein the H13 subtype is influenza A/black headed gull/Sweden/1/99.

30. A chimeric hemagglutinin (HA) polypeptide comprising:
a. a hemagglutinin ectodomain from an influenza B virus with one, two, three or all of the following
i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA;
ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA;
iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA; and
iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA; and
b. a signal peptide, a transmembrane domain and a cytoplasmic tail domain from an influenza A virus.

31. A chimeric hemagglutinin (HA) polypeptide comprising:
a. a hemagglutinin ectodomain from an influenza B virus with one, two, three or all of the following
i. 2, 3, 4, 5, 6, 7, 8, 9, 10, 11, 12, 13, 14, 15, 16, 17, 18, 19, 20 or more amino acid substitutions within the 120 loop of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the influenza B virus HA with amino acid residues found in a corresponding region of an influenza A virus HA;
ii. 2, 3, 4, 5, 6, 7, 8, 9 or more amino acid substitutions within the 150 loop of the influenza B virus HA, wherein the amino acid substitutions substitute 2, 3, 4, 5, 6, 7, 8, 9 or more amino acid residues in the 150 loop of the influenza B virus HA with amino acid residues found in a corresponding region of an influenza A virus HA;
iii. 2, 3, 4, 5 or more amino acid substitutions within the 160 loop of the influenza B virus HA, wherein the amino acid substitutions substitute 2, 3, 4, 5 or more amino acid residues in the 160 loop of the influenza B virus HA with amino acid residues found in a corresponding region of an influenza A virus HA; and
iv. 2, 3, 4, 5, 6, 7, 8 or more amino acid substitutions within the 190 helix of the influenza B virus HA, wherein the amino acid substitutions substitute 2, 3, 4, 5, 6, 7, 8 or more amino acid residues in the 190 helix of the influenza B virus HA with amino acid residues found in a corresponding region of an influenza A virus HA; and
b. a signal peptide, a transmembrane domain and a cytoplasmic tail domain from an influenza A virus.

32. The chimeric HA polypeptide of claim 30 or 31, wherein the influenza B virus is of the Yamagata lineage or of the Victoria lineage.

33. The chimeric HA polypeptide of claim 32, wherein the influenza B virus is influenza B/Yamagata/16/88.

34. The chimeric HA polypeptide of any one of claims 30 to 33, wherein the influenza A virus is an influenza A virus of an H4, H5, H6, H7, H8, H9, H10, H11, H12, H13, H14, H15, H16, H17, or H18.

35. The chimeric HA polypeptide of any one of claims 30 to 33, wherein the influenza A virus HA is an H5 HA subtype.

36. The chimeric HA polypeptide of claim 35, wherein
a. the following amino acid residues in the 120 loop of influenza B virus B/Yamagata/16/88 TIP and NIRLSTHNVINAERAPGGPYRL (SEQ ID NO: 1) are substituted with amino acid residues FIP and KIQLSTKNVINAEHAPGGPYRL (SEQ ID NO: 2);
b. the following amino acid residues in the 150 loop of influenza B virus B/Yamagata/16/88 PNVTSRNG (SEQ ID NO: 18) are substituted with amino acid residues PYQGKSS (SEQ ID NO:19);
c. the following acid residues in the 160 loop of influenza B virus B/Yamagata/16/88 RDNKTA (SEQ ID NO: 5) are substituted with amino acid residues KKNSTY (SEQ ID NO: 6); and/or
d. the following amino acid residues in the 190 helix of influenza B virus B/Yamagata/16/88 NKNQMKN (SEQ ID NO: 7) are substituted with amino acid residues NDAAMQT (SEQ ID NO: 8).

37. The chimeric HA polypeptide of claim 35 or 36, wherein the chimeric HA comprises one, two, or more amino acid substitutions outside of one, two, three, or all of the following: the 120 loop, the 150 loop, the 160 loop, and the 190 helix.

38. The chimeric HA polypeptide of any one of claims 35 to 37, wherein the H5 subtype is influenza A/Vietnam/1203/04 (HALo) virus.

39. The chimeric HA polypeptide of any one of claims 30 to 33, wherein the influenza A virus HA is an H8 HA subtype.

40. The chimeric HA polypeptide of claim 39, wherein
a. the following amino acid residues in influenza B virus B/Yamagata/16/88 TIP and NIRLSTHNVINAERAPGGPYRL (SEQ ID NO: 1) are substituted with amino acid residues HIP and RIRLSTYNVINAETAPGGPYRL (SEQ ID NO: 51);
b. the following amino acid residues in the 150 loop of influenza B virus B/Yamagata/16/88 PNVTSRNG (SEQ ID NO: 18) are substituted with amino acid residues NASTGGQS (SEQ ID NO: 52);
c. the following amino acid residues in the 160 loop of influenza B virus KKKADTY (SEQ ID NO: 53); and/or
d. the following amino acid residues in the 190 helix of influenza B virus B/Yamagata/16/88 NKNQMKN (SEQ ID NO: 7) are substituted with amino acid residues ADAKMQT (SEQ ID NO: 54).

41. The chimeric HA polypeptide of claim 39 or 40, wherein the chimeric HA comprises one, two, or more amino acid substitutions outside of one, two, three, or all of the following: the 120 loop, the 150 loop, the 160 loop, and the 190 helix.

42. The chimeric HA polypeptide of any one of claims 39 to 41, wherein the H8 subtype is influenza A/Mallard/Sweden/24/2002 virus.

43. The chimeric HA polypeptide of any one of claims 30 to 33, wherein the influenza A virus HA is an H11 HA subtype.

44. The chimeric HA polypeptide of claim 43, wherein
a. the following amino acid residues in influenza B virus B/Yamagata/16/88 TIP and NIRLSTHNVINAERAPGGPYRL (SEQ ID NO: 1) are substituted with amino acid residues LIP and KIELSTSNVINAEVAPGGPYRL (SEQ ID NO: 55);
b. following amino acid residues in the 150 loop of influenza B virus B/Yamagata/16/88 PNVTSRNG (SEQ ID NO: 18) are substituted with amino acid residues PFGSSNS (SEQ ID NO:56);
c. the following amino acid residues in the 160 loop of influenza B virus B/Yamagata/16/88 RDNKTA (SEQ ID NO: 5) are substituted with amino acid residues HQSGTY (SEQ ID NO: 57); and/or
d. the following amino acid residues in the 190 helix of influenza B virus B/Yamagata/16/88 NKNQMKN (SEQ ID NO: 7) are substituted with amino acid residues TTLKMHQ (SEQ ID NO: 58) or ATLKMHQ (SEQ ID NO: 70).

45. The chimeric HA polypeptide of claim 43 or 44, wherein the chimeric HA comprises one, two, or more amino acid substitutions outside of one, two, three, or all of the following: the 120 loop, the 150 loop, the 160 loop, and the 190 helix.

46. The chimeric HA polypeptide of any one of claims 43 to 45, wherein the H11 subtype is influenza A/northern shoveler/Netherlands/18/99.

47. The chimeric HA polypeptide of any one of claims 30 to 33, wherein the influenza A virus HA is an H12 HA subtype.

48. The chimeric HA polypeptide of claim 47, wherein
a. the following amino acid residues in influenza B virus B/Yamagata/16/88 TIP and NIRLSTHNVINAERAPGGPYRL (SEQ ID NO: 1) are substituted with amino acid residues YIP and RIKLSTENVINAETAPGGPYRL (SEQ ID NO: 63);
b. the following amino acid residues in the 150 loop of influenza B virus B/Yamagata/16/88 PNVTSRNG (SEQ ID NO: 18) are substituted with amino acid residues NNTSNQGS (SEQ ID NO:64);
c. the following amino acid residues in the 160 loop of influenza B virus B/Yamagata/16/88 RDNKTA (SEQ ID NO: 5) are substituted with amino acid residues LKSGQF (SEQ ID NO: 65); and/or
d. the following amino acid residues in the 190 helix of influenza B virus B/Yamagata/16/88 NKNQMKN (SEQ ID NO: 7) are substituted with amino acid residues PTSDMQI (SEQ ID NO: 66).

49. The chimeric HA polypeptide of claim 47 or 48, wherein the chimeric HA comprises one, two, or more amino acid substitutions outside of one, two, three, or all of the following: the 120 loop, the 150 loop, the 160 loop, and the 190 helix.

50. The chimeric HA polypeptide of any one of claims 47 to 49, wherein the H12 subtype is influenza A/mallard/interior Alaska/7MP0167/2007.

51. The chimeric HA polypeptide of any one of claims 30 to 33, wherein the influenza A virus HA is an H13 HA subtype.

52. The chimeric HA polypeptide of claim 51, wherein
a. the following amino acid residues in influenza B virus B/Yamagata/16/88 TIP and NIRLSTHNVINAERAPGGPYRL (SEQ ID NO: 1) are substituted with amino acid residues NIP and RIELSTHNVINAEVAPGGPYRL (SEQ ID NO: 59);
b. the following amino acid residues in the 150 loop of influenza B virus B/Yamagata/16/88 PNVTSRNG (SEQ ID NO: 18) are substituted with amino acid residues PDKGASS (SEQ ID NO:60);
c. the following amino acid residues in the 160 loop of influenza B virus B/Yamagata/16/88 RDNKTA (SEQ ID NO: 5) are substituted with amino acid residues KRGNQY (SEQ ID NO: 61); and/or
d. the following amino acid residues in the 190 helix of influenza B virus B/Yamagata/16/88 NKNQMKN (SEQ ID NO: 7) are substituted with amino acid residues VSTNMAK (SEQ ID NO: 62).

53. The chimeric HA polypeptide of claim 51 or 52, wherein the chimeric HA comprises one, two, or more amino acid substitutions outside of one, two, three, or all of the following: the 120 loop, the 150 loop, the 160 loop, and the 190 helix.

54. The chimeric HA polypeptide of any one of claims 51 to 53, wherein the H13 subtype is influenza A/black headed gull/Sweden/1/99.

55. A chimeric hemagglutinin (HA) polypeptide comprising the amino acid sequence set forth in SEQ ID NO: 21 or SEQ ID NO: 27, or the amino acid set forth in SEQ ID NO: 21 or SEQ ID NO: 27 without the signal peptide.

56. A chimeric hemagglutinin (HA) polypeptide comprising the amino acid sequence set forth in SEQ ID NO: 23 or SEQ ID NO: 25, or the amino acid sequence set forth in SEQ ID NO: 23 or SEQ ID NO: 25 without the signal peptide.

57. A chimeric hemagglutinin (HA) polypeptide comprising the amino acid sequence of the influenza virus HA ectodomain forth in SEQ ID NO: 21 or SEQ ID NO: 27.

58. A chimeric hemagglutinin (HA) polypeptide comprising the amino acid sequence of the influenza virus HA ectodomain forth in SEQ ID NO: 23 or SEQ ID NO: 25.

59. A chimeric hemagglutinin (HA) polypeptide comprising the amino acid sequence set forth in SEQ ID NO: 44, SEQ ID NO: 46, SEQ ID NO: 48, or SEQ ID NO: 50, or the amino acid sequence set forth in SEQ ID NO: 44, SEQ ID NO: 46, SEQ ID NO: 48, or SEQ ID NO: 50 without the signal peptide.

60. A chimeric hemagglutinin (HA) polypeptide comprising the amino acid sequence of the influenza virus HA ectodomain forth in SEQ ID NO: 44, SEQ ID NO: 46, SEQ ID NO: 48, or SEQ ID NO: 50.

61. The chimeric HA polypeptide of any one of claims 1 to 60, which is isolated.

62. A nucleic acid sequence encoding the chimeric HA polypeptide of any one of claim 1 to 13, 15 to 18, 20 to 29, 59, or 60.

63. A nucleic acid sequence encoding the chimeric HA polypeptide of any one of claim 30 to 55, or 57.

64. A nucleic acid sequence encoding the chimeric HA polypeptide of claim 14, 18, 56 or 58.

65. A nucleic acid sequence comprising the nucleotide sequence set forth in SEQ ID NO: 20 or SEQ ID NO: 26, or a complement thereof.

66. A nucleic acid sequence comprising the nucleotide sequence set forth in SEQ ID NO: 22 or SEQ ID NO: 24, or a complement thereof.

67. A nucleic acid sequence comprising the nucleotide sequence set forth in SEQ ID NO: 43, SEQ ID NO: 45, SEQ ID NO: 47, or SEQ ID NO: 49, or a complement thereof.

68. The nucleic acid sequence of claim 62 or 67, further comprising a nucleotide sequence comprising the 5′ and 3′ non-coding regions of an influenza B virus.

69. The nucleic acid sequence of any one of claims 62 to 68, wherein the nucleic acid sequence is cDNA.

70. The nucleic acid sequence of any one of claims 62 to 69, which is isolated.

71. An expression vector comprising the nucleic acid sequence of any one of claims 62 to 69.

72. A viral vector comprising the nucleic acid sequence of any one of claims 62 to 69.

73. An influenza B virus engineered to express the nucleic acid sequence of claim 62, 67 or 68.

74. An influenza A virus engineered to express the nucleic acid sequence of claim 63 or 65.

75. An influenza virus engineered to express the chimeric HA polypeptide of any one of claim 1 to 13, 15 to 18, 20 to 29, 59 or 60.

76. The influenza virus of claim 75, wherein the influenza virus is an influenza B virus.

77. The influenza virus of claim 76, wherein the chimeric HA polypeptide is encoded by a nucleotide sequence comprising 5′ and 3′ noncoding regions of an influenza B virus.

78. An influenza B virus comprising the chimeric HA polypeptide of any one of claim 1 to 13, 15 to 18, 20 to 29, 59 or 60.

79. An influenza A virus engineered to express the chimeric HA polypeptide of any one of claim 1, 2, 30 to 55, or 57.

80. An influenza A virus comprising the chimeric HA polypeptide of any one of claim 1, 2, 30 to 55, or 57.

81. The influenza virus of claim 74 or 79, wherein the chimeric HA polypeptide is encoded by a nucleotide sequence comprising 5′ and 3′ noncoding regions of an influenza A virus.

82. The influenza virus of any one of claims 73 to 81, which is attenuated.

83. The influenza virus of claim 78 or 80, which is inactivated.

84. A virus-like particle comprising the chimeric HA polypeptide of any one of claims 1 to 60.

85. A subunit vaccine comprising the chimeric HA polypeptide of any one of claims 1 to 61.

86. A split vaccine comprising the chimeric HA polypeptide of any one of claims 1 to 61.

87. An immunogenic composition comprising the chimeric HA polypeptide of any one of claims 1 to 61.

88. An immunogenic composition comprising the influenza virus of any one of claims 73 to 83.

89. An immunogenic composition comprising the virus-like particle of claim 84.

90. The immunogenic composition of any one of claims 87 to 89, comprising an adjuvant.

91. The subunit vaccine of claim 85, comprising an adjuvant.

92. The split vaccine of claim 86, comprising an adjuvant.

93. A method of inducing an immune response against influenza B virus in a subject, comprising administering to the subject the immunogenic composition of any one of claims 87 to 90.

94. A method of inducing an immune response against influenza B virus in a subject, comprising administering to the subject the subunit vaccine of claim 85 or 91.

95. A method of inducing an immune response against influenza B virus in a subject, comprising administering to the subject the split vaccine of claim 86 or 92.

96. A method of inducing an immune response against influenza B virus in a subject comprising
a. administering to the subject a first immunogenic composition comprising a first chimeric HA, which is the chimeric HA of any one of claims 1 to 61; and
b. a certain period of time after the administration of the first immunogenic composition, administering to the subject a second immunogenic composition comprising a second chimeric HA, which is the chimeric HA of any one of claims 1 to 61,
wherein the first and second chimeric HAs are not the same.

97. A method of inducing an immune response against influenza B virus in a subject that has been administered a first immunogenic composition, comprising administering to the subject a second immunogenic composition comprising a second chimeric HA, which is the chimeric HA of any one of claims 1 to 61, wherein the first immunogenic composition comprising a first chimeric HA, which is the chimeric HA of any one of claims 1 to 61, and
wherein the first and second chimeric HAs are not the same.

98. The method of claim 96 or 97, wherein the first and second chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

99. The method of claims 96 to 98, wherein the method further comprises administering to the subject a third immunogenic composition comprising a third chimeric HA, which is the chimeric HA of any one of claims 1 to 61, wherein the first, second, and third chimeric HAs are not the same.

100. The method of claim 99, wherein the first, second, and third chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

101. The method of claim 99 or 100, wherein the third immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the second immunogenic composition.

102. The method of any one of claims 96 to 101, wherein the second immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the first immunogenic composition.

103. A method of inducing an immune response against influenza B virus in a subject, comprising:
a. a first administration of a first immunogenic composition comprising a first chimeric HA polypeptide to the subject, wherein the first chimeric HA polypeptide comprises a first ectodomain of a first influenza B virus comprising:
i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a first subtype;
ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the first subtype;
iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the first subtype; and
iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the first subtype; and
b. a second administration of a second immunogenic composition comprising a second chimeric HA polypeptide to the subject a first period of time after the first administration, wherein the second chimeric HA polypeptide comprises a second ectodomain of a second influenza B virus comprising:
i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a second subtype;
ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the second subtype;
iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the second subtype; and
iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the second subtype; and
c. a third administration of a third immunogenic composition comprising a third chimeric HA polypeptide to the subject a second period of time after the second administration, wherein the third chimeric HA polypeptide comprises a third ectodomain of a third influenza B virus comprising:
i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a third subtype;
ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the third subtype;
iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the third subtype; and
iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the third subtype.

104. The method of claim 103, wherein first chimeric HA polypeptide is administered to the subject as a nucleic acid encoding the polypeptide.

105. The method of claim 103, wherein the first chimeric HA polypeptide is administered to the subject as part of an influenza virus.

106. The method of any one of claims 103 to 105, wherein the first, second and third subtypes are different.

107. The method of claim 106, wherein the subtypes are selected from the group consisting of H4, H5, H6, H7, H8, H9, H10, H11, H12, H13, H14, H15, H16, H17 and H18.

108. The method of claim 106, wherein the subtypes are selected from the group consisting of H5, H8, H11, H12, and H13.

109. The method of any one of claims 103 to 105, wherein the first period of time is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the first administration.

110. The method of any one of claims 103 to 105, wherein the second period of time is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the second administration.

111. The method of any one of claims 93 to 110, wherein the subject is human.

112. An immunogenic composition of any one of claims 87 to 90 for use in a method of inducing an immune response against influenza B virus in a subject, wherein the method comprises administering to the subject said immunogenic composition.

113. A subunit vaccine of claim 85 or 91 for use in a method of inducing an immune response against influenza B virus in a subject, wherein the method comprises administering to the subject said subunit vaccine.

114. A split vaccine of claim 86 or 92 for use in a method of inducing an immune response against influenza B virus in a subject, wherein the method comprises administering to the subject said split vaccine.

115. A first immunogenic composition for use in a method of inducing an immune response against influenza B virus in a subject, wherein the method comprises:
a. administering to the subject said first immunogenic composition, wherein said first immunogenic composition comprises a first chimeric HA, which is the chimeric HA of any one of claims 1 to 61; and
b. a certain period of time after the administration of the first immunogenic composition, administering to the subject a second immunogenic composition comprising a second chimeric HA, which is the chimeric HA of any one of claims 1 to 61,
wherein the first and second chimeric HAs are not the same.

116. A second immunogenic composition for use in a method of inducing an immune response against influenza B virus in a subject that has been administered a first immunogenic composition, wherein the method comprises administering to the subject said second immunogenic composition, wherein said second immunogenic composition comprises a second chimeric HA, which is the chimeric HA of any one of claims 1 to 61, wherein the first immunogenic composition comprising a first chimeric HA, which is the chimeric HA of any one of claims 1 to 61, and wherein the first and second chimeric HAs are not the same.

117. The first immunogenic composition for use of claim 115, wherein the first and second chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

118. The second immunogenic composition for use of claim 116, wherein the first and second chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

119. The first immunogenic composition for use of claim 115 or 117, wherein the method further comprises administering to the subject a third immunogenic composition comprising a third chimeric HA, which is the chimeric HA of any one of claims 1 to 61, wherein the first, second, and third chimeric HAs are not the same.

120. The second immunogenic composition of claim 116 or 118, wherein the method further comprises administering to the subject a third immunogenic composition comprising a third chimeric HA, which is the chimeric HA of any one of claims 1 to 61, wherein the first, second, and third chimeric HAs are not the same.

121. The first immunogenic composition for use of claim 119, wherein the first, second, and third chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

122. The second immunogenic composition for use of claim 120, wherein the first, second, and third chimeric HAs comprise an ectodomain of an influenza B virus comprising the same stem domain but with one, two, three, or all of the following: (i) different 120 loops, (ii) different 150 loops, (iii) different 160 loops, and/or (iv) different 190 helices.

123. The first immunogenic composition for use of claim 119 or 121, wherein the third immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the second immunogenic composition.

124. The second immunogenic composition for use of claim 120 or 122, wherein the third immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the second immunogenic composition.

125. The first immunogenic composition for use of claim 115, 117, 119 or 121, wherein the second immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the first immunogenic composition.

126. The second immunogenic composition for use of claim 116, 118, 120 or 122, wherein the second immunogenic composition is administered 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the administration of the first immunogenic composition.

127. A first immunogenic composition for use in a method of inducing an immune response against influenza B virus in a subject, wherein the method comprises:
a. a first administration of the first immunogenic composition, wherein the first immunogenic composition comprises a first chimeric HA polypeptide to the subject, and wherein the first chimeric HA polypeptide comprises a first ectodomain of a first influenza B virus comprising:
i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a first subtype;
ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the first subtype;
iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the first subtype; and
iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the first subtype; and
b. a second administration of a second immunogenic composition comprising a second chimeric HA polypeptide to the subject a first period of time after the first administration, wherein the second chimeric HA polypeptide comprises a second ectodomain of a second influenza B virus comprising:
i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a second subtype;
ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the second subtype;
iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the second subtype; and
iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the second subtype; and
c. a third administration of a third immunogenic composition comprising a third chimeric HA polypeptide to the subject a second period of time after the second administration, wherein the third chimeric HA polypeptide comprises a third ectodomain of a third influenza B virus comprising:
i. 2, 3, 4, 5, 6, 7, 8, 9, 10, 11, 12, 13, 14, 15, 16, 17, 18, 19, 20 or more amino acid substitutions within the 120 loop of the globular head domain of the influenza B virus HA, wherein the amino acid substitutions substitute 2, 3, 4, 5, 6, 7, 8, 9, 10, 11, 12, 13, 14, 15, 16, 17, 18, 19, 20 or more amino acid residues in the 120 loop of the globular head of the influenza B virus HA with amino acid residues found in a corresponding region of the globular domain of an influenza A virus HA of a third subtype;
ii. 2, 3, 4, 5, 6, 7, 8, 9 or more amino acid substitutions within the 150 loop of the globular head domain of the influenza B virus HA, wherein the amino acid substitutions substitute 2, 3, 4, 5, 6, 7, 8, 9 or more amino acid residues in the 150 loop of the globular head of the influenza B virus HA with amino acid residues found in a corresponding region of the globular domain of an influenza A virus HA of the third subtype;
iii. 2, 3, 4, 5 or more amino acid substitutions within the 160 loop of the globular head domain of the influenza B virus HA, wherein the amino acid substitutions substitute 2, 3, 4, 5 or more amino acid residues in the 160 loop of the globular head of the influenza B virus HA with amino acid residues found in a corresponding region of the globular domain of an influenza A virus HA of the third subtype; and
iv. 2, 3, 4, 5, 6, 7, 8 or more amino acid substitutions within the 190 helix of the globular head domain of the influenza B virus HA, wherein the amino acid substitutions substitute 2, 3, 4, 5, 6, 7, 8 or more amino acid residues in the 190 helix of the globular head of the influenza B virus HA with amino acid residues found in a corresponding region of the globular domain of an influenza A virus HA of the third subtype.

128. The first immunogenic composition for use of claim 127, wherein first chimeric HA polypeptide is administered to the subject as a nucleic acid encoding the polypeptide.

129. The first immunogenic composition for use of claim 127, wherein the first chimeric HA polypeptide is administered to the subject as part of an influenza virus.

130. The first immunogenic composition for use of any one of claims 127 to 129, wherein the first, second and third subtypes are different.

131. The first immunogenic composition for use of claim 130, wherein the subtypes are selected from the group consisting of H4, H5, H6, H7, H8, H9, H10, H11, H12, H13, H14, H15, H16, H17 and H18.

132. The first immunogenic composition for use of claim 130, wherein the subtypes are selected from the group consisting of H5, H8, H11, H12, and H13.

133. The first immunogenic composition for use of any one of claims 127 to 132, wherein the first period of time is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the first administration.

134. The first immunogenic composition for use of any one of claims 127 to 133, wherein the second period of time is 1 week to 9 months, 3 weeks to 8 months, 6 weeks to 12 weeks, 4 weeks to 6 months, 5 weeks to 5 months, 6 weeks to 4 months, 7 weeks to 4 months, 8 weeks to 4 months, 8 weeks to 3 months, 3 months to 6 months, 3 months to 9 months, or 6 months to 9 months after the second administration.

135. The immunogenic composition for use of any one of claims 112 or 115 to 134, or the vaccine for use of 113 or 114, wherein the subject is human.


SEQUENCE LISTING (95 sequences)

SEQ ID NO: 1 - protein, 22 aa, synthetic construct
  source 1..22: note = B/Yamagata/16/88 120 loop
NIRLSTHNVINAERAPGGPYRL

SEQ ID NO: 2 - protein, 22 aa, synthetic construct
  source 1..22: note = Exemplary B/Yamagata/16/88 120 loop substituted with amino acids from A/Vietnam/1203/04
KIQLSTKNVINAEHAPGGPYRL

SEQ ID NO: 3 - protein, 7 aa, synthetic construct
  source 1..7: note = B/Yamagata/16/88 150 loop
NVTSRNG

SEQ ID NO: 4 - protein, 6 aa, synthetic construct
  source 1..6: note = Exemplary B/Yamagata/16/88 150 loop substituted with amino acids from A/Vietnam/1203/04
YQGKSS

SEQ ID NO: 5 - protein, 6 aa, synthetic construct
  source 1..6: note = B/Yamagata/16/88 160 loop
RDNKTA

SEQ ID NO: 6 - protein, 6 aa, synthetic construct
  source 1..6: note = Exemplary B/Yamagata/16/88 160 loop substituted with amino acids from A/Vietnam/1203/04
KKNSTY

SEQ ID NO: 7 - protein, 7 aa, synthetic construct
  source 1..7: note = B/Yamagata/16/88 190 helix
NKNQMKN

SEQ ID NO: 8 - protein, 7 aa, synthetic construct
  source 1..7: note = Exemplary B/Yamagata/16/88 190 helix substituted with amino acids from A/Vietnam/1203/04
NDAAMQT

SEQ ID NO: 9 - protein, 15 aa, synthetic construct
  source 1..15: note = B/Hong Kong/8/73 HA signal peptide
MKAIIVLLMVVTSNA

SEQ ID NO: 10 - protein, 344 aa, synthetic construct
  source 1..344: note = B/Hong Kong/8/73 HA HA1 polypeptide (no signal peptide)
DRICTGITSSNSPHVVKTATQGEVNVTGVIPLTTTPTKSHFANLKGTQTRGKLCPNCLNCTDLDVALGRPKCMGTIPSAKASILHEVKPVTSGCFPIMHDRTKIRQLPNLLRGYENIRLSARNVTNAETAPGGPYIVGTSGSCPNVTNGNGFFATMAWAVPKNKTATNPLTVEVPYICTKGEDQITVWGFHSDDETQMVKLYGDSKPQKFTSSANGVTTHYVSQIGGFPNQAEDEGLPQSGRIVVDYMVQKPGKTGTIAYQRGVLLPQKVWCASGRSKVIKGSLPLIGEADCLHEKYGGLNKSKPYYTGEHAKAIGNCPIWVKTPLKLANGTKYRPPAKLLKER

SEQ ID NO: 11 - protein, 223 aa, synthetic construct
  source 1..223: note = B/Hong Kong/8/73 HA HA2 polypeptide (no stop codon)
GFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 12 - protein, 530 aa, synthetic construct
  source 1..530: note = B/Hong Kong/8/73 HA Ectodomain (excludes signal peptide, transmembrane domain, cytoplasmic tail domain)
DRICTGITSSNSPHVVKTATQGEVNVTGVIPLTTTPTKSHFANLKGTQTRGKLCPNCLNCTDLDVALGRPKCMGTIPSAKASILHEVKPVTSGCFPIMHDRTKIRQLPNLLRGYENIRLSARNVTNAETAPGGPYIVGTSGSCPNVTNGNGFFATMAWAVPKNKTATNPLTVEVPYICTKGEDQITVWGFHSDDETQMVKLYGDSKPQKFTSSANGVTTHYVSQIGGFPNQAEDEGLPQSGRIVVDYMVQKPGKTGTIAYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHT

SEQ ID NO: 13 - protein, 27 aa, synthetic construct
  source 1..27: note = B/Hong Kong/8/73 HA Transmembrane domain
ILLYYSTAASSLAVTLMIAIFIVYMVS

SEQ ID NO: 14 - protein, 10 aa, synthetic construct
  source 1..10: note = B/Hong Kong/8/73 HA Cytoplasmic domain (no stop codon)
RDNVSCSICL

SEQ ID NO: 15 - protein, 42 aa, synthetic construct
  source 1..42: note = B/Hong Kong/8/73 HA Stem domain (encompasses alanines at interface; no signal peptide or stop codon) Piece 1
DRICTGITSSNSPHVVKTATQGEVNVTGVIPLTTTPTKSHFA

SEQ ID NO: 16 - protein, 241 aa, synthetic construct
  source 1..241: note = B/Hong Kong/8/73 HA Stem domain (encompasses alanines at interface; no signal peptide or stop codon) Piece 2
ADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHT

SEQ ID NO: 17 - protein, 247 aa, synthetic construct
  source 1..247: note = B/Hong Kong/8/73 HA Globular head domain (not including alanines)
NLKGTQTRGKLCPNCLNCTDLDVALGRPKCMGTIPSAKASILHEVKPVTSGCFPIMHDRTKIRQLPNLLRGYENIRLSARNVTNAETAPGGPYIVGTSGSCPNVTNGNGFFATMAWAVPKNKTATNPLTVEVPYICTKGEDQITVWGFHSDDETQMVKLYGDSKPQKFTSSANGVTTHYVSQIGGFPNQAEDEGLPQSGRIVVDYMVQKPGKTGTIAYQRGVLLPQKVWCASGRSKVIKGSLPLIGE

SEQ ID NO: 18 - protein, 8 aa, synthetic construct
  source 1..8: note = influenza B/Yamagata/16/88 virus HA 150 loop (including N terminal P)
PNVTSRNG

SEQ ID NO: 19 - protein, 7 aa, synthetic construct
  source 1..7: note = Sequence from influenza A/Vietnam/1203/04(HALo) virus globular head domain (including N terminal P)
PYQGKSS

SEQ ID NO: 20 - DNA, 1862 bp, synthetic construct
  source 1..1862: mol_type = other DNA; note = Nucleic acid sequence encoding H5-4 loop chimeric HA for rescue in PR8
ccgaagttgggggggagcaaaagcaggggaaaataaaaacaaccaaaatgaaggcaaacctactggtcctgttaagtgcacttgcagctgcagatgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggttcataccttcggcaaaagcttcaatactccacgaagtcagacctgttacatccgggtgctttcctataatgcacgacagaacaaaaatcagacagctacccaatcttctcagaggatatgaaaaaatccagttatcaaccaaaaacgttatcaacgcagaacatgcaccaggaggaccctacagacttggaacctcagaatcttgcccataccagggaaagtcctccttcttcgcaacaatggcttgggctgtcccaaaaaagaacagtacatacacgaatccactaacagtagaagtaccatacatttgcacaaaaggagaagaccaaattactgtttgggggttccattctgataatgatgcggcaatgcagacactctatggagactcaaatcctcaaaagttcacctcatctgccaatggagtaaccacacattatgtttctcagattggtgacttcccaaatcaaacagaagacggagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataattatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctactaataacccggcggcc

SEQ ID NO: 21 - protein, 584 aa, synthetic construct
  source 1..584: note = Amino acid sequence of H5-4 loop chimeric HA for rescue in PR8
MKANLLVLLSALAAADADRICTGITSSNSPHVVKTATQGEVNVTGVIPLTTTPTKSHFANLKGTKTRGKLCPNCLNCTDLDVALGRPMCMGFIPSAKASILHEVRPVTSGCFPIMHDRTKIRQLPNLLRGYEKIQLSTKNVINAEHAPGGPYRLGTSESCPYQGKSSFFATMAWAVPKKNSTYTNPLTVEVPYICTKGEDQITVWGFHSDNDAAMQTLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLLVSLGAISFWMCSNGSLQCRICI

SEQ ID NO: 22 - DNA, 1868 bp, synthetic construct
  source 1..1868: mol_type = other DNA; note = Nucleic acid sequence encoding H8-4 loop chimeric HA for rescue in PR8
ccgaagttgggggggagcaaaagcaggggaaaataaaaacaaccaaaatgaaggcaaacctactggtcctgttaagtgcacttgcagctgcagatgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggcacataccttcggcaaaagcttcaatactccacgaagtcagacctgttacatccgggtgctttcctataatgcacgacagaacaaaaatcagacagctacccaatcttctcagaggatatgaaaggatcagattatcaacctacaacgttatcaacgcagaaaccgcaccaggaggaccctacagacttggaacctcaaaatcttgcaatgcttcaacaggaggccaatctttcttcgcaacaatggcttgggctgtcccaaaaaagaaacccgacacctatacgaatccactaacagtagaagtaccatacatttgcacaaaaggagaagaccaaattactgtttgggggttccattctgatccggatgcaaaaatgcaaacactctatggagactcaaatcctcaaaagttcacctcatctgccaatggagtaaccacacattatgtttctcagattggtgacttcccaaatcaaacagaagacggagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataattatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctactaataacccggcggcc

SEQ ID NO: 23 - protein, 586 aa, synthetic construct
  source 1..586: note = Amino acid sequence of H8-4 loop chimeric HA for rescue in PR8
MKANLLVLLSALAAADADRICTGITSSNSPHVVKTATQGEVNVTGVIPLTTTPTKSHFANLKGTKTRGKLCPNCLNCTDLDVALGRPMCMGHIPSAKASILHEVRPVTSGCFPIMHDRTKIRQLPNLLRGYERIRLSTYNVINAETAPGGPYRLGTSKSCNASTGGQSFFATMAWAVPKKKPDTYTNPLTVEVPYICTKGEDQITVWGFHSDPDAKMQTLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLLVSLGAISFWMCSNGSLQCRICI

SEQ ID NO: 24 - DNA, 1862 bp, synthetic construct
  source 1..1862: mol_type = other DNA; note = Nucleic acid sequence encoding H11-4 loop chimeric HA for rescue in PR8
ccgaagttgggggggagcaaaagcaggggaaaataaaaacaaccaaaatgaaggcaaacctactggtcctgttaagtgcacttgcagctgcagatgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggctaataccttcggcaaaagcttcaatactccacgaagtcagacctgttacatccgggtgctttcctataatgcacgacagaacaaaaatcagacagctacccaatcttctcagaggatatgaaaagatcgaattatcaacctcaaacgttatcaacgcagaagtggcaccaggaggaccctacagacttggaacctcaaaatcttgcaaattcggaagctccaattctttcttcgcaacaatggcttgggctgtcccacatcaatcaggaacatatacgaatccactaacagtagaagtaccatacatttgcacaaaaggagaagaccaaattactgtttgggggttccattctgatgccacactgaaaatgcaccaactctatggagactcaaatcctcaaaagttcacctcatctgccaatggagtaaccacacattatgtttctcagattggtgacttcccaaatcaaacagaagacggagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataattatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctactaataacccggcggcc

SEQ ID NO: 25 - protein, 584 aa, synthetic construct
  source 1..584: note = Amino acid sequence of H11-4 loop chimeric HA for rescue in PR8
MKANLLVLLSALAAADADRICTGITSSNSPHVVKTATQGEVNVTGVIPLTTTPTKSHFANLKGTKTRGKLCPNCLNCTDLDVALGRPMCMGLIPSAKASILHEVRPVTSGCFPIMHDRTKIRQLPNLLRGYEKIELSTSNVINAEVAPGGPYRLGTSKSCKFGSSNSFFATMAWAVPHQSGTYTNPLTVEVPYICTKGEDQITVWGFHSDATLKMHQLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLLVSLGAISFWMCSNGSLQCRICI

SEQ ID NO: 26 - DNA, 1865 bp, synthetic construct
  source 1..1865: mol_type = other DNA; note = Nucleic acid sequence encoding H12-4 loop chimeric HA for rescue in PR8
ccgaagttgggggggagcaaaagcaggggaaaataaaaacaaccaaaatgaaggcaaacctactggtcctgttaagtgcacttgcagctgcagatgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggtatataccttcggcaaaagcttcaatactccacgaagtcagacctgttacatccgggtgctttcctataatgcacgacagaacaaaaatcagacagctacccaatcttctcagaggatatgaaagaatcaagttatcaaccttcaacgttatcaacgcagaaacggcaccaggaggaccctacagacttggaacctcaaaatcttgcaacaatacatcaaaccaaggctcattcttcgcaacaatggcttgggctgtcccattaaaatcaggacaattcacgaatccactaacagtagaagtaccatacatttgcacaaaaggagaagaccaaattactgtttgggggttccattctgatccaacatctgatatgcagatactctatggagactcaaatcctcaaaagttcacctcatctgccaatggagtaaccacacattatgtttctcagattggtgacttcccaaatcaaacagaagacggagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataattatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctactaataacccggcggcc

SEQ ID NO: 27 - protein, 585 aa, synthetic construct
  source 1..585: note = Amino acid sequence of H12-4 loop chimeric HA for rescue in PR8
MKANLLVLLSALAAADADRICTGITSSNSPHVVKTATQGEVNVTGVIPLTTTPTKSHFANLKGTKTRGKLCPNCLNCTDLDVALGRPMCMGYIPSAKASILHEVRPVTSGCFPIMHDRTKIRQLPNLLRGYERIKLSTFNVINAETAPGGPYRLGTSKSCNNTSNQGSFFATMAWAVPLKSGQFTNPLTVEVPYICTKGEDQITVWGFHSDPTSDMQILYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLLVSLGAISFWMCSNGSLQCRICI

SEQ ID NO: 28 - protein, 582 aa, synthetic construct
  source 1..582: note = Influenza B/Hong Kong/8/73 virus HA
MKAIIVLLMVVTSNADRICTGITSSNSPHVVKTATQGEVNVTGVIPLTTTPTKSHFANLKGTQTRGKLCPNCLNCTDLDVALGRPKCMGTIPSAKASILHEVKPVTSGCFPIMHDRTKIRQLPNLLRGYENIRLSARNVTNAETAPGGPYIVGTSGSCPNVTNGNGFFATMAWAVPKNKTATNPLTVEVPYICTKGEDQITVWGFHSDDETQMVKLYGDSKPQKFTSSANGVTTHYVSQIGGFPNQAEDEGLPQSGRIVVDYMVQKPGKTGTIAYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 29 - protein, 565 aa, synthetic construct
  source 1..565: note = Influenza A/Puerto Rico/8/34 virus HA
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 30 - protein, 583 aa, synthetic construct
  source 1..583: note = Influenza B/Yamagata/16/88 virus HA
MKAIIVLLMVVTSNADRICTGITSSNSPHVVKTATQGEVNVTGVIPLTTTPTKSHFANLKGTKTRGKLCPNCLNCTDLDVALGRPMCMGTIPSAKASILHEVRPVTSGCFPIMHDRTKIRQLPNLLRGYENIRLSTHNVINAERAPGGPYRLGTSESCPNVTSRNGFFATMAWAVPRDNKTATNPLTVEVPYICTKGEDQITVWGFHSDNKNQMKNLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 31 - DNA, 1742 bp, synthetic construct
  source 1..1742: mol_type = other DNA; note = Nucleic acid sequence encoding influenza A/mallard/Sweden/24/2002 virus HA
gcaaaagcaggggtcacaatggagaagttcatcataatggcaatgctcttggtgagcacaaatgcatatgataggatatgcattggataccaatcaaacaactccactgacacagtaaacacactcatagagcaaaatgtgccagtcacccagacaatggaactagtggaaaccgagaaacatcctgcctattgcaatactgatttgggtgctccactagagttgcgtgattgcaagattgaggcggtgatctatgggaacccaaaatgtgacatccacttgaaagaccaaggttggtcatacatagtggagagacccagtgcaccagaggggatgtgctaccctggatcagtggagaatctcgaagagttgaggtttgttttctccagtgctgcttcctacaaaaggataagattgtttgattactccagatggaatgttaccagatctgggacaagcaaggcatgcaatgcttcaacaggaggccaatctttctatcggagcatcaattggctaactaaaaagaaacccgacacctatgacttcaatgaggggacttatatcaataatgaagatggggacataattttcttatgggggattcatcacccaccggatgcaaaagagcaaacaacactgtacaagaatgcaaatactttgagtagtgttactaccaacactataaacaggagcttccaacccaatatcgggcccaggccactggtgagagggcaacaagggcggatggactattattggggcattttgaagaggggagaaactttaaaaattagaaccaatgggaacctgatcgcgcctgaattcggttatctgctcaaaggggagagccatggcagaataattcaaaatgaggacataccaattgggagttgtcacacgaaatgtcagacatacgcaggagcaattaatagcagcaaaccctttcagaatrcaagcaggcactacatgggagaatgtcccaagtatgtgaagaaggaaagcttacggctggcagttggacttagaaacactccttctattgaacccagaggcttgtttggagccattgctggttttattgaagggggatggtctgggatgattgatggatggtatgggtttcaccatagtaattcagaaggaacaggaatggcggccgatcagaaatcgacacaagaagcaattgacaaaatcaccaataaggtcaataatatagttgataaaatgaacagggaatttgaagtagtgaaccatgagttttccgaagtcgaaaaaagaataaacatgataaatgacaaaattgatgaccagattgaagatctttgggcctacaatgcagagcttctggtcctcctagagaaccagaaaacactggatgaacacgactccaatgtcaaaaatctctttgatgaagtaagaaggagactttcagccaatgcaatagatactgggaatggctgcttcgacatactccacaaatgtgacaacgagtgcatggagaccataaagaatgggacttataaccataaagagtatgaggaagaggccaagctggaaaggagcaagattaacggagtaaagctagaggaaaataccacctataaaattctcagcatttacagtacggtggcggccagtctttgcttggcaatcctgattgctggaggtctaatcctgggcatgcaaaatggatcttgtagatgcatgttctgtatttaagaaaaaacacccttgtttctact

SEQ ID NO: 32 - protein, 566 aa, synthetic construct
  VARIANT 312: note = X can be any naturally occurring amino acid
  source 1..566: note = Amino acid sequence of influenza A/mallard/Sweden/24/2002 virus HA
MEKFIIMAMLLVSTNAYDRICIGYQSNNSTDTVNTLIEQNVPVTQTMELVETEKHPAYCNTDLGAPLELRDCKIEAVIYGNPKCDIHLKDQGWSYIVERPSAPEGMCYPGSVENLEELRFVFSSAASYKRIRLFDYSRWNVTRSGTSKACNASTGGQSFYRSINWLTKKKPDTYDFNEGTYINNEDGDIIFLWGIHHPPDAKEQTTLYKNANTLSSVTTNTINRSFQPNIGPRPLVRGQQGRMDYYWGILKRGETLKIRTNGNLIAPEFGYLLKGESHGRIIQNEDIPIGSCHTKCQTYAGAINSSKPFQNXSRHYMGECPKYVKKESLRLAVGLRNTPSIEPRGLFGAIAGFIEGGWSGMIDGWYGFHHSNSEGTGMAADQKSTQEAIDKITNKVNNIVDKMNREFEVVNHEFSEVEKRINMINDKIDDQIEDLWAYNAELLVLLENQKTLDEHDSNVKNLFDEVRRRLSANAIDTGNGCFDILHKCDNECMETIKNGTYNHKEYEEEAKLERSKINGVKLEENTTYKILSIYSTVAASLCLAILIAGGLILGMQNGSCRCMFCI

SEQ ID NO: 33 - DNA, 1840 bp, synthetic construct
  source 1..1840: mol_type = other DNA; note = DNA sequence encoding influenza A/Vietnam/1203/04 (HALo) virus HA
cggatgttgcccagccggcgccagcgaggaggctgggaccatgccggccagcaaaagcaggggttcaatctgtcaaaatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcagcgggagacgcggggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttaaatttgtgagttcagattgtagttaaaaacacccttgtttctactaataacccggcggcccaaaatgcc

SEQ ID NO: 34 - protein, 585 aa, synthetic construct
  source 1..585: note = aa sequence of influenza A/Vietnam/1203/04 (HALo) virus HA
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICIICEFRLLKTPLFLLITRRPKM

SEQ ID NO: 35 - DNA, 1745 bp, synthetic construct
  source 1..1745: mol_type = other DNA; note = sequence encoding influenza A/northern shoveler/Netherlands/18/99 virus HA
aaagcaggggttctattaagaaatcaaaatgaagaaagcactgctatttgcagctattttcctttatgcaaaggcagatgaaatctgtatcggctacttaagcaacaactcaacagacaaagttgacacaataatcgagaacaatgtcacagtgactagctcagtggaactagtcgaaacagaacacactgggtcattctgctcaatcaatggaaaacaaccgataagtcttggagactgctcttttgctgggtggatattaggtaatcccatgtgtgatgatctaattggtaagacttcatggtcctacattgtggagaagcccaacccaacaaatggaatttgttaccctggaactttggaggatgaagaagaactgagactgaaattcagtggagtcctggaattcagcaagttcgaagcattcacatcaaatggatggggtgctgtgaattcgggagcaggagtgactgctgcatgcaaattcggaagctccaattctttcttccgaaacatggtatggcttatacatcaatcaggaacatatcctgtaataaagaggacttttaacaataccaaaggaagagatgtgttgatcgtttggggaattcatcaccctgccacactgaaagagcaccaagacctatacaagaaagacagttcctatgtagcagtgggttcagaaacttacaacagaagattcaccccagaaatcagcactaggcccaaagtcaatggacaggctggacggatgacattttattggacaatggtcaaaccaggagagtcaataacatttgagtctaatggtgcattcttggctcctagatatgcttttgagattgtctctgttggaaatgggaaattattcagaagcgaactgagtattgaatcatgctctaccaaatgccaaacagaagtaggagggattaatacaaacaaaagtttccacagtgttcataggaacactattggagactgtcccaaatatgtgaatgtcaaatccctaaagcttgccacaggacttagaaatgttccagcaatagcatcaagaggattgtttggagcaatagccggatttatagaaggtgggtggccagggcttatcaatggatggtacggttttcaacacagaaatgaggaaggaacaggcatagcagcagacagggagtcaactcaaaaggcagtagaccagataacatccaaagtaaacaacatcgtcgacagaatgaatacaaatttcgagtctgtgcaacacgaattcagtgaaatagaggagaggataaaccaattgtcgaaacacgtagatgattctgtggttgacatctggtcatataacgcacaactcctcgtttggcttgaaaatgagaaaactctggatctccacgattccaatgttaggaatcttcatgagaaagtcagaaggatgctaaaggacaatgccaaagatgagggaaatggatgcttcaccttttaccacaagtgtgacaacgaatgcatcgaaaaggttagaaacggaacatatgatcacaaagaattcgaagaagaatcaagaatcaatcgccaggagattgagggagtgagattagattctagtgggaatgtgtataaaatactgtcaatttacagctgcatcgcaagcagtcttgtattagcagcactcatcatgggtttcatcctatgggcgtgcagcaatggatcatgtagatgtaccatttgcatttagaattgtggcaaaaacaccc

SEQ ID NO: 36 - protein, 565 aa, synthetic construct
  source 1..565: note = sequence of influenza A/northern shoveler/Netherlands/18/99 virus HA
MKKALLFAAIFLYAKADEICIGYLSNNSTDKVDTIIENNVTVTSSVELVETEHTGSFCSINGKQPISLGDCSFAGWILGNPMCDDLIGKTSWSYIVEKPNPTNGICYPGTLEDEEELRLKFSGVLEFSKFEAFTSNGWGAVNSGAGVTAACKFGSSNSFFRNMVWLIHQSGTYPVIKRTFNNTKGRDVLIVWGIHHPATLKEHQDLYKKDSSYVAVGSETYNRRFTPEISTRPKVNGQAGRMTFYWTMVKPGESITFESNGAFLAPRYAFEIVSVGNGKLFRSELSIESCSTKCQTEVGGINTNKSFHSVHRNTIGDCPKYVNVKSLKLATGLRNVPAIASRGLFGAIAGFIEGGWPGLINGWYGFQHRNEEGTGIAADRESTQKAVDQITSKVNNIVDRMNTNFESVQHEFSEIEERINQLSKHVDDSVVDIWSYNAQLLVWLENEKTLDLHDSNVRNLHEKVRRMLKDNAKDEGNGCFTFYHKCDNECIEKVRNGTYDHKEFEEESRINRQEIEGVRLDSSGNVYKILSIYSCIASSLVLAALIMGFILWACSNGSCRCTICI

SEQ ID NO: 37 - DNA, 1712 bp, synthetic construct
  source 1..1712: mol_type = other DNA; note = sequence encoding A_mallard_interior Alaska_7MP0167_2007 virus HA
ggtcacaatggaaaaattcatcattttgagcactgtcctggcagtaagctttgcatatgacaaaatttgcattggatatcaaacaaacaactcgactgagacagtaaacacactaatcgagcaaaacgttccggtgacacaggtggaagaactcgtgcatggtgggatcgatccggtcctatgtggaacggagctagggtcaccattagtgcttgatgactgttcactcgaaggtctcattctaggcaatcccaaatgtgatctttatctgaatggcagggaatggtcatacatagtagagaggcccaaagaaatggaaggagtttgctacccaggatcaattgaaaatcaagaggagttgagatctctgttttcttccatcaagaagtatgaaagagtgaagatgtttgatttcaccaaatggaatgtcacgtacactggaaccagcaaggcctgcaacaatacatcaaaccaaggctcattctataggagcatgagatggttaaccttaaaatcaggacaattcccagtccagacagatgagtacaagaacaccagagattcggacattgtcttcacctgggccatccatcacccaccaacatctgatgaacagataaagttatacaaaaatccggataccctctcctcagtcaccactgatgaaatcaataggagtttcaagcctaacataggaccaaggccattagtgagaggacaacaggggagaatggattactactgggctgtcctcaaacctggacaaactgtcaaaatacagaccaatggtaatcttattgcacctgaatatggtcacttaattacagggaaatcacatggcaggatactcaagaataacttgcccataggacagtgtgtgactgagtgccagttgaatgaaggggtgatgaatacaagtaaacctttccagaacactagtaagcactatattgggaaatgtcccaaatacataccatcaggaagcctgaaattggcgatagggctcagaaatgttccacaagttcagaacagaggactatttggagcaaaagcaggtttcatagagggcggatggccaggactagtggctggttggtatggatttcagcatcaaaatgcagaggggacaggcatagccgcagacagagacagcactcagaaggcaatagacaatatgcaaaacaaactcaacaatgtcattgacaaaatgaacaaacaatttgaagtggtgaatcatgagttttcagaagtggaaagcagaataaacatgattaattcccaaattgatgatcaaataactgacatatgggcatacaatgctgaactgcttgtcctattggaaaatcagaagacattagatgagcatgatgctaatgtaagaaacttacacgatagagtcagaagagtcctaagagaaaatgcaattgatacaggagatggttgctttgaaattctgcataaatgtgacaacaattgcatggacacaatcagaaatgggacatacaatcacaaggagtatgaggaagaaagcaaaatcgaacgacagaaaattaatggtgtcaaacttgaggagaattctacatataaaattctgagcatctacagcagtgttgcctcaagcttagttctactgctcatgattattgggggtttcattttcgggtgtcaaaatggaaatgttcgttgtactttctgtatttaattaaaaacac

SEQ ID NO: 38 - protein, 564 aa, synthetic construct
  source 1..564: note = sequence of A_mallard_interior Alaska_7MP0167_2007 virus HA
MEKFIILSTVLAVSFAYDKICIGYQTNNSTETVNTLIEQNVPVTQVEELVHGGIDPVLCGTELGSPLVLDDCSLEGLILGNPKCDLYLNGREWSYIVERPKEMEGVCYPGSIENQEELRSLFSSIKKYERVKMFDFTKWNVTYTGTSKACNNTSNQGSFYRSMRWLTLKSGQFPVQTDEYKNTRDSDIVFTWAIHHPPTSDEQIKLYKNPDTLSSVTTDEINRSFKPNIGPRPLVRGQQGRMDYYWAVLKPGQTVKIQTNGNLIAPEYGHLITGKSHGRILKNNLPIGQCVTECQLNEGVMNTSKPFQNTSKHYIGKCPKYIPSGSLKLAIGLRNVPQVQNRGLFGAKAGFIEGGWPGLVAGWYGFQHQNAEGTGIAADRDSTQKAIDNMQNKLNNVIDKMNKQFEVVNHEFSEVESRINMINSQIDDQITDIWAYNAELLVLLENQKTLDEHDANVRNLHDRVRRVLRENAIDTGDGCFEILHKCDNNCMDTIRNGTYNHKEYEEESKIERQKINGVKLEENSTYKILSIYSSVASSLVLLLMIIGGFIFGCQNGNVRCTFCI

SEQ ID NO: 39 - DNA, 1775 bp, synthetic construct
  source 1..1775: mol_type = other DNA; note = sequence encoding influenza A/Puerto Rico/8/34 virus HA
agcaaaagcaggggaaaataaaaacaaccaaaatgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 40 - protein, 560 aa, synthetic construct
  source 1..560: note = sequence of influenza A/Puerto Rico/8/34 virus HA
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQ

SEQ ID NO: 41 - DNA, 1879 bp, synthetic construct
  source 1..1879: mol_type = other DNA; note = sequence encoding influenza B/Yamagata/16/88 virus HA
agcagaagcagagcattttctaatatccacaaaatgaaggcaataattgtactactcatggtagtaacatccaacgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggaccataccttcggcaaaagcttcaatactccacgaagtcagacctgttacatccgggtgctttcctataatgcacgacagaacaaaaatcagacagctacccaatcttctcagaggatatgaaaatatcagattatcaacccataacgttatcaacgcagaaagggcaccaggaggaccctacagacttggaacctcagaatcttgccctaacgttaccagtagaaacggattcttcgcaacaatggcttgggctgtcccaagggacaacaaaacagcaacgaatccactaacagtagaagtaccatacatttgcacaaaaggagaagaccaaattactgtttgggggttccattctgataacaaaaaccaaatgaaaaacctctatggagactcaaatcctcaaaagttcacctcatctgccaatggagtaaccacacattatgtttctcagattggtgacttcccaaatcaaacagaagacggagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataatcatactatactgctctactactcaactgctgcttctagtttggccgtaacattgatgatagctatttttattgtttatatggtctccagagacaatgtttcttgctccatctgtctataaggaaaattaagccctgtattttcctttattgtagtgcttgtttgcttgttaccattacaaagaaacgttattgaaaaatgctcttgttactact

SEQ ID NO: 42 - protein, 583 aa, synthetic construct; identical to SEQ ID NO: 30
  source 1..583: note = sequence of influenza B/Yamagata/16/88 virus HA

SEQ ID NO: 43 - DNA, 1876 bp, synthetic construct
  source 1..1876: mol_type = other DNA; note = sequence encoding mH5/B chimeric HA for rescue in B/Yamagata/16/88
agcagaagcagagcattttctaatatccacaaaatgaaggcaataattgtactactcatggtagtaacatccaacgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggttcataccttcggcaaaagcttcaatactccacgaagtcagacctgttacatccgggtgctttcctataatgcacgacagaacaaaaatcagacagctacccaatcttctcagaggatatgaaaaaatccagttatcaaccaaaaacgttatcaacgcagaacatgcaccaggaggaccctacagacttggaacctcaaaatcttgcccataccagggaaagtcctccttcttcgcaacaatggcttgggctgtcccaaaaaagaacagtacatacacgaatccactaacagtagaagtaccatacatttgcacaaaaggagaagaccaaattactgtttgggggttccattctgataatgatgcggcaatgcagacactctatggagactcaaatcctcaaaagttcacctcatctgccaatggagtaaccacacattatgtttctcagattggtgacttcccaaatcaaacagaagacggagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataatcatactatactgctctactactcaactgctgcttctagtttggccgtaacattgatgatagctatttttattgtttatatggtctccagagacaatgtttcttgctccatctgtctataaggaaaattaagccctgtattttcctttattgtagtgcttgtttgcttgttaccattacaaagaaacgttattgaaaaatgctcttgttactact

SEQ ID NO: 44 - protein, 582 aa, synthetic construct
  source 1..582: note = sequence of mH5/B chimeric HA for rescue in B/Yamagata/16/88
MKAIIVLLMVVTSNADRICTGITSSNSPHVVKTATQGEVNVTGVIPLTTTPTKSHFANLKGTKTRGKLCPNCLNCTDLDVALGRPMCMGFIPSAKASILHEVRPVTSGCFPIMHDRTKIRQLPNLLRGYEKIQLSTKNVINAEHAPGGPYRLGTSKSCPYQGKSSFFATMAWAVPKKNSTYTNPLTVEVPYICTKGEDQITVWGFHSDNDAAMQTLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 45 - DNA, 1882 bp, synthetic construct
  source 1..1882: mol_type = other DNA; note = sequence encoding mH8/B chimeric HA for rescue in B/Yamagata/16/88
agcagaagcagagcattttctaatatccacaaaatgaaggcaataattgtactactcatggtagtaacatccaacgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggcacataccttcggcaaaagcttcaatactccacgaagtcagacctgttacatccgggtgctttcctataatgcacgacagaacaaaaatcagacagctacccaatcttctcagaggatatgaaaggatcagattatcaacctacaacgttatcaacgcagaaaccgcaccaggaggaccctacagacttggaacctcaaaatcttgcaatgcttcaacaggaggccaatctttcttcgcaacaatggcttgggctgtcccaaaaaagaaagcagacacctatacgaatccactaacagtagaagtaccatacatttgcacaaaaggagaagaccaaattactgtttgggggttccattctgatgcagatgcaaaaatgcaaacactctatggagactcaaatcctcaaaagttcacctcatctgccaatggagtaaccacacattatgtttctcagattggtgacttcccaaatcaaacagaagacggagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataatcatactatactgctctactactcaactgctgcttctagtttggccgtaacattgatgatagctatttttattgtttatatggtctccagagacaatgtttcttgctccatctgtctataaggaaaattaagccctgtattttcctttattgtagtgcttgtttgcttgttaccattacaaagaaacgttattgaaaaatgctcttgttactact

SEQ ID NO: 46 - protein, 584 aa, synthetic construct
  source 1..584: note = sequence of mH8/B chimeric HA for rescue in B/Yamagata/16/88
MKAIIVLLMVVTSNADRICTGITSSNSPHVVKTATQGEVNVTGVIPLTTTPTKSHFANLKGTKTRGKLCPNCLNCTDLDVALGRPMCMGHIPSAKASILHEVRPVTSGCFPIMHDRTKIRQLPNLLRGYERIRLSTYNVINAETAPGGPYRLGTSKSCNASTGGQSFFATMAWAVPKKKADTYTNPLTVEVPYICTKGEDQITVWGFHSDADAKMQTLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 47 - DNA, 1972 bp, synthetic construct
  source 1..1972: mol_type = other DNA; note = sequence encoding mH11/B chimeric HA for rescue in B/Yamagata/16/88
agcagaagcagagcattttctaatatccacaaaatgaaggcaataattgtactactcatggtagtaacatccaacgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggctaataccttcggcaaaagcttcaatactccacgaagtcagacctgttacatccgggtgctttcctataatgcacgacagaacaaaaatcagacagctacccaatcttctcagaggatatgaaaagatcgaattatcaacctcaaacgttatcaacgcagaagtggcaccaggaggaccctacagacttggaacctcagaatcttgccctttcggaagctccaattctttcttcgcaacaatggcttgggctgtcccacatcaatcaggaacatatacgaatccactaacagtagaagtaccatacatttgcacaaaaggagaagaccaaattactgtttgggggttccattctgataccacactgaaaatgcaccaactctatggagactcaaatcctcaaaagttcacctcatctgccaatggagtaaccacacattatgtttctcagattggtgacttcccaaatcaaacagaagacgaagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataatcatactatactgctctactactcaactgctgcttctagtttggccgtaacattgatgatagctatttttattgtttatatggtctccagagacaatgtttcttgctccatctgtctataaggaaaattaagccctgtattttcctttattgtagtgcttgtttgcttgttaccattacaaagaaacgttattgaaaaatgctcttgtttactactaggaaaattaagccctgtattttcctttattgtagtgcttgtttgcttgttaccattacaaagaaacgttattgaaaaatgctcttgttactact

SEQ ID NO: 48 - protein, 582 aa, synthetic construct
  source 1..582: note = sequence of mH11/B chimeric HA for rescue in B/Yamagata/16/88
MKAIIVLLMVVTSNADRICTGITSSNSPHVVKTATQGEVNVTGVIPLTTTPTKSHFANLKGTKTRGKLCPNCLNCTDLDVALGRPMCMGLIPSAKASILHEVRPVTSGCFPIMHDRTKIRQLPNLLRGYEKIELSTSNVINAEVAPGGPYRLGTSESCPFGSSNSFFATMAWAVPHQSGTYTNPLTVEVPYICTKGEDQITVWGFHSDTTLKMHQLYGDSNPQKFTSSANGVTTHYVSQIGDFPNQTEDEGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 49 - DNA, 1870 bp, synthetic construct
  source 1..1870: mol_type = other DNA; note = sequence encoding mH13/B chimeric HA for rescue in B/Yamagata/16/88
agcagaagcagagcattttctaatatccacaaaatgaaggcaataattgtactactcatggtagtaacatccaacgcagatcgaatctgcactgggataacatcttcaaactcacctcatgtggtcaaaacagctactcaaggggaagttaatgtgactggtgtgataccactgacaacaacaccaacaaaatctcattttgcaaatctcaaaggaacaaagaccagagggaaactatgcccaaactgtctcaactgcacagatctggatgtggccttgggcagaccaatgtgtatggggaacataccttcggcaaaagcttcaatactccacgaagtcagacctgttacatccgggtgctttcctataatgcacgacagaacaaaaatcagacagctacccaatcttctcagaggatatgaaagaatcgagttatcaacccataacgttatcaacgcagaagtggcaccaggaggaccctacagacttggaacctcaaaatcttgcccagataaaggagccagcagcttcttcgcaacaatggcttgggctgtcccaaagagaggaaatcagtatacgaatccactaacagtagaagtaccatacatttgcacaaaaggagaagaccaaattactgtttgggggttccattctgatgtttccacaaacatggcgaaactctatggagactcaaatcctcaaaagttcacctcatctgccaatggagtaaccacacattatgtttctcagattggtgacttcccaaatcaaacagaagacggagggctaccacaaagcggcagaattgttgttgattacatggtgcaaaaacctgggaaaacaggaacaattgtctatcaaagaggtgttttgttgcctcaaaaggtgtggtgcgcaagtggcaggagcaaggtaataaaagggtccttgcctttaattggtgaagcagattgccttcacgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcttagagggaggatgggaaggaatgattgcaggttggcacggatacacatctcatggagcacatggagtggcagtggcagcagaccttaagagcacgcaagaagccataaacaagataacaaaaaatctcaattctttgagtgagctagaagtaaagaatcttcaaagactaagtggtgccatggatgaactccacaacgaaatactcgagctggatgagaaagtggatgatctcagagctgacacaataagctcgcaaatagagcttgcagtcttgctttccaacgaaggaataataaacagtgaagatgagcatctattggcacttgagagaaaactaaagaaaatgctgggtccctctgctgtagacatagggaatggatgcttcgaaaccaaacacaagtgcaaccagacctgcttagacaggatagctgctggcacctttaatgcaggagaattttctcttcccacttttgattcactgaatattactgctgcatctttaaatgatgatggattggataatcatactatactgctctactactcaactgctgcttctagtttggccgtaacattgatgatagctatttttattgtttatatggtctccagagacaatgtttcttgctccatctgtctataaggaaaattaagccctgtattttcctttattgtagtgcttgtttgcttgttaccattacaaagaaacgttattgaaaaatgctcttgtt

SEQ ID NO: 50 - protein, 582 aa, synthetic construct
  source 1..582: note = sequence of mH13/B chimeric HA for rescue in amino acids from B/Yamagata/16/88
MKAIIVLLMVVTSNADRICTGITSSNSPHVVKTATQGEVNVTGVIPLTTTPTKSHFANLKGTKTRGKLCPNCLNCTDLDVALGRPMCMGNIPSAKASILHEVRPVTSGCFPIMHDRTKIRQLPNLLRGYERIELSTHNVINAEVAPGGPYRLGTSKSCPDKGASSFFATMAWAVPKRGNQYTNPLTVEVPYICTKGEDQITVWGFHSDVSTNMAKLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 51 - protein, 22 aa, synthetic construct
  source 1..22: note = Exemplary B/Yamagata/16/88 120 loop substituted with amino acids from A/Mallard/Sweden/24/2002 virus (H8)
RIRLSTYNVINAETAPGGPYRL

SEQ ID NO: 52 - protein, 8 aa, synthetic construct
  source 1..8: note = Exemplary B/Yamagata/16/88 150 loop substituted with amino acids from A/Mallard/Sweden/24/2002 virus (H8)
NASTGGQS

SEQ ID NO: 53 - protein, 7 aa, synthetic construct
  source 1..7: note = Exemplary B/Yamagata/16/88 160 loop substituted with amino acids from A/Mallard/Sweden/24/2002 virus (H8)
KKKADTY

SEQ ID NO: 54 - protein, 7 aa, synthetic construct
  source 1..7: note = Exemplary B/Yamagata/16/88 190 helix substituted with amino acids from A/Mallard/Sweden/24/2002 virus (H8)
ADAKMQT

SEQ ID NO: 55 - protein, 22 aa, synthetic construct
  source 1..22: note = Exemplary B/Yamagata/16/88 120 loop substituted with amino acids from A/northern shoveler/Netherlands/18/99
KIELSTSNVINAEVAPGGPYRL

SEQ ID NO: 56 - protein, 7 aa, synthetic construct
  source 1..7: note = Exemplary B/Yamagata/16/88 150 loop substituted with amino acids from A/northern shoveler/Netherlands/18/99
PFGSSNS

SEQ ID NO: 57 - protein, 6 aa, synthetic construct
  source 1..6: note = Exemplary B/Yamagata/16/88 160 loop substituted with amino acids from A/northern shoveler/Netherlands/18/99
HQSGTY

SEQ ID NO: 58 - protein, 7 aa, synthetic construct
  source 1..7: note = Exemplary B/Yamagata/16/88 190 helix substituted with amino acids from A/northern shoveler/Netherlands/18/99
TTLKMHQ

SEQ ID NO: 59 - protein, 22 aa, synthetic construct
  source 1..22: note = Exemplary B/Yamagata/16/88 120 loop substituted with amino acids from A/black headed gull/Sweden/1/99
RIELSTHNVINAEVAPGGPYRL

SEQ ID NO: 60 - protein, 7 aa, synthetic construct
  source 1..7: note = Exemplary B/Yamagata/16/88 150 loop substituted with amino acids from A/black headed gull/Sweden/1/99
PDKGASS

SEQ ID NO: 61 - protein, 6 aa, synthetic construct
  source 1..6: note = Exemplary B/Yamagata/16/88 160 loop substituted with amino acids from A/black headed gull/Sweden/1/99
KRGNQY

SEQ ID NO: 62 - protein, 7 aa, synthetic construct
  source 1..7: note = Exemplary B/Yamagata/16/88 190 helix substituted with amino acids from A/black headed gull/Sweden/1/99
VSTNMAK

SEQ ID NO: 63 - protein, 22 aa, synthetic construct
  source 1..22: note = Exemplary B/Yamagata/16/88 120 loop substituted with amino acids from A/mallard/interior Alaska/7MP0167/2007
RIKLSTFNVINAETAPGGPYRL

SEQ ID NO: 64 - protein, 8 aa, synthetic construct
  source 1..8: note = Exemplary B/Yamagata/16/88 150 loop substituted with amino acids from A/mallard/interior Alaska/7MP0167/2007
NNTSNQGS

SEQ ID NO: 65 - protein, 6 aa, synthetic construct
  source 1..6: note = Exemplary B/Yamagata/16/88 160 loop substituted with amino acids from A/mallard/interior Alaska/7MP0167/2007
LKSGQF

SEQ ID NO: 66 - protein, 7 aa, synthetic construct
  source 1..7: note = Exemplary B/Yamagata/16/88 190 helix substituted with amino acids from A/mallard/interior Alaska/7MP0167/2007
PTSDMQI

SEQ ID NO: 67 - protein, 7 aa, synthetic construct
  source 1..7: note = Exemplary B/Yamagata/16/88 150 loop substituted with amino acids from A/northern shoveler/Netherlands/18/99
KFGSSNS

SEQ ID NO: 68 - protein, 7 aa, synthetic construct
  source 1..7: note = Exemplary B/Yamagata/16/88 160 loop substituted with amino acids from A/mallard/Sweden/24/2002
KKKPDTY

SEQ ID NO: 69 - protein, 7 aa, synthetic construct
  source 1..7: note = Exemplary B/Yamagata/16/88 190 helix substituted with amino acids from A/mallard/Sweden/24/2002
PDAKMQT

SEQ ID NO: 70 - protein, 7 aa, synthetic construct
  source 1..7: note = Exemplary B/Yamagata/16/88 190 helix substituted with amino acids from A/northern shoveler/Netherlands/18/99
ATLKMHQ

SEQ ID NO: 71 - DNA, 1765 bp, synthetic construct
  source 1..1765: mol_type = other DNA; note = Nucleic acid sequence encoding influenza A/black headed gull/Sweden/1/99 virus HA
agcaaaagcaggggaaaatttcaacaaactgatacaagagaaatggacatcccagtagtcgcattcttgatattaaccagtacatgcgtacaggctgataggatatgtgttgggtacttaagcaccaactcatcagaaaaggttgacacactgttagaaaacgatgttccggttacaagctctgttgatctggttgagactaaccacacaggaacatattgttctttgggtggaatcagtccggtgcacctgggagactgtagcttcgagggctggattgtagggaaccctgcctgtgccagcaacctggggatcagagaatggtcatacttgattgaagatccttctgctcctcatggattgtgctacccaggggagttagacaacaatggagaattgaggcacttgttcagtggaatcagatctttcagtagaacagagttgattgcacctacttcttggggggcagtgaatgatggagtatcgtcggcctgtccagataaaggagccagcagcttttaccggaacttggtatggtttgtgaagagaggaaatcagtatcctgtaatccgcgggacctacaacaacactactggcagagatgttttggttatatggggtatacatcaccctgtttccacagacgaagcgaaacaactatatgtcaataacaacccatacacgttggtatctaccagttcatggagtaggaaatacaacttagagactggaacccggcctggatacaatggccaaaagagttggatgaagatttactggtatttgatgcacccaggggagtcaatcagtttcgaaagcaatggaggattattggcacccagatatggttatattattgaggaatatggaaaagggcgaattttccaaagccgcattcgaattgctaaatgcaatactaagtgccaaacatctgtcggtgggataaataccaacaaaacatttcaaaacatagagagaaatgcacttggggattgcccgaaatacataaaatctggacagctcaagttagccaccggacttaggaatgtacctgccatatcaaacagagggttgttcggggctattgcaggcttcatagaaggtggttggccaggattaataaatggttggtatggattccaacatcagaacgaacaaggagtgggcatggctgcagacaaagagtcaacacaaaaggctattgatcaaataacaaccaagataaacaatatcattgaaaaaatgaatgggaattatgactccatacgaggtgaattcaatcaggtggaacaaagaataaatatgcttgcagacagaatagatgatgctgtaactgatgtatggtcatacaatgcaaagcttcttgtgttactagagaacgataaaactctagacatgcatgatgctaatgttagaaacctgcatgatcaagtccgtagagcactaaagaccaatgcaattgatgagggaaatggatgtttcgaactcctccataaatgcaatgactcttgcatggagacaataagaaatggaacgtacaatcatacagaatatgaggaagaatccaaattaaagagacaggaaatagaaggaataaagctgaagtcagacgatagtgtttataaagcactatcgatttacagctgcattgcaagcagtattgtattggtaggactcatacttacattcatcatgtgggcatgcagcagtggcaattgccggttcaatatttgtatataagtagaaaaaacacccttgttctact

SEQ ID NO: 72 - protein, 565 aa, synthetic construct
  source 1..565: note = sequence of influenza A/black headed gull/Sweden/1/99 virus HA
MDIPVVAFLILTSTCVQADRICVGYLSTNSSEKVDTLLENDVPVTSSVDLVETNHTGTYCSLGGISPVHLGDCSFEGWIVGNPACASNLGIREWSYLIEDPSAPHGLCYPGELDNNGELRHLFSGIRSFSRTELIAPTSWGAVNDGVSSACPDKGASSFYRNLVWFVKRGNQYPVIRGTYNNTTGRDVLVIWGIHHPVSTDEAKQLYVNNNPYTLVSTSSWSRKYNLETGTRPGYNGQKSWMKIYWYLMHPGESISFESNGGLLAPRYGYIIEEYGKGRIFQSRIRIAKCNTKCQTSVGGINTNKTFQNIERNALGDCPKYIKSGQLKLATGLRNVPAISNRGLFGAIAGFIEGGWPGLINGWYGFQHQNEQGVGMAADKESTQKAIDQITTKINNIIEKMNGNYDSIRGEFNQVEQRINMLADRIDDAVTDVWSYNAKLLVLLENDKTLDMHDANVRNLHDQVRRALKTNAIDEGNGCFELLHKCNDSCMETIRNGTYNHTEYEEESKLKRQEIEGIKLKSDDSVYKALSIYSCIASSIVLVGLILTFIMWACSSGNCRFNICI

SEQ ID NO: 73 - protein, 585 aa, synthetic construct
  VARIANT 212: note = X = Asn or Ser
  VARIANT 214: note = X = Asn, Ile, Thr or Ser
  source 1..585: note = Amino acid sequence of mouse adapted Influenza B/Malaysia/2506/2004 virus HA
MKAIIVLLMVVTSNADRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDXEXQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 74 - protein, 15 aa, synthetic construct
  source 1..15: note = Signal peptide of mouse adapted Influenza B/Malaysia/2506/2004 virus HA
MKAIIVLLMVVTSNA

SEQ ID NO: 75 - protein, 347 aa, synthetic construct
  VARIANT 197: note = X = Asn or Ser
  VARIANT 199: note = X = Asn, Ile, Thr or Ser
  source 1..347: note = HA1 of mouse adapted Influenza B/Malaysia/2506/2004 virus HA
DRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDXEXQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKER

SEQ ID NO: 76 - protein, 223 aa, synthetic construct
  source 1..223: note = HA2 of mouse adapted Influenza B/Malaysia/2506/2004 virus HA
GFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 77 - protein, 250 aa, synthetic construct
  VARIANT 155: note = X = Asn or Ser
  VARIANT 157: note = X = Asn, Ile, Thr or Ser
  source 1..250: note = Head Sequence of mouse adapted Influenza B/Malaysia/2506/2004 virus HA
NLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDXEXQMAKLYGDSKPQKFTSSANGVTTHYVSQIGGFPNQTEDGGLPQSGRIVVDYMVQKSGKTGTITYQRGILLPQKVWCASGRSKVIKGSLPLIGE

SEQ ID NO: 78 - protein, 42 aa, synthetic construct; identical to SEQ ID NO: 15
  source 1..42: note = N-terminal segment of Stalk of mouse adapted Influenza B/Malaysia/2506/2004 virus HA

SEQ ID NO: 79 - protein, 241 aa, synthetic construct
  source 1..241: note = C-terminal segment of Stalk of mouse adapted Influenza B/Malaysia/2506/2004 virus HA
ADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHT

SEQ ID NO: 80 - DNA, 2312 bp, synthetic construct
  source 1..2312: mol_type = other DNA; note = Nucleotide sequence of mouse adapted Influenza B/Malaysia/2506/2004 virus PB1
aatataaatccttatttcctcttcatagatgtgcccatacaggcagcaatttcaacaacattcccatacactggtgttcccccttattcccatggaacgggaacaggctacacaatagacaccgtgatcagaacacatgagtactcaarcaaggggaaacagtacatttctgatgttacaggatgcacaatggtagatccaacaaatggcccattacccgaagataatgagccgagtgcctatgcgcaattagattgcgttttagaggctttggatagaatggatgaagaacacccaggtctgtttcaagcagcctcacagaatgctatggaggccctaatggtcacaactgtagacaaattaacccaggggagacagacttttgattggacagtatgcagaaaccaacctgctgcaacggcactgaacacaacaataacctcttttaggttgaatgatttaaatggagccgacaaaggtggattagtacctttttgccaggatatcattgattcattagacagacctgaaatgactttcttctcagtaaagaatataaagaaaaaattgcctgccaaaaacagaaagggtttcctcataaagaggataccaatgaaggtaaaggacaaaataaccaaagtggaatacatcaaaagagcattatcattaaacacaatgacaaaagacgctgaaagaggcaaactaaaaagaagagcgattgccaccgctggaatacaaatcagagggtttgtattagtagttgaaaacttggctaaaaatatatgtgaaaatctagaacaaagtggtttaccagtaggtggaaacgagaagaaagccaaactgtcaaacgcagtggcaaaaatgctcagtaactgcccacctggagggatcagcatgacagtaacaggagacaatactaaatggaatgaatgtttaaacccaagaatctttttggctatgactgaaagaataaccagagacagcccaatttggttcagggatttttgtagtatagcaccggtcctgttctccaataagatagccagattggggaaagggtttatgataacaagcaaaacaaagagactgaaggctcaaataccttgtcctgatctgtttagtatatcattagaaagatataatgaagaaacaagggcaaaattgaaaaagctaaaaccattcttcaatgaagaaggaactgcatctttgtcacctgggatgatgatgggaatgtttaatatgctatctaccgtgttgggggtagctgcactaggtatcaagaacattggaaacaaagaatacttatgggatggactgcaatcttctgatgattttgctctatttgttaatgcaaaggatgaagaaacatgtatggaaggaataaacgacttttaccgaacatgtaaattattgggaataaacatgagcaaaaagaaaagttactgtaatgagactggaatgtttgaatttacaagcatgttctacagagatggatttgtatctaattttgcaatggaactcccttcatttggggttgctggagtaaatgaatcagcagatatggcaatagggatgacaataataaagaacaacatgatcaacaatggaatgggtccggcaacagcacaaacagccatacagttattcatagctgattatagatacacctacaaatgccacaggggagattccaaagtagagggaaagagaatgaaaatcataaaggagttatgggaaaacactaaaggaagagatggtctattagtagcagatggtgggcccaacatttacaatttgagaaacttgcatattccagaaatagtattaaagtataacttaatggaccctgaatacaaagggcggttacttcatcctcaaaatccctttgtgggacatttgtctattgagggcatcaaagaggcagatataaccccagcacatggtccagtaaagaaaatggactacgatgcggtgtctggaactcatagttggagaaccaaaagaaacagatctatactaaacactgatcagaggaacatgattcttgaggaacaatgctacgctaagtgttgcaacctatttgaggcctgttttaacagtgcatcatacaggaagccagtgggtcaacatagcatgcttgaggctatggcccacagattaagaatggatgcacgattagattatgaatcaggaagaatgtcaaaggatgattttgagaaagcaatggctcaccttggtgagattgggtacatataagcttcgaagatgtctatggggttattggtcatcattgaatacatgcgatacacaaa

SEQ ID NO: 81 - DNA, 2358 bp, synthetic construct
  source 1..2358: mol_type = other DNA; note = Nucleotide sequence of mouse adapted Influenza B/Malaysia/2506/2004 virus PB2
ttcaagatgacattggccaaaattgaattgttaaaacaactgctaagggacaatgaagccaaaacagttttgaagcaaacaacggtagaccaatataacataataagaaaattcaatacatcaaggattgaaaagaatccttcactaaggatgaaatgggccatgtgttctaattttcccttggctctaaccaagggcgacatggcaaatagaatccccttggaatacaaaggaatacaacttaaaacaaatgctgaagacataggaaccaaaggccaaatgtgctcaatagcagcagttacttggtggaatacatatggaccaataggagatactgaaggtttcgaaagggtctacgaaagcttttttctcagaaaaatgagacttgacaatgccacttggggccgaataacttttggcccagttgaaagagtgagaaaaagggtactgctaaaccctctcaccaaggaaatgcctccagatgaggcgagcaatgtgataatggaaatattgttccctaaagaagcaggaataccaagagaatccacttggatacatagrgaactgataaaagaaaaaagagaaaaattgaaaggaacaatgataactccaatcgtactggcatacatgcttgaaagagaactggttgctcgaagaagattcttgccagtggcaggagcaacatcagctgagttcatagaaatgctacactgcttacaaggtgaaaattggagacaaatatatcacccaggagggaataaattaactgagtctaggtctcaatcaatgatagtagcttgtagaaaaataatcagaagatcaatagtcgcttcaaacccactggagctagctgtagaaattgcaaacaagactgtgatagatactgaacctttaaagtcatgtctggcagccatagacggaggtgatgtagcttgtgacataataagagctgcattaggactaaagatcagacaaagacaaagatttggacggcttgagttaaaaagaatatcaggaagaggattcaaaaatgatgaagaaatattaatagggaacggaacaatacagaagattggaatatgggacggggaagaggagttccatgtaagatgtggtgaatgcaggggaatattaaaaaagagtaaaatgaaactggaaaaactactaataaattcagccaaaaaggaggacatgagagatttaataatcttatgcatggtattttctcaagacactaggatgttccaaggggtgagaggagaaataaattttcttaatcgagcaggccaacttttatctccaatgtaccaactccaacgatattttttgaatagaagcaacgacctttttgatcaatgggggtatgaggaatcacccaaagcaagtgaactatatgggataaatgaatcaatgaatgcatctgactatacattgaaaggggttgtagtgacaagaaatgtaattgacgactttagctctactgaaacagaaaaagtatccataacaaaaaatcttagtttaataaaaaggactggggaagtcataatgggagctaatgacgtgagtgaattagaatcacaagcacagctgatgataacatatgatacacctaagatgtgggaaatgggaacaaccaaagaactggtgcaaaacacttatcaatgggtgctaaaaaacttggtaacactgaaggctcagtttcttctaggaaaagaggacatgttccaatgggatgcatttgaagcatttgagagcataattcctcagaagatggctggtcagtacagtggatttgcaagagcagtgctcaaacaaatgagagaccaggaggttatgaaaactgaccagttcataaagttgttgcctttttgtttctcaccaccaaaattaaggagcaatggggagccttatcaattcttaaaacttgtattgaaaggaggaggggaaaatttcatcgaagtaaggaaagggtcccctctattttcctataatccacaaacagaagtcctaaccatatgcggcagaatgatgtcattaaaagggaaaattgaagatgaagaaaggaatagatcaatggggaatgcagtattagcaggctttctcgttagtggcaagtatgacccagatcttggagatttcaaaactattgaagaacttgaaaggctgaaaccgggggaaaaggcaaatatcttactttatcaaggaaagccagttaaagtagttaaaaggaaaaggtatagtgctttgtccaatgacatttcacaaggaattaagagacaaagaatgacagttgagtccatggggtgggccttgagctaatataaatttatccattaattcaatgaacgcaattgagtg

SEQ ID NO: 82 - DNA, 2248 bp, synthetic construct
  source 1..2248: mol_type = other DNA; note = Nucleotide sequence of mouse adapted Influenza B/Malaysia/2506/2004 virus PA
atacttttattacaagaaacttccaaactacaataatacaaaaggccagaaacacaatggcagaatttagtgaagatcctgaattacaaccagcaatgctattcaatatctgcgtccatctagaggtttgctatgtaataagtgacatgaattttcttgacgaagaaggaaaagcatatacagcattagaaggacaagggaaagaacaaaatttgagaccacaatatgaagtaattgagggaatgccaagaaccatagcatggatggtccaaagatccttagctcaagagcatggaatagagactcccaagtatctggctgatttgtttgattataaaaccaagagatttatagaagttggaataacaaaaggattggctgatgattacttttggaaaaagaaagaaaagttgggaaatagcatggaactgatgatattcagctacaatcaagactactcgttaagtaatgaatcctcattggatgaggaagggaaagggagagtgctaagcagactcacagaacttcaggctgaattaagtctgaaaaacctatggcaagttctcataggagaagaagatgttgaaaagggaattgactttaaacttggacaaacaatatctagactaagggatatatctgttcccgctggtttctccaattttgaaggaatgaggagctacatagacaatatagacccaaaaggagcaatagagagaaatctagcaaggatgtctcccttagtatcagtcacacctaaaaagttgacatgggaggacctaagaccaatagggcctcacatttacaaccatgagctaccagaagttccatataatgcctttcttctaatgtctgatgaactggggctggccaatatgactgagggaaagtccaaaaaaccgaagacattagccaaagaatgtctagaaaagtactcaacactacgggatcaaactracccaatattaataatgaaaagcgaaaaagctaatgaaaatttcctatggaagctttggagagactgtgtaaatacaataagtaatgaggaaatgagtaacgagttacagaaaaccaattatgccaagtgggccacaggggatggattaacataccagaaaataatgaaagaagtagcaatagatgacgaaacaatgtgccaagaagagcctaaaatccctaataaatgtagagtggctgcttgggttcaaacagagatgaatctattgagcactctgacaagtaaaagagctctggacctaccagaaatagggccagacgtagcacccgtggagcatgtagggagtgaaagaaggaaatactttgttaatgaaatcaactactgtaaggcctctacagttatgatgaagtatgtgctttttcacacttcattgttgaatgaaagcaatgccagcatgggaaaatacaaagtaataccaataaccaatagagtagtaaatgaaacaggagaaagtttcgacatgctttatggtctggcggttaaaggacaatctcatctgaggggagatactgatgttgtaacagttgtaactttcgaatttagtagtacagacccaagagtggactcaggaaagtggccaaaatatactgtgtttaggattggctccctatttgtgagtgggagggaaaaatctgtgtacctgtattgccgagtgaatggcacaaataagatccaaatgaaatggggaatggaagctagaagatgtctgcttcaatcaatgcaacaaatggaagcaattgttgaacaggaatcatcgatacagggatatgacatgaccaaggcttgtttcaagggagacagagtaaatagccccaaaactttcagtattggaactcaagaagggaaactagtaaaaggatcctttggaaaagcactaagagtaatatttactaaatgtttgatgcactatgtatttggaaatgcccaattggaggggtttagtgccgagtctaggagacttctactgttgatccaagcattaaaggacagaaagggcccttgggtgttcgacttagagggaatgtattctggaatagaagaatgtattagcaacaacccttgggtaatacagagtgcatactggtttaatgaatggttgggctttgaaaaggaggggagtaaagtgttagaatcagtggatgaaataatggatgaataaaaggacatggtactcaatttggtactattttgttcattatgtatctaaacatccaataaaaagaaccaaga

SEQ ID NO: 83 - DNA, 1824 bp, synthetic construct
  misc_feature 641: note = n is a, c, g, or t
  source 1..1824: mol_type = other DNA; note = Nucleotide sequence of mouse adapted Influenza B/Malaysia/2506/2004 virus HA
atgaaggcaataattgtactactcatggtagtaacatccaatgcagatcgaatctgcactgggataacatcgtcaaactcaccacatgttgtcaaaactgctactcaaggggaggtcaatgtgactggtgtaataccactgacaacaacacccaccaaatctcattttgcaaatctcaaaggaacagaaaccagagggaaactatgcccaaaatgcctcaactgcacagatctggacgtggccttgggcagaccaaaatgcacggggaacataccctcggcaagagtttcaatactccatgaagtcagacctgttacatctgggtgctttcctataatgcacgacagaacaaaaattagacagctgcctaaccttctcagaggatacgaacatatcaggttatcaactcataacgttatcaatgcagaaaatgcaccaggaggaccctacaaaattggaacctcagggtcttgccctaacgttaccaatggaaacggatttttcgcaacaatggcttgggccgtcccaaaaaacgacaacaacaaaacagcaacaaattcattaacaatagaagtaccatacatttgtacagaaggagaagaccaaattaccgtttgggggttccactctgatarcgaaanccaaatggcaaagctctatggggactcaaagccccagaagttcacctcatctgccaacggagtgaccacacattacgtttcacagattggtggcttcccaaatcaaacagaagacggaggactaccacaaagtggtagaattgttgttgattacatggtgcaaaaatctgggaaaacaggaacaattacctatcaaagaggtattttattgcctcaaaaagtgtggtgcgcaagtggcaggagcaaggtaataaaaggatccttgcctttaattggagaagcagattgcctccacgaaaaatacggtggattaaacaaaagcaagccttactacacaggggaacatgcaaaggccataggaaattgcccaatatgggtgaaaacacccttgaagctggccaatggaaccaaatatagacctcctgcaaaactattaaaggaaaggggtttcttcggagctattgctggtttcttagaaggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagacgagaaagtggatgatctcagagctgatacaataagctcacaaatagaactcgcagtcctgctttccaatgaaggaataataaacagtgaagatgagcatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacttttgattcactgaatattactgctgcatctttaaatgacgatggattggataatcatactatactgctttactactcaactgctgcctccagtttggctgtaacattgatgatagctatctttgttgtttatatggtctccagagacaatgtttcttgctccatctgtctataaggaaagttaaaccctgtattttcctttattgtagtgcttgtttgcttgttaccattacaaaaaacg

SEQ ID NO: 84 - DNA, 1772 bp, synthetic construct
  source 1..1772: mol_type = other DNA; note = Nucleotide sequence of mouse adapted Influenza B/Malaysia/2506/2004 virus NP
caacaaaagaactgaaaatcaaaatgtccaacatggatattgacggtatcaacactgggacaattgacaaagcaccggaagaaataacttctggaaccagtgggacaaccagaccaatcatcagaccagcaacccttgccccaccaagcaacaaacgaacccggaacccatccccggaaagagcaaccacaatcagtgaagctgatgtcggaaggaaaacccaaaagaaacagaccccgacagagataaagaagagcgtctacaatatggtagtgaaactgggtgaattctataaccagatgatggtcaaagctggacttaacgatgacatggagagaaacctaattcaaaatgcgcatgctgtggaaagaattctattggctgccactgatgacaagaaaactgaattccagaagaaaaagaatgccagagatgtcaaagaagggaaagaagaaatagatcacaacaaaacagggggcaccttttacaagatggtaagagatgataaaaccatctacttcagccctataagagtcacctttttaaaagaagaagtaaaaacaatgtacaaaaccaccatggggagtgatggcttcagcggactaaatcacataatgattgggcattcacagatgaatgatgtctgtttccaaagatcaaaggcactaaaaagagttggacttgacccttcattaatcagtacctttgcaggaagcacactccccagaagatcaggtgcaactggtgttgcgatcaaaggaggtggaactctagtggctgaagccattcgatttataggaagagcaatggcagacagagggctattgagagacatcaaagccaagactgcgtatgaaaagattcttctgaacctaaaaaacaaatgctctgcgccccaacagaaggctctagttgatcaagtgatcggaagtagaaatccagggattgcagacattgaagacctaaccctacttgctcgtagtatggtcgttgttaggccctctgtggcgagcaaagtagtgcttcccataagcatttacgccaaaatacctcaactagggttcaacgttgaagagtactctatggttgggtatgaagccatggctctttacaatatggcaacacctgtttccatattaagagtgggagatgatgcaaaggacaaatcacaattattcttcatgtcttgcttcggagctgcctatgaagacctgagagttttgtctgcattaacaggcacagaattcaagcctagatcagcattaaaatgcaagggtttccatgttccagcaaaggaacaggtggaaggaatgggggcagctctgatgtccatcaagctccagttttgggctccaatgaccagatctgggggaaacgaagtaggtggagacggggggtctggccaaataagttgcagcccagtgtttgcagtagaaagacctattgctctaagcaagcaagctgtaagaagaatgctgtcaatgaatattgagggacgtgatgcagatgtcaaaggaaatctactcaagatgatgaatgactcaatggctaagaaaaccaatggaaatgctttcattgggaagaaaatgtttcaaatatcagacaaaaacaaaaccaatcccgttgaaattccaattaagcaaaccatccccaatttcttctttgggagggacacagcagaggattatgatgacctcgattattaaagcaacaaaatagacactatgactgtgattgtttcaatacgtttggaatgtgggtgtttactctta

SEQ ID NO: 85 - DNA, 1483 bp, synthetic construct
  source 1..1483: mol_type = other DNA; note = Nucleotide sequence of mouse adapted Influenza B/Malaysia/2506/2004 virus NA
aaatgaacaatgctaccttcaactatacaaacgttaaccctatttctcacatcagggggagtattattatcactatatgtgtcagcttcattgtcatacttactatattcggatatattgctaaaattcccatcaacagaaattactgcaccaacaatgccattggattgtgcaaacgcatcaaatgttcaggctgtgaaccgttctgcaacaaaaggggtgacacttcttctcccagaaccggagtggacatacccgcgtttatcttgcccgggctcaacctttcagaaagcactcctaattagccctcatagattcggagaaaccaaaggaaactcagctcccttgataataagggaaccttttattgcttgtggaccaaaggaatgcaaacactttgctctaacccactatgcagcccaaccagggggatactacaatggaacaagaggagacagaaacaagctgaggcatctaatttcagtcaaattgggcaaaatcccaacagtagaaaactccattttccacatggcagcatggagcgggtccgcatgccatgatggtaaggaatggacatatatcggagttgatggccctgacaataatgcattgctcaaaataaaatatggagaagcatatactgacacataccattcctatgcaaacaacatcctaagaacacaagaaagtgcctgcaattgcatcgggggaaattgttatcttatgataactgatggctcagcttcaggtgttagtgaatgcagatttcttaagattcgagagggccgaataataaaagaaatatttccaacaggaagaataaaacatactgaagaatgcacatgcggatttgctagcaataaaaccatagaatgtgcctgtagagataacagttacacagcaaaaagaccctttgtcaaattaaacgtggagactgatacagcagaaataagattgatgtgcacagagacttatttggacacccccagaccagatgatggaagcataacagggccttgtgaatctaatggggacaaagggagtggaggcatcaagggaggatttgtccatcaaagaatggcatccaagattggaaggtggtactctcgaacgatgtctaaaactaaaaggatggggatggggctgtatgtcaartatgatggagacccatgggctgacagtgatgcccttgcttttagtggagtaatggtttcaatggaagaacctggttggtactcctttggcttcgaaataaaagacaagaaatgtgatgtcccctgtattgggatagagatggtacatgatggtggaaaagagacttggcactcagcagctacagccatttactgtttaatgggctcaggacagctgctgtgggacactgtcacaggtgttaatatggctctgtaatggaggaatggttgagtctgttctaaaccctttgttcctattttgtttgaacaattgtccttactgaacttaa

SEQ ID NO: 86 - DNA, 1140 bp, synthetic construct
  source 1..1140: mol_type = other DNA; note = Nucleotide sequence of mouse adapted Influenza B/Malaysia/2506/2004 virus M
cgctgtttggagacacaattgcctacctgctttcattgacagaagatggagaaggcaaagcagaactagcagaaaaattacactgttggtttggtgggaaagaatttgacctagactctgccttggaatggataaaaaacaaaagatgcttaactgatatacaaaaagcactaattggtgcctctatatgctttttaaaacccaaagaccaggaaagaaaaagaagattcatcacagaacccttatcaggaatgggaacaacagcaacaaaaaagaaaggcctgattctggctgagagaaaaatgagaagatgtgtgagctttcatgaagcatttgaaatagcagaaggccatgaaagctcagcgctactatactgtctcatggtcatgtacctgaatcctggaaattattcaatgcaagtaaaactaggaacgctctgtgctttatgcgagaaacaagcatcacattcacacagggctcatagcagagcagcgagatcttcagtgcctggagtgagacgagaaatgcagatggtctcagctatgaacacagcgaaaacaatgaatggaatgggaaaaggagaagacgtccaaaagctggcagaagagctgcaaagcaacattggagtgctgagatctcttggggcaagtcaaaagaatggggaaggaattgcaaargatgtaatggaagtgctaaagcagagctctatgggaaattcagctcttgtgaagaaatatctataatgctcgaaccatttcagattctttcaatttgttcttttatcttatcagctctccatttcatggcttggacaatagggcatttgaatcaaataaaaagaggaataaacatgaaaatacgaataaaaggtccaaacaaagagacaataaacagagaggtatcaattttgagacacagttaccaaaaagaaatccaggccaaagaaacaatgaaggaagtactctctgacaacatggaggtattgagtgaccacataataattgaggggctttctgccgaagagataataaaaatgggtgaaacagttttggagatagaagaattgcattaaattcaatttttactgtatttcttactatgcatttaagcaaattgtaatcaatgtcagcaaataaactg

SEQ ID NO: 87 - DNA, 1033 bp, synthetic construct
  source 1..1033: mol_type = other DNA; note = Nucleotide sequence of mouse adapted Influenza B/Malaysia/2506/2004 virus NS
cgaacaacatgaccacaacacaaattgaggtgggtccgggagcaaccaatgccaccataaactttgaagcaggaattctggagtgctatgaaaggctttcatggcaaagagcccttgactaccctggtcaagaccgcctaaacagactaaagagaaaattagagtcaagaataaagactcacaacaaaagtgagcctgaaagtaaaaggatgtcccttgaagagagaaaagcaattggagtaaaaatgatgaaagtactcctatttatggatccgtctgctggaattgaagggtttgagccatactgtatgaaaagttcctcaaatagcaactgtacgaaatacaattggaccgattacccttcaacaccagggaggtgccttgatgacatagaagaagaaccagaggatgttgatggcccaactgaaatagtattaagggacatgaacaacaaagatgcaaggcaaaagataaaggaggaagtaaacactcagaaagaagggaagttccgtttgacaataaaaagggatatgcgtaatgtattgtccttgagagtgttggtaaacggaacattcctcaaacatcccaatggatacaagtccttatcaactctgcatagattgaatgcatatgaccagagtggaaggcttgttgctaaacttgttgctactgatgatcttacagtggaggatgaagaagatggccatcggatcctcaactcactcttcgagcgtcttaatgaaggacattcaaagccaattcgagcagctgaaactgcggtgggagtcttatcccaatttggtcaagagcaccgattatcaccagaagagggagacaattagactggtcacggaagaactttatcttttaagtaaaagaattgatgataacatattgttccacaaaacagtaatagctaacagctccataatagctgacatggttgtatcattatcattattagaaacattgtatgaaatgaaggatgtagttgaagtgtacagcaggcagtgcttgtgaatttaaaataaaa

SEQ ID NO: 88 - protein, 585 aa, Influenza B virus
  VARIANT 214: note = X = Ile or Thr
  source 1..585: note = full length amino acid sequence for influenza B/Malaysia/2506/20/03 HA
MKAIIVLLMVVTSNADRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNEXQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 89 - protein, 15 aa, Influenza B virus
  source 1..15: note = Signal Peptide for influenza B/Malaysia/2506/20/03 HA
MKAIIVLLMVVTSNA

SEQ ID NO: 90 - protein, 347 aa, Influenza B virus
  VARIANT 199: note = X = Ile or Thr
  source 1..347: note = HA1 Domain for influenza B/Malaysia/2506/20/03 HA
DRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNEXQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKER

SEQ ID NO: 91 - protein, 223 aa, Influenza B virus; identical to SEQ ID NO: 76
  source 1..223: note = HA2 Domain for influenza B/Malaysia/2506/20/03 HA

SEQ ID NO: 92 - protein, 42 aa, Influenza B virus; identical to SEQ ID NO: 15
  source 1..42: note = N-Terminal Segment of Stalk (encompasses alanine at interface with globular head domain) for influenza B/Malaysia/2506/20/03 HA

SEQ ID NO: 93 - protein, 241 aa, Influenza B virus; identical to SEQ ID NO: 79
  source 1..241: note = C-Terminal Segment of Stalk (encompasses alanine at interface with globular head domain) for influenza B/Malaysia/2506/20/03 HA

SEQ ID NO: 94 - protein, 250 aa, Influenza B virus
  VARIANT 157: note = X = Ile or Thr
  source 1..250: note = Globular Head Domain for influenza B/Malaysia/2506/20/03 HA
NLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNEXQMAKLYGDSKPQKFTSSANGVTTHYVSQIGGFPNQTEDGGLPQSGRIVVDYMVQKSGKTGTITYQRGILLPQKVWCASGRSKVIKGSLPLIGE

SEQ ID NO: 95 - protein, 533 aa, Influenza B virus
  source 1..533: note = Ectodomain for influenza B/Malaysia/2506/20/03 HA
  VARIANT 199: note = X = Ile or Thr
DRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNEXQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHT